(19) United States
(12) Patent Application Publication
(10) Pub. No.: US 2006/0111357 A1
(43) Pub. Date: May 25, 2006

(54) Quinoline compounds for use in mch receptor related disorders

(76) Inventors: Thomas Michael Frimurer, COPENHAGEN S (DK); Trond Ulven, Copenhagen K (DK); Thomas Hogberg, Akarp (SE); Pia Karina Norregaard, Lyngby (DK); Paul Brian Little, Naestved (DK); Jean-Marie Receveur, Frederiksberg C (DK)

Correspondence Address: EDWARDS & ANGELL, LLP, P.O. BOX 55874, BOSTON, MA, 02205, US

(21) Appl. No.: 10/538,455

(22) PCT Filed: Dec. 11, 2003
(86) PCT No.: PCT/DK03/00857

§ 371 Date: Sep. 2, 2005

(51) Int. Cl. A61K 31/519; A61K 31/517; A61K 31/498; A61K 31/4745; A61K 31/525; A61K 31/4709

(52) U.S. Cl. 514/249; 514/264.11; 514/251; 514/300; 514/314; 514/266.2

(57) ABSTRACT

The present invention relates to the use of quinoline compounds for the preparation of a pharmaceutical and/or a cosmetic composition for the treatment, prophylaxis and/or diagnosis of a condition caused by or involving a melanin-concentrating hormone. The invention also relates to novel quinoline compounds per se. The quinoline compounds have been found to interact with a melanin-concentrating hormone receptor, a MCH receptor. The compounds have modulating activity on the MCH receptor such as e.g. antagonistic, agonistic or allosteric activity and are useful for medicinal or cosmetic purposes such as, e.g. in the treatment or prevention of feeding disorders like obesity, metabolic syndrome, Type II diabetes, bulimia, etc. or in the treatment or prevention of depression.

### FIELD OF THE INVENTION

The present invention relates to the use of quinoline compounds for the preparation of a pharmaceutical and/or a cosmetic composition for the treatment, prophylaxis and/or diagnosis of a condition caused by or involving a melanin-concentrating hormone. The invention also relates to novel quinoline compounds per se. The quinoline compounds have been found to interact with a melanin-concentrating hormone receptor, a MCH receptor. The compounds have modulating activity on the MCH receptor such as e.g. antagonistic, agonistic or allosteric activity and are useful for medicinal or cosmetic purposes such as, e.g. in the treatment or prevention of feeding disorders like obesity, metabolic syndrome, Type II diabetes, bulimia etc. or in the treatment or prevention of depression.

The invention also relates to therapeutic and/or prophylactic use of the compounds, to novel compounds and to processes for the preparation of the novel compounds, to pharmaceutical compositions comprising the compounds, to the manufacture of such compositions and to methods for the treatment and/or prevention of MCH receptor related disorders.

The invention is characterised by compounds with favourable physicochemical features, which are of importance for manufacturing of pharmaceutical preparations and for providing efficient delivery of the drug to the target organ. The favourable properties include a sufficient aqueous solubility of the compounds provided by a basic aliphatic nitrogen.

Additionally, the invention relates to a group of compounds displaying a reduced propensity to block HERG channels and accordingly are less likely to induce prolonged QT interval on the ECG that is associated with tachyarrhythmias known as ventricular tachycardia, torsades de pointes ventricular tachycardia, and ventricular fibrillation, which could lead to sudden death. The problem of medication-induced long QT syndrome is a significant issue to the pharmaceutical industry. (Molecular and Cellular Mechanisms of Cardiac Arrhythmias, Mark T. Keating and Michael C. Sanguinetti (2001) Cell, Vol. 104, 569-580).

### BACKGROUND OF THE INVENTION

Melanin-concentrating hormone (MCH) is a cyclic peptide that originally was isolated from salmoid pituitaries. In the fish, the 17 amino acid peptide causes aggregation of melanin and inhibits the release of ACTH. Mammalian MCH (19 amino acids) is highly conserved between rat, mouse and human exhibiting 100% amino acid identity. In the last decades there has been increasing activity in the research in the physiologic roles of MCH. It has been reported that MCH is involved in the feeding or body weight regulation, in energy balance, in response to stress, in water balance, in energy metabolism, in the general arousal/attention state, memory and cognitive functions and in psychiatric disorders. The biological effects of MCH are believed to be mediated by specific MCH receptors, and the MCH1 and MCH2 receptors have been described. Antagonists of MCH receptor (e.g. MCH1 receptor) may be suitable for use as obesity or weight reducing agents and they are also believed to have antidepressant and/or anxiolytic properties.

The present invention provides novel compounds as well as novel use of compounds that have been found to possess MCH modulating activity, i.e. antagonistic, inverse agonistic/negative antagonism, allosteric modulator, partial agonist or agonistic action.

### DETAILED DESCRIPTION OF THE INVENTION

The term “alkenyl” is intended to indicate an unsaturated alkyl group having one or more double bonds and containing from 2-10 carbon atoms, such as e.g. 2-8,2-6 or 2-4 carbon atoms.

The term “alkynyl” is intended to indicate an unsaturated alkyl group having one or more triple bonds and containing from 2-10 carbon atoms, such as e.g. 2-8,2-6 or 2-4 carbon atoms.

The term “alkyl” or “Alk” is intended to denote a cyclic or acyclic, branched or non-branched, saturated alkyl group of 1-10 carbon atoms, such as e.g. 1-8,1-6 or 1-4 carbon atoms.

The term “cycloalkyl” is intended to denote a cyclic, saturated alkyl group of 3-7 carbon atoms.

The term “cycloalkenyl” is intended to denote a cyclic, unsaturated alkyl group of 5-7 carbon atoms having one or more double bonds.

The term “alkoxy” is intended to indicate the group alkyl-O—.

The term “aryl” is intended to denote an aromatic (unsaturated), typically 6-membered, ring, which may be a single ring (e.g. phenyl) or fused with other 5- or 6-membered rings (e.g. naphthyl or indole).

The term “heteroaryl” is intended to denote an aromatic (unsaturated), 5- or 6-membered, ring, which may be a single ring (e.g. pyridyl) or fused with other 5- or 6-membered rings (e.g. quinoline or indole).

The term “heterocyclyl” is intended to indicate a cyclic unsaturated (heteroalkenyl), aromatic (“heteroaryl”) or saturated (“heterocycloalkyl”) group comprising at least one heteroatom.

The present invention relates to the use of a compound with the following structure (Formula 1a) 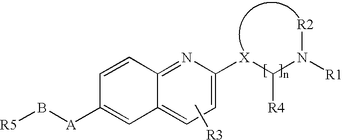
wherein the quinoline moiety may contain more than one nitrogen atom such as, e.g. 2 or 3 nitrogen atoms, and wherein -A- is a linker, which is selected from the group consisting of 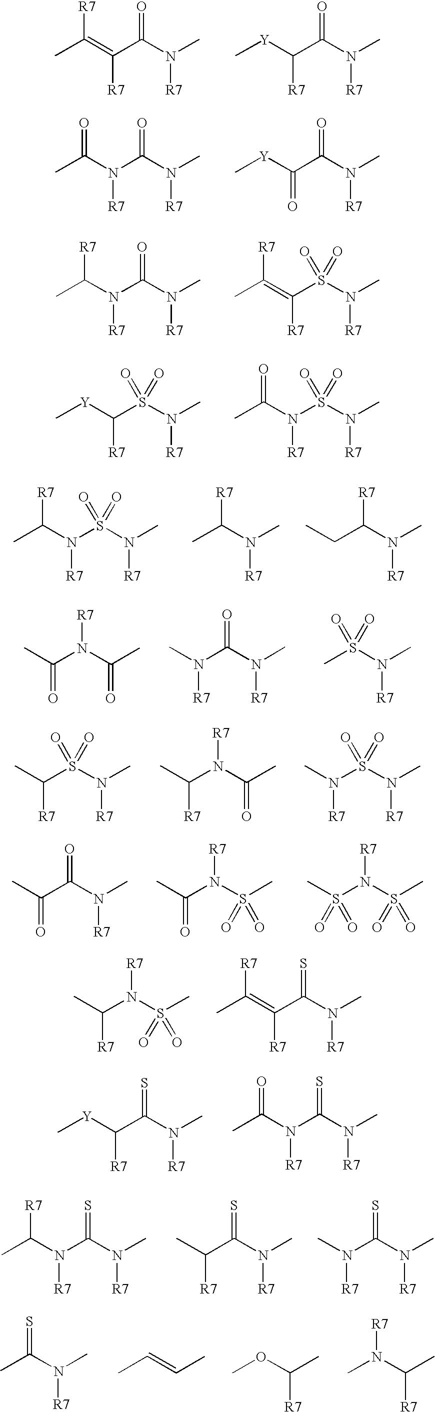 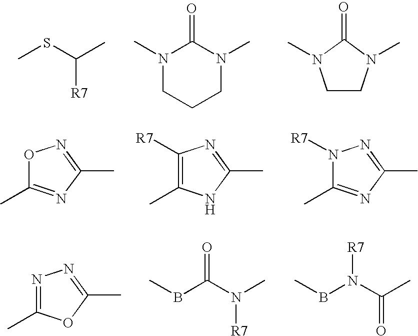
in which B is defined below, and, wherein the linker may be attached via either of the two free bonds to the B group;
and Y being CHR7, O, S, NR7;
and R7 is the same or different and is hydrogen or a straight or branched C₁-C₄ alkyl or alkenyl group; R7 can be linked direct or via hetero atoms to B or the quinoline ring system when chemically feasible;
and X being nitrogen, carbon, oxygen or sulphur and X being restricted to nitrogen or carbon when X linked to R2 as indicated in formula Ia;
B is an aryl or heteroaryl group such as, e.g. phenyl, pyridine, pyrimidine, pyrazine, thiophene, oxazole, isothiazole, pyrazole, pyrrole, imidazole, indole, benzimidazole, quinoline, isoquinoline, furan, benzofuran, benzothiophene, benzothiazole, indazole, thiazole, isoxazole, oxadiazole, indan;
R1 and R2 are the same or different selected from hydrogen, straight or branched alkyl, alkenyl or alkynyl groups with 1-6 carbon atoms; cycloalkyl groups with 3-7 carbons; alkylcycloalkyl with 4-8 carbons atoms; alkylaryl groups such as benzyl, 2-ethylphenyl, 3-propylphenyl; alkylheteroaryl groups; the alkyl, aryl and heteroaryl groups may be substituted with substituents such as Alk-CONH—, Alk-O—, HO—, NC—, AlkNH—, Alk₂N—, —CONH₂, —CONHAlk, —CONAlk₂, or the aryl and heteroaryl groups fused with moieties such as —O—CH₂O—, —N═CH—NH—, —O—CH═N—; R2 may be further substituted with one or more R4 groups in any position;
Alk is the same or a different alkyl, alkenyl or alkynyl group;
R4 is the same or different and is hydrogen or a straight or branched C₁-C₄ alkyl group; and may be substituted with one or two C₁-C₄ alkyl groups;
R3 may be selected from hydrogen, alkyl, alkenyl or alkynyl groups, halogen atoms, alkoxy groups (AlkO—), hydroxy, alkylamino groups (AlkNH—), dialkylamino groups (Alk₂N—), hydroxylalkyl groups, carboxamido groups (—CONH₂, —CONHAlk, —CONAlk₂), acylamido groups (—NHCO-Alk), acyl groups (—CO-Alk), —CHO, nitrile, —SCH₃, partially or fully fluorinated alkyl, alkoxy or thioalkoxy groups such as —CH₂CF₃, —CF₂CF₃, —CF₃, —OCF₃, —SCF₃; —SO₂NH₂, —SO₂NHAlk, —SO₂NAlk₂, —SO₂Alk;
R1, R2, R3 or R4 may optionally be linked to each other, or to the carbon chain linking the two nitrogen atoms, when possible; and O or NR1 may be inserted in the chain or ring in a chemically stable position; R4 may optionally be linked to X;
R5 is hydrogen, halogen atoms, alkyl, alkenyl or alkynyl groups, cycloalkyl groups with 3-7 carbons, aryl groups (Ar), heteroaryl groups, heterocyclyl groups, alkylcycloalkyl groups, alkylaryl groups, alkylheterocyclyl groups, alkylheteroaryl groups, arylalkoxy groups (e.g. ArCH₂O—), aryloxy groups (ArO—), arylamino groups (Ar—NR7—, ArNH—), arylalkylamino groups (ArAlkNH—, ArAlkNR7—, ArCH₂NR7—, ArCH₂NH—), alkoxy groups (AlkO—), alkylamino groups (AlkNH—), dialkylamino groups (Alk₂N—), —CONH₂, —CONHAlk, —CONHAr —CONAlk₂, —NHCO-Alk, —NHCO—Ar, —CO-Alk, —CO—Ar, —CF₂—Ar, —N(CF₃)₂, —SCH₃, partially or fully fluorinated alkyl, alkoxy or thioalkoxy groups such as —CH₂CF₃, —CF₂CF₃, —CF₃, —OCF₃, —SCF₃;
optionally, one or more R5 may be present on B; and
n is 0, 1, 2 or 3 with the proviso that when n is 0 or 1 then X is C and when n is 2 or 3, then X is C, O, S or N
for the preparation of a pharmaceutical composition for the treatment, prophylaxis and/or diagnosis of a condition caused by or involving a melanin-concentration hormone.

The structure of the compounds according to the invention may vary within the scope defined above. This variation may occur at different parts of the molecule, and certain structures are of higher interest than others. In the following are given structural variations which describe the scope of the invention more clearly and define those compounds which are of most interest in the uses or methods described herein.

According to one embodiment, the nitrogen-containing chain may have the structure: 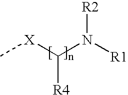
wherein X, R1, R2, R4 and n are as defined above.

Additionally, the nitrogen-containing chain may have the structure: 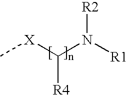
while the quinoline moiety has one of the following structures: 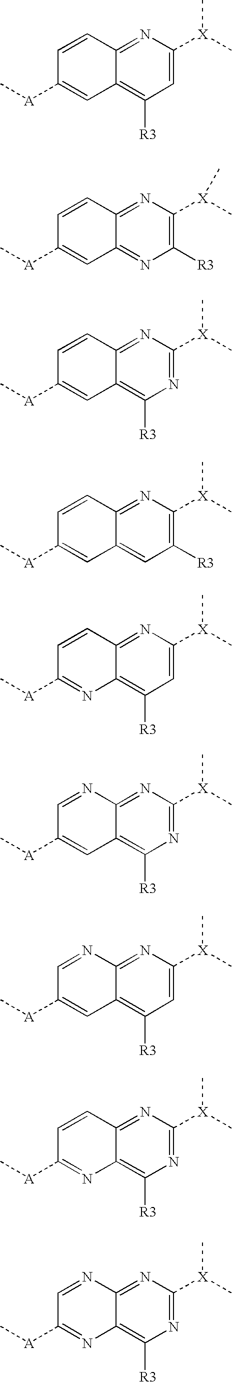 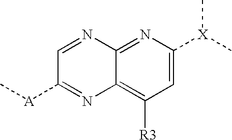
wherein A, B, R1, R2, R3, R4, R5, R7, Y, X and n are as defined above.

In a particular aspect, a cyclic group is formed between R2 and the nitrogen in the 2-position of the quinoline ring, giving a ring system with both nitrogen atoms endo to the ring. Therefore, the invention relates to use of a compound as described above, wherein the nitrogen-containing chain has the structure: 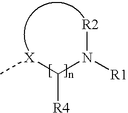
wherein X, R1, R2, R4 and n are as defined above.

Combinations of certain sub-structures give compounds which have the desired properties (i.e. interaction with an MCH receptor). Therefore, the invention relates to use as described herein, wherein the nitrogen-containing chain has the structure: 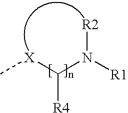
and the quinoline moiety has one of the following structures: 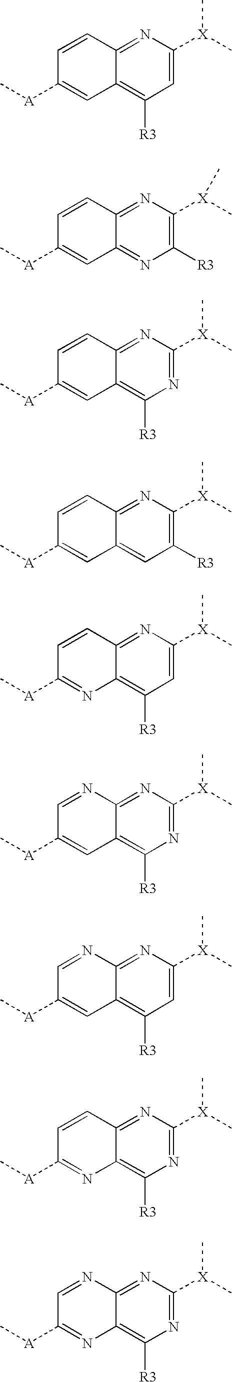 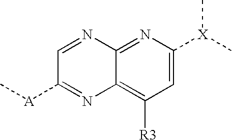
wherein X, A, B, R1, R2, R3, R4, R5, R7, Y and n are as defined above.

The size of the ring on the Eastern portion of the molecule is important, and it has been discovered that compounds in which the chain length is 2 are of particular interest. Therefore, as a development of the above structure, the invention also relates to use of a compound as described above, wherein the nitrogen-containing chain has the structure: 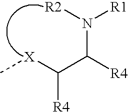
wherein X, R1, R2 and R4 are as defined above.

As before, combinations may be made of Eastern portions and quinoline moieties, so that the invention relates to use of a compound as previously described, wherein the nitrogen-containing chain has the structure: 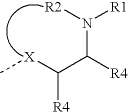
and the quinoline moiety has one of the following structures: 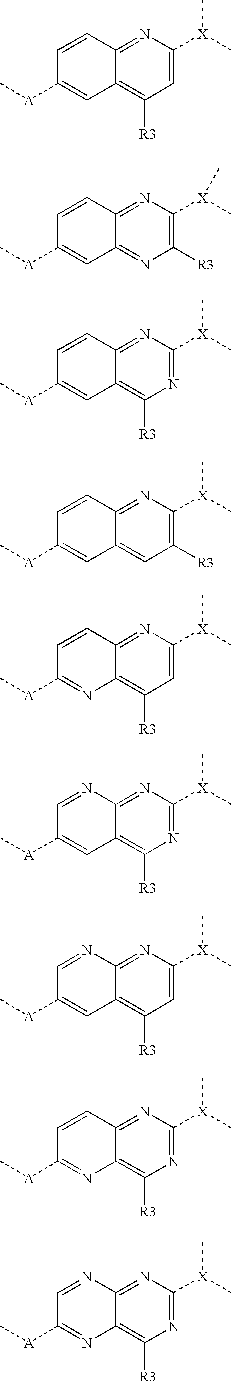 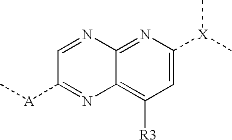
wherein A, B, R1, R2, R3, R4, R5, R7, Y, X and n are as defined above.

As a development of this, it has been found that 6-membered rings are of particular interest on the Eastern portions. Therefore, the nitrogen-containing chain may have the structure: 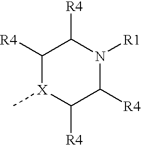
wherein X, R1 and R4 are as defined above.

Again, combining this feature with the quinoline moieties means that the nitrogen-containing chain may have the structure: 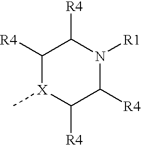
and the quinoline moiety may have one of the following structures: 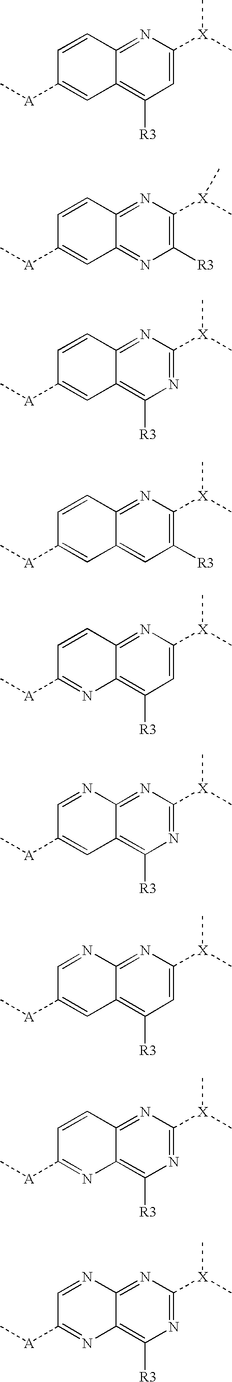 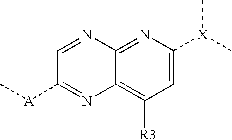
wherein X, A, B, R1, R2, R3, R4, R5, R7, Y and n are as defined above.

As well as being cyclic, the Eastern portion may contain bridged moieties, which are comprised of combinations of R1, R2 and R4. Therefore, in one embodiment of the present invention, the nitrogen-containing chain has the structure: 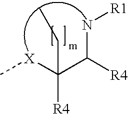
wherein X, R1 and R4 are as defined above and m is 1 or 2.

Accordingly, combination of this feature with the quinoline moieties allows the nitrogen-containing chain to have the structure: 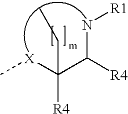
and the quinoline moiety to have one of the following structures: 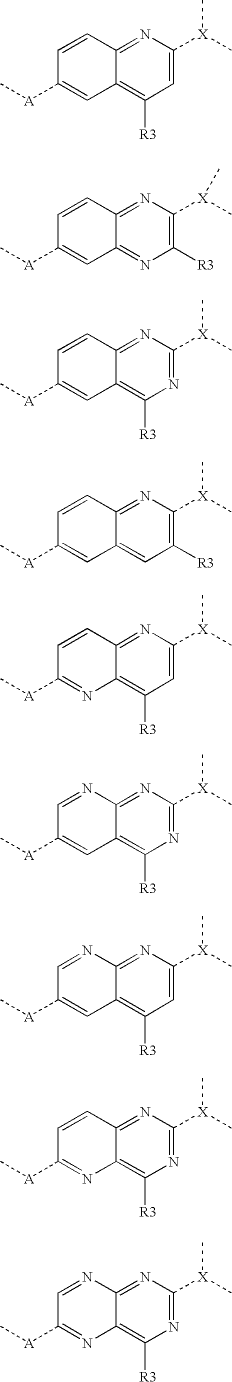 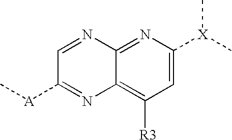
wherein X, A, B, R1, R2, R3, R4, R5, R7, Y and n are as defined in above, and m is 1 or 2.

As an alternative to the cyclic structures described above, a ring may be formed between R4 and the nitrogen which is bound to the 2-position of the quinoline, giving a structure in which one N atom is exo to the ring. In this form, the nitrogen-containing chain may have the structure: 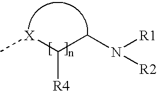
wherein X, R1, R2, R4 and n are as defined above.

Such sub-structures can also be combined with specific quinoline systems to give compounds with the most interesting properties. Therefore, the invention relates to use of a compound, wherein the nitrogen-containing chain has the structure: 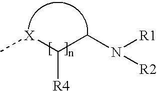
and the quinoline moiety has one of the following structures: 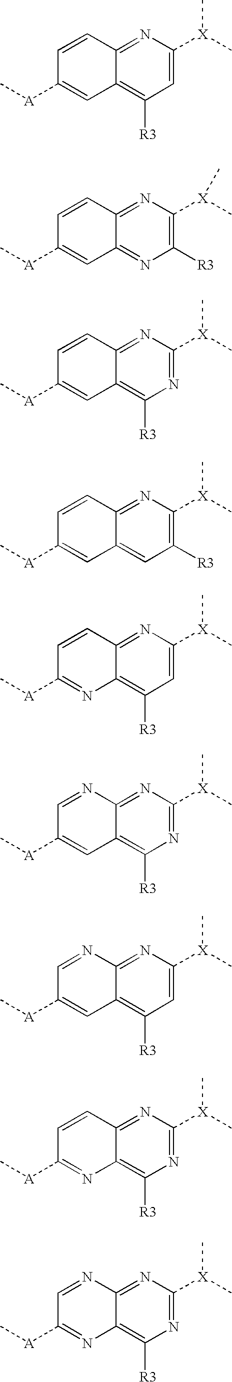 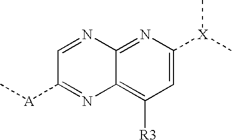
wherein X, A, B, R1, R2, R3, R4, R5, R7, Y and n are as defined above.

As previously, the ring may take various sizes, but it has been found that the presence of two R4 groups gives compounds with the most interesting properties. Hence, the nitrogen-containing chain may have the structure: 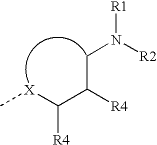
wherein X, R1, R2 and R4 are as defined above.

Combinations of this sub-structure with certain quinoline moieties means that the nitrogen-containing chain may have the structure: 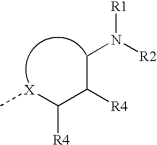
and the quinoline moiety may have one of the following structures: 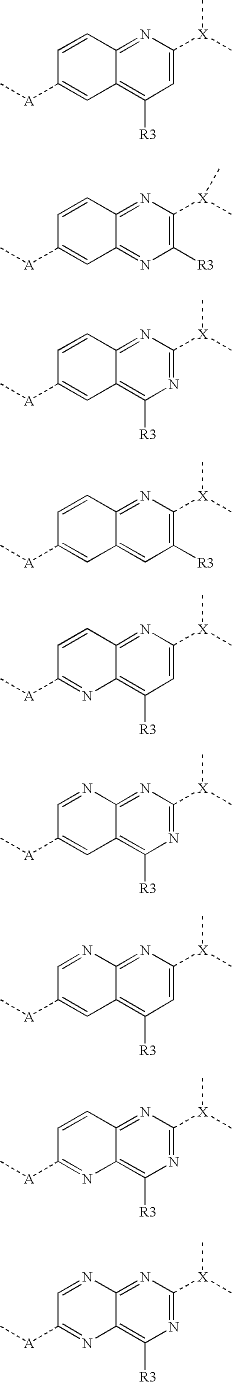 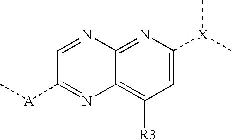
wherein X, A, B, R1, R2, R3, R4, R5, R7, Y and n are as defined above.

As before, the cyclic sub-structure may also be bridged. Interesting nitrogen-containing chains according to the invention have the structure: 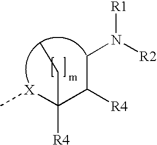
wherein X, R1, R2 and R4 are as defined above and m is 1 or 2.

Combinations of this sub-structure with certain quinoline moieties means that the nitrogen-containing chain may have the structure: 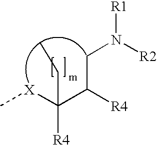
and the quinoline moiety may have one of the following structures: 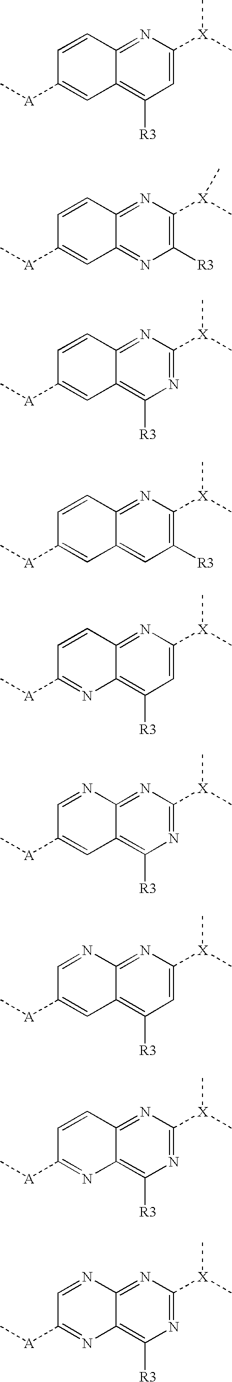 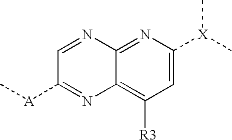
wherein X, A, B, R1, R2, R3, R4, R5, R7, Y and n are as defined above and m is 1 or 2.

As previously, it is particularly of interest when the nitrogen-containing chain (Eastern portion) contains a 6-membered ring. Therefore, the invention relates to use of a compound as described above, wherein the nitrogen-containing chain has the structure: 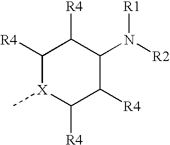
wherein X, R1, R2 and R4 are as defined above.

This sub-structure can also be combined with the quinoline moieties of interest.

Therefore, the invention relates to use as described herein, wherein the nitrogen-containing chain has the structure: 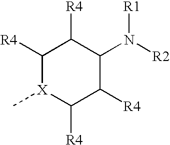
and the quinoline moiety has one of the following structures: 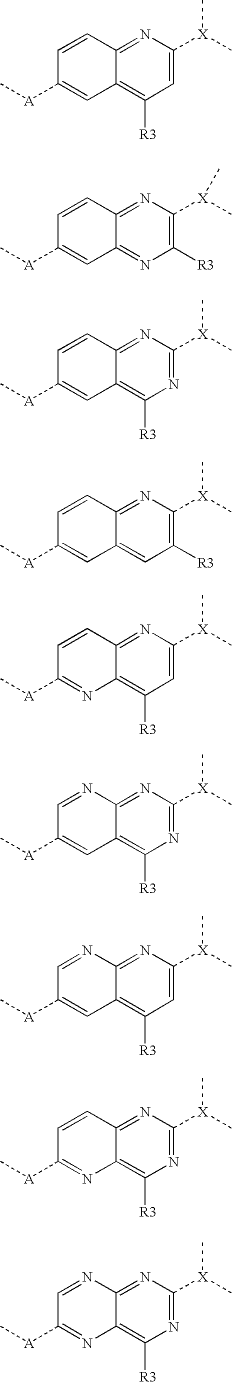 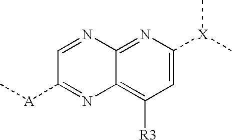
wherein X, A, B, R1, R2, R3, R4, R5, R7, Y and n are as defined above.

As well as variations in the Eastern portion, it is also of interest to vary the linker A. In a particular embodiment, the invention relates to use of a compound as described above, wherein A is selected from the group consisting of:

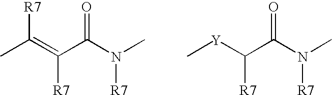

Combinations can be made between the linker A and other parts of the molecule. For example, A may have the structure 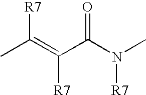
while the nitrogen-containing chain has the structure: 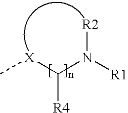
where X, R1, R2, R4, R7 and n are as defined above.

Interesting combinations of A, the Eastern portion and the quinoline moiety may be made. Examples of these are the instances where the compound has one of the following structures: 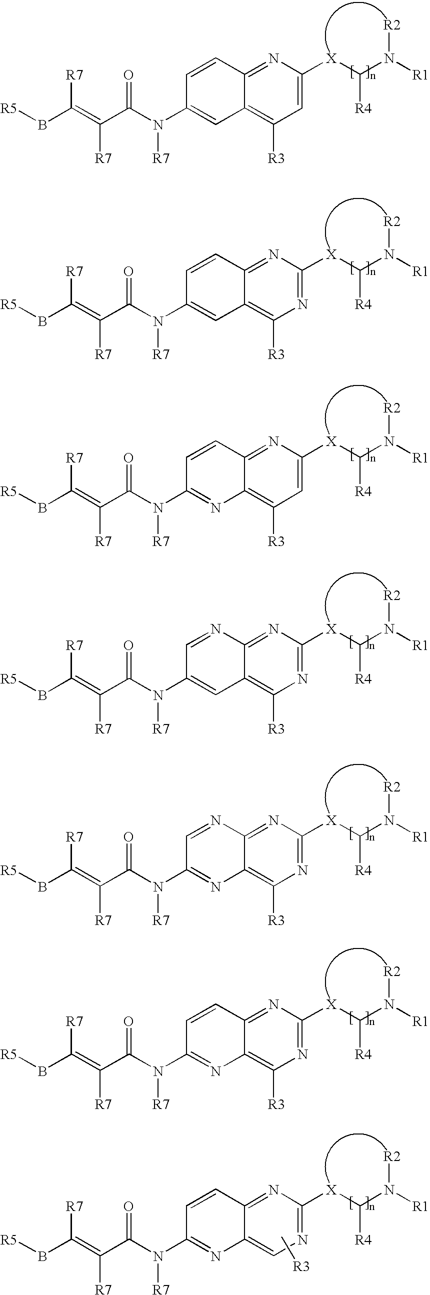
wherein X, B, R1, R2, R3, R4, R5, R7 and n are as defined above.

In a further limitation, the compounds according to the invention may have one of the following structures: 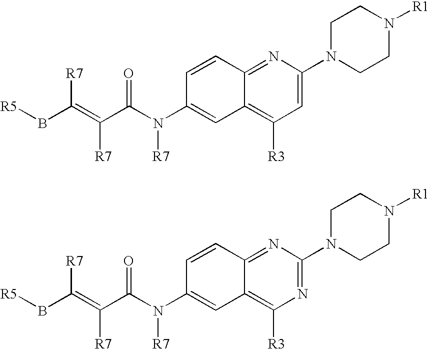
wherein B, R1, R2, R3, R4, R5 and R7 are as defined above.

An alternative combination of sub-structures is that where A has the structure 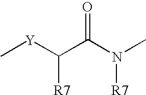
and the nitrogen-containing chain has the structure:

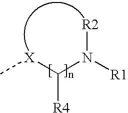

where X, R1, R2, R4, R7, n, and Y are as defined above.

Combinations of this linker A with quinoline moieties and Eastern portions of interest give compounds with one of the following structures: 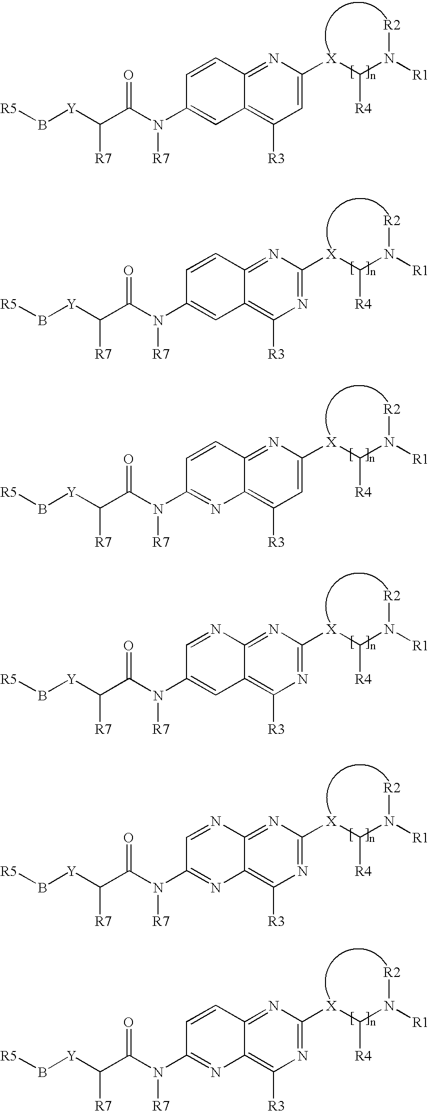
wherein X, B, R1, R2, R3, R4, R5, R7, Y and n are as defined above.

In a particular embodiment of interest, the compound may have one of the following structures: 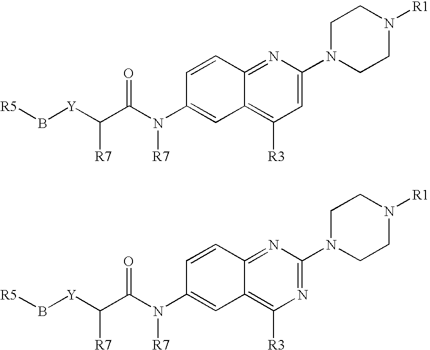
wherein B, R1, R2, R3, R4, R5, Y and R7 are as defined above.

An alternative combination of a particular A with a particular Eastern portion is that in which A has the structure 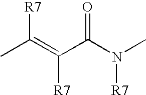
and the nitrogen-containing chain has the structure: 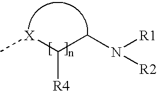
where X, R1, R2, R4, R7, n, and Y are as defined above.

According to this combination, compound according to the invention may have one of the following structures: 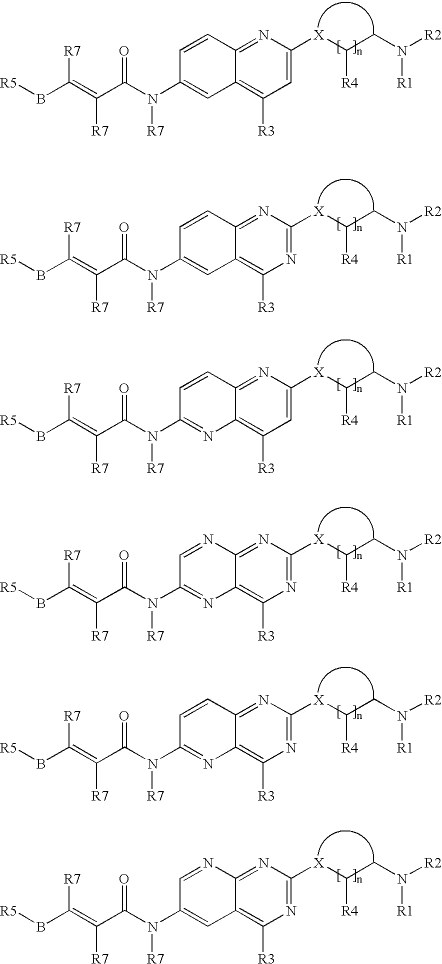
wherein X, B, R1, R2, R3, R4, R5, R7 and n are as defined above.

More precisely, the compound may have one of the following structures: 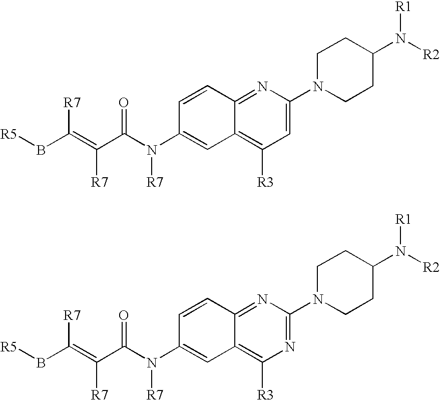
wherein X, B, R1, R2, R3, R4, R5 and R7 are as defined above.

The linker A may alternatively have the structure 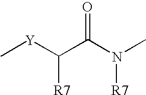
while the nitrogen-containing chain has the structure:

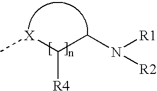

In this case, the compounds the invention may have one of the following structures: 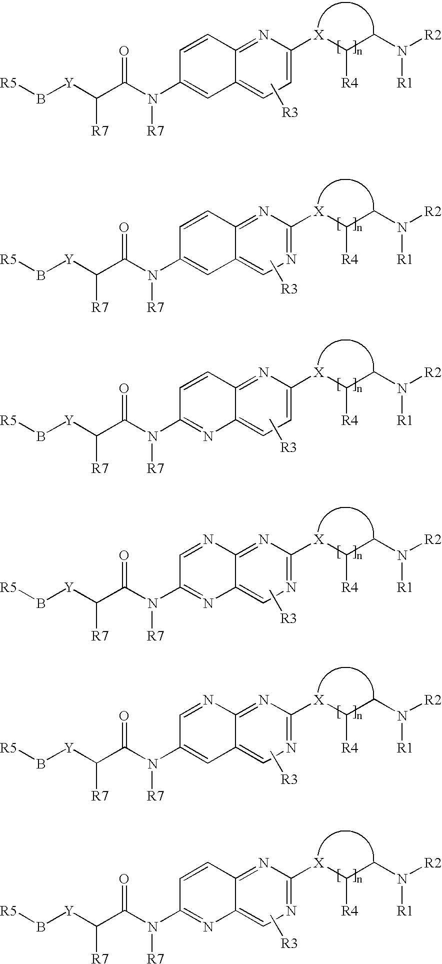
wherein X, B, R1, R2, R3, R4, R5, R7, Y and n are as defined above.

More precisely, the compound may have one of the following structures: 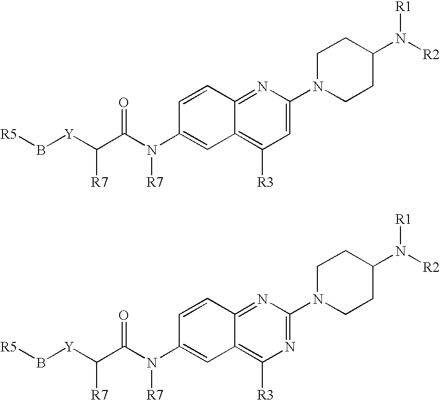
wherein B, R1, R2, R3, R4, R5, R7 and Y are as defined above.

Variations in the structure of Formula 1a lead to different effects on the MCH receptor. In particular, it is of interest when X is nitrogen. Groups R1-R7, Y and B may also be varied to provide a compound which has a desired effect. In all of the above structures, those of particular interest are obtained when R3 is methyl. Another interesting variation is that when R7 if hydrogen. Alternatively, R4 may be hydrogen.

As regards R1, it may be hydrogen or a lower straight, branched or cyclic alkyl group with 1-6 carbon atoms such as, e.g., methyl, ethyl, propyl, butyl, isopropyl, isobutyl, cyclopentyl, which may be substituted with OH. Alternatively R1 may be hydrogen, methyl, ethyl, propyl, iso-propyl, butyl, iso-butyl, sec-butyl, tert-butyl or 2-hydroxyethyl. more precisely, R1 may be methyl, ethyl or 2-hydroxyethyl.

An interesting variation with regard to Y is oxygen. In addition, B may be phenyl or pyridine.

The R5 substituent may be selected from a fairly broad range. In one interesting embodiment, R5 is halogen atoms, alkyl or alkenyl groups, cycloalkyl groups with 3-7 carbons, heterocyclyl groups, alkylcycloalkyl groups, alkoxy groups (AlkO—), alkylamino groups (AlkNH—), dialkylamino groups (Alk₂N—), —CONHAlk, —CONAlk₂, —NHCO-Alk, —CO-Alk, —N(CF₃)₂, —SCH₃, partially or fully fluorinated alkyl, alkoxy or thioalkoxy groups such as —CH₂CF₃, —CF₂CF₃, —CF₃, —OCF₃, —SCF₃. Additionally, R5 may be halogen atoms, alkyl groups, —SCH₃, partially or fully fluorinated alkyl, alkoxy or thioalkoxy groups such as —CH₂CF₃, —CF₂CF₃, —CF₃, —OCF₃, —SCF₃.

The invention also relates to those novel compounds per se, which have the structures described above, as well as the limitations described above. Particular novel compounds are those in which the quinoline moiety contains more than one nitrogen atom, such as e.g. 2 or 3 nitrogen atoms. Such novel compounds are to be used in the same methods, applications and treatments as the described compounds. Other interesting embodiments appear from the appended claims.

Synthetic Routes

Compounds according to the above-mentioned structures may be commercially available or may be prepared along the lines outlined below.

Compounds of formula I are preferably made by connecting an appropriately functionalised (A″) quinoline moiety III with a suitably functionalised (A″) aryl moiety II using well-known synthetic routes according to the following general scheme:

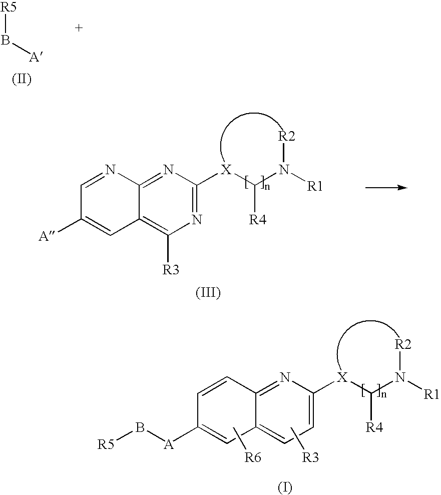

For example, urea bonds -A- can be formed by reaction of II having A′ as isocyanate with III having A″ equal to NH—R7 using appropriate catalysis by base or acid. The reverse use of III having A″ as isocyanate with II having A′ equal to NH—R7 can also be applied. Analogously, carbamates can for example be made by reaction of II having A′ as isocyanate with III having A″ equal to OH or the reverse use of OH and isocyanate in A′ and A″.

Preparation of amide and sulphonamide bonds 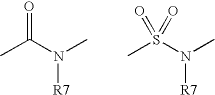
in the connecting A-linkage can be made via reaction of A″ in compound III being NH—R7 with activated forms, e.g. acid chlorides or active esters, of A′ in compound II being COOH or SO₂OH. Alternatively, the conversion can be made directly with the acids having A′ as COOH using suitable coupling reagents such as dicyclohexylcarbodiimide (DCC), and promoters such as 1-hydroxybenzotriazole. The reverse use of A′ and A″ in II and III can be applied as well to form the linker in the opposite direction.

Formation of the connecting A-linkage to form 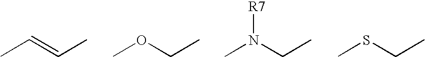
bonds in either direction between B and the quinoline can be made by N—, O— or S-alkylations of compound II with A′ being OH, NH—R7, or SH with compound III with A″ being a CH₂-Lg wherein Lg being a suitable leaving group such as halogen (Cl, Br, I), tosyl or mesyl using appropriate catalysts and conditions, or by a Mitsunobu reaction with Lg being OH. The alkene linkage can be made by a Wittig reaction with compound II with A′ being CHO and compound III with A″ being CH₂—PPh₃. The reverse use of A′ and A″ in II and III can be applied as well to form the linker in the opposite direction.

Formation of the connecting A-linkage to form 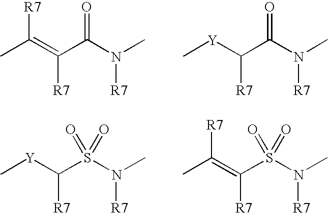
bonds in either direction between B and the quinoline can be made by N—, O— or S-alkylations of compound II with A′ being OH, NH—R7, or SH with compound III with A″ being a —NR7-CO—CHR7-Lg or —NR7-SO₂—CHR7-Lg wherein Lg being a suitable leaving group such as halogen (Cl, Br, I), tosyl or mesyl using appropriate catalysts and conditions, or by a Mitsunobu reaction with Lg being OH. The alkene linkage can be made by a Homer-Emmons-Wadsworth reaction with compound II with A′ being CHO. The reverse use of A′ and A″ in II and III can be applied as well to form the linker in the opposite direction.

The 5-membered heterocyclic linkers 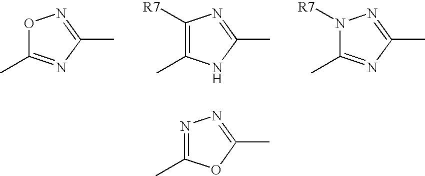
can be made according to standard cyclisation procedures using appropriate solvents, catalysts and temperatures. For example, formation of 1,2,4-triazole can be made from II with A′ being acylhydrazide with III with A″ being amide or thioamide or the reverse orientation of A′ and A″. 1,2,4-Oxadiazole can be formed from II with A′ being amidoxime with III with A″ being carboxylic ester or the reverse orientation of A′ and A″. 1,3,4-Oxadiazole can be formed from II with A′ being acylhydrazide with III with A″ being carboxylic ester or the reverse orientation of A′ and A″.

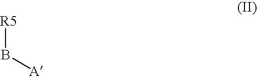

Aromatic substituents R3, R5 and R6 are preferably introduced prior to formation of the A- or B-linkage either direct or via a masked functionality that is compatible with the subsequent synthetic steps.

Compounds of formula I can also be made by reacting a quinoline with a leaving group in the 2-position (IV) with a nucleophilic or activated fragment (V), e.g. in an aromatic nucleophilic substitution or a metal catalyzed coupling reaction.

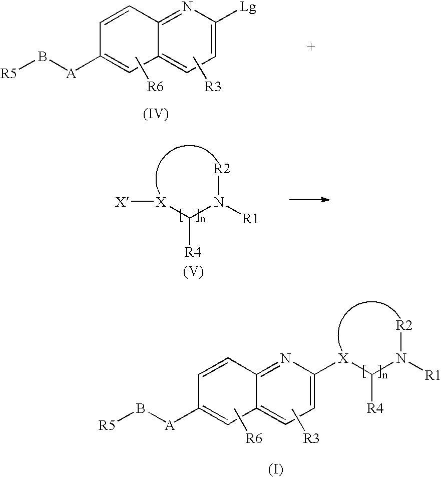

Alternatively, compounds of formula I can be made by N-alkylation of compounds of formula I having R1 or R2 being hydrogen using well-known synthetic routes such as reductive alkylation or alkylation with alkyl halides in case the functionalisation of the molecule is compatible with this type of reactions. For example amines VI can be reacted with reagents R1-Lg wherein Lg being a leaving group according to the following general scheme: 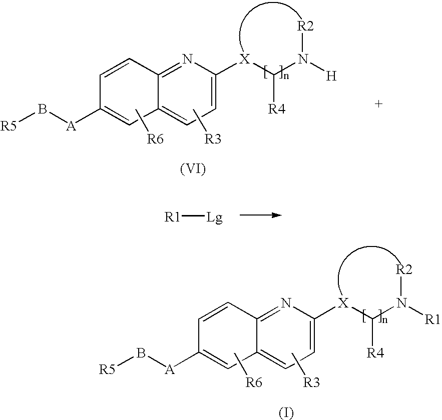
Examples of Specific Synthetic Methods

Thus, compound I having NHCON—R7 as linker A with R7 defined as hydrogen or lower alkyl or alkenyl group, can be produced, for instance, by the following urea reaction, or by the corresponding inverse reaction, analogous to formation of the thiourea below.

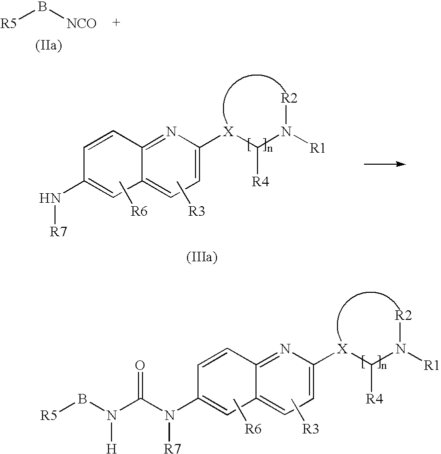

Compounds of formula I containing thioureas can be made from reactions of thioisocyanates with amines, analogous to the metods exemplified for ureas.

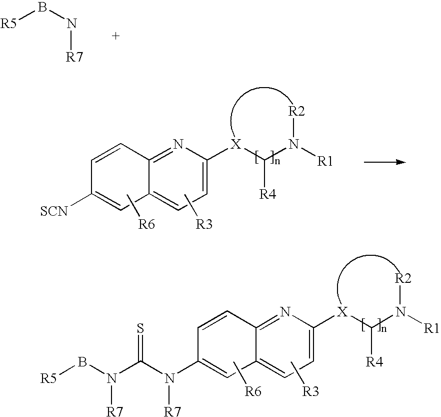

Compound IIa and compound IIIa are reacted in an inert solvent in accordance with standard procedures. Typically, inert solvents can be ether solvents, halogenated hydrocarbon solvents, nitrile solvents, aromatic solvents and amide solvents. Reaction temperature is usually room temperature and the reaction time is 2 hours to 1 day.

Compound IIa can be produced from the corresponding carboxylic acid. For instance, 4-phenoxyphenylisocyanate can be produced in accordance with methods such as described in “Comprehensive Organic Transformation”, 2^{nd} Edition (Wiley); R. C.

Larock.

Compound I having NAlk-CO-NR7 as linker A with R7 defined as hydrogen or lower alkyl or alkenyl group, can be produced, for instance, by the following urea reaction.

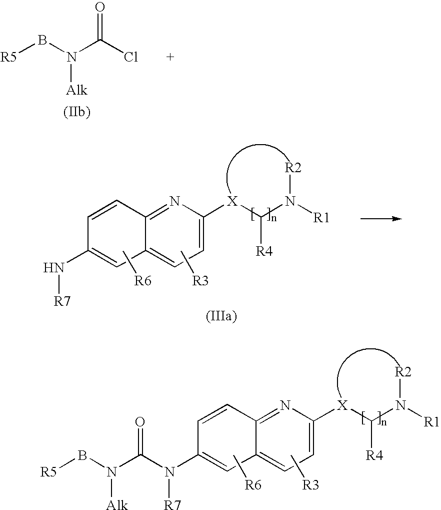

Compound IIIa and 1 equivalent of compound IIb are reacted in an inert solvent, usually in the presence of an excess of a base in accordance with known procedures (e.g. WO 9205174; J. Med. Chem. 43(20), 3653-3664, 2000). Suitable inert solvents can be ether solvents, halogenated hydrocarbon solvents, nitrile solvents, aromatic solvents and amide solvents. As a base can be used for instance triethylamine, diisopropylethylamine and sodium carbonate. Typically, the reaction temperature is 0° C. to room temperature and the reaction time is 1 hour to 1 day.

Compound I having CON—R7 as linker A with R7 defined as hydrogen or lower alkyl or alkenyl group, can be produced by the following amidation reaction.

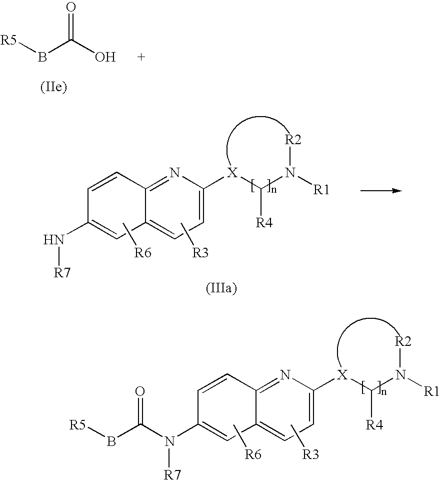

The amide bonds are formed by reacting a suitably activated carboxylic acid IIe (acid chloride, mixed anhydrides, esters with phenol bearing electron withdrawing substituents, 1-hydroxybenzotriazole, N-hydroxysuccinimide, 2-hydroxypyridine) with anilines IIIa in an inert solvent. As inert solvents can be used ether solvents, amide solvents and halogenated hydrocarbon solvents. If required the reaction is performed in the presence of a base. Suitable bases that can be used are triethylamine, diiisopropylethylamine, pyridine, 4-dimethylaminopyridine (DMAP) and sodium carbonate. The reaction temperature is usually between 0° C. to 30° C. and reaction time is 1 hour to 1 day.

The coupling can also be performed directly from IIe using suitable coupling reagents such as dicyclohexylcarbodiimide (DCC), 1-(3-dimethylaminopropyl)-3-ethyl-cabodiimide (EDCI), N-ethoxycarbonyl-2-ethoxy-1,2-dihydroquinoline (EEDQ) preferably in presence of promoting agents capable of forming an active ester such as 1-hydroxybenzotriazole, N-hydroxysuccinimide, 2-hydroxypyridine in an inert solvent. As inert solvents can be used ether solvents, amide solvents and halogenated hydrocarbon solvents. If required the reaction is performed in the presence of a base. Suitable bases that can be used are triethylamine, diiisopropylethylamine, pyridine, N-ethyldiisopropylamine, and 4-methylmorpholine. The reaction temperature is usually between 0° C. to 30° C. and reaction time is 1 hour to 1 day.

Analogously, a sulphonamide group, as the connecting A-linkage to form 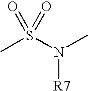
bonds can be made via the corresponding reaction of Ar—NH—R7 (IIIa) with activated forms of sulphonic acids, such sulphonyl chlorides, in the presence of base.

Compound I having 1,2,4-oxadiazole (X═O) or 1,2,4-triazole (X═NH) heterocyclic rings as linker A can be produced, for instance, by the following cyclodehydratation reaction.

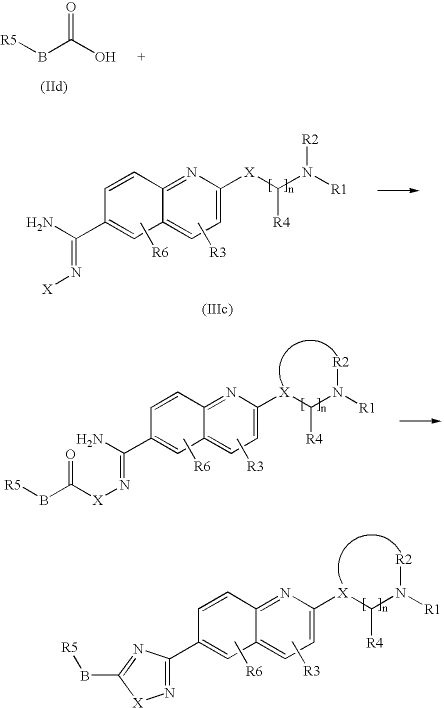

The ring closure is done in an inert solvent with or without the presence of a suitable base or acid (e.g. N-tetrabutyl ammonium fluoride, sodium hydride, sodium ethoxide or polyphosphoric acid) in accordance with standard methods such as described in Tetrahedron Lett. 42, 1441-1443, 2001; Tetrahedron Lett. 42, 1495-1498, 2001. Suitable, inert solvents can be ether solvents, amide solvents and aromatic solvents. The reaction temperature is usually room temperature to 100° C. and the reaction time is 1 hour to 3 days.

The intermediate can be produced by reaction of an activated derivative of compound IId with 1 equivalent of compound IIIc in an inert solvent in the presence of a base. As inert solvents can be used ether solvents, amide solvents and halogenated hydrocarbon solvents. Suitable bases that can be used are triethylamine, diiisopropylethylamine, pyridine and sodium carbonate.

Appropriate examples of the activated derivatives of compound IId include active esters (e.g. esters with phenol bearing electron withdrawing substituents, 1-hydroxybenzo-triazole, N-hydroxysuccinamide), acid chlorides, symmetrical or unsymmetrical anhydrides and orthoesters. The reaction temperature is usually between 0° C. to 30° C. and reaction time is 1 hour to 1 day.

Compounds of the type le can be made e.g. by reacting α-halo-amides of type IIIe with alcohols or phenols of type IIe.

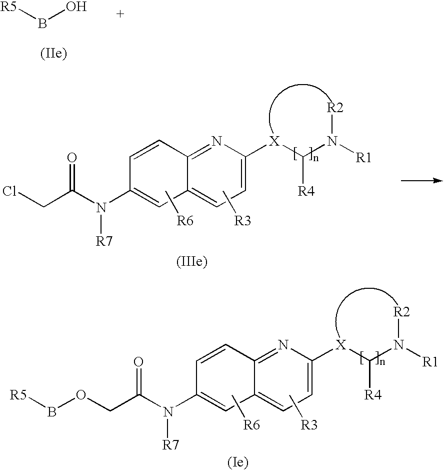

The reaction may be performed by heating a solution of IIe (2.5 equiv) with IIIe in acetone, in the presence of excess of a base, such as potassium carbonate (5 equiv). The reaction temperature is usually between 20 and 60° C., and the reaction time is usually between 0.5 and 24 hours.

Connection of the Eastern portion to the quinoline moiety can be carried out according to the methods described in the examples. Based on this knowledge, a person skilled in the art will be able to adapt the processes so as to be able to synthesise the compounds of interest.

Compounds

Below follows some examples of specific compounds for use according to the invention. In the compounds mentioned the different parts of the compounds, i.e. the linker -A-, the B group, the R1, R2, R3, R4, R5, R6 groups and the chain length are specified. Though not shown nor specifically mentioned, the invention also includes all compounds wherein all the mentioned variations in one part of the molecule, e.g. linker -A- is combined with all variations of the other features mentioned in the examples.
N-(4-Methyl-2-piperazin-1-yl-quinolin-6-yl)-2-(4-trifluoromethoxy-phenoxy)-acetamide
N-[4-Methyl-2-(4-methyl-piperazin-1-yl)-quinolin-6-yl]-2-(4-trifluoromethoxy-phenoxy)-acetamide
N-[4-Methyl-2-(4-pyrrolidin-1-yl-piperidin-1-yl)-quinolin-6-yl]-2-(4-trifluoromethoxy-phenoxy)-acetamide
N-[4-Methyl-2-(4-methyl-[1,4]diazepan-1-yl)-quinolin-6-yl]-2-(4-trifluoromethoxy-phenoxy)-acetamide
N{2-[4-(2-Hydroxy-ethyl)-piperazin-1-yl]-4-methyl-quinolin-6-yl}-2-(4-trifluoromethoxy-phenoxy)-acetamide
N-[2-(2,5-Diaza-bicyclo[2.2.1]hept-2-yl)-4-methyl-quinolin-6-yl]-2-(4-trifluoromethoxy-phenoxy)-acetamide
N-[2-(4-Dimethylamino-piperidin-1-yl)-4-methyl-quinolin-6-yl]-2-(4-trifluoromethoxy-phenoxy)-acetamide
2-(2-Chloro-4-trifluoromethoxy-phenoxy)-N-[4-methyl-2-(4-methyl-[1,4]diazepan-1-yl)-quinolin-6-yl]-acetamide
2-(2-Chloro-4-trifluoromethoxy-phenoxy)-N-(4-methyl-2-piperazin-1-yl-quinolin-6-yl)-acetamide
2-(2-Chloro-4-trifluoromethoxy-phenoxy)-N-[4-methyl-2-(4-methyl-piperazin-1-yl)-quinolin-6-yl]-acetamide
2-(2-Chloro-4-trifluoromethoxy-phenoxy)-N-{2-[4-(2-hydroxy-ethyl)-piperazin-1-yl]-4-methyl-quinolin-6-yl}-acetamide
2-(2-Chloro-4-trifluoromethoxy-phenoxy)-N-[2-(2,5-diaza-bicyclo[2.2.1]hept-2-yl)-4-methyl-quinolin-6-yl]-acetamide
2-(2-Chloro-4-trifluoromethoxy-phenoxy)-N-[4-methyl-2-(4-pyrrolidin-1-yl-piperidin-1-yl)-quinolin-6-yl]-acetamide
2-(2-Chloro-4-trifluoromethoxy-phenoxy)-N-[2-(4-dimethylamino-piperidin-1-yl)-4-methyl-quinolin-6-yl]-acetamide
2-(4-Chloro-phenoxy)-N-(4-methyl-2-piperazin-1-yl-quinolin-6-yl)-acetamide
2-(4-Chloro-phenoxy)-N-[4-methyl-2-(4-methyl-[1,4]diazepan-1-yl)-quinolin-6-yl]-acetamide
2-(4-Chloro-phenoxy)-N-[4-methyl-2-(4-methyl-piperazin-1-yl)-quinolin-6-yl]-acetamide
2-(4-Chloro-phenoxy)-N-[4-methyl-2-(4-pyrrolidin-1-yl-piperidin-1-yl)-quinolin-6-yl]-acetamide
2-(4-Chloro-phenoxy)-N{2-[4-(2-hydroxy-ethyl)-piperazin-1-yl]-4-methyl-quinolin-6-yl}-acetamide
2-(4-Chloro-phenoxy)-N-[2-(4-dimethylamino-piperidin-1-yl)-4-methyl-quinolin-6-yl]-acetamide
2-(4-Chloro-phenoxy)-N-[2-(2,5-diaza-bicyclo[2.2.1]hept-2-yl)-4-methyl-quinolin-6-yl]-acetamide
N-(4-Methyl-2-piperazin-1-yl-quinolin-6-yl)-2-p-tolyloxy-acetamide
N-{2-[4-(2-Hydroxy-ethyl)-piperazin-1-yl]-4-methyl-quinolin-6-yl}-2-p-tolyloxy-acetamide
N-[4-Methyl-2-(4-methyl-piperazin-1-yl)-quinolin-6-yl]-2-p-tolyloxy-acetamide
N-[4-Methyl-2-(4-methyl-[1,4]diazepan-1-yl)-quinolin-6-yl]-2-p-tolyloxy-acetamide
N-[4-Methyl-2-(4-pyrrolidin-1-yl-piperidin-1-yl)-quinolin-6-yl]-2-p-tolyloxy-acetamide
N-[2-(2,5-Diaza-bicyclo[2.2.1]hept-2-yl)-4-methyl-quinolin-6-yl]-2-p-tolyloxy-acetamide
N-[2-(4-Dimethylamino-piperidin-1-yl)-4-methyl-quinolin-6-yl]-2-p-tolyloxy-acetamide
N-(4-Methyl-2-piperazin-1-yl-quinolin-6-yl)-2-(4-trifluoromethyl-phenoxy)-acetamide
N-[4-Methyl-2-(4-methyl-[1,4]diazepan-1-yl)-quinolin-6-yl]-2-(4-trifluoromethyl-phenoxy)-acetamide
N-[4-Methyl-2-(4-methyl-piperazin-1-yl)-quinolin-6-yl]-2-(4-trifluoromethyl-phenoxy)-acetamide
N-{2-[4-(2-Hydroxy-ethyl)-piperazin-1-yl]-4-methyl-quinolin-6-yl}-2-(4-trifluoromethyl-phenoxy)-acetamide
N-[4-Methyl-2-(4-pyrrolidin-1-yl-piperidin-1-yl)-quinolin-6-yl]-2-(4-trifluoromethyl-phenoxy)-acetamide
N-[2-(2,5-Diaza-bicyclo[2.2.1]hept-2-yl)-4-methyl-quinolin-6-yl]-2-(4-trifluoromethyl-phenoxy)-acetamide
N-[2-(4-Dimethylamino-piperidin-1-yl)-4-methyl-quinolin-6-yl]-2-(4-trifluoromethyl-phenoxy)-acetamide
2-(4-Bromo-phenoxy)-N-(4-methyl-2-piperazin-1-yl-quinolin-6-yl)-acetamide
2-(4-Bromo-phenoxy)-N-[4-methyl-2-(4-methyl-[1,4]diazepan-1-yl)-quinolin-6-yl]-acetamide
2-(4-Bromo-phenoxy)-N-[4-methyl-2-(4-methyl-piperazin-1-yl)-quinolin-6-yl]-acetamide
2-(4-Bromo-phenoxy)-N-[4-methyl-2-(4-pyrrolidin-1-yl-piperidin-1-yl)-quinolin-6-yl]-acetamide
2-(4-Bromo-phenoxy)-N-{2-[4-(2-hydroxy-ethyl)-piperazin-1-yl]-4-methyl-quinolin-6-yl}-acetamide
2-(4-Bromo-phenoxy)-N-[2-(2,5-diaza-bicyclo[2.2.1]hept-2-yl)-4-methyl-quinolin-6-yl]-acetamide
2-(4-Bromo-phenoxy)-N-[2-(4-dimethylamino-piperidin-1-yl)-4-methyl-quinolin-6-yl]-acetamide
N-(4-Methyl-2-piperazin-1-yl-quinolin-6-yl)-3-(4-trifluoromethoxy-phenyl)-propionamide
N-[4-Methyl-2-(4-methyl-[1,4]diazepan-1-yl)-quinolin-6-yl]-3-(4-trifluoromethoxy-phenyl)-propionamide
N-[4-Methyl-2-(4-methyl-piperazin-1-yl)-quinolin-6-yl]-3-(4-trifluoromethoxy-phenyl)-propionamide
N-{2-[4-(2-Hydroxy-ethyl)-piperazin-1-yl]4-methyl-quinolin-6-yl}-3-(4-trifluoromethoxy-phenyl)-propionamide
N-[4-Methyl-2-(4-pyrrolidin-1-yl-piperidin-1-yl)-quinolin-6-yl]-3-(4-trifluoromethoxy-phenyl)-propionamide
N-{2-(2,5-Diaza-bicyclo[2.2.1]hept-2-yl)-4-methyl-quinolin-6-yl}-3-(4 trifluoromethoxy-phenyl)-propionamide
N-[2-(4-Dimethylamino-piperidin-1-yl)-4-methyl-quinolin-6-yl]-3-(4-trifluoromethoxy-phenyl)-propionamide
(E)-N-(4-Methyl-2-piperazin-1-yl-quinolin-6-yl)-3-(4-trifluoromethoxy-phenyl)-acrylamide
(E)-N-[4-Methyl-2-(4-methyl-[1,4]diazepan-1-yl)-quinolin-6-yl]-3-(4-trifluoromethoxy-phenyl)-acrylamide
(E)-N-[4-Methyl-2-(4-methyl-piperazin-1-yl)-quinolin-6-yl]-3-(4-trifluoromethoxy-phenyl)-acrylamide
(E)-N-{2-[4-(2-Hydroxy-ethyl)-piperazin-1-yl]-4-methyl-quinolin-6-yl}-3-(4-trifluoromethoxy-phenyl)-acrylamide
(E)-N-[2-(4-Dimethylamino-piperidin-1-yl)-4-methyl-quinolin-6-yl]-3-(4-trifluoromethoxy-phenyl)-acrylamide
(E)-N-[2-(2,5-Diaza-bicyclo[2.2.1]hept-2-yl)-4-methyl-quinolin-6-yl]-3-(4-trifluoromethoxy-phenyl)-acrylamide
(E)-N-[4-Methyl-2-(4-pyrrolidin-1-yl-piperidin-1-yl)-quinolin-6-yl]-3-(4-trifluoromethoxy-phenyl)-acrylamide
3-(4-Chloro-phenyl)-N-(4-methyl-2-piperazin-1-yl-quinolin-6-yl)-propionamide
3-(4-Chloro-phenyl)-N-[4-methyl-2-(4-methyl-[1,4]diazepan-1-yl)-quinolin-6-yl]-propionamide
3-(4-Chloro-phenyl)-N-[4-methyl-2-(4-methyl-piperazin-1-yl)-quinolin-6-yl]-propionamide
3-(4-Chloro-phenyl)-N-{2-[4-(2-hydroxy-ethyl)-piperazin-1-yl]-4-methyl-quinolin-6-yl}-propionamide
3-(4-Chloro-phenyl)-N-[4-methyl-2-(4-pyrrolidin-1-yl-piperidin-1-yl)-quinolin-6-yl]-propionamide
3-(4-Chloro-phenyl)-N-[2-(2,5-diaza-bicyclo[2.2.1]hept-2-yl)-4-methyl-quinolin-6-yl]-propionamide
3-(4-Chloro-phenyl)-N-[2-(4-dimethylamino-piperidin-1-yl)-4-methyl-quinolin-6-yl]-propionamide
2-(2,4-Dichloro-phenoxy)-N-(4-methyl-2-piperazin-1-yl-quinolin-6-yl)-acetamide
2-(2,4-Dichloro-phenoxy)-N-[4-methyl-2-(4-methyl-[1,4]diazepan-1-yl)-quinolin-6-yl]-acetamide
2-(2,4-Dichloro-phenoxy)-N-[4-methyl-2-(4-methyl-piperazin-1-yl)-quinolin-6-yl-1]-acetamide
2-(2,4-Dichloro-phenoxy)-N-{2-[4-(2-hydroxy-ethyl)-piperazin-1-yl]-4-methyl-quinolin-6-yl}acetamide
2-(2,4-Dichloro-phenoxy)-N-[2-(4-dimethylamino-piperidin-1-yl)-4-methyl-quinolin-6-yl]-acetamide
N-[2-(2,5-Diaza-bicyclo[2.2.1]hept-2-yl)-4-methyl-quinolin-6-yl]-2-(2,4-dichloro-phenoxy)-acetamide
2-(2,4-Dichloro-phenoxy)-N-[4-methyl-2-(4-pyrrolidin-1-yl-piperidin-1-yl)-quinolin-6-yl]-acetamide
N-(4-Methyl-2-piperazin-1-yl-quinazolin-6-yl)-2-(4-trifluoromethoxy-phenoxy)-acetamide
N-[4-Methyl-2-(4-methyl-[1,4]diazepan-1-yl)-quinazolin-6-yl]-2-(4-trifluoromethoxy-phenoxy)-acetamide
N-[4-Methyl-2-(4-methyl-piperazin-1-yl)-quinazolin-6-yl]-2-(4-trifluoromethoxy-phenoxy)-acetamide
N-{2-[4-(2-Hydroxy-ethyl)-piperazin-1-yl]-4-methyl-quinazolin-6-yl}-2-(4-trifluoromethoxy-phenoxy)-acetamide
N-[4-Methyl-2-(4-pyrrolidin-1-yl-piperidin-1-yl)-quinazolin-6-yl]-2-(4-trifluoromethoxy-phenoxy)-acetamide
N-[2-(2,5-Diaza-bicyclo[2.2.1]hept-2-yl)-4-methyl-quinazolin-6-yl]-2-(4-trifluoromethoxy-phenoxy)-acetamide
N-[2-(4-Dimethylamino-piperidin-1-yl)-4-methyl-quinazolin-6-yl]-2-(4-trifluoromethoxy-phenoxy)-acetamide
2-(2-Chloro-4-trifluoromethoxy-phenoxy)-N-(4-methyl-2-piperazin-1-yl-quinazolin-6-yl)-acetamide
2-(2-Chloro-4-trifluoromethoxy-phenoxy)-N-[4-methyl-2-(4-methyl-[1,4]diazepan-1-yl)-quinazolin-6-yl]-acetamide
2-(2-Chloro-4-trifluoromethoxy-phenoxy)-N-[4-methyl-2-(4-methyl-piperazin-1-yl)-quinazolin-6-yl]-acetamide
2-(2-Chloro-4-trifluoromethoxy-phenoxy)-N-{2-[4-(2-hydroxy-ethyl)-piperazin-1-yl]4-methyl-quinazolin-6-yl}-acetamide
2-(2-Chloro-4-trifluoromethoxy-phenoxy)-N-[2-(4-dimethylamino-piperidin-1-yl)-4-methyl-quinazolin-6-yl]-acetamide
2-(2-Chloro-4-trifluoromethoxy-phenoxy)-N-[2-(2,5-diaza-bicyclo[2.2.1]hept-2-yl)-4-methyl-quinazolin-6-yl]-acetamide
2-(2-Chloro-4-trifluoromethoxy-phenoxy)-N-[4-methyl-2-(4-pyrrolidin-1-yl-piperidin-1-yl)-quinazolin-6-yl]-acetamide
2-(4-Chloro-phenoxy)-N-(4-methyl-2-piperazin-1-yl-quinazolin-6-yl)-acetamide
2-(4-Chloro-phenoxy)-N-[4-methyl-2-(4-methyl-[1,4]diazepan-1-yl)-quinazolin-6-yl]-acetamide
2-(4-Chloro-phenoxy)-N-[4-methyl-2-(4-methyl-piperazin-1-yl)-quinazolin-6-yl]-acetamide
2-(4-Chloro-phenoxy)-N-{2-[4-(2-hydroxy-ethyl)-piperazin-1-yl]-4-methyl-quinazolin-6-yl}-acetamide
2-(4-Chloro-phenoxy)-N-[4-methyl-2-(4-pyrrolidin-1-yl-piperidin-1-yl)-quinazolin-6-yl]-acetamide
2-(4-Chloro-phenoxy)-N-[2-(2,5-diaza-bicyclo[2.2.1]hept-2-yl)-4-methyl-quinazolin-6-yl]-acetamide
2-(4-Chloro-phenoxy)-N-[2-(4-dimethylamino-piperidin-1-yl)-4-methyl-quinazolin-6-yl]-acetamide
N-(4-Methyl-2-piperazin-1-yl-quinazolin-6-yl)-2-p-tolyloxy-acetamide
N-[4-Methyl-2-(4-methyl-[1,4]diazepan-1-yl)-quinazolin-6-yl]-2-p-tolyloxy-acetamide
N-[4-Methyl-2-(4-methyl-piperazin-1-yl)-quinazolin-6-yl]-2-p-tolyloxy-acetamide
N-{2-[4-(2-Hydroxy-ethyl)-piperazin-1-yl]-4-methyl-quinazolin-6-yl}2-p-tolyloxy-acetamide
N-[4-Methyl-2-(4-pyrrolidin-1-yl-piperidin-1-yl)-quinazolin-6-yl]-2-p-tolyloxy-acetamide
N-[2-(2,5-Diaza-bicyclo[2.2.1]hept-2-yl)-4-methyl-quinazolin-6-yl]-2-p-tolyloxy-acetamide
N-[2-(4-Dimethylamino-piperidin-1-yl)-4-methyl-quinazolin-6-yl]-2-p-tolyloxy-acetamide
N-[4-Methyl-2-piperazin-1-yl-quinazolin-6-yl]-2-(4-trifluoromethyl-phenoxy)-acetamide
N-[4-Methyl-2-(4-methyl-[1,4]diazepan-1-yl)-quinazolin-6-yl]-2-(4-trifluoromethyl-phenoxy)-acetamide
N-[4-Methyl-2-(4-methyl-piperazin-1-yl)-quinazolin-6-yl]-2-(4-trifluoromethyl-phenoxy)-acetamide
N-{2-[(4-(2-Hydroxy-ethyl)-piperazin-1-yl]-4-methyl-quinazolin-6-yl}-2-(4-trifluoromethyl-phenoxy)-acetamide
N-[4-Methyl-2-(4-pyrrolidin-1-yl-piperidin-1-yl)-quinazolin-6-yl]-2-(4-trifluoromethyl-phenoxy)-acetamide
N-[2-(2,5-Diaza-bicyclo[2.2.1]hept-2-yl)-4-methyl-quinazolin-6-yl]-2-(4-trifluoromethyl-phenoxy)-acetamide
2-(4-Bromo-phenoxy)-N-(4-methyl-2-piperazin-1-yl-quinazolin-6-yl)-acetamide
2-(4-Bromo-phenoxy)-N-(4-methyl-2-piperazin-1-yl-quinazolin-6-yl)-acetamide
2-(4-Bromo-phenoxy)-N-[4-methyl-2-(4-methyl-piperazin-1-yl)-quinazolin-6-yl]-acetamide
2-(4-Bromo-phenoxy)-N-{2-[4-(2-hydroxy-ethyl)-piperazin-1-yl]-4-methyl-quinazolin-6-yl}acetamide
2-(4-Bromo-phenoxy)-N-[4-methyl-2-(4-pyrrolidin-1-yl-piperidin-1-yl)-quinazolin-6-yl]-acetamide
2-(4-Bromo-phenoxy)-N-[2-(2,5-diaza-bicyclo[2.2.1]hept-2-yl)-4-methyl-quinazolin-6-yl]-acetamide
2-(4-Bromo-phenoxy)-N-[2-(4-dimethylamino-piperidin-1-yl)-4-methyl-quinazolin-6-yl]-acetamide
N-(4-Methyl-2-piperazin-1-yl-quinazolin-6-yl)-3-(4-trifluoromethoxy-phenyl)-propionamide
N-[4-Methyl-2-(4-methyl-[1,4]diazepan-1-yl)-quinazolin-6-yl]-3-(4-trifluoromethoxy-phenyl)-propionamide
N-[4-Methyl-2-(4-methyl-piperazin-1-yl)-quinazolin-6-yl]-3-(4-trifluoromethoxy-phenyl)-propionamide
N-{2-[4-(2-Hydroxy-ethyl)-piperazin-1-yl]-4-methyl-quinazolin-6-yl}3-(4-trifluoromethoxy-phenyl)-propionamide
N-[4-Methyl-2-(4-pyrrolidin-1-yl-piperidin-1-yl)-quinazolin-6-yl]-3-(4-trifluoromethoxy-phenyl)-propionamide
N-[2-(2,5-Diaza-bicyclo[2.2.1]hept-2-yl-4-methyl-quinazolin-6-yl]-3-(4-trifluoromethoxy-phenyl)-propionamide
N-[2-(4-Dimethylamino-piperidin-1-yl)-4-methyl-quinazolin-6-yl]-3-(4-trifluoromethoxy-phenyl)-propionamide
(E)-N-(4-Methyl-2-piperazin-1-yl-quinazolin-6-yl)-3-(4-trifluoromethoxy-phenyl)-acrylamide
(E)-N-[4-Methyl-2-(4-methyl-[1,4]diazepan-1-yl)-quinazolin-6-yl]-3-(4-trifluoromethoxy-phenyl)-acrylamide
(E)-N-[4-Methyl-2-(4-methyl-piperazin-1-yl)-quinazolin-6-yl]-3-(4-trifluoromethoxy-phenyl)-acrylamide
(E)-N-{2-[4-(2-Hydroxy-ethyl)-piperazin-1-yl]-4-methyl-quinazolin-6-yl}3-(4-trifluoromethoxy-phenyl)-acrylamide
(E)-N-[4-Methyl-2-(4-pyrrolidin-1-yl-piperidin-1-yl)-quinazolin-6-yl]-3-(4-trifluoromethoxy-phenyl)-acrylamide
(E)-N-[2-(2,5-Diaza-bicyclo[2.2.1]hept-2-yl)-4-methyl-quinazolin-6-yl]-3-(4-trifluoromethoxy-phenyl)-acrylamide
(E)-N-[2-(4-Dimethylamino-piperidin-1-yl)-4-methyl-quinazolin-6-yl]-3-(4-trifluoromethoxy-phenyl)-acrylamide
3-(4-Chloro-phenyl)-N-(4-methyl-2-piperazin-1-yl-quinazolin-6-yl)-propionamide
3-(4-Chloro-phenyl)-N-[4-methyl-2-(4-methyl-[1,4]diazepan-1-yl)-quinazolin-6-yl]-propionamide
3-(4-Chloro-phenyl)-N-[4-methyl-2-(4-methyl-piperazin-1-yl)-quinazolin-6-yl]-propionamide
3-(4-Chloro-phenyl)-N-{2-[4-(2-hydroxy-ethyl)-piperazin-1-yl]-4-methyl-quinazolin-6-yl}-propionamide
3-(4-Chloro-phenyl)-N-[4-methyl-2-(4-pyrrolidin-1-yl-piperidin-1-yl)-quinazolin-6-yl]-propionamide
3-(4-Chloro-phenyl)-N-[2-(2,5-diaza-bicyclo[2.2.1]hept-2-yl)-4-methyl-quinazolin-6-yl]-propionamide
3-(4-Chloro-phenyl)-N-[2-(4-dimethylamino-piperidin-1-yl)-4-methyl-quinazolin-6-yl]-propionamide
2-(2,4-Dichloro-phenoxy)-N-(4-methyl-2-piperazin-1-yl-quinazolin-6-yl)-acetamide
2-(2,4-Dichloro-phenoxy)-N-[4-methyl-2-(4-methyl-[1,4]diazepan-1-yl)-quinazolin-6-yl]-acetamide
2-(2,4-Dichloro-phenoxy)-N-[4-methyl-2-(4-methyl-piperazin-1-yl)-quinazolin-6-yl]-acetamide
2-(2,4-Dichloro-phenoxy)-N-{2-[4-(2-hydroxy-ethyl)-piperazin-1-yl]4-methyl-quinazolin-6-yl}-acetamide
2-(2,4-Dichloro-phenoxy)-N-[4-methyl-2-(4-pyrrolidin-1-yl-piperidin-1-yl)-quinazolin-6-yl]-acetamide
N-[2-(2,5-Diaza-bicyclo[2.2.1]hept-2-yl)-4-methyl-quinazolin-6-yl]-2-(2,4-dichloro-phenoxy)-acetamide
2-(2,4-Dichloro-phenoxy)-N-[2-(4-dimethylamino-piperidin-1-yl)-4-methyl-quinazolin-6-yl]-acetamide
N-(4-Methyl-2-piperazin-1-yl-quinazolin-6-yl)-2-(4-trifluoromethoxy-phenoxy)-acetamide
N-[2-(4-Methyl-[1,4]diazepan-1-yl)-quinazolin-6-yl]-2-(4-trifluoromethoxy-phenoxy)-acetamide
N-[2-(4-Methyl-piperazin-1-yl)-quinazolin-6-yl]-2-(4-trifluoromethoxy-phenoxy)-acetamide
N-{2-[4-(2-Hydroxy-ethyl)-piperazin-1-yl)-quinazolin-6-yl}-2-(4-trifluoromethoxy-phenoxy)-acetamide
N-[2-(4-Pyrrolidin-1-yl-piperidin-1-yl)-quinazolin-6-yl]-2-(4-trifluoromethoxy-phenoxy)-acetamide
N-[2-(2,5-Diaza-bicyclo[2.2.1]hept-2-yl)quinazolin-6-yl]-2-(4-trifluoromethoxy-phenoxy)-acetamide
N-[2-(4-Dimethylamino-piperidin-1-yl)-quinazolin-6-yl]-2-(4-trifluoromethoxy-phenoxy)-acetamide
2-(2-Chloro-4-trifluoromethoxy-phenoxy)-N-(2-piperazin-1-yl-quinazolin-6-yl)-acetamide
2-(2-Chloro-4-trifluoromethoxy-phenoxy)-N-[2-(4-methyl-[1,4]diazepan-1-yl)-quinazolin-6-yl]-acetamide
2-(2-Chloro-4-trifluoromethoxy-phenoxy)-N-[2-(4-methyl-piperazin-1-yl)-quinazolin-6-yl]-acetamide
2-(2-Chloro-4-trifluoromethoxy-phenoxy)-N-{2-[4-(2-hydroxy-ethyl)-piperazin-1-yl]-quinazolin-6-yl}-acetamide
2-(2-Chloro-4-trifluoromethoxy-phenoxy)-N-[2-(4-dimethylamino-piperidin-1-yl)-quinazolin-6-yl]-acetamide
2-(2-Chloro-4-trifluoromethoxy-phenoxy)-N-[2-(2,5-diaza-bicyclo[2.2.1]hept-2-yl)-quinazolin-6-yl]-acetamide
2-(2-Chloro-4-trifluoromethoxy-phenoxy)-N-[2-(4-pyrrolidin-1-yl-piperidin-1-yl)-quinazolin-6-yl]-acetamide
2-(4-Chloro-phenoxy)-N-(2-piperazin-1-yl-quinazolin-6-yl)-acetamide
2-(4-Chloro-phenoxy)-N-[2-(4-methyl-[1,4]diazepan-1-yl)-quinazolin-6-yl]-acetamide
2-(4-Chloro-phenoxy)-N-[2-(4-methyl-piperazin-1-yl)-quinazolin-6-yl]-acetamide
2-(4-Chloro-phenoxy)-N-{2-[4-(2-hydroxy-ethyl)-piperazin-1-yl}-quinazolin-6-yl]acetamide
2-(4-Chloro-phenoxy)-N-[2-(4-pyrrolidin-1-yl-piperidin-1-yl-quinazolin-6-yl]-acetamide
2-(4-Chloro-phenoxy)-N-[2-(2,5-diaza-bicyclo[2.2.1]hept-2-yl)-quinazolin-6-yl]-acetamide
2-(4-Chloro-phenoxy)-N-[2-(4-dimethylamino-piperidin-1-yl)-quinazolin-6-yl]-acetamide
N-(2-piperazin-1-yl-quinazolin-6-yl)-2-p-tolyloxy-acetamide
N-[2-(4-Methyl-[1,4]diazepan-1-yl)-quinazolin-6-yl]-2-p-tolyloxy-acetamide
N-[2-(4-Methyl-piperazin-1-yl)-quinazolin-6-yl]-2-p-tolyloxy-acetamide
N-{2-[4-(2-Hydroxy-ethyl)-piperazin-1-yl]-quinazolin-6-yl}-2-p-tolyloxy-acetamide
N-[2-(4-Pyrrolidin-1-yl-piperidin-1-yl)-quinazolin-6-yl]-2-p-tolyloxy-acetamide
N-[2-(2,5-Diaza-bicyclo[2.2.1]hept-2-yl)-quinazolin-6-yl]-2-p-tolyloxy-acetamide
N-[2-(4-Dimethylamino-piperidin-1-yl)-quinazolin-6-yl]-2-p-tolyloxy-acetamide
N-(2-piperazin-1-yl-quinazolin-6-yl)-2-(4-trifluoromethyl-phenoxy)-acetamide
N-[2-(4-Methyl-[1,4]diazepan-1-yl)-quinazolin-6-yl]-2-(4-trifluoromethyl-phenoxy)-acetamide
N-[2-(4-Methyl-piperazin-1-yl)-quinazolin-6-yl]-2-(4-trifluoromethyl-phenoxy)-acetamide
N-{2-[4-(2-Hydroxy-ethyl)-piperazin-1-yl]-quinazolin-6-yl}-2-(4-trifluoromethyl-phenoxy)-acetamide
N-[2-(4-Pyrrolidin-1-yl-piperidin-1-yl)-quinazolin-6-yl]-2-(4-trifluoromethyl-phenoxy)-acetamide
N-[2-(2,5-Diaza-bicyclo[2.2.1]hept-2-yl)-quinazolin-6-yl]-2-(4-trifluoromethyl-phenoxy)-acetamide
N-[2-(4-Dimethylamino-piperidin-1-yl)-quinazolin-6-yl]-2-(4-trifluoromethyl-phenoxy)-acetamide
2-(4-Bromo-phenoxy)-N-(2-piperazin-1-yl-quinazolin-6-yl]-acetamide
2-(4-Bromo-phenoxy)-N-[2-(4-methyl-piperazin-1-yl)-quinazolin-6-yl]-acetamide
2-(4-Bromo-phenoxy)-N-[2-(4-pyrrolidin-1-yl-piperidin-1-yl)-quinazolin-6-yl]-acetamide
2-(4-Bromo-phenoxy)-N-[2-(4-methyl-[1,4]diazepan-1-yl)-quinazolin-6-yl]-acetamide
2-(4-Bromo-phenoxy)-N-{2-[4-(2-hydroxy-ethyl)-piperazin-1-yl]-quinazolin-6-yl}acetamide
2-(4-Bromo-phenoxy)-N-[2-(2,5-diaza-bicyclo[2.2.1]hept-2-yl)-quinazolin-6-yl]-acetamide
2-(4-Bromo-phenoxy)-N-[2-(4-dimethylamino-piperidin-1-yl)-quinazolin-6-yl]-acetamide
N-(2-piperazin-1-yl-quinazolin-6-yl)-3-(4-trifluoromethoxy-phenyl)-propionamide
N-[2-(4-Methyl-[1,4]diazepan-1-yl)-quinazolin-6-yl]-3-(4-trifluoromethoxy-phenyl)-propionamide
N-[2-(4-Methyl-piperazin-1-yl)-quinazolin-6-yl]-3-(4-trifluoromethoxy-phenyl)-propionamide
N{2-[4-(2-Hydroxy-ethyl)-piperazin-1-yl]-quinazolin-6-yl}-3-(4-trifluoromethoxy-phenyl)-propionamide
N-[2-(4-Pyrrolidin-1-yl-piperidin-1-yl)-quinazolin-6-yl]-3-(4-trifluoromethoxy-phenyl)-propionamide
N-[2-(2,5-Diaza-bicyclo[2.2.1]hept-2-yl)-quinazolin-6-yl]-3-(4-trifluoromethoxy-phenyl)-propionamide
N-[2-(4-Dimethylamino-piperidin-1-yl)-quinazolin-6-yl]-3-(4-trifluoromethoxy-phenyl)-propionamide
(E)-N-(2-piperazin-1-yl-quinazolin-6-yl)-3-(4-trifluoromethoxy-phenyl)-acrylamide
(E)-N-[2-(4-Methyl-[1,4]diazepan-1-yl)-quinazolin-6-yl]-3-(4-trifluoromethoxy-phenyl)-acrylamide
(E)-N-[2-(4-Methyl-piperazin-1-yl)-quinazolin-6-yl]-3-(4-trifluoromethoxy-phenyl)-acrylamide
(E)-N-{2-[4-(2-Hydroxy-ethyl)-piperazin-1-yl]-quinazolin-6-yl}-3-(4-trifluoromethoxy-phenyl)-acrylamide
(E)-N-[2-(4-Pyrrolidin-1-yl-piperidin-1-yl)-quinazolin-6-yl]-3-(4-trifluoromethoxy-phenyl)-acrylamide
(E)-N-[2-(2,5-Diaza-bicyclo[2.2.1]hept-2-yl)-quinazolin-6-yl]-3-(4-trifluoromethoxy-phenyl)-acrylamide
(E)-N-[2-(4-Dimethylamino-piperidin-1-yl)-quinazolin-6-yl]-3-(4-trifluoromethoxy-phenyl)-acrylamide
3-(4-Chloro-phenyl)-N-(2-piperazin-1-yl-quinazolin-6-yl)-propionamide
3-(4-Chloro-phenyl)-N-[2-(4-methyl-[1,4]diazepan-1-yl-quinazolin-6-yl]-propionamide
3-(4-Chloro-phenyl)-N-[2-(4-methyl-piperazin-1-yl)-quinazolin-6-yl]-propionamide
3-(4-Chloro-phenyl)-N-{2-[4-(2-hydroxy-ethyl)-piperazin-1-yl]-quinazolin-6-yl}-propionamide
3-(4-Chloro-phenyl)-N-[2-(4-pyrrolidin-1-yl-piperidin-1-yl)-quinazolin-6-yl]-propionamide
3-(4-Chloro-phenyl)-N-[2-(2,5-diaza-bicyclo[2.2.1]hept-2-yl)-quinazolin-6-yl]-propionamide
3-(4-Chloro-phenyl)-N-[2-(4-dimethylamino-piperidin-1-yl)-quinazolin-6-yl]-propionamide
2-(2,4-Dichloro-phenoxy)-N-(2-piperazin-1-yl-quinazolin-6-yl)-acetamide
2-(2,4-Dichloro-phenoxy)-N-[2-(4-methyl-[1,4]diazepan-1-yl)-quinazolin-6-yl]-acetamide
2-(2,4-Dichloro-phenoxy)-N-[2-(4-methyl-piperazin-1-yl)-quinazolin-6-yl]-acetamide
2-(2,4-Dichloro-phenoxy)-N-{2-[4-(2-hydroxy-ethyl)-piperazin-1-yl]-quinazolin-6-yl}-acetamide
2-(2,4-Dichloro-phenoxy)-N-[2-(4-pyrrolidin-1-yl-piperidin-1-yl)-quinazolin-6-yl]-acetamide
N-[2-(2,5-Diaza-bicyclo[2.2.1]hept-2-yl)-quinazolin-6-yl]-2-(2,4-dichloro-phenoxy)-acetamide
2-(2,4-Dichloro-phenoxy)-N-[2-(4-dimethylamino-piperidin-1-yl)-quinazolin-6-yl]-acetamide
N-(4-Amino-piperidin-1-yl)-4-methyl-quinolin-6-yl)-2-(4-trifluoromethoxy-phenoxy)-acetamide
2-(2-Chloro-4-trifluoromethoxy-phenoxy)-N-(4-amino-piperidin-1-yl)-4-methyl-quinolin-6-yl)-acetamide
2-(4-Chloro-phenoxy)-N-(4-amino-piperidin-1-yl)-4-methyl-quinolin-6-yl)-acetamide
N-(4-Amino-piperidin-1-yl)-4-methyl-quinolin-6-yl)-2-p-tolyloxy-acetamide
N-(4-Amino-piperidin-1-yl)-4-methyl-quinolin-6-yl)-2-(4-trifluoromethyl-phenoxy)-acetamide
2-(4-Bromo-phenoxy)-N-(4-amino-piperidin-1-yl)-4-methyl-quinolin-6-yl)-acetamide
N-(4-Amino-piperidin-1-yl)-4-methyl-quinolin-6-yl)-3-(4-trifluoromethoxy-phenyl)-propionamide
(E)-N-(4-Amino-piperidin-1-yl)+methyl-quinolin-6-yl)-3-(4-trifluoromethoxy-phenyl)-acrylamide
3-(4-Chloro-phenyl)-N-(4-amino-piperidin-1-yl)-4-methyl-quinolin-6-yl)-propionamide
2-(2,4-Dichloro-phenoxy)-N-(4-amino-piperidin-1-yl)-4-methyl-quinolin-6-yl)-acetamide
N-(4-Methyl-2-(4-methylamino-piperidin-1-yl-quinolin-6-yl)-2-(4-trifluoromethoxy-phenoxy)-acetamide
2-(2-Chloro-4-trifluoromethoxy-phenoxy)-N-(4-methyl-2-(4-methylamino-piperidin-1-yl-quinolin-6-yl)-acetamide
2-(4-Chloro-phenoxy)-N-(4-methyl-2-(4-methylamino-piperidin-1-yl-quinolin-6-yl)-acetamide
N-(4-Methyl-2-(4-methylamino-piperidin-1-yl-quinolin-6-yl)-2-p-tolyloxy-acetamide
N-(4-Methyl-2-(4-methylamino-piperidin-1-yl-quinolin-6-yl)-2-(4-trifluoromethyl-phenoxy)-acetamide
2-(4-Bromo-phenoxy)-N-(4-methyl-2-(4-methylamino-piperidin-1-yl-quinolin-6-yl)-acetamide
N-(4-Methyl-2-(4-methylamino-piperidin-1-yl-quinolin-6-yl)-3-(4-trifluoromethoxy-phenyl)-propionamide
(E)-N-(4-Methyl-2-(4-methylamino-piperidin-1-yl-quinolin-6-yl)-3-(4-trifluoromethoxy-phenyl)-acrylamide
3-(4-Chloro-phenyl)-N-(4-methyl-2-(4-methylamino-piperidin-1-yl-quinolin-6-yl)-propionamide
2-(2,4-Dichloro-phenoxy)-N-(4-methyl-2-(4-methylamino-piperidin-1-yl-quinolin-6-yl)-acetamide
N-(4-Methyl-2-(4-isopropyl-piperazin-1-yl)-quinolin-6-yl)-2-(4-trifluoromethoxy-phenoxy)-acetamide
2-(2-Chloro-4-trifluoromethoxy-phenoxy)-N-(4-methyl-2-(4-isopropyl-piperazin-1-yl)-quinolin-6-yl)-acetamide
2-(4-Chloro-phenoxy)-N-(4-methyl-2-(4-isopropyl-piperazin-1-yl)-quinolin-6-yl)-acetamide
N-(4-Methyl-2-(4-isopropyl-piperazin-1-yl)-quinolin-6-yl)-2-p-tolyloxy-acetamide
N-(4-Methyl-2-(4-isopropyl-piperazin-1-yl)-quinolin-6-yl)-2-(4-trifluoromethyl-phenoxy)-acetamide
2-(4-Bromo-phenoxy)-N-(4-methyl-2-(4-isopropyl-piperazin-1-yl)-quinolin-6-yl)-acetamide
N-(4-Methyl-2-(4-isopropyl-piperazin-1-yl)-quinolin-6-yl)-3-(4-trifluoromethoxy-phenyl)-propionamide
(E)-N-(4-Methyl-2-(4-isopropyl-piperazin-1-yl)-quinolin-6-yl)-3-(4-trifluoromethoxy-phenyl)-acrylamide
3-(4-Chloro-phenyl)-N-(4-methyl-2-(4-isopropyl-piperazin-1-yl)-quinolin-6-yl)-propionamide
2-(2,4-Dichloro-phenoxy)-N-(4-methyl-2-(4-isopropyl-piperazin-1-yl)-quinolin-6-yl)-acetamide
N-{2-[4-(2-Hydroxy-ethyl)-[1,4]diazepan-1-yl]-4-methyl-quinolin-6-yl}-2-(4-trifluoromethoxy-phenoxy)-acetamide
2-(2-Chloro-4-trifluoromethoxy-phenoxy)-N-{2-[4-(2-hydroxy-ethyl)-[1,4]diazepan-1-yl]-4-methyl-quinolin-6-yl}-acetamide
2-(4-Chloro-phenoxy)-N-{2-[4-(2-hydroxy-ethyl)-[1,4]diazepan-1-yl]-4-methyl-quinolin-6-yl}-acetamide
N-{2-[4-(2-Hydroxy-ethyl)-[1,4]diazepan-1-yl]-4-methyl-quinolin-6-yl}-2-p-tolyloxy-acetamide
N{2-[4-(2-Hydroxy-ethyl)-[1,4]diazepan-1-yl]-4-methyl-quinolin-6-yl}-2-(4-trifluoromethyl-phenoxy)-acetamide
2-(4-Bromo-phenoxy)-N-{2-[4-(2-hydroxy-ethyl)-[1,4]diazepan-1-yl]-4-methyl-quinolin-6-yl}-acetamide
N-{2-[4-(2-Hydroxy-ethyl)-[1,4]diazepan-1-yl]-4-methyl-quinolin-6-yl}-3-(4-trifluoromethoxy-phenyl)-propionamide
(E)-N-{2-[4-(2-Hydroxy-ethyl)-[1,4]diazepan-1-yl]-4-methyl-quinolin-6-yl}-3-(4-trifluoromethoxy-phenyl)-acrylamide
3-(4-Chloro-phenyl)-N-{2-[4-(2-hydroxy-ethyl)-[1,4]diazepan-1-yl]-4-methyl-quinolin-6-yl}-propionamide
2-(2,4-Dichloro-phenoxy)-N-{2-[4-(2-hydroxy-ethyl)-[1,4]diazepan-1-yl]-4-methyl-quinolin-6-yl}acetamide
N-(2-[1,4]Diazepan-1-yl-4-methyl-quinolin-6-yl)-2-(4-trifluoromethoxy-phenoxy)-acetamide
2-(2-Chloro-4-trifluoromethoxy-phenoxy)-N-(2-[1,4]diazepan-1-yl-4-methyl-quinolin-6-yl)-acetamide
2-(4-Chloro-phenoxy)-N-(2-[1,4]diazepan-1-yl-4-methyl-quinolin-6-yl)-acetamide
N-(2-[1,4]Diazepan-1-yl-4-methyl-quinolin-6-yl)-2-p-tolyloxy-acetamide
N-(2-[1,4]diazepan-1-yl-4-methyl-quinolin-6-yl)-2-(4-trifluoromethyl-phenoxy)-acetamide
2-(4-Bromo-phenoxy)-N-(2-[1,4]diazepan-1-yl-4-methyl-quinolin-6-yl)-acetamide
N-(2-[1,4]Diazepan-1-yl-4-methyl-quinolin-6-yl)-3-(4-trifluoromethoxy-phenyl)-propionamide
(E)-N-(2-[1,4]Diazepan-1-yl-4-methyl-quinolin-6-yl)-3-(4-trifluoromethoxy-phenyl)-acrylamide
3-(4-Chloro-phenyl)-N-(2-[1,4]diazepan-1-yl-4-methyl-quinolin-6-yl)-propionamide
2-(2,4-Dichloro-phenoxy)-N-(2-[1,4]diazepan-1-yl-4-methyl-quinolin-6-yl)-acetamide
N-(4-Amino-piperidin-1-yl)-4-methyl-quinazolin-6-yl)-2-(4-trifluoromethoxy-phenoxy)-acetamide
2-(2-Chloro-4-trifluoromethoxy-phenoxy)-N-(4-amino-piperidin-1-yl)-4-methyl-quinazolin-6-yl)-acetamide
2-(4-Chloro-phenoxy)-N-(4-amino-piperidin-1-yl)-4-methyl-quinazolin-6-yl)-acetamide
N-(4-Amino-piperidin-1-yl)-4-methyl-quinazolin-6-yl)-2-p-tolyloxy-acetamide
N-(4-Amino-piperidin-1-yl)-4-methyl-quinazolin-6-yl)-2-(4-trifluoromethyl-phenoxy)-acetamide
2-(4-Bromo-phenoxy)-N-(4-amino-piperidin-1-yl)-4-methyl-quinazolin-6-yl)-acetamide
N-(4-Amino-piperidin-1-yl)-4-methyl-quinazolin-6-yl)-3-(4-trifluoromethoxy-phenyl)-propionamide
(E)-N-(4-Amino-piperidin-1-yl)-4-methyl-quinazolin-6-yl)-3-(4-trifluoromethoxy-phenyl)-acrylamide
3-(4-Chloro-phenyl)-N-(4-amino-piperidin-1-yl)-4-methyl-quinazolin-6-yl)-propionamide
2-(2,4-Dichloro-phenoxy)-N-(4-amino-piperidin-1-yl)-4-methyl-quinazolin-6-yl)-acetamide
N-(4-Methyl-2-(4-methylamino-piperidin-1-yl-quinazolin-6-yl)-2-(4-trifluoromethoxy-phenoxy)-acetamide
2-(2-Chloro-4-trifluoromethoxy-phenoxy)-N-(4-methyl-2-(4-methylamino-piperidin-1-yl-quinazolin-6-yl)-acetamide
2-(4-Chloro-phenoxy)-N-(4-methyl-2-(4-methylamino-piperidin-1-yl-quinazolin-6-yl)-acetamide
N-(4-Methyl-2-(4-methylamino-piperidin-1-yl-quinazolin-6-yl)-2-p-tolyloxy-acetamide
N-(4-Methyl-2-(4-methylamino-piperidin-1-yl-quinazolin-6-yl)-2-(4-trifluoromethyl-phenoxy)-acetamide
2-(4-Bromo-phenoxy)-N-(4-methyl-2-(4-methylamino-piperidin-1-yl-quinazolin-6-yl)-acetamide
N-(4-Methyl-2-(4-methylamino-piperidin-1-yl-quinazolin-6-yl)-3-(4-trifluoromethoxy-phenyl)-propionamide
(E)-N-(4-Methyl-2-(4-methylamino-piperidin-1-yl-quinazolin-6-yl)-3-(4-trifluoromethoxy-phenyl)-acrylamide
3-(4-Chloro-phenyl)-N-(4-methyl-2-(4-methylamino-piperidin-1-yl-quinazolin-6-yl)-propionamide
2-(2,4-Dichloro-phenoxy)-N-(4-methyl-2-(4-methylamino-piperidin-1-yl-quinazolin-6-yl)-acetamide
N-(4-Methyl-2-(4-isopropyl-piperazin-1-yl)-quinazolin-6-yl)-2-(4-trifluoromethoxy-phenoxy)-acetamide
2-(2-Chloro trifluoromethoxy-phenoxy)-N-(4-methyl-2-(4-isopropyl-piperazin-1-yl)-quinazolin-6-yl)-acetamide
2-(4-Chloro-phenoxy)-N-(4-methyl-2-(4-isopropyl-piperazin-1-yl)-quinazolin-6-yl)-acetamide
N-(4-Methyl-2-(4-isopropyl-piperazin-1-yl)-quinazolin-6-yl)-2-p-tolyloxy-acetamide
N-(4-Methyl-2-(4-isopropyl-piperazin-1-yl)-quinazolin-6-yl)-2-(4-trifluoromethyl-phenoxy)-acetamide
2-(4-Bromo-phenoxy)-N-(4-methyl-2-(4-isopropyl-piperazin-1-yl)-quinazolin-6-yl)-acetamide
N-(4-Methyl-2-(4-isopropyl-piperazin-1-yl)-quinazolin-6-yl)-3-(4-trifluoromethoxy-phenyl)-propionamide
(E)-N-(4-Methyl-2-(4-isopropyl-piperazin-1-yl)-quinazolin-6-yl)-3-(4-trifluoromethoxy-phenyl)-acrylamide
3-(4-Chloro-phenyl)-N-(4-methyl-2-(4-isopropyl-piperazin-1-yl)-quinazolin-6-yl)-propionamide
2-(2,4-Dichloro-phenoxy)-N-(4-methyl-2-(4-isopropyl-piperazin-1-yl)-quinazolin-6-yl)-acetamide
N-{2-[4-(2-Hydroxy-ethyl)-[1,4]diazepan-1-yl]-4-methyl-quinazolin-6-yl}-2-(4-trifluoromethoxy-phenoxy)-acetamide
2-(2-Chloro-4-trifluoromethoxy-phenoxy)-N-{2-[4-(2-hydroxy-ethyl)-[1,4]diazepan-1-yl]-4-methyl-quinazolin-6-yl}acetamide
2-(4-Chloro-phenoxy)-N-{2-[4-(2-hydroxy-ethyl)-[1,4]diazepan-1-yl]4-methyl-quinazolin-6-yl}-acetamide
N-{2-[4-(2-Hydroxy-ethyl)-[1,4]diazepan-1-yl]4-methyl-quinazolin-6-yl}-2-p-tolyloxy-acetamide
N-{2-[4-(2-Hydroxy-ethyl)-[1,4]diazepan-1-yl]4-methyl-quinazolin-6-yl}-2-(4-trifluoromethyl-phenoxy)-acetamide
2-(4-Bromo-phenoxy)-N-{2-[4-(2-hydroxy-ethyl)-[1,4]diazepan-1-yl]4-methyl-quinazolin-6-yl}acetamide
N-{2-[4-(2-Hydroxy-ethyl)-[1,4]diazepan-1-yl]4-methyl-quinazolin-6-yl}-3-(4-trifluoromethoxy-phenyl)-propionamide
(E)-N-{2-[4-(2-Hydroxy-ethyl)-[1,4]diazepan-1-yl]4-methyl-quinazolin-6-yl}-3-(4-trifluoromethoxy-phenyl)-acrylamide
3-(4-Chloro-phenyl)-N-{2-[4-(2-hydroxy-ethyl)-[1,4]diazepan-1-yl]-4-methyl-quinazolin-6-yl}propionamide
2-(2,4-Dichloro-phenoxy)-N-{2-[4-(2-hydroxy-ethyl)-[1,4]diazepan-1-yl]-4-methyl-quinazolin-6-yl}-acetamide
N-(2-[1,4]Diazepan-1-yl-4-methyl-quinazolin-6-yl)-2-(4-trifluoromethoxy-phenoxy)-acetamide
2-(2-Chloro-4-trifluoromethoxy-phenoxy)-N-(2-[1,4]diazepan-1-yl-4-methyl-quinazolin-6-yl)-acetamide
2-(4-Chloro-phenoxy)-N-(2-[1,4]diazepan-1-yl-4-methyl-quinazolin-6-yl)-acetamide
N-(2-[1,4]Diazepan-1-yl-4-methyl-quinazolin-6-yl)-2-p-tolyloxy-acetamide
N-(2-[1,4]Diazepan-1-yl-4-methyl-quinazolin-6-yl)-2-(4-trifluoromethyl-phenoxy)-acetamide
2-(4-Bromo-phenoxy)-N-(2-[1,4]diazepan-1-yl-4-methyl-quinazolin-6-yl)-acetamide
N-(2-[1,4]Diazepan-1-yl-4-methyl-quinazolin-6-yl)-3-(4-trifluoromethoxy-phenyl)-propionamide
(E)-N-(2-[1,4]Diazepan-1-yl-4-methyl-quinazolin-6-yl)-3-(4-trifluoromethoxy-phenyl)-acrylamide
3-(4-Chloro-phenyl)-N-(2-[1,4]diazepan-1-yl-4-methyl-quinazolin-6-yl)-propionamide
2-(2,4-Dichloro-phenoxy)-N-(2-[1,4]diazepan-1-yl-4-methyl-quinazolin-6-yl)-acetamide
N-(4-Methyl-2-piperazin-1-yl-quinolin-6-yl)-3-(4-chloro-phenyl)-acrylamide
N-[4-Methyl-2-(4-methyl-piperazin-1-yl)-quinolin-6-yl]-3-(4-chloro-phenyl)-acrylamide
N-[4-Methyl-2-(4-pyrrolidin-1-yl-piperidin-1-yl)-quinolin-6-yl]-3-(4-chloro-phenyl)-acrylamide
N-[4-Methyl-2-(4-methyl-[1,4]diazepan-1-yl)-quinolin-6-yl]-3-(4-chloro-phenyl)-acrylamide
N-{2-[4-(2-Hydroxy-ethyl)-piperazin-1-yl]-4-methyl-quinolin-6-yl}-2-(4-trifluoromethoxy-phenoxy)-acetamide
N-[2-(2,5-Diaza-bicyclo[2.2.1]hept-2-yl)-4-methyl-quinolin-6-yl]-3-(4-chloro-phenyl)-acrylamide
N-[2-(4-Dimethylamino-piperidin-1-yl)-4-methyl-quinolin-6-yl]-3-(4-chloro-phenyl)-acrylamide
N-(4-Methyl-2-piperazin-1-yl-quinolin-6-yl)-2-(2-chloro-4-trifluoromethyl-phenoxy)-acetamide
N-[4-Methyl-2-(4-methyl-piperazin-1-yl)-quinolin-6-yl]-2-(2-chloro-4-trifluoromethyl-phenoxy)-acetamide
N-[4-Methyl-2-(4-pyrrolidin-1-yl-piperidin-1-yl)-quinolin-6-yl]-2-(2-chloro-4-trifluoromethyl-phenoxy)-acetamide
N-[4-Methyl-2-(4-methyl-[1,4]diazepan-1-yl)-quinolin-6-yl]-2-(2-chloro-4-trifluoromethyl-phenoxy)-acetamide
N-{2-[4-(2-Hydroxy-ethyl)-piperazin-1-yl]4-methyl-quinolin-6-yl}-2-(4-trifluoromethoxy-phenoxy)-acetamide
N-[2-(2,5-Diaza-bicyclo[2.2.1]hept-2-yl)-4-methyl-quinolin-6-yl]-2-(2-chloro-4-trifluoromethyl-phenoxy)-acetamide
N-[2-(4-Dimethylamino-piperidin-1-yl)-4-methyl-quinolin-6-yl]-2-(2-chloro-4-trifluoromethyl-phenoxy)-acetamide
N-(4-Methyl-2-piperazin-1-yl-quinazolin-6-yl)-3-(4-chloro-phenyl)-acrylamide
N-[4-Methyl-2-(4-methyl-piperazin-1-yl)-quinazolin-6-yl]-3-(4-chloro-phenyl)-acrylamide
N-[4-Methyl-2-(4-pyrrolidin-1-yl-piperidin-1-yl)-quinazolin-6-yl]-3-(4-chloro-phenyl)-acrylamide
N-[4-Methyl-2-(4-methyl-[1,4]diazepan-1-yl)-quinazolin-6-yl]-3-(4-chloro-phenyl)-acrylamide
N-{2-[4-(2-Hydroxy-ethyl)-piperazin-1-yl]4-methyl-quinazolin-6-yl}-2-(4-trifluoromethoxy-phenoxy)-acetamide
N-[2-(2,5-Diaza-bicyclo[2.2.1]hept-2-yl)-4-methyl-quinazolin-6-yl]-3-(4-chloro-phenyl)-acrylamide
N-[2-(4-Dimethylamino-piperidin-1-yl)-4-methyl-quinazolin-6-yl]-3-(4-chloro-phenyl)-acrylamide
N-(4-Methyl-2-piperazin-1-yl-quinazolin-6-yl)-2-(2-chloro-4-methyl-phenoxy)-acetamide
N-(4-Methyl-2-piperazin-1-yl-quinazolin-6-yl)-2-(2-chloro-4-methyl-phenoxy)-acetamide
N-[4-Methyl-2-(4-methyl-piperazin-1-yl)-quinazolin-6-yl]-2-(2-chloro-4-methyl-phenoxy)-acetamide
N-[4-Methyl-2-(4-pyrrolidin-1-yl-piperidin-1-yl)-quinazolin-6-yl]-2-(2-chloro-4-methyl-phenoxy)-acetamide
N-[4-Methyl-2-(4-methyl-[1,4]diazepan-1-yl)-quinazolin-6-yl]-2-(2-chloro-4-methyl-phenoxy)-acetamide
N-{2-[4-(2-Hydroxy-ethyl)-piperazin-1-yl]-4-methyl-quinazolin-6-yl)-2-(2-chloro-4-methyl-phenoxy)-acetamide
N-[2-(2,5-Diaza-bicyclo[2.2.1]hept-2-yl)-4-methyl-quinazolin-6-yl]-2-(2-chloro-4-methyl-phenoxy)-acetamide
N-[2-(4-Dimethylamino-piperidin-1-yl)-4-methyl-quinazolin-6-yl]-2-(2-chloro-4-methyl-phenoxy)-acetamide
N-(4-methyl-2-piperazin-1-yl-quinazolin-6-yl)-2-(2-chloro-4-trifluoromethyl-phenoxy)-acetamide
N-(4-Methyl-2-piperazin-1-yl-quinazolin-6-yl)-2-(2-chloro-4-trifluoromethyl-phenoxy)-acetamide
N-[4-Methyl-2-(4-methyl-piperazin-1-yl)-quinazolin-6-yl]-2-(2-chloro-4-trifluoromethyl-phenoxy)-acetamide
N-[4-Methyl-2-(4-pyrrolidin-1-yl-piperidin-1-yl)-quinazolin-6-yl]-2-(2-chloro-4-trifluoromethyl-phenoxy)-acetamide
N-[4-Methyl-2-(4-methyl-[1,4]diazepan-1-yl)-quinazolin-6-yl]-2-(2-chloro-4-trifluoromethyl-phenoxy)-acetamide
N-{2-[4-(2-Hydroxy-ethyl)-piperazin-1-yl]-methyl-quinazolin-6-yl}-2-(4-trifluoromethoxy-phenoxy)-acetamide
N-[2-(2,5-Diaza-bicyclo[2.2.1]hept-2-yl)-4-methyl-quinazolin-6-yl]-2-(2-chloro-4-trifluoromethyl-phenoxy)-acetamide
N-[2-(4-Dimethylamino-piperidin-1-yl)-4-methyl-quinazolin-6-yl]-2-(2-chloro-4-trifluoromethyl-phenoxy)-acetamide
N-(4-Methyl-2-piperazin-1-yl-pyrido[3,2-d]pyrimidin-6-yl)-2-(4-trifluoromethoxy-phenoxy)-acetamide
N-[4-Methyl-2-(4-methyl-piperazin-1-yl)-pyrido[3,2-d]pyrimidin-6-yl]-2-(4-trifluoromethoxy-phenoxy)-acetamide
N-[4-Methyl-2-(4-pyrrolidin-1-yl-piperidin-1-yl)-pyrido[3,2-d]pyrimidin-6-yl]-2-(4-trifluoromethoxy-phenoxy)-acetamide
N-[4-Methyl-2-(4-methyl-[1,4]diazepan-1-yl)-pyrido[3,2-d]pyrimidin-6-yl]-2-(4-trifluoromethoxy-phenoxy)-acetamide
N-{2-[4-(2-Hydroxy-ethyl)-piperazin-1-yl]-4-methyl-pyrido[3,2-d]pyrimidin-6-yl}-2-(4-trifluoromethoxy-phenoxy)-acetamide
N-[2-(2,5-Diaza-bicyclo[2.2.1]hept-2-yl)-4-methyl-pyrido[3,2-d]pyrimidin-6-yl]-2-(4-trifluoromethoxy-phenoxy)-acetamide
N-[2-(4-Dimethylamino-piperidin-1-yl)-4-methyl-pyrido[3,2-d]pyrimidin-6-yl]-2-(4-trifluoromethoxy-phenoxy)-acetamide
2-(2-Chloro-4-trifluoromethoxy-phenoxy)-N-[4-methyl-2-(4-methyl-[1,4]diazepan-1-yl)-pyrido[3,2-d]pyrimidin-6-yl]-acetamide
2-(2-Chloro-4-trifluoromethoxy-phenoxy)-N-(4-methyl-2-piperazin-1-yl-pyrido[3,2-d]pyrimidin-6-yl)-acetamide
2-(2-Chloro-4-trifluoromethoxy-phenoxy)-N-[4-methyl-2-(4-methyl-piperazin-1-yl)-pyrido[3,2-d]pyrimidin-6-yl]-acetamide
2-(2-Chloro-4-trifluoromethoxy-phenoxy)-N-{2-[4-(2-hydroxy-ethyl)-piperazin-1-yl]4-methyl-pyrido[3,2-d]pyrimidin-6-yl}-acetamide
2-(2-Chloro-4-trifluoromethoxy-phenoxy)-N-[2-(2,5-diaza-bicyclo[2.2.1]hept-2-yl)-4-methyl-pyrido[3,2-d]pyrimidin-6-yl]-acetamide
2-(2-Chloro-4-trifluoromethoxy-phenoxy)-N-[4-methyl-2-(4-pyrrolidin-1-yl-piperidin-1-yl)-pyrido[3,2-d]pyrimidin-6-yl]-acetamide
2-(2-Chloro-4-trifluoromethoxy-phenoxy)-N-[2-(4-dimethylamino-piperidin-1-yl)-4-methyl-pyrido[3,2-d]pyrimidin-6-yl]-acetamide
2-(4-Chloro-phenoxy)-N-(4-methyl-2-piperazin-1-yl-pyrido[3,2-d]pyrimidin-6-yl)-acetamide
2-(4-Chloro-phenoxy)-N-[4-methyl-2-(4-methyl-[1,4]diazepan-1-yl)-pyrido[3,2-d]pyrimidin-6-yl]-acetamide
2-(4-Chloro-phenoxy)-N-[4-methyl-2-(4-methyl-piperazin-1-yl)-pyrido[3,2-d]pyrimidin-6-yl]-acetamide
2-(4-Chloro-phenoxy)-N-[4-methyl-2-(4-pyrrolidin-1-yl-piperidin-1-yl)-pyrido[3,2-d]pyrimidin-6-yl]-acetamide
2-(4-Chloro-phenoxy)-N-{2-[4-(2-hydroxyethyl)-piperazin-1-yl]-4-methyl-pyrido[3,2-d]pyrimidin-6-yl}-acetamide
2-(4-Chloro-phenoxy)-N-[2-(4-dimethylamino-piperidin-1-yl)-4-methyl-pyrido[3,2-d]pyrimidin-6-yl]-acetamide
2-(4-Chloro-phenoxy)-N-[2-(2,5-diaza-bicyclo[2.2.1]hept-2-yl)-4-methyl-pyrido[3,2-d]pyrimidin-6-yl]-acetamide
N-(4-Methyl-2-piperazin-1-yl-pyrido[3,2-d]pyrimidin-6-yl)-2-p-tolyloxy-acetamide
N{2-[4-(2-Hydroxy-ethyl)-piperazin-1-yl]-4-methyl-pyrido[3,2-d]pyrimidin-6-yl}-2-p-tolyloxy-acetamide
N-[4-Methyl-2-(4-methyl-piperazin-1-yl)-pyrido[3,2-d]pyrimidin-6-yl]-2-p-tolyloxy-acetamide
N-[4-Methyl-2-(4-methyl-[1,4]diazepan-1-yl)-pyrido[3,2-d]pyrimidin-6-yl]-2-p-tolyloxy-acetamide
N-[4-Methyl-2-(4-pyrrolidin-1-yl-piperidin-1-yl)-pyrido[3,2-d]pyrimidin-6-yl]-2-p-tolyloxy-acetamide
N-[2-(2,5-Diaza-bicyclo[2.2.1]hept-2-yl)-4-methyl-pyrido[3,2-d]pyrimidin-6-yl]-2-p-tolyloxy-acetamide
N-[2-(4-Dimethylamino-piperidin-1-yl)-4-methyl-pyrido[3,2-d]pyrimidin-6-yl]-2-p-tolyloxy-acetamide
N-(4-Methyl-2-piperazin-1-yl-pyrido[3,2-d]pyrimidin-6-yl)-2-(4-trifluoromethyl-phenoxy)-acetamide
N-[4-Methyl-2-(4-methyl-[1,4]diazepan-1-yl)-pyrido[3,2-d]pyrimidin-6-yl]-2-(4-trifluoromethyl-phenoxy)-acetamide
N-[4-Methyl-2-(4-methyl-piperazin-1-yl)-pyrido[3,2-d]pyrimidin-6-yl-2-(4-trifluoromethyl-phenoxy)-acetamide
N-(2-[4-(2-Hydroxy-ethyl)-piperazin-1-y]-4-methyl-pyrido[3,2-d]pyrimidin-6-yl}2-(4-trifluoromethyl-phenoxy)-acetamide
N-[4-Methyl-2-(4-pyrrolidin-1-yl-piperidin-1-yl)-pyrido[3,2-d]pyrimidin-6-yl]-2-(4-trifluoromethyl-phenoxy)-acetamide
N-[2-(2,5-Diaza-bicyclo[2.2.1]hept-2-yl)-4-methyl-pyrido[3,2-d]pyrimidin-6-yl]-2-(4-trifluoromethyl-phenoxy)-acetamide
N-[2-(4-Dimethylamino-piperidin-1-yl)-4-methyl-pyrido[3,2-d]pyrimidin-6-yl]-2-(4-trifluoromethyl-phenoxy)-acetamide
2-(4-Bromo-phenoxy)-N-(4-methyl-2-piperazin-1-yl-pyrido[3,2-d]pyrimidin-6-yl)-acetamide
2-(4-Bromo-phenoxy)-N-[4-methyl-2-(4-methyl-[1,4]diazepan-1-yl)-pyrido[3,2-d]pyrimidin-6-yl]-acetamide
2-(4-Bromo-phenoxy)-N-[4-methyl-2-(4-methyl-piperazin-1-yl)-pyrido[3,2-d)pyrimidin-6-yl]-acetamide
2-(4-Bromo-phenoxy)-N-[4-methyl-2-(4-pyrrolidin-1-yl-piperidin-1-yl)-pyrido[3,2-d]pyrimidin-6-yl]-acetamide
2-(4-Bromo-phenoxy)-N-{2-[4-(2-hydroxy-ethyl)-piperazin-1-yl]-4-methyl-pyrido[3,2-d]pyrimidin-6-yl}-acetamide
2-(4-Bromo-phenoxy)-N-[2-(2,5-diaza-bicyclo[2.2.1]hept-2-yl)-4-methyl-pyrido[3,2-d]pyrimidin-6-yl]-acetamide
2-(4-Bromo-phenoxy)-N-[2-(4-dimethylamino-piperidin-1-yl)-4-methyl-pyrido[3,2-d]pyrimidin-6-yl]-acetamide
N-(4-Methyl-2-piperazin-1-yl-pyrido[3,2-d]pyrimidin-6-yl)-3-(4-trifluoromethoxy-phenyl)-propionamide
N-[4-Methyl-2-(4-methyl-(1,4]diazepan-1-yl)-pyrido[3,2-d]pyrimidin-6-yl]-3-(4-trifluoromethoxy-phenyl)-propionamide
N-[4-Methyl-2-(4-methyl-piperazin-1-yl)-pyrido[3,2-d]pyrimidin-6-yl]-3-(4-trifluoromethoxy-phenyl)-propionamide
N-{2-[4-(2-Hydroxy-ethyl)-piperazin-1-yl]-4-methyl-pyrido[3,2-d]pyrimidin-6-yl}-3-(4-trifluoromethoxy-phenyl)-propionamide
N-[4-Methyl-2-(4-pyrrolidin-1-yl-piperidin-1-yl)-pyrido[3,2-d]pyrimidin-6-yl]-3-(4-trifluoromethoxy-phenyl)-propionamide
N-[2-(2,5-Diaza-bicyclo[2.2.1]hept-2-yl)-4-methyl-pyrido[3,2-d]pyrimidin-6-yl]-3-(4-trifluoromethoxy-phenyl)-propionamide
N-[2-(4-Dimethylamino-piperidin-1-yl)-4-methyl-pyrido[3,2-d]pyrimidin-6-yl]-3-(4-trifluoromethoxy-phenyl)-propionamide
(E)-N-(4-Methyl-2-piperazin-1-yl-pyrido[3,2-d]pyrimidin-6-yl)-3-(4-trifluoromethoxy-phenyl)-acrylamide
(E)-N-[4-Methyl-2-(4-methyl-[1,4]diazepan-1-yl)-pyrido[3,2-d]pyrimidin-6-yl]-3-(4-trifluoromethoxy-phenyl)-acrylamide
(E)-N-[4-Methyl-2-(4-methyl-piperazin-1-yl)-pyrido[3,2-d]pyrimidin-6-yl]-3-(4-trifluoromethoxy-phenyl)-acrylamide
(E)-N-{2-[4-(2-Hydroxy-ethyl)-piperazin-1-yl]-4-methyl-pyrido[3,2-d]pyrimidin-6-yl}-3-(4-trifluoromethoxy-phenyl)-acrylamide
(E)-N-[2-(4-Dimethylamino-piperidin-1-yl)-4-methyl-pyrido[3,2-d]pyrimidin-6-yl]-3-(4-trifluoromethoxy-phenyl)-acrylamide
(E)-N-[2-(2,5-Diaza-bicyclo[2.2.1]hept-2-yl)-4-methyl-pyrido[3,2-d]pyrimidin-6-yl]-3-(4-trifluoromethoxy-phenyl)-acrylamide
(E)-N-[4-Methyl-2-(4-pyrrolidin-1-yl-piperidin-1-yl)-pyrido[3,2-d]pyrimidin-6-yl]-3-(4-trifluoromethoxy-phenyl)-acrylamide
3-(4-Chloro-phenyl)-N-(4-methyl-2-piperazin-1-yl-pyrido[3,2-d]pyrimidin-6-yl)-propionamide
3-(4-Chloro-phenyl)-N-[4-methyl-2-(4-methyl-[1,4]diazepan-1-yl)-pyrido[3,2-d]pyrimidin-6-yl]-propionamide
3-(4-Chloro-phenyl)-N-[4-methyl-2-(4-methyl-piperazin-1-yl)-pyrido[3,2-d]pyrimidin-6-yl]-propionamide
3-(4-Chloro-phenyl)-N-{2-[4-(2-hydroxy-ethyl)-piperazin-1-yl]4-methyl-pyrido[3,2-d]pyrimidin-6-yl}-propionamide
3-(4-Chloro-phenyl)-N-[4-methyl-2-(4-pyrrolidin-1-yl-piperidin-1-yl)-pyrido[3,2-d]pyrimidin-6-yl]-propionamide
3-(4-Chloro-phenyl)-N-[2-(2,5-diaza-bicyclo[2.2.1]hept-2-yl)-4-methyl-pyrido[3,2-d]pyrimidin-6-yl]-propionamide
3-(4-Chloro-phenyl)-N-[2-(4-dimethylamino-piperidin-1-yl)-4-methyl-pyrido[3,2-d]pyrimidin-6-yl]-propionamide
2-(2,4-Dichloro-phenoxy)-N-(4-methyl-2-piperazin-1-yl-pyrido[3,2-d]pyrimidin-6-yl)-acetamide
2-(2,4-Dichloro-phenoxy)-N-[4-methyl-2-(4-methyl-[1,4]diazepan-1-yl)-pyrido[3,2-d]pyrimidin-6-yl]-acetamide
2-(2,4-Dichloro-phenoxy)-N-[4-methyl-2-(4-methyl-piperazin-1-yl)-pyrido[3,2-d]pyrimidin-6-yl]-acetamide
2-(2,4-Dichloro-phenoxy)-N-{2-[4-(2-hydroxy-ethyl)-piperazin-1-yl]-4-methyl-pyrido[3,2-d]pyrimidin-6-yl}acetamide
2-(2,4-Dichloro-phenoxy)-N-[2-(4-dimethylamino-piperidin-1-yl)-4-methyl-pyrido[3,2-d]pyrimidin-6-yl]-acetamide
N-[2-(2,5-Diaza-bicyclo[2.2.1]hept-2-yl)-4-methyl-pyrido[3,2-d]pyrimidin-6-yl]-2-(2,4-dichloro-phenoxy)-acetamide
2-(2,4-Dichloro-phenoxy)-N-[4-methyl-2-(4-pyrrolidin-1-yl-piperidin-1-yl)-pyrido[3,2-d]pyrimidin-6-yl]-acetamide
N-(4-Methyl-2-piperazin-1-yl-pyrido[2,3-d]pyrimidin-6-yl)-2-(4-trifluoromethoxy-phenoxy)-acetamide
N-(4-Methyl-2-piperazin-1-yl-pyrido[2,3-d]pyrimidin-6-yl)-2-(4-trifluoromethoxy-phenoxy)-acetamide
N-[4-Methyl-2-(4-methyl-piperazin-1-yl)-pyrido[2,3-d]pyrimidin-6-yl]-2-(4-trifluoromethoxy-phenoxy)-acetamide
N-[4-Methyl-2-(4-pyrrolidin-1-yl-piperidin-1-yl)-pyrido[2,3-d]pyrimidin-6-yl]-2-(4-trifluoromethoxy-phenoxy)-acetamide
N-[4-Methyl-2-(4-methyl-[1,4]diazepan-1-yl)-pyrido[2,3-d]pyrimidin-6-yl]-2-(4-trifluoromethoxy-phenoxy)-acetamide
N-{2-[4-(2-Hydroxy-ethyl)-piperazin-1-yl]-4-methyl-pyrido[2,3-d]pyrimidin-6-yl}-2-(4-trifluoromethoxy-phenoxy)-acetamide
N-[2-(2,5-Diaza-bicyclo[2.2.1]hept-2-yl)-4-methyl-pyrido[2,3-d]pyrimidin-6-yl]-2-(4-trifluoromethoxy-phenoxy)-acetamide
N-[2-(4-Dimethylamino-piperidin-1-yl)-4-methyl-pyrido[2,3-d]pyrimidin-6-yl]-2-(4-trifluoromethoxy-phenoxy)-acetamide
2-(2-Chloro-4-trifluoromethoxy-phenoxy)-N-[4-methyl-2-(4-methyl-[1,4]diazepan-1-yl)-pyrido[2,3-d]pyrimidin-6-yl]-acetamide
2-(2-Chloro-4-trifluoromethoxy-phenoxy)-N-(4-methyl-2-piperazin-1-yl-pyrido[2,3-d]pyrimidin-6-yl)-acetamide
2-(2-Chloro-4-trifluoromethoxy-phenoxy)-N-[4-methyl-2-(4-methyl-piperazin-1-yl)-pyrido[2,3-d]pyrimidin-6-yl]-acetamide
2-(2-Chloro-4-trifluoromethoxy-phenoxy)-N-{2-[4-(2-hydroxy-ethyl)-piperazin-1-yl]4-methyl-pyrido[2,3-d]pyrimidin-6-yl}-acetamide
2-(2-Chloro-4-trifluoromethoxy-phenoxy)-N-[2-(2,5-diaza-bicyclo[2.2.1]hept-2-yl)-4-methyl-pyrido[2,3-d]pyrimidin-6-yl]-acetamide
2-(2-Chloro-4-trifluoromethoxy-phenoxy)-N-[4-methyl-2-(4-pyrrolidin-1-yl-piperidin-1-yl)-pyrido[2,3-d]pyrimidin-6-yl]-acetamide
2-(2-Chloro-4-trifluoromethoxy-phenoxy)-N-[2-(4-dimethylamino-piperidin-1-yl)-4-methyl-pyrido[2,3-d]pyrimidin-6-yl]-acetamide
2-(4-Chloro-phenoxy)-N-(4-methyl-2-piperazin-1-yl-pyrido[2,3-d]pyrimidin-6-yl)-acetamide
2-(4-Chloro-phenoxy)-N-[4-methyl-2-(4-methyl-[1,4]diazepan-1-yl)-pyrido[2,3-d]pyrimidin-6-yl]-acetamide
2-(4-Chloro-phenoxy)-N-[4-methyl-2-(4-methyl-piperazin-1-yl)-pyrido[2,3-d]pyrimidin-6-yl]-acetamide
2-(4-Chloro-phenoxy)-N-[4-methyl-2-(4-pyrrolidin-1-yl-piperidin-1-yl)-pyrido[2,3-d]pyrimidin-6-yl]-acetamide
2-(4-Chloro-phenoxy)-N-{2-[4-(2-hydroxy-ethyl)-piperazin-1-yl]-4-methyl-pyrido[2,3-d]pyrimidin-6-yl}acetamide
2-(4-Chloro-phenoxy)-N-[2-(4-dimethylamino-piperidin-1-yl)-4-methyl-pyrido[2,3-d]pyrimidin-6-yl]-acetamide
2-(4-Chloro-phenoxy)-N-[2-(2,5-diaza-bicyclo[2.2.1]hept-2-yl)-4-methyl-pyrido[2,3-d]pyrimidin-6-yl]-acetamide
N-(4-Methyl-2-piperazin-1-yl-pyrido[2,3-d]pyrimidin-6-yl)-2-p-tolyloxy-acetamide
N-{2-[4-(2-Hydroxy-ethyl)-piperazin-1-yl]4-methyl-pyrido[2,3-d]pyrimidin-6-yl}2-p-tolyloxy-acetamide
N-[4-Methyl-2-(4-methyl-piperazin-1-yl)-pyrido[2,3-d]pyrimidin-6-yl]-2-p-tolyloxy-acetamide
N-[4-Methyl-2-(4-methyl-[1,4]diazepan-1-yl)-pyrido[2,3-d]pyrimidin-6-yl]-2-p-tolyloxy-acetamide
N-[4-Methyl-2-(4-pyrrolidin-1-yl-piperidin-1-yl)-pyrido[2,3-d]pyrimidin-6-yl]-2-p-tolyloxy-acetamide
N-[2-(2,5-Diaza-bicyclo[2.2.1]hept-2-yl)-4-methyl-pyrido[2,3-d]pyrimidin-6-yl]-2-p-tolyloxy-acetamide
N-[2-(4-Dimethylamino-piperidin-1-yl)-4-methyl-pyrido[2,3-d]pyrimidin-6-yl]-2-p-tolyloxy-acetamide
N-(4-Methyl-2-piperazin-1-yl-pyrido[2,3-d]pyrimidin-6-yl)-2-(4-trifluoromethyl-phenoxy)-acetamide
N-[4-Methyl-2-(4-methyl-[1,4]diazepan-1-yl)-pyrido[2,3-d]pyrimidin-6-yl]-2-(4-trifluoromethyl-phenoxy)-acetamide
N-[4-Methyl-2-(4-methyl-piperazin-1-yl)-pyrido[2,3-d]pyrimidin-6-yl]-2-(4-trifluoromethyl-phenoxy)-acetamide
N-{2-[4-(2-Hydroxy-ethyl)-piperazin-1-yl]-4-methyl-pyrido[2,3-d]pyrimidin-6-yl}-2-(4-trifluoromethyl-phenoxy)-acetamide
N-[4-Methyl-2-(4-pyrrolidin-1-yl-piperidin-1-yl)-pyrido[2,3-d]pyrimidin-6-yl]-2-(4-trifluoromethyl-phenoxy)-acetamide
N-[2-(2,5-Diaza-bicyclo[2.2.1]hept-2-yl)-4-methyl-pyrido[2,3-d]pyrimidin-6-yl]-2-(4-trifluoromethyl-phenoxy)-acetamide
N-[2-(4-Dimethylamino-piperidin-1-yl)-4-methyl-pyrido[2,3-d]pyrimidin-6-yl]-2-(4-trifluoromethyl-phenoxy)-acetamide
2-(4-Bromo-phenoxy)-N-(4-methyl-2-piperazin-1-yl-pyrido[2,3-d]pyrimidin-6-yl)-acetamide
2-(4-Bromo-phenoxy)-N-[4-methyl-2-(4-methyl-[1,4]diazepan-1-yl)-pyrido[2,3-d]pyrimidin-6-yl]-acetamide
2-(4-Bromo-phenoxy)-N-[4-methyl-2-(4-methyl-piperazin-1-yl)-pyrido[2,3-d]pyrimidin-6-yl]-acetamide
2-(4-Bromo-phenoxy)-N-[4-methyl-2-(4-pyrrolidin-1-yl-piperidin-1-yl)-pyrido[2,3-d]pyrimidin-6-yl]-acetamide
2-(4-Bromo-phenoxy)-N-{2-[4-(2-hydroxy-ethyl)-piperazin-1-yl]-4-methyl-pyrido[2,3-d]pyrimidin-6-yl}acetamide
2-(4-Bromo-phenoxy)-N-[2-(2,5-diaza-bicyclo[2.2.1]hept-2-yl)-4-methyl-pyrido[2,3-d]pyrimidin-6-yl]-acetamide
2-(4-Bromo-phenoxy)-N-[2-(4-dimethylamino-piperidin-1-yl)-4-methyl-pyrido[2,3-d]pyrimidin-6-yl]-acetamide
N-(4-Methyl-2-piperazin-1-yl-pyrido[2,3-d]pyrimidin-6-yl)-3-(4-trifluoromethoxy-phenyl)-propionamide
N-[4-Methyl-2-(4-methyl-[1,4]diazepan-1-yl)-pyrido[2,3-d]pyrimidin-6-yl]-3-(4-trifluoromethoxy-phenyl)-propionamide
N-[4-Methyl-2-(4-methyl-piperazin-1-yl)-pyrido[2,3-d]pyrimidin-6-yl]-3-(4-trifluoromethoxy-phenyl)-propionamide
N-{2-[4-(2-Hydroxy-ethyl)-piperazin-1-yl]-4-methyl-pyrido[2,3-d]pyrimidin-6-yl}-3-(4-trifluoromethoxy-phenyl)-propionamide
N-[4-Methyl-2-(4-pyrrolidin-1-yl-piperidin-1-yl)-pyrido[2,3-d]pyrimidin-6-yl]-3-(4-trifluoromethoxy-phenyl)-propionamide
N-[2-(2,5-Diaza-bicyclo[2.2.1]hept-2-yl)-4-methyl-pyrido[2,3-d]pyrimidin-6-yl]-3-(4-trifluoromethoxy-phenyl)-propionamide
N-[2-(4-Dimethylamino-piperidin-1-yl)-4-methyl-pyrido[2,3-d]pyrimidin-6-yl]-3-(4-trifluoromethoxy-phenyl)-propionamide
(E)-N-(4-Methyl-2-piperazin-1-yl-pyrido[2,3-d]pyrimidin-6-yl)-3-(4-trifluoromethoxy-phenyl)-acrylamide
(E)-N-[4-Methyl-2-(4-methyl-[1,4]diazepan-1-yl)-pyrido[2,3-d]pyrimidin-6-yl]-3-(4-trifluoromethoxy-phenyl)-acrylamide
(E)-N-[4-Methyl-2-(4-methyl-piperazin-1-yl)-pyrido[2,3-d]pyrimidin-6-yl]-3-(4-trifluoromethoxy-phenyl)-acrylamide
(E)-N-{2-[4-(2-Hydroxy-ethyl)-piperazin-1-yl]-4-methyl-pyrido[2,3-d]pyrimidin-6-yl}3-(4-trifluoromethoxy-phenyl)-acrylamide
(E)-N-[2-(4-Dimethylamino-piperidin-1-yl)-4-methyl-pyrido[2,3-d]pyrimidin-6-yl]-3-(4-trifluoromethoxy-phenyl)-acrylamide
(E)-N-[2-(2,5-Diaza-bicyclo[2.2.1]hept-2-yl)-4-methyl-pyrido[2,3-d]pyrimidin-6-yl]-3-(4-trifluoromethoxy-phenyl)-acrylamide
(E)-N-[4-Methyl-2-(4-pyrrolidin-1-yl-piperidin-1-yl)-pyrido[2,3-d]pyrimidin-6-yl]-3-(4-trifluoromethoxy-phenyl)-acrylamide
3-(4-Chloro-phenyl)-N-(4-methyl-2-piperazin-1-yl-pyrido[2,3-d]pyrimidin-6-yl)-propionamide
3-(4-Chloro-phenyl)-N-[4-methyl-2-(4-methyl-[1,4]diazepan-1-yl)-pyrido[2,3-d]pyrimidin-6-yl]-propionamide 3-(4-Chloro-phenyl)-N-[4-methyl-2-(4-methyl-piperazin-1-yl)-pyrido[2,3-d]pyrimidin-6-yl]-propionamide
3-(4-Chloro-phenyl)-N{2-[4-(2-hydroxy-ethyl)-piperazin-1-yl]-4-methyl-pyrido[2,3-d]pyrimidin-6-yl}-propionamide
3-(4-Chloro-phenyl)-N-[4-methyl-2-(4-pyrrolidin-1-yl-piperidin-1-yl)-pyrido[2,3-d]pyrimidin-6-yl]-propionamide
3-(4-Chloro-phenyl)-N-[2-(2,5-diaza-bicyclo[2.2.1]hept-2-yl)-4-methyl-pyrido[2,3-d]pyrimidin-6-yl]-propionamide
3-(4-Chloro-phenyl)-N-[2-(4-dimethylamino-piperidin-1-yl)-4-methyl-pyrido[2,3-d]pyrimidin-6-yl]-propionamide
2-(2,4-Dichloro-phenoxy)-N-(4-methyl-2-piperazin-1-yl-pyrido[2,3-d]pyrimidin-6-yl)-acetamide 2-(2,4-Dichloro-phenoxy)-N-[4-methyl-2-(4-methyl-[1,4]diazepan-1-yl)-pyrido[2,3-d]pyrimidin-6-yl]-acetamide
2-(2,4-Dichloro-phenoxy)-N-[4-methyl-2-(4-methyl-piperazin-1-yl)-pyrido[2,3-d]pyrimidin-6-yl]-acetamide
2-(2,4-Dichloro-phenoxy)-N-{2-[4-(2-hydroxy-ethyl)-piperazin-1-yl]-4-methyl-pyrido[2,3-d]pyrimidin-6-yl}-acetamide
2-(2,4-Dichloro-phenoxy)-N-[2-(4-dimethylamino-piperidin-1-yl)-4-methyl-pyrido[2,3-d]pyrimidin-6-yl]-acetamide
N-[2-(2,5-Diaza-bicyclo[2.2.1]hept-2-yl)-4-methyl-pyrido[2,3-d]pyrimidin-6-yl]-2-(2,4-dichloro-phenoxy)-acetamide
2-(2,4-Dichloro-phenoxy)-N-[4-methyl-2-(4-pyrrolidin-1-yl-piperidin-1-yl)-pyrido[2,3-d]pyrimidin-6-yl]-acetamide
N-(4-Methyl-2-piperazin-1-yl-pteridin-6-yl)-2-(4-trifluoromethoxy-phenoxy)-acetamide
N-(4-Methyl-2-piperazin-1-yl-pteridin-6-yl)-2-(4-trifluoromethoxy-phenoxy)-acetamide
N-[4-Methyl-2-(4-methyl-piperazin-1-yl)-pteridin-6-yl]-2-(4-trifluoromethoxy-phenoxy)-acetamide
N-[4-Methyl-2-(4-pyrrolidin-1-yl-piperidin-1-yl)-pteridin-6-yl]-2-(4-trifluoromethoxy-phenoxy)-acetamide
N-[4-Methyl-2-(4-methyl-[1,4]diazepan-1-yl)-pteridin-6-yl]-2-(4-trifluoromethoxy-phenoxy)-acetamide
N-{2-[4-(2-Hydroxy-ethyl)-piperazin-1-yl]-4-methyl-pteridin-6-yl)-2-(4-trifluoromethoxy-phenoxy)-acetamide
N-[2-(2,5-Diaza-bicyclo[2.2.1]hept-2-yl)-4-methyl-pteridin-6-yl]-2-(4-trifluoromethoxy-phenoxy)-acetamide
N-[2-(4-Dimethylamino-piperidin-1-yl)-4-methyl-pteridin-6-yl]-2-(4-trifluoromethoxy-phenoxy)-acetamide
2-(2-Chloro-4-trifluoromethoxy-phenoxy)-N-[4-methyl-2-(4-methyl-[1,4]diazepan-1-yl)-pteridin-6-yl]-acetamide
2-(2-Chloro-4-trifluoromethoxy-phenoxy)-N-(4-methyl-2-piperazin-1-yl-pteridin-6-yl)-acetamide
2-(2-Chloro-4-trifluoromethoxy-phenoxy)-N-[4-methyl-2-(4-methyl-piperazin-1-yl)-pteridin-6-yl]-acetamide
2-(2-Chloro-4-trifluoromethoxy-phenoxy)-N-{2-[4-(2-hydroxy-ethyl)-piperazin-1-yl]-4-methyl-pteridin-6-yl}acetamide
2-(2-Chloro-4-trifluoromethoxy-phenoxy)-N-[2-(2,5-diaza-bicyclo[2.2.1]hept-2-yl)-4-methyl-pteridin-6-yl]-acetamide
2-(2-Chloro-4-trifluoromethoxy-phenoxy)-N-[4-methyl-2-(4-pyrrolidin-1-yl-piperidin-1-yl)-pteridin-6-yl]-acetamide
2-(2-Chloro-4-trifluoromethoxy-phenoxy)-N-[2-(4-dimethylamino-piperidin-1-yl)-4-methyl-pteridin-6-yl]-acetamide
2-(4-Chloro-phenoxy)-N-(4-methyl-2-piperazin-1-yl-pteridin-6-yl)-acetamide
2-(4-Chloro-phenoxy)-N-[4-methyl-2-(4-methyl-[1,4]diazepan-1-yl)-pteridin-6-y]-acetamide
2-(4-Chloro-phenoxy)-N-[4-methyl-2-(4-methyl-piperazin-1-yl)-pteridin-6-yl]-acetamide
2-(4-Chloro-phenoxy)-N-[4-methyl-2-(4-pyrrolidin-1-yl-piperidin-1-yl)-pteridin-6-yl]-acetamide
2-(4-Chloro-phenoxy)-N-{2-[4-(2-hydroxy-ethyl)-piperazin-1-yl]-4-methyl-pteridin-6-yl}-acetamide
2-(4-Chloro-phenoxy)-N-[2-(4-dimethylamino-piperidin-1-yl)-4-methyl-pteridin-6-yl]-acetamide
2-(4-Chloro-phenoxy)-N-[2-(2,5-diaza-bicyclo[2.2.1]hept-2-yl)-4-methyl-pteridin-6-yl]-acetamide
N-(4-Methyl-2-piperazin-1-yl-pteridin-6-yl)-2-p-tolyloxy-acetamide
N-{2-[4-(2-Hydroxy-ethyl)-piperazin-1-yl]-4-methyl-pteridin-6-yl}-2-p-tolyloxy-acetamide
N-[4-Methyl-2-(4-methyl-piperazin-1-yl)-pteridin-6-yl]-2-p-tolyloxy-acetamide
N-[4-Methyl-2-(4-methyl-[1,4]diazepan-1-yl)-pteridin-6-yl]-2-p-tolyloxy-acetamide
N-[4-Methyl-2-(4-pyrrolidin-1-yl-piperidin-1-yl)-pteridin-6-yl]-2-p-tolyloxy-acetamide
N-[2-(2,5-Diaza-bicyclo[2.2.1]hept-2-yl)-4-methyl-pteridin-6-yl]-2-p-tolyloxy-acetamide
N-[2-(4-Dimethylamino-piperidin-1-yl)-4-methyl-pteridin-6-yl]-2-p-tolyloxy-acetamide
N-(4-Methyl-2-piperazin-1-yl-pteridin-6-yl)-2-(4-trifluoromethyl-phenoxy)-acetamide
N-[4-Methyl-2-(4-methyl-[1,4]diazepan-1-yl)-pteridin-6-yl]-2-(4-trifluoromethyl-phenoxy)-acetamide
N-[4-Methyl-2-(4-methyl-piperazin-1-yl)-pteridin-6-yl]-2-(4-trifluoromethyl-phenoxy)-acetamide
N-{2-[4-(2-Hydroxy-ethyl)-piperazin-1-yl]-4-methyl-pteridin-6-yl}-2-(4-trifluoromethyl-phenoxy)-acetamide
N-[4-Methyl-2-(4-pyrrolidin-1-yl-piperidin-1-yl)-pteridin-6-yl]-2-(4-trifluoromethyl-phenoxy)-acetamide
N-[2-(2,5-Diaza-bicyclo[2.2.1]hept-2-yl)-4-methyl-pteridin-6-yl]-2-(4-trifluoromethyl-phenoxy)-acetamide
N-[2-(4-Dimethylamino-piperidin-1-yl)-4-methyl-pteridin-6-yl]-2-(4-trifluoromethyl-phenoxy)-acetamide
2-(4-Bromo-phenoxy)-N-(4-methyl-2-piperazin-1-yl-pteridin-6-yl)-acetamide
2-(4-Bromo-phenoxy)-N-[4-methyl-2-(4-methyl-[1,4]diazepan-1-yl)-pteridin-6-yl]-acetamide
2-(4-Bromo-phenoxy)-N-[4-methyl-2-(4-methyl-piperazin-1-yl)-pteridin-6-yl]-acetamide
2-(4-Bromo-phenoxy)-N-[4-methyl-2-(4-pyrrolidin-1-yl-piperidin-1-yl)-pteridin-6-yl]-acetamide
2-(4-Bromo-phenoxy)-N-{2-[4-(2-hydroxy-ethyl)-piperazin-1-yl]-4-methyl-pteridin-6-yl}-acetamide
2-(4-Bromo-phenoxy)-N-[2-(2,5-diaza-bicyclo[2.2.1]hept-2-yl)+methyl-pteridin-6-yl]-acetamide
2-(4-Bromo-phenoxy)-N-[2-(4-dimethylamino-piperidin-1-yl)-4-methyl-pteridin-6-yl]-acetamide
N-(4-Methyl-2-piperazin-1-yl-pteridin-6-yl)-3-(4-trifluoromethoxy-phenyl)-propionamide
N-[4-Methyl-2-(4-methyl-[1,4]diazepan-1-yl)-pteridin-6-yl]-3-(4-trifluoromethoxy-phenyl)-propionamide
N-[4-Methyl-2-(4-methyl-piperazin-1-yl)-pteridin-6-yl]-3-(4-trifluoromethoxy-phenyl)-propionamide
N-{2-[4-(2-Hydroxy-ethyl)-piperazin-1-yl]-4-methyl-pteridin-6-yl}3-(4-trifluoromethoxy-phenyl)-propionamide
N-[4-Methyl-2-(4-pyrrolidin-1-yl-piperidin-1-yl)-pteridin-6-yl]-3-(4-trifluoromethoxy-phenyl)-propionamide
N-[2-(2,5-Diaza-bicyclo[2.2.1]hept-2-yl)-4-methyl-pteridin-6-yl]-3-(4trifluoromethoxy-phenyl)-propionamide
N-[2-(4-Dimethylamino-piperidin-1-yl)-4-methyl-pteridin-6-yl]-3-(4-trifluoromethoxy-phenyl)-propionamide
(E)-N-(4-Methyl-2-piperazin-1-yl-pteridin-6-yl)-3-(4-trifluoromethoxy-phenyl)-acrylamide
(E)-N-[4-Methyl-2-(4-methyl-[1,4]diazepan-1-yl)-pteridin-6-yl]-3-(4-trifluoromethoxy-phenyl)-acrylamide
(E)-N-[4-Methyl-2-(4-methyl-piperazin-1-yl)-pteridin-6-yl]-3-(4-trifluoromethoxy-phenyl)-acrylamide
(E)-N-{2-[4-(2-Hydroxy-ethyl)-piperazin-1-yl]-4-methyl-pteridin-6-yl}-3-(4-trifluoromethoxy-phenyl)-acrylamide
(E)-N-[2-(4-Dimethylamino-piperidin-1-yl)-4-methyl-pteridin-6-yl]-3-(4-trifluoromethoxy-phenyl)-acrylamide
(E)-N-[2-(2,5-Diaza-bicyclo[2.2.1]hept-2-yl)-4-methyl-pteridin-6-yl]-3-(4-trifluoromethoxy-phenyl)-acrylamide
(E)-N-[4-Methyl-2-(4-pyrrolidin-1-yl-piperidin-1-yl)-pteridin-6-yl]-3-(4-trifluoromethoxy-phenyl)-acrylamide
3-(4-Chloro-phenyl)-N-(4-methyl-2-piperazin-1-yl-pteridin-6-yl)-propionamide
3-(4-Chloro-phenyl)-N-[4-methyl-2-(4-methyl-[1,4]diazepan-1-yl)-pteridin-6-yl]-propionamide
3-(4-Chloro-phenyl)-N-[4-methyl-2-(4-methyl-piperazin-1-yl)-pteridin-6-yl]-propionamide
3-(4-Chloro-phenyl)-N-{2-[4-(2-hydroxy-ethyl)-piperazin-1-yl]-4-methyl-pteridin-6-yl}-propionamide
3-(4-Chloro-phenyl)-N-[4-methyl-2-(4-pyrrolidin-1-yl-piperidin-1-yl)-pteridin-6-yl]-propionamide
3-(4-Chloro-phenyl)-N-[2-(2,5-diaza-bicyclo[2.2.1]hept-2-yl)-4-methyl-pteridin-6-yl]-propionamide
3-(4-Chloro-phenyl)-N-[2-(4-dimethylamino-piperidin-1-yl)-4-methyl-pteridin-6-yl]-propionamide
2-(2,4-Dichloro-phenoxy)-N-(4-methyl-2-piperazin-1-yl-pteridin-6-yl)-acetamide
2-(2,4-Dichloro-phenoxy)-N-[4-methyl-2-(4-methyl-[1,4]diazepan-1-yl)-pteridin-6-yl]-acetamide
2-(2,4-Dichloro-phenoxy)-N-[4-methyl-2-(4-methyl-piperazin-1-yl)-pteridin-6-yl]-acetamide
2-(2,4-Dichloro-phenoxy)-N-{2-[4-(2-hydroxy-ethyl)-piperazin-1-yl]4-methyl-pteridin-6-yl}acetamide
2-(2,4-Dichloro-phenoxy)-N-[2-(4-dimethylamino-piperidin-1-yl)-4-methyl-pteridin-6-yl]-acetamide
N-[2-(2,5-Diaza-bicyclo[2.2.1]hept-2-yl)-4-methyl-pteridin-6-yl]-2-(2,4-dichloro-phenoxy)-acetamide
2-(2,4-Dichloro-phenoxy)-N-[4-methyl-2-(4-pyrrolidin-1-yl-piperidin-1-yl)-pteridin-6-yl]-acetamide
2-(4-Thiomethoxy-phenoxy)-N-[4-methyl-2-(4-methyl-piperazin-1-yl)-quinolin-6-yl]-acetamide,
2-(4-Propyl-phenoxy)-N-[4-methyl-2-(4-methyl-piperazin-1-yl)-quinolin-6-yl]-acetamide,
2-(4-Trifluorothiomethoxy-phenoxy)-N-[4-methyl-2-(4-methyl-piperazin-1-yl)-quinolin-6-yl]-acetamide,
2-(4-Bromo-2-fluoro-phenoxy)-N-[4-methyl-2-(4-methyl-piperazin-1-yl)-quinolin-6-yl]-acetamide,
2-(4-Methoxy-2-fluoro-phenoxy)-N-[4-methyl-2-(4-methyl-piperazin-1-yl)-quinolin-6-yl]-acetamide,
2-(4-Isopropoxy-phenoxy)-N-[4-methyl-2-(4-methyl-piperazin-1-yl)-quinolin-6-yl]-acetamide,
2-(2-Chloro-4-ethoxy-phenoxy)-N-[4-methyl-2-(4-methyl-piperazin-1-yl)-quinolin-6-yl]-acetamide,
2-(4-Trifluoromethoxy-2-fluoro-phenoxy)-N-[4-methyl-2-(4-methyl-piperazin-1-yl)-quinolin-6-yl]-acetamide,
2-(4-Chloro-2-fluoro-phenoxy)-N-[4-methyl-2-(4-methyl-piperazin-1-yl)-quinolin-6-yl]-acetamide,
2-(2-Chloro-4-isopropoxy-phenoxy)-N-[4-methyl-2-(4-methyl-piperazin-1-yl)-quinolin-6-yl]-acetamide,
2-(2-Chloro-4-ethyl-phenoxy)-N-[4-methyl-2-(4-methyl-piperazin-1-yl)-quinolin-6-yl]-acetamide,
2-(4-Trifluoromethyl-2-fluoro-phenoxy)-N-[4-methyl-2-(4-methyl-piperazin-1-yl)-quinolin-6-yl]-acetamide,
3-(4-Thiomethoxy-phenyl)-N-[4-methyl-2-(4-methyl-piperazin-1-yl)-quinolin-6-yl]-acrylamide,
3-(4-Propyl-phenyl)-N-[4-methyl-2-(4-methyl-piperazin-1-yl)-quinolin-6-yl]-acrylamide,
3-(4-Trifluorothiomethoxy-phenyl)-N-[4-methyl-2-(4-methyl-piperazin-1-yl)-quinolin-6-yl]-acrylamide,
3-(4-Bromo-2-fluoro-phenyl)-N-[4-methyl-2-(4-methyl-piperazin-1-yl)-quinolin-6-yl]-acrylamide,
3-(4-Methoxy-2-fluoro-phenyl)-N-[4-methyl-2-(4-methyl-piperazin-1-yl)-quinolin-6-yl]-acrylamide,
3-(4-Isopropoxy-phenyl)-N-[4-methyl-2-(4-methyl-piperazin-1-yl)-quinolin-6-yl]-acrylamide,
3-(2-Chloro-4-ethoxy-phenyl)-N-[4-methyl-2-(4-methyl-piperazin-1-yl)-quinolin-6-yl]-acrylamide,
3-(4-Trifluoromethoxy-2-fluoro-phenyl)-N-[4-methyl-2-(4-methyl-piperazin-1-yl)-quinolin-6-yl]-acrylamide,
3-(4-Chloro-2-fluoro-phenyl)-N-[4-methyl-2-(4-methyl-piperazin-1-yl)-quinolin-6-yl]-acrylamide,
3-(2-Chloro-4-isopropoxy-phenyl)-N-[4-methyl-2-(4-methyl-piperazin-1-yl)-quinolin-6-yl]-acrylamide,
3-(2-Chloro-4-ethyl-phenyl)-N-[4-methyl-2-(4-methyl-piperazin-1-yl)-quinolin-6-yl]-acrylamide,
3-(4-Trifluoromethyl-2-fluoro-phenyl)-N-[4-methyl-2-(4-methyl-piperazin-1-yl)-quinolin-6-yl]-acrylamide,
3-(4-Thiomethoxy-phenyl)-N-[4-methyl-2-(4-methyl-piperazin-1-yl)-quinolin-6-yl]-propionamide,
3-(4-Propyl-phenyl)-N-[4-methyl-2-(4-methyl-piperazin-1-yl)-quinolin-6-yl]-propionamide,
3-(4-Trifluorothiomethoxy-phenyl)-N-[4-methyl-2-(4-methyl-piperazin-1-yl)-quinolin-6-yl]-propionamide,
3-(4-Bromo-2-fluoro-phenyl)-N-[4-methyl-2-(4-methyl-piperazin-1-yl)-quinolin-6-yl]-propionamide,
3-(4-Methoxy-2-fluoro-phenyl)-N-[4-methyl-2-(4-methyl-piperazin-1-yl)-quinolin-6-yl]-propionamide,
3-(4-Isopropoxy-phenyl)-N-[4-methyl-2-(4-methyl-piperazin-1-yl)-quinolin-6-yl]-propionamide,
3-(2-Chloro-4-ethoxy-phenyl)-N-[4-methyl-2-(4-methyl-piperazin-1-yl)-quinolin-6-yl]-propionamide,
3-(4-Trifluoromethoxy-2-fluoro-phenyl)-N-[4-methyl-2-(4-methyl-piperazin-1-yl)-quinolin-6-yl]-propionamide,
3-(4-Chloro-2-fluoro-phenyl)-N-[4-methyl-2-(4-methyl-piperazin-1-yl)-quinolin-6-yl]-propionamide,
3-(2-Chloro-4-isopropoxy-phenyl)-N-[4-methyl-2-(4-methyl-piperazin-1-yl)-quinolin-6-yl]propionamide,
3-(2-Chloro-4-ethyl-phenyl)-N-[4-methyl-2-(4-methyl-piperazin-1-yl)-quinolin-6-yl]-propionamide,
3-(4-Trifluoromethyl-2-fluoro-phenyl)-N-[4-methyl-2-(4-methyl-piperazin-1-yl)-quinolin-6-yl]-propionamide,
2-(4-Thiomethoxy-phenoxy)-N-[4-methyl-2-(4-pyrrolidino-piperidin-1-yl)-quinolin-6-yl]-acetamide,
2-(4-Propyl-phenoxy)-N-[4-methyl-2-(4-pyrrolidino-piperidin-1-yl)-quinolin-6-yl]-acetamide,
2-(4-Trifluorothiomethoxy-phenoxy)-N-[4-methyl-2-(4-pyrrolidino-piperidin-1-yl)-quinolin-6-yl]-acetamide,
2-(4-Bromo-2-fluoro-phenoxy)-N-[4-methyl-2-(4-pyrrolidino-piperidin-1-yl)-quinolin-6-yl]-acetamide,
2-(4-Methoxy-2-fluoro-phenoxy)-N-[4-methyl-2-(4-pyrrolidino-piperidin-1-yl)-quinolin-6-yl]-acetamide,
2-(4-Isopropoxy-phenoxy)-N-[4-methyl-2-(4-pyrrolidino-piperidin-1-yl)-quinolin-6-yl]-acetamide,
2-(2-Chloro-4-ethoxy-phenoxy)-N-[4-methyl-2-(4-pyrrolidino-piperidin-1-yl)-quinolin-6-yl]-acetamide,
2-(4-Trifluoromethoxy-2-fluoro-phenoxy)-N-[4-methyl-2-(4-pyrrolidino-piperidin-1-yl)-quinolin-6-yl]-acetamide,
2-(4-Chloro-2-fluoro-phenoxy)-N-[4-methyl-2-(4-pyrrolidino-piperidin-1-yl)-quinolin-6-yl]-acetamide,
2-(2-Chloro-4-isopropoxy-phenoxy)-N-[4-methyl-2-(4-pyrrolidino-piperidin-1-yl)-quinolin-6-yl]-acetamide,
2-(2-Chloro-4-ethyl-phenoxy)-N-[4-methyl-2-(4-pyrrolidino-piperidin-1-yl)-quinolin-6-yl]-acetamide,
2-(4-Trifluoromethyl-2-fluoro-phenoxy)-N-[4-methyl-2-(4-pyrrolidino-piperidin-1-yl)-quinolin-6-yl]-acetamide,
3-(4-Thiomethoxy-phenyl)-N-[4-methyl-2-(4-pyrrolidino-piperidin-1-yl)-quinolin-6-yl]-acrylamide,
3-(4-Propyl-phenyl)-N-[4-methyl-2-(4-pyrrolidino-piperidin-1-yl)-quinolin-6-yl]-acrylamide,
3-(4-Trifluorothiomethoxy-phenyl)-N-[4-methyl-2-(4-pyrrolidino-piperidin-1-yl)-quinolin-6-yl]-acrylamide,
3-(4-Bromo-2-fluoro-phenyl)-N-[4-methyl-2-(4-pyrrolidino-piperidin-1-yl)-quinolin-6-yl]-acrylamide,
3-(4-Methoxy-2-fluoro-phenyl)-N-[4-methyl-2-(4-pyrrolidino-piperidin-1-yl)-quinolin-6-yl]-acrylamide,
3-(4-Isopropoxy-phenyl)-N-[4-methyl-2-(4-pyrrolidino-piperidin-1-yl)-quinolin-6-yl]-acrylamide,
3-(2-Chloro-4-ethoxy-phenyl)-N-[4-methyl-2-(4-pyrrolidino-piperidin-1-yl)-quinolin-6-yl]-acrylamide,
3-(4-Trifluoromethoxy-2-fluoro-phenyl)-N-[4-methyl-2-(4-pyrrolidino-piperidin-1-yl)-quinolin-6-yl]-acrylamide,
3-(4-Chloro-2-fluoro-phenyl)-N-[4-methyl-2-(4-pyrrolidino-piperidin-1-yl)-quinolin-6-yl]-acrylamide,
3-(2-Chloro-4-isopropoxy-phenyl)-N-[4-methyl-2-(4-pyrrolidino-piperidin-1-yl)-quinolin-6-yl]-acrylamide,
3-(2-Chloro-4-ethyl-phenyl)-N-[4-methyl-2-(4-pyrrolidino-piperidin-1-yl)-quinolin-6-yl]-acrylamide,
3-(4-Trifluoromethyl-2-fluoro-phenyl)-N-[4-methyl-2-(4-pyrrolidino-piperidin-1-yl)-quinolin-6-yl]-acrylamide,
3-(4-Thiomethoxy-phenyl)-N-[4-methyl-2-(4-pyrrolidino-piperidin-1-yl)-quinolin-6-yl]-propionamide,
3-(4-Propyl-phenyl)-N-[4-methyl-2-(4-pyrrolidino-piperidin-1-yl)-quinolin-6-yl]-propionamide,
3-(4-Trifluorothiomethoxy-phenyl)-N-[4-methyl-2-(4-pyrrolidino-piperidin-1-yl)-quinolin-6-yl]-propionamide,
3-(4-Bromo-2-fluoro-phenyl)-N-[4-methyl-2-(4-pyrrolidino-piperidin-1-yl)-quinolin-6-yl]-propionamide,
3-(4-Methoxy-2-fluoro-phenyl)-N-[4-methyl-2-(4-pyrrolidino-piperidin-1-y)-quinolin-6-yl]-propionamide,
3-(4-isopropoxy-phenyl)-N-[4-methyl-2-(4-pyrrolidino-piperidin-1-yl)-quinolin-6-yl]-propionamide,
3-(2-Chloro-4-ethoxy-phenyl)-N-[4-methyl-2-(4-pyrrolidino-piperidin-1-yl)-quinolin-6-yl]-propionamide,
3-(4-Trifluoromethoxy-2-fluoro-phenyl)-N-[4-methyl-2-(4-pyrrolidino-piperidin-1-yl)-quinolin-6-yl]-propionamide,
3-(4-Chloro-2-fluoro-phenyl)-N-[4-methyl-2-(4-pyrrolidino-piperidin-1-yl)-quinolin-6-yl]-propionamide,
3-(2-Chloro-4-isopropoxy-phenyl)-N-[4-methyl-2-(4-pyrrolidino-piperidin-1-yl)-quinolin-6-yl]-propionamide,
3-(2-Chloro-4-ethyl-phenyl)-N-[methyl-2-(4-pyrrolidino-piperidin-1-yl)-quinolin-6-yl]-propionamide,
3-(4-Trifluoromethyl-2-fluoro-phenyl)-N-[4-methyl-2-(4-pyrrolidino-piperidin-1-yl)-quinolin-6-yl]-propionamide,
2-(4-Thiomethoxy-phenoxy)-N-[4-methyl-2-(4-dimethylamino-piperidin-1-yl)-quinolin-6-yl]-acetamide,
2-(4-Propyl-phenoxy)-N-[4-methyl-2-(4-dimethylamino-piperidin-1-yl)-quinolin-6-yl]-acetamide,
2-(4-Trifluorothiomethoxy-phenoxy)-N-[4-methyl-2-(4-dimethylamino-piperidin-1-yl)-quinolin-6-yl]-acetamide,
2-(4-Bromo-2-fluoro-phenoxy)-N-[4-methyl-2-(4-dimethylamino-piperidin-1-yl)-quinolin-6-yl]-acetamide,
2-(4-Methoxy-2-fluoro-phenoxy)-N-[4-methyl-2-(4-dimethylamino-piperidin-1-yl)-quinolin-6-yl]-acetamide,
2-(4-Isopropoxy-phenoxy)-N-[4-methyl-2-(4-dimethylamino-piperidin-1-yl)-quinolin-6-yl]-acetamide,
2-(2-Chloro-4-ethoxy-phenoxy)-N-[4-methyl-2-(4-dimethylamino-piperidin-1-yl)-quinolin-6-yl]-acetamide,
2-(4-Trifluoromethoxy-2-fluoro-phenoxy)-N-[4-methyl-2-(4-dimethylamino-piperidin-1-yl)-quinolin-6-yl]-acetamide,
2-(4-Chloro-2-fluoro-phenoxy)-N-[4-methyl-2-(4-dimethylamino-piperidin-1-yl)-quinolin-6-yl]-acetamide,
2-(2-Chloro-4-isopropoxy-phenoxy)-N-[4-methyl-2-(4-dimethylamino-piperidin-1-yl)-quinolin-6-yl]-acetamide,
2-(2-Chloro-4-ethyl-phenoxy)-N-[4-methyl-2-(4-dimethylamino-piperidin-1-yl)-quinolin-6-yl]-acetamide,
2-(4-Trifluoromethyl-2-fluoro-phenoxy)-N-[4-methyl-2-(4-dimethylamino-piperidin-1-yl)-quinolin-6-yl]-acetamide,
3-(4-Thiomethoxy-phenyl)-N-[4-methyl-2-(4-dimethylamino-piperidin-1-yl)-quinolin-6-yl]-acrylamide,
3-(4-Propyl-phenyl)-N-[4-methyl-2-(4-dimethylamino-piperidin-1-yl)-quinolin-6-yl]-acrylamide,
3-(4-Trifluorothiomethoxy-phenyl)-N-[4-methyl-2-(4-dimethylamino-piperidin-1-yl)-quinolin-6-yl]-acrylamide,
3-(4-Bromo-2-fluoro-phenyl)-N-[4-methyl-2-(4-dimethylamino-piperidin-1-yl)-quinolin-6-yl]-acrylamide,
3-(4-Methoxy-2-fluoro-phenyl)-N-[4-methyl-2-(4-dimethylamino-piperidin-1-yl)-quinolin-6-yl]-acrylamide,
3-(4-Isopropoxy-phenyl)-N-[4-methyl-2-(4-dimethylamino-piperidin-1-yl)-quinolin-6-yl]-acrylamide,
3-(2-Chloro-4-ethoxy-phenyl)-N-[4-methyl-2-(4-dimethylamino-piperidin-1-yl)-quinolin-6-yl]-acrylamide,
3-(4-Trifluoromethoxy-2-fluoro-phenyl)-N-[4-methyl-2-(4-dimethylamino-piperidin-1-yl)-quinolin-6-yl]-acrylamide,
3-(4-Chloro-2-fluoro-phenyl)-N-[4-methyl-2-(4-dimethylamino-piperidin-1-yl)-quinolin-6-yl]-acrylamide,
3-(2-Chloro-4-isopropoxy-phenyl)-N-[4-methyl-2-(4-dimethylamino-piperidin-1-yl)-quinolin-6-yl]-acrylamide,
3-(2-Chloro-4-ethyl-phenyl)-N-[4-methyl-2-(4-dimethylamino-piperidin-1-yl)-quinolin-6-yl]-acrylamide,
3-(4-Trifluoromethyl-2-fluoro-phenyl)-N-[4-methyl-2-(4-dimethylamino-piperidin-1-yl)-quinolin-6-yl]-acrylamide,
3-(4-Thiomethoxy-phenyl)-N-[4-methyl-2-(4-dimethylamino-piperidin-1-yl)-quinolin-6-yl]-propionamide,
3-(4-Propyl-phenyl)-N-[4-methyl-2-(4-dimethylamino-piperidin-1-yl)-quinolin-6-yl]-propionamide,
3-(4-Trifluorothiomethoxy-phenyl)-N-[4-methyl-2-(4-dimethylamino-piperidin-1-yl)-quinolin-6-yl]-propionamide,
3-(4-Bromo-2-fluoro-phenyl)-N-[4-methyl-2-(4-dimethylamino-piperidin-1-yl)-quinolin-6-yl]-propionamide,
3-(4-Methoxy-2-fluoro-phenyl)-N-[4-methyl-2-(4-dimethylamino-piperidin-1-yl)-quinolin-6-yl]-propionamide,
3-(4-Isopropoxy-phenyl)-N-[4-methyl-2-(4-dimethylamino-piperidin-1-yl)-quinolin-6-yl]-propionamide,
3-(2-Chloro-4-ethoxy-phenyl)-N-[4-methyl-2-(4-dimethylamino-piperidin-1-yl)-quinolin-6-yl]-propionamide,
3-(4-Trifluoromethoxy-2-fluoro-phenyl)-N-[4-methyl-2-(4-dimethylamino-piperidin-1-yl)-quinolin-6-yl]-propionamide,
3-(4-Chloro-2-fluoro-phenyl)-N-[4-methyl-2-(4-dimethylamino-piperidin-1-yl)-quinolin-6-yl]-propionamide,
3-(2-Chloro-4-isopropoxy-phenyl)-N-[4-methyl-2-(4-dimethylamino-piperidin-1-yl)-quinolin-6-yl-propionamide,
3-(2-Chloro-4-ethyl-phenyl)-N-[4-methyl-2-(4-dimethylamino-piperidin-1-yl)-quinolin-6-yl]-propionamide,
3-(4-Trifluoromethyl-2-fluoro-phenyl)-N-[4-methyl-2-(4-dimethylamino-piperidin-1-yl)-quinolin-6-yl]-propionamide,
2-(4-Thiomethoxy-phenoxy)-N-[4-methyl-2-(4-methyl-piperazin-1-yl)-quinazolin-6-yl]-acetamide,
2-(4-Propyl-phenoxy)-N-[4-methyl-2-(4-methyl-piperazin-1-yl)-quinazolin-6-yl]-acetamide,
2-(4-Trifluorothiomethoxy-phenoxy)-N-[4-methyl-2-(4-methyl-piperazin-1-yl)-quinazolin-6-yl]-acetamide,
2-(4-Bromo-2-fluoro-phenoxy)-N-[4-methyl-2-(4-methyl-piperazin-1-yl)-quinazolin-6-yl]-acetamide,
2-(4-Methoxy-2-fluoro-phenoxy)-N-[4-methyl-2-(4-methyl-piperazin-1-yl)-quinazolin-6-yl]-acetamide,
2-(4-Isopropoxy-phenoxy)-N-[4-methyl-2-(4-methyl-piperazin-1-yl)-quinazolin-6-yl]-acetamide,
2-(2-Chloro-4-ethoxy-phenoxy)-N-[4-methyl-2-(4-methyl-piperazin-1-yl)-quinazolin-6-yl]-acetamide,
2-(4-Trifluoromethoxy-2-fluoro-phenoxy)-N-[4-methyl-2-(4-methyl-piperazin-1-yl)-quinazolin-6-yl]-acetamide,
2-(4-Chloro-2-fluoro-phenoxy)-N-[4-methyl-2-(4-methyl-piperazin-1-yl)-quinazolin-6-yl]-acetamide,
2-(2-Chloro-4-isopropoxy-phenoxy)-N-[4-methyl-2-(4-methyl-piperazin-1-yl)-quinazolin-6-yl]-acetamide,
2-(2-Chloro-4-ethyl-phenoxy)-N-[4-methyl-2-(4-methyl-piperazin-1-yl)-quinazolin-6-yl]-acetamide,
2-(4-Trifluoromethyl-2-fluoro-phenoxy)-N-[4-methyl-2-(4-methyl-piperazin-1-yl)-quinazolin-6-yl]-acetamide,
3-(4-Thiomethoxy-phenyl)-N-[4-methyl-2-(4-methyl-piperazin-1-yl)-quinazolin-6-yl]-acrylamide,
3-(4-Propyl-phenyl)-N-[4-methyl-2-(4-methyl-piperazin-1-yl)-quinazolin-6-yl]-acrylamide,
3-(4-Trifluorothiomethoxy-phenyl)-N-[4-methyl-2-(4-methyl-piperazin-1-yl)-quinazolin-6-yl]-acrylamide,
3-(4-Bromo-2-fluoro-phenyl)-N-[4-methyl-2-(4-methyl-piperazin-1-yl)-quinazolin-6-yl]-acrylamide,
3-(4-Methoxy-2-fluoro-phenyl)-N-[4-methyl-2-(4-methyl-piperazin-1-yl)-quinazolin-6-yl]-acrylamide,
3-(4-Isopropoxy-phenyl)-N-[4-methyl-2-(4-methyl-piperazin-1-yl)-quinazolin-6-yl]-acrylamide,
3-(2-Chloro-4-ethoxy-phenyl)-N-[4-methyl-2-(4-methyl-piperazin-1-yl)-quinazolin-6-y]-acrylamide,
3-(4-Trifluoromethoxy-2-fluoro-phenyl)-N-[4-methyl-2-(4-methyl-piperazin-1-yl)-quinazolin-6-yl]-acrylamide,
3-(4-Chloro-2-fluoro-phenyl)-N-[4-methyl-2-(4-methyl-piperazin-1-yl)-quinazolin-6-yl]-acrylamide,
3-(2-Chloro-4-isopropoxy-phenyl)-N-[4-methyl-2-(4-methyl-piperazin-1-yl)-quinazolin-6-yl]-acrylamide,
3-(2-Chloro-4-ethyl-phenyl)-N-[4-methyl-2-(4-methyl-piperazin-1-yl)-quinazolin-6-yl]-acrylamide,
3-(4-Trifluoromethyl-2-fluoro-phenyl)-N-[4-methyl-2-(4-methyl-piperazin-1-yl)-quinazolin-6-yl]-acrylamide,
3-(4-Thiomethoxy-phenyl)-N-[4-methyl-2-(4-methyl-piperazin-1-yl)-quinazolin-6-yl]-propionamide,
3-(4-Propyl-phenyl)-N-[4-methyl-2-(4-methyl-piperazin-1-yl)-quinazolin-6-yl]-propionamide,
3-(4-Trifluorothiomethoxy-phenyl)-N-[4-methyl-2-(4-methyl-piperazin-1-yl)-quinazolin-6-yl]-propionamide,
3-(4-Bromo-2-fluoro-phenyl)-N-[4-methyl-2-(4-methyl-piperazin-1-yl)-quinazolin-6-yl]-propionamide,
3-(4-Methoxy-2-fluoro-phenyl)-N-[4-methyl-2-(4-methyl-piperazin-1-yl)-quinazolin-6-yl]-propionamide,
3-(4-Isopropoxy-phenyl)-N-[4-methyl-2-(4-methyl-piperazin-1-yl)-quinazolin-6-yl]-propionamide,
3-(2-Chloro-4-ethoxy-phenyl)-N-[4-methyl-2-(4-methyl-piperazin-1-yl)-quinazolin-6-yl]-propionamide,
3-(4-Trifluoromethoxy-2-fluoro-phenyl)-N-[4-methyl-2-(4-methyl-piperazin-1-yl)-quinazolin-6-yl]-propionamide,
3-(4-Chloro-2-fluoro-phenyl)-N-[4-methyl-2-(4-methyl-piperazin-1-yl)-quinazolin-6-yl]-propionamide,
3-(2-Chloro-4-isopropoxy-phenyl)-N-[4-methyl-2-(4-methyl-piperazin-1-yl)-quinazolin-6-yl]-propionamide,
3-(2-Chloro-4-ethyl-phenyl)-N-[4-methyl-2-(4-methyl-piperazin-1-yl)-quinazolin-6-yl]-propionamide,
3-(4-Trifluoromethyl-2-fluoro-phenyl)-N-[4-methyl-2-(4-methyl-piperazin-1-yl)-quinazolin-6-yl]-propionamide,
2-(4-Thiomethoxy-phenoxy)-N-[4-methyl-2-(4-pyrrolidino-piperidin-1-yl)-quinazolin-6-yl]-acetamide,
2-(4-Propyl-phenoxy)-N-[4-methyl-2-(4-pyrrolidino-piperidin-1-yl)-quinazolin-6-yl]-acetamide,
2-(4-Trifluorothiomethoxy-phenoxy)-N-[4-methyl-2-(4-pyrrolidino-piperidin-1-yl)-quinazolin-6-yl]-acetamide,
2-(4-Bromo-2-fluoro-phenoxy)-N-[4-methyl-2-(4-pyrrolidino-piperidin-1-yl)-quinazolin-6-yl]-acetamide,
2-(4-Methoxy-2-fluoro-phenoxy)-N-[4-methyl-2-(4-pyrrolidino-piperidin-1-yl)-quinazolin-6-yl]-acetamide,
2-(4-Isopropoxy-phenoxy)-N-[4-methyl-2-(4-pyrrolidino-piperidin-1-yl)-quinazolin-6-yl]-acetamide,
2-(2-Chloro-4-ethoxy-phenoxy)-N-[4-methyl-2-(4-pyrrolidino-piperidin-1-yl)-quinazolin-6-yl]-acetamide,
2-(4-Trifluoromethoxy-2-fluoro-phenoxy)-N-[4-methyl-2-(4-pyrrolidino-piperidin-1-yl)-quinazolin-6-yl]-acetamide,
2-(4-Chloro-2-fluoro-phenoxy)-N-[4-methyl-2-(4-pyrrolidino-piperidin-1-y)-quinazolin-6-yl]-acetamide,
2-(2-Chloro-4-isopropoxy-phenoxy)-N-[4-methyl-2-(4-pyrrolidino-piperidin-1-yl)-quinazolin-6-yl]-acetamide,
2-(2-Chloro-4-ethyl-phenoxy)-N-[4-methyl-2-(4-pyrrolidino-piperidin-1-yl)-quinazolin-6-yl]-acetamide,
2-(4-Trifluoromethyl-2-fluoro-phenoxy)-N-[4-methyl-2-(4-pyrrolidino-piperidin-1-yl)-quinazolin-6-yl]-acetamide,
3-(4-Thiomethoxy-phenyl)-N-[4-methyl-2-(4-pyrrolidino-piperidin-1-yl)-quinazolin-6-yl]-acrylamide,
3-(4-Propyl-phenyl)-N-[4-methyl-2-(4-pyrrolidino-piperidin-1-yl)-quinazolin-6-yl]-acrylamide,
3-(4-Trifluorothiomethoxy-phenyl)-N-[4-methyl-2-(4-pyrrolidino-piperidin-1-yl)-quinazolin-6-yl]-acrylamide,
3-(4-Bromo-2-fluoro-phenyl)-N-[4-methyl-2-(4-pyrrolidino-piperidin-1-yl)-quinazolin-6-yl]-acrylamide,
3-(4-Methoxy-2-fluoro-phenyl)-N-[4-methyl-2-(4-pyrrolidino-piperidin-1-yl)-quinazolin-6-yl]-acrylamide,
3-(4-Isopropoxy-phenyl)-N-[4-methyl-2-(4-pyrrolidino-piperidin-1-yl)-quinazolin-6-yl]-acrylamide,
3-(2-Chloro-4-ethoxy-phenyl)-N-[4-methyl-2-(4-pyrrolidino-piperidin-1-yl)-quinazolin-6-yl]-acrylamide,
3-(4-Trifluoromethoxy-2-fluoro-phenyl)-N-[4-methyl-2-(4-pyrrolidino-piperidin-1-yl)-quinazolin-6-yl]-acrylamide,
3-(4-Chloro-2-fluoro-phenyl)-N-[4-methyl-2-(4-pyrrolidino-piperidin-1-yl)-quinazolin-6-yl]-acrylamide,
3-(2-Chloro-4-isopropoxy-phenyl)-N-[4-methyl-2-(4-pyrrolidino-piperidin-1-yl)-quinazolin-6-yl]-acrylamide,
3-(2-Chloro-4-ethyl-phenyl)-N-[4-methyl-2-(4-pyrrolidino-piperidin-1-yl)-quinazolin-6-yl]-acrylamide,
3-(4-Trifluoromethyl-2-fluoro-phenyl)-N-[4-methyl-2-(4-pyrrolidino-piperidin-1-yl)-quinazolin-6-yl]-acrylamide,
3-(4-Thiomethoxy-phenyl)-N-[4-methyl-2-(4-pyrrolidino-piperidin-1-yl)-quinazolin-6-yl]-propionamide,
3-(4-Propyl-phenyl)-N-[4-methyl-2-(4-pyrrolidino-piperidin-1-yl)-quinazolin-6-yl]-propionamide,
3-(4-Trifluorothiomethoxy-phenyl)-N-[4-methyl-2-(4-pyrrolidino-piperidin-1-yl)-quinazolin-6-yl]-propionamide,
3-(4-Bromo-2-fluoro-phenyl)-N-[4-methyl-2-(4-pyrrolidino-piperidin-1-yl)-quinazolin-6-yl]-propionamide,
3-(4-Methoxy-2-fluoro-phenyl)-N-[4-methyl-2-(4-pyrrolidino-piperidin-1-yl)-quinazolin-6-yl]-propionamide,
3-(4-Isopropoxy-phenyl)-N-[4-methyl-2-(4-pyrrolidino-piperidin-1-yl)-quinazolin-6-yl]-propionamide,
3-(2-Chloro-4-ethoxy-phenyl)-N-[4-methyl-2-(4-pyrrolidino-piperidin-1-yl)-quinazolin-6-yl]-propionamide,
3-(4-Trifluoromethoxy-2-fluoro-phenyl)-N-[4-methyl-2-(4-pyrrolidino-piperidin-1-yl)-quinazolin-6-yl]-propionamide,
3-(4-Chloro-2-fluoro-phenyl)-N-[4-methyl-2-(4-pyrrolidino-piperidin-1-yl)-quinazolin-6-yl]-propionamide,
3-(2-Chloro-4-isopropoxy-phenyl)-N-[4-methyl-2-(4-pyrrolidino-piperidin-1-yl)-quinazolin-6-yl]-propionamide,
3-(2-Chloro-4-ethyl-phenyl)-N-[4-methyl-2-(4-pyrrolidino-piperidin-1-yl)-quinazolin-6-yl]-propionamide,
3-(4-Trifluoromethyl-2-fluoro-phenyl)-N-[4-methyl-2-(4-pyrrolidino-piperidin-1-yl)-quinazolin-6-yl]-propionamide,
2-(4-Thiomethoxy-phenoxy)-N-[4-methyl-2-(4-dimethylamino-piperidin-1-yl)-quinazolin-6-yl]-acetamide,
2-(4-Propyl-phenoxy)-N-[4-methyl-2-(4-dimethylamino-piperidin-1-yl)-quinazolin-6-yl]-acetamide,
2-(4-Trifluorothiomethoxy-phenoxy)-N-[4-methyl-2-(4-dimethylamino-piperidin-1-yl)-quinazolin-6-yl]-acetamide,
2-(4-Bromo-2-fluoro-phenoxy)-N-[4-methyl-2-(4-dimethylamino-piperidin-1-yl)-quinazolin-6-yl]-acetamide,
2-(4-Methoxy-2-fluoro-phenoxy)-N-[4-methyl-2-(4-dimethylamino-piperidin-1-yl)-quinazolin-6-yl]-acetamide,
2-(4-Isopropoxy-phenoxy)-N-[4-methyl-2-(4-dimethylamino-piperidin-1-yl)-quinazolin-6-yl]-acetamide,
2-(2-Chloro-4-ethoxy-phenoxy)-N-[4-methyl-2-(4-dimethylamino-piperidin-1-yl)-quinazolin-6-yl]-acetamide,
2-(4-Trifluoromethoxy-2-fluoro-phenoxy)-N-[4-methyl-2-(4-dimethylamino-piperidin-1-yl)-quinazolin-6-yl]-acetamide,
2-(4-Chloro-2-fluoro-phenoxy)-N-[4-methyl-2-(4-dimethylamino-piperidin-1-yl)-quinazolin-6-yl]-acetamide,
2-(2-Chloro-4-isopropoxy-phenoxy)-N-[4-methyl-2-(4-dimethylamino-piperidin-1-yl)-quinazolin-6-yl]-acetamide,
2-(2-Chloro-4-ethyl-phenoxy)-N-[4-methyl-2-(4-dimethylamino-piperidin-1-yl)-quinazolin-6-yl]-acetamide,
2-(4-Trifluoromethyl-2-fluoro-phenoxy)-N-[4-methyl-2-(4-dimethylamino-piperidin-1-yl)-quinazolin-6-yl]-acetamide,
3-(4-Thiomethoxy-phenyl)-N-[4-methyl-2-(4-dimethylamino-piperidin-1-yl)-quinazolin-6-yl]-acrylamide,
3-(4-Propyl-phenyl)-N-[4-methyl-2-(4-dimethylamino-piperidin-1-yl)-quinazolin-6-yl]-acrylamide,
3-(4-Trifluorothiomethoxy-phenyl)-N-[4-methyl-2-(4-dimethylamino-piperidin-1-yl)-quinazolin-6-yl]-acrylamide,
3-(4-Bromo-2-fluoro-phenyl)-N-[4-methyl-2-(4-dimethylamino-piperidin-1-yl)-quinazolin-6-yl]-acrylamide,
3-(4-Methoxy-2-fluoro-phenyl)-N-[4-methyl-2-(4-dimethylamino-piperidin-1-yl)-quinazolin-6-yl]-acrylamide,
3-(4-Isopropoxy-phenyl)-N-[4-methyl-2-(4-dimethylamino-piperidin-1-yl)-quinazolin-6-yl]-acrylamide,
3-(2-Chloro-4-ethoxy-phenyl)-N-[4-methyl-2-(4-dimethylamino-piperidin-1-yl)-quinazolin-6-yl]-acrylamide,
3-(4-Trifluoromethoxy-2-fluoro-phenyl)-N-[4-methyl-2-(4-dimethylamino-piperidin-1-yl)-quinazolin-6-yl]-acrylamide,
3-(4-Chloro-2-fluoro-phenyl)-N-[4-methyl-2-(4-dimethylamino-piperidin-1-yl)-quinazolin-6-yl]-acrylamide,
3-(2-Chloro-4-isopropoxy-phenyl)-N-[4-methyl-2-(4-dimethylamino-piperidin-1-yl)-quinazolin-6-yl]-acrylamide,
3-(2-Chloro-4-ethyl-phenyl)-N-[4-methyl-2-(4-dimethylamino-piperidin-1-yl)-quinazolin-6-yl]-acrylamide,
3-(4-Trifluoromethyl-2-fluoro-phenyl)-N-[4-methyl-2-(4-dimethylamino-piperidin-1-yl)-quinazolin-6-yl]-acrylamide,
3-(4-Thiomethoxy-phenyl)-N-[4-methyl-2-(4-dimethylamino-piperidin-1-yl)-quinazolin-6-yl]-propionamide,
3-(4-Propyl-phenyl)-N-[4-methyl-2-(4-dimethylamino-piperidin-1-yl)-quinazolin-6-yl]-propionamide,
3-(4-Trifluorothiomethoxy-phenyl)-N-[4-methyl-2-(4-dimethylamino-piperidin-1-yl)-quinazolin-6-yl]-propionamide,
3-(4-Bromo-2-fluoro-phenyl)-N-[4-methyl-2-(4-dimethylamino-piperidin-1-yl)-quinazolin-6-yl]-propionamide,
3-(4-Methoxy-2-fluoro-phenyl)-N-[4-methyl-2-(4-dimethylamino-piperidin-1-yl)-quinazolin-6-yl]-propionamide,
3-(4-Isopropoxy-phenyl)-N-[4-methyl-2-(4-dimethylamino-piperidin-1-yl)-quinazolin-6-yl]-propionamide,
3-(2-Chloro-4-ethoxy-phenyl)-N-[4-methyl-2-(4-dimethylamino-piperidin-1-yl)-quinazolin-6-yl]-propionamide,
3-(4-Trifluoromethoxy-2-fluoro-phenyl)-N-[4-methyl-2-(4-dimethylamino-piperidin-1-yl)-quinazolin-6-yl]-propionamide,
3-(4-Chloro-2-fluoro-phenyl)-N-[4-methyl-2-(4-dimethylamino-piperidin-1-yl)-quinazolin-6-yl]-propionamide,
3-(2-Chloro isopropoxy-phenyl)-N-[4-methyl-2-(4-dimethylamino-piperidin-1-yl)-quinazolin-6-yl]-propionamide,
3-(2-Chloro-4-ethyl-phenyl)-N-[4-methyl-2-(4-dimethylamino-piperidin-1-yl)-quinazolin-6-yl]-propionamide,
3-(4-Trifluoromethyl-2-fluoro-phenyl)-N-[4-methyl-2-(4-dimethylamino-piperidin-1-yl)-quinazolin-6-yl]-propionamide,
3-(4-Chloro-2-thienyl)-N-[4-methyl-2-(4-methyl-piperazin-1-yl)-quinolin-6-yl]-propionamide,
3-(5-Chloro-2-thienyl)-N-[4-methyl-2-(4-methyl-piperazin-1-yl)-quinolin-6-yl]-propionamide,
3-(5-Chloro-3-thienyl)-N-[4-methyl-2-(4-methyl-piperazin-1-yl)-quinolin-6-yl]-propionamide,
3-(5-Trifluoromethyl-3-thienyl)-N-[4-methyl-2-(4-methyl-piperazin-1-yl)-quinolin-6-yl]-propionamide,
2-(4-Chloro-2-thienyloxy)-N-[4-methyl-2-(4-methyl-piperazin-1-yl)-quinolin-6-yl]-acetamide,
2-(5-Chloro-2-thienyloxy)-N-[4-methyl-2-(4-methyl-piperazin-1-yl)-quinolin-6-yl]-acetamide,
2-(5-Chloro-3-thienyloxy)-N-[4-methyl-2-(4-methyl-piperazin-1-yl)-quinolin-6-yl]-acetamide,
2-(5-Trifluoromethyl-3-thienyloxy)-N-[4-methyl-2-(4-methyl-piperazin-1-yl)-quinolin-6-yl]-acetamide,
3-(4-Methyl-2-thienyl)-N-[4-methyl-2-(4-methyl-piperazin-1-yl)-quinolin-6-yl]-propionamide,
3-(5-Methyl-2-thienyl)-N-[4-methyl-2-(4-methyl-piperazin-1-yl)-quinolin-6-yl]-propionamide,
3-(5-Methyl-3-thienyl)-N-[4-methyl-2-(4-methyl-piperazin-1-yl)-quinolin-6-yl]-propionamide,
3-(5-Trifluoromethoxy-3-thienyl)-N-[4-methyl-2-(4-methyl-piperazin-1-yl)-quinolin-6-yl]-propionamide,
2-(4-Methyl-2-thienyloxy)-N-[4-methyl-2-(4-methyl-piperazin-1-yl)-quinolin-6-yl]-acetamide,
2-(5-Methyl-2-thienyl)oxy-N-[4-methyl-2-(4-methyl-piperazin-1-yl)-quinolin-6-yl]-acetamide,
2-(5-Methyl-3-thienyloxy)-N-(4-methyl-2-(4-methyl-piperazin-1-yl)-quinolin-6-yl]-35 acetamide,
2-(5-Trifluoromethoxy-3-thienyloxy)-N-[4-methyl-2-(4-methyl-piperazin-1-yl)-quinolin-6-yl]-acetamide,
3-(4-Chloro-2-thienyl)-N-[4-methyl-2-(4-pyrrolidino-piperidin-1-yl)-quinolin-6-yl]-propionamide,
3-(5-Chloro-2-thienyl)-N-(4-methyl-2-(4-pyrrolidino-piperidin-1-yl l)-quinolin-6-yl]-propionamide,
3-(5-Chloro-3-thienyl)-N-[4-methyl-2-(4-pyrrolidino-piperidin-1-yl)-quinolin-6-yl]-propionamide,
3-(5-Trifluoromethyl-3-thienyl)-N-[4-methyl-2-(4-pyrrolidino-piperidin-1-yl)-quinolin-6-yl]-propionamide,
2-(4-Chloro-2-thienyloxy)-N-[4-methyl-2-(4-pyrrolidino-piperidin-1-yl l)-quinolin-6-yl]-acetamide,
2-(5-Chloro-2-thienyloxy)-N-[4-methyl-2-(4-pyrrolidino-piperidin-1-yl)-quinolin-6-yl]-acetamide,
2-(5-Chloro-3-thienyloxy)-N-[4-methyl-2-(4-pyrrolidino-piperidin-1-yl)-quinolin-6-yl]-acetamide,
2-(5-Trifluoromethyl-3-thienyloxy)-N-[4-methyl-2-(4-pyrrolidino-piperidin-1-yl)-quinolin-6-yl]-acetamide,
3-(4-Methyl-2-thienyl)-N-[4-methyl-2-(4-pyrrolidino-piperidin-1-yl)-quinolin-6-yl]-propionamide,
3-(5-Methyl-2-thienyl)-N-[4-methyl-2-(4-pyrrolidino-piperidin-1-yl)-quinolin-6-yl]-propionamide,
3-(5-Methyl-3-thienyl)-N-[4-methyl-2-(4-pyrrolidino-piperidin-1-yl)-quinolin-6-yl]-propionamide,
3-(5-Trifluoromethoxy-3-thienyl)-N-[4-methyl-2-(4-pyrrolidino-piperidin-1-yl)-quinolin-6-yl]-propionamide,
2-(4-Methyl-2-thienyloxy)-N-[4-methyl-2-(4-pyrrolidino-piperidin-1-yl)-quinolin-6-yl]-acetamide,
2-(5-Methyl-2-thienyloxy)-N-[4-methyl-2-(4-pyrrolidino-piperidin-1-yl)-quinolin-6-yl]-acetamide,
2-(5-Methyl-3-thienyloxy)-N-[4-methyl-2-(4-pyrrolidino-piperidin-1-yl)-quinolin-6-yl]-acetamide,
2-(5-Trifluoromethoxy-3-thienyloxy)-N-[4-methyl-2-(4-pyrrolidino-piperidin-1-yl)-quinolin-6-yl]-acetamide,
3-(4-Chloro-2-thienyl)-N-[4-methyl-2-(4-dimethylamino-piperidin-1-yl)-quinolin-6-yl]-propionamide,
3-(5-Chloro-2-thienyl)-N-[4-methyl-2-(4-dimethylamino-piperidin-1-yl l)-quinolin-6-yl]-propionamide,
3-(5-Chloro-3-thienyl)-N-[4-methyl-2-(4-dimethylamino-piperidin-1-yl)-quinolin-6-yl]-propionamide,
3-(5-Trifluoromethyl-3-thienyl)-N-[4-methyl-2-(4-dimethylamino-piperidin-1-yl)-quinolin-6-yl]-propionamide,
2-(4-Chloro-2-thienyloxy)-N-[4-methyl-2-(4-dimethylamino-piperidin-1-yl l)-quinolin-6-yl]-acetamide,
2-(5-Chloro-2-thienyloxy)-N-[4-methyl-2-(4-dimethylamino-piperidin-1-yl)-quinolin-6-yl]-acetamide,
2-(5-Chloro-3-thienyloxy)-N-[4-methyl-2-(4-dimethylamino-piperidin-1-yl)-quinolin-6-yl]-acetamide,
2-(5-Trifluoromethyl-3-thienyloxy)-N-[4-methyl-2-(4-dimethylamino-piperidin-1-yl)-quinolin-6-yl]-acetamide,
3-(4-Methyl-2-thienyl)-N-[4-methyl-2-(4-dimethylamino-piperidin-1-yl)-quinolin-6-yl]-propionamide,
3-(5-Methyl-2-thienyl)-N-[4-methyl-2-(4-dimethylamino-piperidin-1-yl)-quinolin-6-yl]-propionamide,
3-(5-Methyl-3-thienyl)-N-[4-methyl-2-(4-dimethylamino-piperidin-1-yl)-quinolin-6-yl]-propionamide,
3-(5-Trifluoromethoxy-3-thienyl)-N-[4-methyl-2-(4-dimethylamino-piperidin-1-yl)-quinolin-6-yl]-propionamide,
2-(4-Methyl-2-thienyloxy)-N-[4-methyl-2-(4-dimethylamino-piperidin-1-yl)-quinolin-6-yl]-acetamide,
2-(5-Methyl-2-thienyloxy)-N-[4-methyl-2-(4-dimethylamino-piperidin-1-yl)-quinolin-6-yl]-acetamide,
2-(5-Methyl-3-thienyloxy)-N-[4-methyl-2-(4-dimethylamino-piperidin-1-yl)-quinolin-6-yl]-acetamide,
2-(5-Trifluoromethoxy-3-thienyloxy)-N-[4-methyl-2-(4-dimethylamino-piperidin-1-yl)-quinolin-6-yl]-acetamide,
3-(4-Chloro-2-thienyl)-N-[4-methyl-2-(4-methyl-piperazin-1-yl)-quinazolin-6-yl]-propionamide,
3-(5-Chloro-2-thienyl)-N-[4-methyl-2-(4-methyl-piperazin-1-yl)-quinazolin-6-yl]-propionamide,
3-(5-Chloro-3-thienyl)-N-[4-methyl-2-(4-methyl-piperazin-1-yl)-quinazolin-6-yl]-propionamide,
3-(5-Trifluoromethyl-3-thienyl)-N-[4-methyl-2-(4-methyl-piperazin-1-yl)-quinazolin-6-yl]-propionamide,
2-(4-Chloro-2-thienyloxy)-N-[4-methyl-2-(4-methyl-piperazin-1-yl)-quinazolin-6-yl]-acetamide,
2-(5-Chloro-2-thienyloxy)-N-[4-methyl-2-(4-methyl-piperazin-1-yl)-quinazolin-6-yl]-acetamide,
2-(5-Chloro-3-thienyloxy)-N-[4-methyl-2-(4-methyl-piperazin-1-yl)-quinazolin-6-yl]-acetamide,
2-(5-Trifluoromethyl-3-thienyloxy)-N-[4-methyl-2-(4-methyl-piperazin-1-yl)-quinazolin-6-yl]-acetamide,
3-(4-Methyl-2-thienyl)-N-[4-methyl-2-(4-methyl-piperazin-1-yl)-quinazolin-6-yl]-propionamide,
3-(5-Methyl-2-thienyl)-N-[4-methyl-2-(4-methyl-piperazin-1-yl)-quinazolin-6-yl]-propionamide,
3-(5-Methyl-3-thienyl)-N-[4-methyl-2-(4-methyl-piperazin-1-yl)-quinazolin-6-yl]-propionamide,
3-(5-Trifluoromethoxy-3-thienyl)-N-[4-methyl-2-(4-methyl-piperazin-1-yl)-quinazolin-6-yl]-propionamide,
2-(4-Methyl-2-thienyloxy)-N-[4-methyl-2-(4-methyl-piperazin-1-yl)-quinazolin-6-yl]-acetamide,
2-(5-Methyl-2-thienyl)oxy-N-[4-methyl-2-(4-methyl-piperazin-1-yl)-quinazolin-6-yl]-acetamide,
2-(5-Methyl-3-thienyloxy)-N-[4-methyl-2-(4-methyl-piperazin-1-yl)-quinazolin-6-yl]-acetamide,
2-(5-Trifluoromethoxy-3-thienyloxy)-N-[4-methyl-2-(4-methyl-piperazin-1-yl)-quinazolin-6-yl]-acetamide,
3-(4-Chloro-2-thienyl)-N-[4-methyl-2-(4-pyrrolidino-piperidin-1-yl)-quinazolin-6-yl]-propionamide,
3-(5-Chloro-2-thienyl)-N-[4-methyl-2-(4-pyrrolidino-piperidin-1-yl l)-quinazolin-6-yl]-propionamide,
3-(5-Chloro-3-thienyl)-N-[4-methyl-2-(4-pyrrolidino-piperidin-1-yl)-quinazolin-6-yl]-propionamide,
3-(5-Trifluoromethyl-3-thienyl)-N-[4-methyl-2-(4-pyrrolidino-piperidin-1-yl)-quinazolin-6-yl]-propionamide,
2-(4-Chloro-2-thienyloxy)-N-[4-methyl-2-(4-pyrrolidino-piperidin-1-yl l)-quinazolin-6-yl]-acetamide,
2-(5-Chloro-2-thienyloxy)-N-[4-methyl-2-(4-pyrrolidino-piperidin-1-yl)-quinazolin-6-yl]-acetamide,
2-(5-Chloro-3-thienyloxy)-N-[4-methyl-2-(4-pyrrolidino-piperidin-1-yl)-quinazolin-6-yl]-acetamide,
2-(5-Trifluoromethyl-3-thienyloxy)-N-[4-methyl-2-(4-pyrrolidino-piperidin-1-yl)-quinazolin-6-yl]-acetamide,
3-(4-Methyl-2-thienyl)-N-[4-methyl-2-(4-pyrrolidino-piperidin-1-yl)-quinazolin-6-yl]-propionamide,
3-(5-Methyl-2-thienyl)-N-[4-methyl-2-(4-pyrrolidino-piperidin-1-yl)-quinazolin-6-yl]-propionamide,
3-(5-Methyl-3-thienyl)-N-[4-methyl-2-(4-pyrrolidino-piperidin-1-yl)-quinazolin-6-yl]-propionamide,
3-(5-Trifluoromethoxy-3-thienyl)-N-[4-methyl-2-(4-pyrrolidino-piperidin-1-yl)-quinazolin-6-yl]-propionamide,
2-(4-Methyl-2-thienyloxy)-N-[4-methyl-2-(4-pyrrolidino-piperidin-1-yl)-quinazolin-6-yl]-acetamide,
2-(5-Methyl-2-thienyloxy)-N-[4-methyl-2-(4-pyrrolidino-piperidin-1-yl)-quinazolin-6-yl]-acetamide,
2-(5-Methyl-3-thienyloxy)-N-[4-methyl-2-(4-pyrrolidino-piperidin-1-yl)-quinazolin-6-yl]-acetamide,
2-(5-Trifluoromethoxy-3-thienyloxy)-N-[4-methyl-2-(4-pyrrolidino-piperidin-1-yl)-quinazolin-6-yl]-acetamide,
3-(4-Chloro-2-thienyl)-N-[4-methyl-2-(4-dimethylamino-piperidin-1-yl)-quinazolin-6-yl]-propionamide,
3-(5-Chloro-2-thienyl)-N-[4-methyl-2-(4-dimethylamino-piperidin-1-yl l)-quinazolin-6-yl]-propionamide,
3-(5-Chloro-3-thienyl)-N-[4-methyl-2-(4-dimethylamino-piperidin-1-yl)-quinazolin-6-yl]-propionamide,
3-(5-Trifluoromethyl-3-thienyl)-N-[4-methyl-2-(4-dimethylamino-piperidin-1-yl)-quinazolin-6-yl]-propionamide,
2-(4-Chloro-2-thienyloxy)-N-[4-methyl-2-(4-dimethylamino-piperidin-1-yl l)-quinazolin-6-yl]-acetamide,
2-(5-Chloro-2-thienyloxy)-N-[4-methyl-2-(4-dimethylamino-piperidin-1-yl)-quinazolin-6-yl]-acetamide,
2-(5-Chloro-3-thienyloxy)-N-[4-methyl-2-(4-dimethylamino-piperidin-1-yl)-quinazolin-6-yl]-acetamide,
2-(5-Trifluoromethyl-3-thienyloxy)-N-[4-methyl-2-(4-dimethylamino-piperidin-1-yl)-quinazolin-6-yl]-acetamide,
3-(4-Methyl-2-thienyl)-N-[4-methyl-2-(4-dimethylamino-piperidin-1-yl)-quinazolin-6-yl]-propionamide,
3-(5-Methyl-2-thienyl)-N-[4-methyl-2-(4-dimethylamino-piperidin-1-yl)-quinazolin-6-yl]-propionamide,
3-(5-Methyl-3-thienyl)-N-[4-methyl-2-(4-dimethylamino-piperidin-1-yl)-quinazolin-6-yl]-propionamide,
3-(5-Trifluoromethoxy-3-thienyl)-N-[4-methyl-2-(4-dimethylamino-piperidin-1-yl)-quinazolin-6-yl]-propionamide,
2-(4-Methyl-2-thienyloxy)-N-[4-methyl-2-(4-dimethylamino-piperidin-1-yl)-quinazolin-6-yl]-acetamide,
2-(5-Methyl-2-thienyloxy)-N-[4-methyl-2-(4-dimethylamino-piperidin-1-yl)-quinazolin-6-yl]-acetamide,
2-(5-Methyl-3-thienyloxy)-N-[4-methyl-2-(4-dimethylamino-piperidin-1-yl)-quinazolin-6-yl]-acetamide,
2-(5-Trifluoromethoxy-3-thienyloxy)-N-[4-methyl-2-(4-dimethylamino-piperidin-1-yl)-quinazolin-6-yl]-acetamide.
2-(2,4-Dichloro-phenoxy)-N-[4-methyl-2-(4-methyl-piperazin-1-yl)-quinolin-6-yl]-acetamide,
3-(3-Chloro-phenyl)-N-[4-methyl-2-(4-methyl-piperazin-1-yl)-quinolin-6-yl]-acrylamide,
N-[4-Methyl-2-(4-methyl-piperazin-1-yl)-quinolin-6-yl]-3-p-tolyl-acrylamide
3-(2,5-Dimethoxy-phenyl)-N-[4-methyl-2-(4-methyl-piperazin-1-yl)-quinolin-6-yl]-acrylamide,
N-[4-Methyl-2-(4-methyl-piperazin-1-yl)-quinolin-6-yl]-2-m-tolyloxy-acetamide,
3-(2,3-Dimethoxy-phenyl)-N-[4-methyl-2-(4-methyl-piperazin-1-yl)-quinolin-6-yl]-acrylamide,
3-[4-(3-Methyl-butoxy)-phenyl]-N-[4-methyl-2-(4-methyl-piperazin-1-yl)-quinolin-6-yl]-acrylamide,
3-(4-Ethoxy-3-methoxy-phenyl)-N-[4-methyl-2-(4-methyl-piperazin-1-yl)-quinolin-6-yl]-acrylamide,
2-(2,4-Dichlorophenoxy)-N-[4-methyl-2-(4-methyl-piperazin-1-yl)-quinolin-6-yl]-propionamide,
N-[2-(4-Ethyl-piperazin-1-yl)-4-methyl-quinolin-6-yl]-3,4-dimethyl-benzamide,
N-[2-(4-Ethyl-piperazin-1-yl)-4-methyl-quinolin-6-yl]4-methoxy-benzamide,
4-Butyl-N-[2-(4-ethyl-piperazin-1-yl)-4-methyl-quinolin-6-yl]-benzamide,
5-Bromo-furan-2-carboxylic acid [2-(4-ethyl-piperazin-1-yl)-4-methyl-quinolin-6-yl]-amide,
5-Chloro-N-[2-(4-ethyl-piperazin-1-yl)-4-methyl-quinolin-6-yl]-2-methoxy-benzamide,
N-[2-(4-Ethyl-piperazin-1-yl)-4-methyl-quinolin-6-yl]-3,5-dimethoxy-benzamide,
N-[2-(4-Ethyl-piperazin-1-yl)-4-methyl-quinolin-6-yl]-2-(3-methoxy-phenyl)-acetamide,
N-[2-(4-Ethyl-piperazin-1-yl)-4-methyl-quinolin-6-yl]-2,4-dimethoxy-benzamide,
N-[2-(4-Ethyl-piperazin-1-yl)-4-methyl-quinolin-6-yl]-3,4-dimethoxy-benzamide,
4-Bromo-N-[2-(4-ethyl-piperazin-1-yl)-4-methyl-quinolin-6-yl]-3-methoxy-benzamide,
2-(3,4-Dimethoxy-phenyl)-N-[2-(4-ethyl-piperazin-1-yl)-4-methyl-quinolin-6-yl]-acetamide,
3-(3-Chloro-phenyl)-N-[2-(4-ethyl-piperazin-1-yl)-4-methyl-quinolin-6-yl]-acrylamide,
3-(3-Chloro-phenyl)-N-[2-(4-ethyl-piperazin-1-yl)-4-methyl-quinolin-6-yl]-acrylamide,
N-[2-(4-Ethyl-piperazin-1-yl)-4-methyl-quinolin-6-yl]-3-p-tolyl-acrylamide,
2-(3,4-Dichloro-phenyl)-N-[2-(4-ethyl-piperazin-1-yl)-4-methyl-quinolin-6-yl]-acetamide,
N-[2-(4-Ethyl-piperazin-1-yl)-4-methyl-quinolin-6-yl]-2-(3-trifluoromethyl-phenyl)-acetamide,
N-[2-(4-Ethyl-piperazin-1-yl)-4-methyl-quinolin-6-yl]-3-(4-methoxy-phenyl)-propionamide,
3-(2,5-Dimethoxy-phenyl)-N-[2-(4-ethyl-piperazin-1-yl)-4-methyl-quinolin-6-yl]-acrylamide,
6-Chloro-N-[2-(4-ethyl-piperazin-1-yl)-4-methyl-quinolin-6-yl]-nicotinamide,
N-[2-(4-Ethyl-piperazin-1-yl)-4-methyl-quinolin-6-yl]-3-trifluoromethyl-benzamide,
N-[2-(4-Ethyl-piperazin-1-yl)-4-methyl-quinolin-6-yl]-3-trifluoromethyl-benzamide,
2-(4-Bromo-5-methyl-3-trifluoromethyl-pyrazol-1-yl)-N-[2-(4-ethyl-piperazin-1-yl)-4-methyl-quinolin-6-yl]-acetamide,
2-(4-Bromo-5-methyl-3-trifluoromethyl-pyrazol-1-yl)-N-[2-(4-ethyl-piperazin-1-yl)-4-methyl-quinolin-6-yl]-acetamide,
2-(4-Chloro-phenoxy)-N-[2-(4-ethyl-piperazin-1-yl)-4-methyl-quinolin-6-yl]-acetamide,
2-(4-Chloro-phenoxy)-N-[2-(4-ethyl-piperazin-1-yl)-4-methyl-quinolin-6-yl]-acetamide,
2-(4-Chloro-phenoxy)-N-[2-(4-ethyl-piperazin-1-yl)-4-methyl-quinolin-6-yl]-acetamide,
N-[2-(4-Ethyl-piperazin-1-yl)-4-methyl-quinolin-6-yl]-3-(4-isopropoxy-phenyl)-acrylamide,
N-[2-(4-Ethyl-piperazin-1-yl)-4-methyl-quinolin-6-yl]-3-(2-isopropoxy-phenyl)-acrylamide,
N-[2-(4-Ethyl-piperazin-1-yl)-4-methyl-quinolin-6-yl]-3-(2-isopropoxy-phenyl)-acrylamide,
N-[2-(4-Ethyl-piperazin-1-yl)-4-methyl-quinolin-6-yl]-3-(2-thioxo-benzooxazol-3-yl)-propionamide,
2-Thiophen-2-yl-quinoline-4-carboxylic acid [2-(4-ethyl-piperazin-1-yl)-4-methyl-quinolin-6-yl]-amide,
Pyrazine-2-carboxylic acid [2-(4-ethyl-piperazin-1-yl)-4-methyl-quinolin-6-yl]-amide,
N-[2-(4-Ethyl-piperazin-1-yl)-4-methyl-quinolin-6-yl]-2,6-difluoro-benzamide,
2-Methyl-N-[4-methyl-2-(4-methyl-piperazin-1-yl)-quinolin-6-yl]-benzamide,
2-Chloro-N-[4-methyl-2-(4-methyl-piperazin-1-yl)-quinolin-6-yl]-nicotinamide,
N-[4-Methyl-2-(4-methyl-piperazin-1-yl)-quinolin-6-yl]-isonicotinamide,
Benzo[1,3]dioxole-5-carboxylic acid [4-methyl-2-(4-pyrimidin-2-yl-piperazin-1-yl)-quinolin-6-yl]-amide,
N-[4-Methyl-2-(4-methyl-piperazin-1-yl)-quinolin-6-yl]-nicotinamide,
N-[4-Methyl-2-(4-pyrimidin-2-yl-piperazin-1-yl)-quinolin-6-yl]-3-trifluoromethyl-benzamide,
3-Dimethylamino-N-[4-methyl-2-(4-pyrimidin-2-yl-piperazin-1-yl)-quinolin-6-yl]-benzamide,
4-Ethoxy-N-[4-methyl-2-(4-pyrimidin-2-yl-piperazin-1-yl)-quinolin-6-yl]-benzamide,
2-Chloro-4-fluoro-N-[4-methyl-2-(4-methyl-piperazin-1-yl)-quinolin-6-yl]-benzamide,
2-Fluoro-N-[4-methyl-2-(4-methyl-piperazin-1-yl)-quinolin-6-yl]-benzamide,
4-tert-Butyl-N-[4-methyl-2-(4-methyl-piperazin-1-yl)-quinolin-6-yl]-benzamide,
4-Butyl-N-[4-methyl-2-(4-methyl-piperazin-1-yl)-quinolin-6-yl]-benzamide,
4-Fluoro-N-[4-methyl-2-(4-methyl-piperazin-1-yl)-quinolin-6-yl]-benzamide,
2-Methoxy-N-[4-methyl-2-(4-methyl-piperazin-1-yl)-quinolin-6-yl]-benzamide,
3,4,5-Trimethoxy-N-[4-methyl-2-(4-methyl-piperazin-1-yl)-quinolin-6-yl]-benzamideN-[4-Methyl-2-(4-methyl-piperazin-1-yl)-quinolin-6-yl]-2-nitro-benzamide,
3-Chloro-N-[4-methyl-2-(4-methyl-piperazin-1-yl)-quinolin-6-yl]-benzamide,
2-Chloro-N-[4-methyl-2-(4-methyl-piperazin-1-yl)-quinolin-6-yl]-benzamide,
4-Bromo-3-methoxy-N-[4-methyl-2-(4-methyl-piperazin-1-yl)-quinolin-6-yl]-benzamide,
4-Diethylamino-N-[4-methyl-2-(4-methyl-piperazin-1-yl)-quinolin-6-yl]-benzamide,
2-Chloro-N-[4-methyl-2-(4-methyl-piperazin-1-yl)-quinolin-6-yl]-4-nitro-benzamide,
N-[4-Methyl-2-(4-methyl-piperazin-1-yl)-quinolin-6-yl]-3-nitro-benzamide,
2,4-Dimethoxy-N-[4-methyl-2-(4-methyl-piperazin-1-yl)-quinolin-6-yl]-benzamide,
3,4-Dimethoxy-N-[4-methyl-2-(4-methyl-piperazin-1-yl)-quinolin-6-yl]-benzamide,
3-Methyl-N-[4-methyl-2-(4-methyl-piperazin-1-yl)-quinolin-6-yl]4-nitro-benzamide,
Pyrazine-2-carboxylic acid [4-methyl-2-(4-methyl-piperazin-1-yl)-quinolin-6-yl]-amide,
3-Methoxy-N-[4-methyl-2-(4-methyl-piperazin-1-yl)-quinolin-6-yl]-benzamide,
5-Chloro-2-methoxy-N-[4-methyl-2-(4-methyl-piperazin-1-yl)-quinolin-6-yl]-benzamide,
2-Thiophen-2-yl-quinoline-4-carboxylic acid [4-methyl-2-(4-methyl-piperazin-1-yl)-quinolin-6-yl]-amide,
2-Ethoxy-N-[4-methyl-2-(4-methyl-piperazin-1-yl)-quinolin-6-yl]-benzamide,
3,4,5-Triethoxy-N-[4-methyl-2-(4-methyl-piperazin-1-yl)-quinolin-6-yl]-benzamide,
5-Bromo-2-chloro-N-[4-methyl-2-(4-methyl-piperazin-1-yl)-quinolin-6-yl]-benzamide,
2,3-Dimethoxy-N-[4-methyl-2-(4-methyl-piperazin-1-yl)-quinolin-6-yl]-benzamide,
3-Dimethylamino-N-[4-methyl-2-(4-methyl-piperazin-1-yl)-quinolin-6-yl]-benzamide,
6-Chloro-N-[4-methyl-2-(4-methyl-piperazin-1-yl)-quinolin-6-yl]-nicotinamide,
3-Fluoro-N-[4-methyl-2-(4-methyl-piperazin-1-yl)-quinolin-6-yl]-benzamide,
N-[4-Methyl-2-(4-methyl-piperazin-1-yl)-quinolin-6-yl]-3-trifluoromethyl-benzamide,
Benzo[1,3]dioxole-5-carboxylic acid [4-methyl-2-(4-methyl-piperazin-1-yl)-quinolin-6-yl]-amide,
Thiophene-2-carboxylic acid [4-methyl-2-(4-methyl-piperazin-1-yl)-quinolin-6-yl]-amide,
Furan-2-carboxylic acid [4-methyl-2-(4-methyl-piperazin-1-yl)-quinolin-6-yl]-amide,
5-Bromo-furan-2-carboxylic acid [4-methyl-2-(4-methyl-piperazin-1-yl)-quinolin-6-yl]-amide,
2-(4-Chloro-phenyl)-N-[2-(4-ethyl-piperazin-1-yl)-4-methyl-quinolin-6-yl]-acetamide,
N-[2-(4-Ethyl-piperazin-1-yl)-4-methyl-quinolin-6-yl]-2-(4-nitro-phenyl)-acetamide,
N-[2-(4-Ethyl-piperazin-1-yl)-4-methyl-quinolin-6-yl]-2-(4-methoxy-phenyl)-acetamide,
N-[2-(4-Ethyl-piperazin-1-yl)-4-methyl-quinolin-6-yl]-2-p-tolyl-acetamide,
2-(2-Chloro-6-fluoro-phenyl)-N-[2-(4-ethyl-piperazin-1-yl)-4-methyl-quinolin-6-yl]-acetamide,
2-(3-Methoxy-phenyl)-N-[4-methyl-2-(4-methyl-piperazin-1-yl)-quinolin-6-yl]-acetamide,
N-[2-(4-Ethyl-piperazin-1-yl)-4-methyl-quinolin-6-yl]-2-pyridin-3-yl-acetamide,
N-[2-(4-Ethyl-piperazin-1-yl)-4-methyl-quinolin-6-yl]-2-(2-methoxy-phenyl)-acetamide,
2-(4-Methoxy-phenyl)-N-[4-methyl-2-(4-methyl-piperazin-1-yl)-quinolin-6-yl]-acetamide,
N-[4-Methyl-2-(4-methyl-piperazin-1-yl)-quinolin-6-yl]-2-m-tolyl-acetamide,
2-(2-Methoxy-phenyl)-N-[4-methyl-2-(4-methyl-piperazin-1-yl)-quinolin-6-yl]-acetamide,
2-(3,4-Dichloro-phenyl)-N-[4-methyl-2-(4-methyl-piperazin-1-yl-quinolin-6-yl]-acetamide,
2-(3-Methoxy-phenyl)-N-[4-methyl-2-(4-methyl-piperazin-1-yl)-quinolin-6-yl]-acetamide,
2-(2-Bromo-phenyl)-N-[2-(4-ethyl-piperazin-1-yl)-4-methyl-quinolin-6-yl]-acetamide,
2-Cyclopentyl-N-[2-(4-ethyl-piperazin-1-yl)-4-methyl-quinolin-6-yl]-2-phenyl-acetamide,
N-[4-Methyl-2-(4-methyl-piperazin-1-y)-quinolin-6-yl]-2-(3-trifluoromethyl-phenyl)-acetamide,
3-(4-Bromo-phenyl)-N-[4-methyl-2-(4-pyrimidin-2-yl-piperazin-1-yl)-quinolin-6-yl]-acrylamide,
N-[2-(4-Ethyl-piperazin-1-yl)-4-methyl-quinolin-6-yl]-3-(4-methoxy-phenyl)-propionamide,
2-(2,4-Dichloro-phenoxy)-N-[2-(4-ethyl-piperazin-1-yl)-4-methyl-quinolin-6-yl]-propionamide,
2-(4-Chloro-phenoxy)-N-[2-(4-ethyl-piperazin-1-yl)-4-methyl-quinolin-6-yl]-acetamide,
N-[2-(4-Ethyl-piperazin-1-yl)-4-methyl-quinolin-6-yl]-2-(pyridin-4-ylsulfanyl)-acetamide,
3-(2,5-Dimethoxy-phenyl)-N-[4-methyl-2-(4-pyrimidin-2-yl-piperazin-1-yl)-quinolin-6-yl]-acrylamide,
N-[2-(4-Ethyl-piperazin-1-yl)-4-methyl-quinolin-6-yl]-2-m-tolyloxy-acetamide,
3-(3,4-Dimethoxy-phenyl)-N-[4-methyl-2-(4-pyrimidin-2-yl-piperazin-1-yl)-quinolin-6-yl]-acrylamide,
3-(4-Methoxy-phenyl)-N-[4-methyl-2-(4-pyrimidin-2-yl-piperazin-1-yl)-quinolin-6-yl]-propionamide,
3-Benzo[1,3]dioxol-5-yl-N-[4-methyl-2-(4-pyrimidin-2-yl-piperazin-1-yl)-quinolin-6-yl]-acrylamide,
3-(2-Isopropoxy-phenyl)-N-[4-methyl-2-(4-pyrimidin-2-yl-piperazin-1-yl)-quinolin-6-yl]-acrylamide,
3-(4-Methoxy-phenyl)-N-[4-methyl-2-(4-pyrimidin-2-yl-piperazin-1-yl)-quinolin-6-yl]-acrylamide,
2-(4-Chloro-phenoxy)-N-[4-methyl-2-(4-pyrimidin-2-yl-piperazin-1-yl)-quinolin-6-yl]-acetamide,
2-Methyl-N-[4-methyl-2-(4-pyrimidin-2-yl-piperazin-1-yl)-quinolin-6-yl]-3-phenyl-acrylamide,
N-[4-Methyl-2-(4-pyrimidin-2-yl-piperazin-1-yl)-quinolin-6-yl]-2-(pyridin-4-ylsulfanyl)-acetamide,
3-(4-Ethoxy-3-methoxy-phenyl)-N-[4-methyl-2-(4-pyrimidin-2-yl-piperazin-1-yl)-quinolin-6-yl]-acrylamide,
3-[4-(3-Methyl-butoxy)-phenyl]-N-[4-methyl-2-(4-pyrimidin-2-yl-piperazin-1-yl)-quinolin-6-yl]-acrylamide,
3-(4-Isopropoxy-phenyl)-N-[4-methyl-2-(4-pyrimidin-2-yl-piperazin-1-yl)-quinolin-6-yl-acrylamide,
3-(2,3-Dimethoxy-phenyl)-N-[4-methyl-2-(4-pyrimidin-2-yl-piperazin-1-yl)-quinolin-6-yl]-acrylamide,
N-[4-Methyl-2-(4-pyrimidin-2-yl-piperazin-1-yl)-quinolin-6-yl]-2-m-tolyloxy-acetamide,
3-(3,4-Dimethoxy-phenyl)-N-[4-methyl-2-(4-methyl-piperazin-1-yl)-quinolin-6-yl]-acrylamide,
3-(2-Chloro-phenyl)-N-[4-methyl-2-(4-methyl-piperazin-1-yl)-quinolin-6-yl]-acrylamide,
2-Methanesulfonyl-1,2,3,4-tetrahydro-isoquinoline-3-carboxylic acid [4-methyl-2-(4-pyrimidin-2-yl-piperazin-1-yl)-quinolin-6-yl]-amide,
N-[4-Methyl-2-(4-methyl-piperazin-1-yl)-quinolin-6-yl]-3-p-tolyl-acrylamide,
3-(2-Ethoxy-phenyl)-N-[4-methyl-2-(4-pyrimidin-2-yl-piperazin-1-yl)-quinolin-6-yl]-acrylamide,
N-[4-Methyl-2-(4-pyrimidin-2-yl-piperazin-1-yl)-quinolin-6-yl]-2-(2-oxo-benzooxazol-3-yl)-acetamide,
N-[4-Methyl-2-(4-methyl-piperazin-1-yl)-quinolin-6-yl]-3-(3,4,5-trimethoxy-phenyl)-acrylamide,
2-(2,4-Dichloro-phenoxy)-N-[4-methyl-2-(4-methyl-piperazin-1-yl)-quinolin-6-yl]-acetamide,
3-(3-Chloro-phenyl)-N-[4-methyl-2-(-methyl-piperazin-1-yl)-quinolin-6-yl]-acrylamide,
2-(2,4-Dichloro-phenoxy)-N-[4-methyl-2-(4-pyrimidin-2-yl-piperazin-1-yl)-quinolin-6-yl]-propionamide,
3-[4-(3-Methyl-butoxy)-phenyl]-N-[4-methyl-2-(4-methyl-piperazin-1-yl)-quinolin-6-yl]-acrylamide,
3-Benzo[1,3]dioxol-5-yl-N-[4-methyl-2-(4-methyl-piperazin-1-yl)-quinolin-6-yl]-acrylamide,
3-(4-Methoxy-phenyl)-N-[4-methyl-2-(4-methyl-piperazin-1-yl)-quinolin-6-yl]-propionamide,
N-[4-Methyl-2-(4-methyl-piperazin-1-yl)-quinolin-6-yl]-3-pyridin-3-yl-acrylamide,
2-Methyl-N-[4-methyl-2-(4-methyl-piperazin-1-yl)-quinolin-6-yl]-3-phenyl-acrylamide,
2-(4-Chloro-phenoxy)-N-[4-methyl-2-(4-methyl-piperazin-1-yl)-quinolin-6-yl]-acetamide,
3-(4-Methoxy-phenyl)-N-[4-methyl-2-(4-methyl-piperazin-1-yl)-quinolin-6-yl]-acrylamide,
3-(2,5-Dimethoxy-phenyl)-N-[4-methyl-2-(4-methyl-piperazin-1-yl)-quinolin-6-yl]-crylamide,
N-[4-Methyl-2-(4-methyl-piperazin-1-yl)-quinolin-6-yl]-2-m-tolyloxy-acetamide,
3-(2,3-Dimethoxy-phenyl)-N-[4-methyl-2-(4-methyl-piperazin-1-yl)-quinolin-6-yl]-acrylamide,

N-[4-Methyl-2-(4-methyl-piperazin-1-yl)-quinolin-6-yl]-2-(2-oxo-benzooxazol-3-yl)-acetamide,
3-(2-Isopropoxy-phenyl)-N-[4-methyl-2-(4-methyl-piperazin-1-yl)-quinolin-6-yl]-acrylamide,
3-(2-Isopropoxy-phenyl)-N-[4-methyl-2-(4-methyl-piperazin-1-yl)-quinolin-6-yl]-acrylamide,
3-(4-Ethoxy-3-methoxy-phenyl)-N-[4-methyl-2-(4-methyl-piperazin-1-yl)-quinolin-6-yl]-acrylamide,
2-(2,4-Dichloro-phenoxy)-N-[4-methyl-2-(4-methyl-piperazin-1-yl)-quinolin-6-yl]-propionamide,
3-Furan-2-yl-N-[4-methyl-2-(4-methyl-piperazin-1-yl)-quinolin-6-yl]-acrylamide,
3-(5-Methyl-furan-2-yl)-N-[4-methyl-2-(4-methyl-piperazin-1-yl)-quinolin-6-yl]-acrylamide,
3-(5-Methyl-furan-2-yl)-N-[4-methyl-2-(4-pyrimidin-2-yl-piperazin-1-yl)-quinolin-6-yl]-acrylamide,
N-[4-Methyl-2-(4-methyl-piperazin-1-yl)-quinolin-6-yl]-3-(3-methyl-thiophen-2-yl)-acrylamide,
N-[4-Methyl-2-(4-pyrimidin-2-yl-piperazin-1-yl)-quinolin-6-yl]-3-thiophen-2-yl-acrylamide,
N-[4-Methyl-2-(4-methyl-piperazin-1-yl)-quinolin-6-yl]-3-thiophen-2-yl-acrylamide,
1-Benzo[1,3]dioxol-5-ylmethyl-3-[2-(4-ethyl-piperazin-1-yl)-4-methyl-quinolin-6-yl]-thiourea,
1-(2-Ethyl-phenyl)-3-[2-(4-ethyl-piperazin-1-yl)-4-methyl-quinolin-6-yl]-1-methyl-thiourea,
1-Ethyl-3-[2-(4-ethyl-piperazin-1-yl)-4-methyl-quinolin-6-yl]-1-(4-fluoro-phenyl)-thiourea,
1-[2-(4-Ethyl-piperazin-1-yl)-4-methyl-quinolin-6-yl]-3-furan-2-ylmethyl-thiourea,
1-[2-(4-Ethyl-piperazin-1-yl)-4-methyl-quinolin-6-yl]-3-(4-fluoro-benzyl)-thiourea,
1-[2-(4-Ethyl-piperazin-1-yl)-4-methyl-quinolin-6-yl]-3-(2-methoxy-benzyl)-thiourea,
1-[2-(4-Ethyl-piperazin-1-yl)-4-methyl-quinolin-6-yl]-3-thiophen-2-ylmethyl-thiourea,
1-(4-Ethoxy-phenyl)-1-ethyl-3-[2-(4-ethyl-piperazin-1-yl)-4-methyl-quinolin-6-yl]-thiourea,
1-Benzyl-3-[2-(4-ethyl-piperazin-1-yl)-4-methyl-quinolin-6-yl]-1-methyl-thiourea,
1-(4-Ethyl-phenyl)-3-[2-(4-ethyl-piperazin-1-yl)-4-methyl-quinolin-6-yl]-1-propyl-thiourea,
2-(2,4-Dichloro-phenoxy)-N-(2-dimethylaminomethyl-quinolin-6-yl)-acetamide,
2-(2,4-Dichloro-phenoxy)-N-{2-[(2-dimethylamino-ethyl)-methyl-amino]-quinolin-6-yl}-acetamide,
2-(2,4-Dichloro-phenoxy)-N-[2-(2-morpholin-4-yl-ethyl)-quinolin-6-yl]-acetamide,
2-(2,4-Dichloro-phenoxy)-N-[2-(2-dimethylamino-ethoxy)-quinolin-6-yl]-acetamide,
2-(2,4-Dichloro-phenoxy)-N-(2-[(1-methyl-pyrrolidin-2-ylmethyl)-amino]-quinolin-6-yl}acetamide,
N-[2-(4-Amino-butyl)-quinolin-6-yl]-2-(2,4-dichloro-phenoxy)-acetamide,
2-(2,4-Dichloro-phenoxy)-N-(6-dimethylaminomethyl-5,6,7,8-tetrahydro-acridin-2-yl)-acetamide,
2-(2,4-Dichloro-phenoxy)-N-{2-[2-(3,4-dihydro-1H-isoquinolin-2-yl)-cyclopentylmethyl]-quinolin-6-yl}-acetamide,
1-(4-Methyl-benzyl)-3-[2-(4-methyl-piperazin-1-yl)-quinolin-6-yl]-urea,
1-(4-Fluoro-benzoyl)-1-methyl-3-[2-(4-methyl-piperazin-1-yl)-quinolin-6-yl]-urea,
1-[2-(4-Methyl-piperazin-1-yl)-quinolin-6-yl]-ethanesulfonic acid [1-(4-chloro-phenyl)-ethyl]-amide,
2,3-Dihydro-benzo[1,4]dioxine-2-carboxylic acid [2-(4-methyl-piperazin-1-yl)-quinolin-4-yl]-amide,
2-Phenyl-propene-1-sulfonic acid [2-(4-methyl-piperazin-1-yl)-quinolin-6-yl]-amide,
Thiophene-2-carboxylic acid methyl-[2-(4-methyl-piperazin-1-yl)-quinoline-6-carbonyl]-amide,
2-[4-(3-Acetylamino-benzyl)-piperazin-1-yl]-quinoline-6-carboxylic acid [1-(4-fluoro-phenyl)-propyl]-methyl-amide,
C-(4-Chloro-phenoxy)-N-methyl-N-[2-(4-methyl-piperazin-1-yl)-quinolin-6-yl]-methanesulfonamide,
1-[2-(4-Methyl-piperazin-1-yl)-quinolin-6-yl]-3-(3-trifluoromethoxy-phenyl)-urea,
2-Phenyl-propene-1-sulfonic acid [4-methyl-2-(4-methyl-piperazin-1-yl)-quinolin-6-y]-amide,
1-[4-Methyl-2-(4-methyl-piperazin-1-yl)-quinolin-6-yl]-3-(4-phenoxy-phenyl)-urea,
1-[4-Methyl-2-(4-methyl-piperazin-1-yl)-quinolin-6-yl]-3-(4-trifluoromethoxy-phenyl)-urea,
1-(5-Methoxy-pyrazin-2-yl)-3-[4-methyl-2-(4-methyl-piperazin-1-yl)-quinolin-6-yl]-urea,
1-[4-Methyl-2-(4-methyl-piperazin-1-yl)-quinolin-6-yl]-3-pyridin-3-ylmethyl-urea,
1-[4-Methyl-2-(4-methyl-piperazin-1-yl)-quinolin-6-yl]-3-thiophen-2-ylmethyl-urea,
2-(1H-Indol-3-yl)-N-[4-methyl-2-(4-methyl-piperazin-1-yl)-quinolin-6-yl]-acetamide,
3,4-Dihydro-1H-isoquinoline-2-carboxylic acid [4-methyl-2-(4-propyl-piperazin-1-yl)-quinolin-6-yl]-amide,
5-Chloro-2,3-dihydro-benzofuran-2-carboxylic acid [4-methyl-2-(4-methyl-piperazin-1-yl)-quinolin-6-yl]-amide,
2-(4-Methyl-piperazin-1-yl)-6-[(2-phenyl-cyclopropanecarbonyl)-amino]-quinoline-4-carboxylic acid dimethylamide,
N-[2-(2-Diethylamino-ethylsulfanyl)-4-methyl-quinolin-6-yl]-3-furan-2-yl-acrylamide,
N-[2-(4-Ethyl-piperazin-1-yl)-4-methyl-quinolin-6-yl]-2-(1H-indol-3-yl)-2-oxo-acetamide,
3-(2,4-Dichloro-phenyl)-N-(4-methyl-2-morpholin-4-yl-quinolin-6-yl)-acrylamide,
3-Benzofuran-2-yl-N-[2-(3-dimethylamino-piperidin-1-yl)-4-methyl-quinolin-6-yl]-acrylamide,
6-Methyl-4-oxo-chroman-2-carboxylic acid {2-[(3-acetylamino-benzyl)-(2-dimethylamino-ethyl)-amino]-4-methyl-quinolin-6-yl}-amide,
N-[2-(4-Benzyl-piperazin-1-yl)-4-methyl-quinolin-6-yl]-3-(4-trifluoromethoxy-phenyl)-acrylamide,
6-Chloro-4-oxo-chroman-2-carboxylic acid [4-methyl-2-(3-pyrrolidin-1-yl-azepan-1-yl)-quinolin-6-yl]-amide,
6,8-Dichloro-4-oxo-chroman-2-carboxylic acid {2-[ethyl-(3-piperidin-1-yl-propyl)-amino]-4-methyl-quinolin-6-yl}-amide,
4-Oxo-chroman-2-carboxylic acid [2-(4-dimethylamino-piperidin-1-yl)-4-methyl-quinolin-6-yl]-amide,
Benzo[b]thiophene-3-carboxylic acid {4-methyl-2-[methyl-(2-pyrrolidin-1-yl-ethyl)-amino]-quinolin-6-yl}amide,
3-(2-Chloro-phenyl)-N-{2-[(9H-fluoren-9-yl)-methyl-amino]4-methyl-quinolin-6-yl}-acrylamide,
2-Phenyl-cyclopropanecarboxylic acid [2-(4-methyl-piperazin-1-yl)-4-phenyl-quinolin-6-yl]-amide,
N-{4-Ethyl-2-[methyl-(2-methylamino-cyclopentyl)-amino]-quinolin-6-yl}-3-phenyl-propionamide,
2-(4-Chloro-phenoxy)-N-[4-methyl-2-(4-methyl-3-phenyl-piperazin-1-yl)-quinolin-6-yl]-acetamide,
N-[2-(3-Amino-2-phenyl-pyrrolidin-1-yl)-4-methyl-quinolin-6-yl]-2-phenoxy-acetamide,
N-[2-(1-Ethyl-pyrrolidin-3-ylamino)-4-methyl-quinolin-6-yl]-2-methyl-3-phenyl-acrylamide,
N-[2-(2-Amino-ethylamino)-4-methyl-quinolin-6-yl]-2-phenylsulfanyl-acetamide,
N-[4-Methyl-2-(4-methyl-[1,4]diazepan-1-yl)-quinolin-6-yl]-2-pentafluorophenyloxy-acetamide
   Salts, Complexes or Solvates

The invention also relates to the compounds and their uses in the form of their physiologically acceptable salts, complexes, solvates or prodrugs.

When a compound or a compound for use according to the invention possesses a basic functional group it can form a salt with an inorganic or organic acid.

Examples of physiologically acceptable salts of the compounds according to the invention include salts with inorganic acids, salts with organic acids, and salts with basic or acidic amino acids.

Examples of salts with inorganic acids include salts with hydrochloric acid, hydrobromic acid, hydroiodic acid, nitric acid or nitrous acid (to form e.g. a nitrate or a nitrite), sulfuric acid (to form e.g., a H₂SO₃ salt, a sulfate or a H₂SO₅ salt) and phosphoric acid (to form e.g. a H₃PO₃ salt or a H₃PO₄ salt)

Examples of salts with organic acids include salts with formic acid, acetic acid, propionic acid, butyric acid, pentanoic acid, longer saturated or unsaturated fatty acids, oxalic acid, tartaric acid, malonic acid, succinic acid, citric acid, C₄H₈(COOH)₂, C₅H₁₀(COOH)₂, acrylic acid, crotonic acid, maleic acid, malic acid, fumaric acid, H₂CO₃, lactic acid, ascorbic acid, benzoic acid, salicylic acid and phthalic acid, pamoic acid, trifluoroacetic acid, methanesulfonic acid, benzenesulfonic acid, p-toluenesulfonic acid and 3-chlorobenzoic acid.

Examples of salts with acidic amino acids include salts with aspartic acid and glutamic acid.

Optical Isomers

When a compound or a compound for use according to the invention contains optical isomers, diastereomers or other stereroisomers these are included as a compound of the invention as well as the racemate, i.e. mixture of enantiomers. Each of them can be obtained by methods known by a person skilled in the art. For example the optical isomer can be obtained using an optically active synthetic intermediate, an asymmetric synthesis or subjecting the racemic mixture of the final product or a suitable intermediate to optical resolution in accordance with known methods such as, e.g., fractional recrystallisation method, chiral column method, diastereomer method etc.

Other Forms

The invention also encompasses a compound or the use of a compound in amorphous, any polymorphous or any crystalline form.

Disorders

The compounds or the compounds for use according to the invention can be used in medicine and to modulate the activity of a MCH receptor. The compounds may be used as agents for preventing or treating diseases caused by or involving a melanin-concentrating hormone, i.e. they are useful for treating or preventing a MCH or MCH receptor related disorder or abnormality in a subject such as, e.g., an animal or a mammal such as, e.g., a human.

The compounds or the compounds for use according to the invention may have antagonistic, inverse agonistic, agonistic or allosteric activity against a MCH receptor, normally antagonistic activity.

In the present context an agonist is defined as a compound that increases the functional activity of a MCH receptor (e.g. the signal transduction through a receptor). The term “agonist” includes partial agonist, i.e. which increases the functional activity of the receptor to a submaximal level. An inverse agonist (or negative antagonist) is defined as a compound that decreases the basal functional activity of a MCH receptor.

An allosteric compound is defined as a compound that enhances or diminishes the effects of other receptor ligands.

An antagonist is defined as a compound that decreases the functional activity of a MCH receptor either by inhibiting the action of an agonist or by its own intrinsic activity.

The MCH receptors mentioned in the invention include MCH1 and MCH2 receptors. It also includes MCH receptors having at least about 80% such as, e.g. at least about 85% or at least about 90% homology to the amino acid sequences CTLITAMDAN or CTIITSLDTC.

The MCH receptors may be an animal or a mammalian or non-mammalian receptor, such as a human receptor.

Increasing or decreasing the activity of a MCH receptor such as, e.g. a MCH1 receptor alleviates a MCH-related disorder or abnormality. In specific embodiments the disorder is a steroid or pituitary hormone disorder, an epinephrine release disorder, a gastrointestinal disorder, a cardiovascular disorder, an electrolyte balance disorder, hypertension, diabetes, a respiratory disorder, asthma, a reproductive function disorder, a muscoskeletal disorder, a neuroendocrine disorder, a cognitive disorder, a memory disorder such as, e.g., Alzheimer's disease, a sensory modulation and transmission disorder, a motor coordination disorder, a sensory integration disorder, a motor integration disorder, a dopaminergic function disorder such as, e.g. Parkinson's disease, a sensory transmission disorder, an olfaction disorder, a sympathetic innervation disorder, an affective disorder such as, e.g. depression, a stress-related disorder, a fluid-balance disorder, a urinary disorder such as, e.g., urinary incontinence, a seizure disorder, pain, psychotic behaviour such as, e.g., schizophrenia, morphine or opioid tolerance, opiate addiction or migraine.

More specifically, the compounds of the invention are useful for the treatment or prevention of feeding disorders such as, e.g., overweight, adiposity, obesity and bulimia (e.g. malignant mastocytosis, exogeneous obesity, hyperinsulinar obesity, hyperplasmic obesity, hypophyseal adposity, hypoplasmic obesity, hypophysal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity etc.), hyperfagia, emotional disorders, dementia or hormonal disorders.

In the present context the term body mass index or BMI is defined as body weight (kg)/height² (m²), and the term overweight is intended to indicate a BMI in a range from about 25 to about 29.9, whereas obesity is intended to indicate a BMI, which is at least about 30.

A compound of the invention is also useful as an agent for preventing or treating lifestyle diseases such as, e.g., diabetes, diabetic complications (e.g. retinopathy, neuropathy, nephropathy etc.), arteriosclerosis and gonitis.

The present invention further relates to a cosmetic method for reducing overweight and/or for treating of and/or preventing overweight, bulimia, bulimia nervosa, obesity and/or complications thereto, the method comprising administering to an animal such as, e.g. a human in need thereof, an effective amount of a compound according to the invention

The invention also relates to a method for the treatment and/or prophylaxis of diseases caused by a melanin-concentrating hormone, the method comprising administering to a mammal in need thereof an efficient amount of a compound according to the invention.

A mentioned above, the MCH-related disorders may be a feeding disorder. Accordingly, the invention relates to a method for the treatment and/or prophylaxis of diseases caused by feeding disorders, the method comprising administering to a mammal in need thereof an efficient amount of a compound according to the invention.

The invention also relates to a method for modifying the feeding behaviour of a mammal, the method comprising administering to a mammal in need thereof an efficient amount of a compound according to the invention.

Furthermore, the invention relates to a method for the reduction of body mass, the method comprising administering to a mammal in need thereof an efficient amount of a compound according to the invention.

Moreover, the invention relates to a method for the treatment and/or prophylaxis of Syndrome X (metabolic syndrome) or any combination of obesity, insulin resistance, dyslipidemia, impaired glucose tolerance and hypertension, the method comprising administering to a mammal in need thereof an efficient amount of a compound according to the invention.

Another aspect of the invention is a method for the treatment and/or prophylaxis of Type II diabetes or Non Insulin Dependent Diabetes Mellitus (NIDDM), the method comprising administering to a mammal in need thereof an efficient amount of a compound according to the invention.

A still further aspect of the invention is a method for the treatment and/or prophylaxis of bulimia, bulimia nervosa and/or obesity, the method comprising administering to a mammal in need thereof an efficient amount of a compound according to the invention.

Moreover, the invention relates to a method for the treatment and/or prophylaxis of depression and/or anxiety, the method comprising administering to a mammal in need thereof an efficient amount of a compound according to the invention.

Selectivity

As mentioned above, the invention relates to a group of compounds displaying a reduced propensity to block HERG channels. A prolongation of the QT interval measured at the electrocardiogram (ECG) reflects a prolongation of cardiac ventricular repolarization. Excessive prolongation of the QT interval can be proarrhythmic and degenerate into a potentially fatal ventricular arrhythmia known as torsade de pointes (TdP).

Drug-induced prolongation of the QT interval has become a public health concern and attracted considerable regulatory and clinical attention since several non-cardiovascular drugs already on the market have been recognized to have a tendency to produce QT interval prolongation and/or TdP. Drug-induced QT prolongation is mainly associated with inhibition of HERG channels. Experimental data indicates that HERG channels underlie I(Kr), an important K⁺ current component in the repolarization of myocardial cells and the inherited Long QT syndrome type 2 (LQT2) is due to mutations in HERG. Inhibition of HERG channels by drugs intended for non-cardiovascular use is therefore considered as an adverse effect. It has been surprisingly found that compounds as described herein which contain a cyclic nitrogen-containing chain (Eastern portion) have unexpectedly improved properties with respect to HERG over those without a cyclic Eastern portion. Such compounds of interest should have a Ki value above 1 μM, such as e.g. above 2 μM, above 3 μM, above 5 μM, above 10 μM, above 25 μM in the protocol described in the examples so as to avoid the adverse effects associated with inhibition of HERG. Such improvements may also occur in those compounds in which the nitrogen-containing chain (Eastern portion) does not have a cyclic structure.

Solubility

As discussed, the compounds of the present invention have properties which are favourable with regard to pharmaceutical formulation and bioavailability. These include a sufficient aqueous solubility of the compounds provided by a basic aliphatic nitrogen. Solubility of drug substances might lead to an insufficient bio-availability even if no other limitations such as poor permeability or extensive first-pass metabolism are at hand. The finding that introduction of a nitrogen atom in the Eastern portion enhance the solubility of said compounds is supported by the methods given in the Examples. Compounds of interest according to this invention are those which have solubility of at least 25 μM, such as e.g. at least 50 μM, at least 75 μM, at least 100 μM, at least 125 μM, at least 150 μM, at least 200 μM in the experimental method described in the Examples. An additional factor which may be used to distinguish the compounds of the invention is that their solubility is increased by a factor of at least 2, such as e.g. at least 3, at least 5, at least 10, at least 15, at least 20, at least 30, at least 50, over comparable compounds which do not contain such a nitrogen group (e.g. those which contain a morpholine group). It is important that the remainder of the molecule remains unchanged (i.e. comparing “like with like”).

Pharmaceutical Compositions

The compounds or the compounds for use in the methods according to the invention are normally presented in the form of a pharmaceutical or a cosmetic composition comprising the specific compound or a physiologically acceptable salt thereof together with one or more physiologically acceptable excipients.

The compounds may be administered to the animal including a mammal such as, e.g., a human by any convenient administration route such as, e.g., the oral, buccal, nasal, ocular, pulmonary, topical, transdermal, vaginal, rectal, ocular, parenteral (including inter alia subcutaneous, intramuscular, and intravenous), route in a dose that is effective for the individual purposes. A person skilled in the art will know how to chose a suitable administration route.

The pharmaceutical or cosmetic composition comprising a compound according to the invention may be in the form of a solid, semi-solid or fluid composition.

The solid composition may be in the form of tablets such as, e.g. conventional tablets, effervescent tablets, coated tablets, melt tablets or sublingual tablets, pellets, powders, granules, granulates, particulate material, solid dispersions or solid solutions.

A semi-solid form of the composition may be a chewing gum, an ointment, a cream, a liniment, a paste, a gel or a hydrogel.

The fluid form of the composition may be a solution, an emulsion including nano-emulsions, a suspension, a dispersion, a liposomal composition, a spray, a mixture, a syrup or a aerosol.

Fluid compositions, which are sterile solutions or dispersions can be utilized by for example intraveneous, intramuscular, intrathecal, epidural, intraperitoneal or subcutaneous injection of infusion. The compounds may also be prepared as a sterile solid composition, which may be dissolved or dispersed before or at the time of administration using e.g. sterile water, saline or other appropriate sterile injectable medium.

Other suitable dosages forms of the pharmaceutical compositions according to the invention may be vagitories, suppositories, plasters, patches, tablets, capsules, sachets, troches, devices etc.

The dosage form may be designed to release the compound freely or in a controlled manner e.g. with respect to tablets by suitable coatings.

The pharmaceutical composition may comprise a therapeutically effective amount of a compound according to the invention.

The content of a compound of the invention in a pharmaceutical composition of the invention is e.g. from about 0.1 to about 100% w/w of the pharmaceutical composition.

The pharmaceutical or cosmetic compositions may be prepared by any of the method well known to a person skilled in pharmaceutical or cosmetic formulation.

In pharmaceutical or cosmetic compositions, the compounds are normally combined with a pharmaceutical excipient, i.e. a therapeutically inert substance or carrier.

The carrier may take a wide variety of forms depending on the desired dosage form and administration route.

The pharmaceutically or cosmetically acceptable excipients may be e.g. fillers, binders, disintegrants, diluents, glidants, solvents, emulsifying agents, suspending agents, stabilizers, enhancers, flavours, colors, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook.

Dosage

Optimal dosages to be administered may be determined by those skilled in the art, and will vary with the particular compound in use, the strength of the composition, the route of administration, the frequency of administration, the age, weight, gender, diet and condition of the subject to be treated and the condition being treated and the advancement of the disease condition etc.

Suitable dosages may be from about 0.001 mg to about 1 g such as, e.g. from about 0.005 to about 750 mg, from about 0.01 to about 500 mg, from about 0.05 to about 500 mg, from about 0.1 to about 250 mg, from about 0.1 to about 100 mg or from about 0.5 to about 50 mg.

The amounts can be divided into one or several doses for administration daily, every second day, weekly, every two weeks, monthly or with any other suitable frequency. Normally, the administration is daily.

A compound or a pharmaceutical composition according to the invention may be used in combination with other drug substances such as agents for treating disorders like e.g. diabetes, diabetes complications, obesity, hypertension, hyperlipidemia, arteriosclerosis, arthritis, anxiety, and/or depression etc.

Other Aspects of the Invention

The above-mentioned formulas encompass known as well as novel compounds. With respect to the novel compounds, the invention also relates to the compounds per se as well as to the use of the novel compounds in medicine especially the use in the prevention, treatment and/or diagnosis of the above-mentioned conditions. The details and particulars mentioned above apply mutatis mutandis to the other aspects of the invention.

### EXPERIMENTAL

Materials and Methods

Transfections and Tissue Culture—The cDNA encoding the human MCH-1 receptor was cloned from a human brain cDNA library and cloned into the eukaryotic expression vector pcDNA3.1 (Invitrogen). Assays were performed on transiently transfected COS-7 cells or stably transfected CHO (Chinese Hamster Ovary) cells, expressing the human MCH-1 receptor in pcDNA3.1. Stable MCH-1 receptor transfectants of CHO cells were obtained using 5 μg plasmid cDNA and a standard calcium phosphate transfection method (Johansen et al., 1990; Gether et al., 1992) with subsequent selection in 1 mg/ml G418 (Life Technology). Clones were screened by a MCH receptor radioligand binding assay (as described below). Stably transfected CHO cells were maintained in RPMI 1640 culture medium (invitrogen), supplemented with 10% fetal calf serum (Invitrogen), 100 U/ml penicillin, 100 μg/ml streptomycin (Life Technology), and 500 μg/ml G418 (Life Technology). COS-7 cells were grown in Dulbecco's modified Eagle's medium (DMEM) 1885 (Invitrogen) supplemented with 10% fetal calf serum, 100 U/ml penicillin, 100 μg/ml streptomycin, and were transiently transfected by a standard calcium phosphate transfection method (Johansen et al., 1990; Gether et al., 1992) two days before assay.

Radioligand Binding Assay—Transiently transfected COS-7 cells or stably transfected CHO cells, expressing human MCH-1 receptor were seeded in multi-well culture plates one day before the assay. The number of cells per well was determined by the apparent expression efficiency of the cell line aiming at 5-10% binding of the added radioligand. Cells were assayed by competition binding for 3 hours at room temperature using 15 pM [¹²⁵I]-MCH (Amersham Pharmacia Biotech) plus variable amounts of unlabeled ligand in 0.5 ml of a 25 mM Hepes buffer, pH 7.4, supplemented with 10 mM MgCl₂, 5 mM MnCl₂, 10 mM NaCl, 0.1% (w/v) bovine serum albumin (BSA), 100 μg/ml bacitracin. The assay was performed in duplicate. Nonspecific binding was determined as the binding in the presence of 1 μM MCH (Bachem). Binding data were analyzed and IC₅₀ values determined by non-linear regression using the Prism software (GraphPad software, San Diego). Values of the dissociation and inhibition constants (K_{d} and Kᵢ) were estimated from competition binding using the equations K_{d}=IC₅₀-L and Kᵢ=IC₅₀/(1+L/K_{d),} respectively, where L is the concentration of radioligand.

Phosphatidylinositol assay—To assay phosphatidylinositol turnover, transiently transfected COS-7 cells or stably transfected CHO cells, expressing human MCH-1 receptor (2×10⁵ cells/well) were incubated for 24 h with 5 μCi of (³H]-myo-inositol (Amersham Pharmacia Biotech) in 0.5 ml inositol-free culture medium. Cells were washed twice in PI-buffer: 20 mM HEPES, pH 7.4, supplemented with 140 mM NaCl, 5 mM KCl, 1 mM MgSO₄, 1 mM CaCl₂, 10 mM glucose, 0.02% (w/v) bovine serum; and were incubated in 0.5 ml PI-buffer supplemented with 10 mM LiCl at 37° C. for 45 min.

Phosphatidylinositol turnover was stimulated by submaximal concentrations of MCH, i.e. 10 nM in the presence of increasing amounts of ligand. The ligand was added 5 min. before adding the agonist (MCH). Cells were extracted with 10 mM ice-cold Formic acid, and the generated [³H]-inositol phosphates were purified on Bio-Rad AG 1-X8 anion-exchange resin. Determinations were made in duplicate. PI data were analyzed and IC₅₀ values determined by non-linear regression using the Prism software (GraphPad software, San Diego).

Scintillation Proximity Assay (SPA)—Measurement of [¹²⁵I]-MCH binding was performed in duplicates by incubating membranes and beads with tracer in the presences of various concentrations of test compounds (10⁻⁸ to 10⁻⁴M) in DMSO (3 μl) at room temperature for two hours. Membranes and beads were pre-incubated for 20 min. The binding buffer contained 50 mM Tris (pH 7.4), 8 mM MgCl2, 12% glycerol, 0.1% (w/v) bovine serum albumin (BSA), and protease inhibitors (Complete protease inhibitor cocktail tablets, Roche). A final [¹²⁵I]-MCH (2000 CVmmol; Amersham Pharmacia Biotech) concentration of 75.000 cpm/well (33.8 nCi) was applied and PEI-treated WGA-coupled PVT SPA beads, type B from Amersham Pharmacia Biotech were used at a final concentration of 0.4 mg/well. Moreover, CHO-K1 membranes expressing the hMCH receptor were purchased from Euroscreen (ES-370-M) and a final concentration of 2 μg/well were used. Binding data were analyzed and IC₅₀ values determined by non-linear regression using the Prism software (GraphPad software, San Diego). Values of the inhibition constant (Kᵢ) were estimated from competition binding using the equation Kᵢ=IC₅₀/(1+L/K_{d}), where L and K_{d} are the concentration and affinity constant, respectively, of the radioligand.

### REFERENCES

Gether, U., Marray, T., Schwartz, T. W, and Johansen, T. E. (1992). Stable expression of high affinity NK₁ (substance P) and NK₂ (neurokinin A) receptors but low affinity NK₃ (neurokinin B) receptors in transfected CHO cells. FEBS Lett., 296, 241-244.
Johansen, T. E., Schøller, M. S., Tolstoy, S. and Schwartz, T W (1990). Biosynthesis of peptide precursors and protease inhibitors using new constitutive and inducible eukaryotic expressions vectors. FEBS Lett., 267, 289-294.
   HERG Selectivity
   Method:

Plasmids: The human ERG (KCNH2) and KCNE1 were subcloned into the mammalian expression vectors pNS1 n and pNS1z, respectively, to give the plasmid constructs pNS1n_hERG and pNS1Z_minK.

HEK 293 cells stably expressing HERG+KCNE1: HEK 293 tissue culture cells were grown in DMEM (Dulbecco's Modified Eagle Medium) supplemented with 10% foetal calf serum at 37° C. in 5% CO₂. One day prior to transfection, 10⁶ cells were plated in a cell culture T25 flask. The following day, cells were transfected with equal amounts of the plasmids pNS1n_hERG and pNS1Z_mink using lipofection (Lipofectamin, Life Technologies). The cells were incubated with the lipofection mixture for 5 hours, rinsed with regular media, and grown for 72 hours before successfully co-transfected cells were selected in media supplemented with 0.25 mg/ml Zeocin and 0.5 mg/ml geneticin (G418) (Life Technologies). Single clones were picked and propagated in selection media until sufficient cells for freezing were available. Hereafter the cells were cultured in regular medium without selection agent. Expression of functional HERG channels was verified by patch-clamp measurements. After propagation, aliquots of the cells were frozen and since then experiments have been conducted on cells that have been passaged from 10-70 times since the transfection.

Whole-cell recordings: Cells plated on cover slips (Ø3.5 mm) were placed in a 15 μl perfusion chamber (flowrate ˜1 ml/min=full exchange every 1 sec). All experiments were performed at room temperature (20-22° C.) using an EPC-9 patch-clamp amplifier (HEKA-electronics, Lambrecht, Germany) connected to a Macintosh G4 computer via an ITC-16 interface. Data were stored directly on the hard disk and analysed by the IGOR software (Wavemetrics, Lake Oswego, USA). Series resistances as well as capacitance compensation were updated before each stimulus. The cell capacitances were 9.6-15.4 pF and the uncompensated series resistances were 1.5-2.2 MΩ in the seven experiments conducted in this study. A voltage-protocol simulating a human cardiac action potential (holding potential −90 mV, peak +30 mV, duration 315 mseconds) was applied to a cell every 5 seconds. A stable baseline current was obtained within a period of 1-2 minutes and a compound was then applied by changing to an extracellular solution containing the compound to be tested. After washout the next compound was added if the current returned to the baseline level.

Solutions: The intracellular (pipette) solution had the following composition (conc. in mM): 144 KCl, 10 EGTA, 1.42 MgCl₂, 5.17 CaCl₂, 4 Na-ATP and 10 HEPES (pH=7.2). The extracellular (bath) solution contained (conc. in mM): 144 KCl, 2 CaCl₂, 1 MgCl₂, 10 HEPES (pH=7.4).

Compounds: Compounds as 10 mM stock solutions in DMSO. All compounds were diluted at least 1000 fold in the extracellular solution. When tested the presence of up to 0.1% DMSO in the extracellular solution is without effect on the recorded currents.

Analysis: The peak of the tail-current obtained at the end of the action potential was measured as a function of time, and this analysis was exported to IGOR (Wavemetrics, Lake Oswego, USA), for further analysis. If a block of the HERG current was observed the blocker-induced decrease in current versus time was fitted to equation (1) to give the rate constants kₒₙ and k_{off}, and thereby Kᵢ
Iₜ=Iₒ*(1−(C/C+(k_{off}/kₒₙ)))*(1-exp(−(C*kₒₙ+k_{off})*t))) (1)
where:
Iₜ=current at time t k_{off}=off-rate
Iₒ=unblocked current kₒₙ=on-rate
C=drug concentration Kᵢ=k_{off}/kₒₙ

The Kᵢ value obtained is equal to the IC₅₀ value obtained from a fit to the Michaelis Menten equation. This can be visualized by solving equation (1) for t=∞. The analysis is based on the assumption that the drugs (D) interact with a receptor (R) on the HERG channels in the following way:

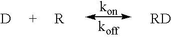

This is a simple bimolecular reaction, which integrated under non-equilibrium conditions are described by equation (1).

Verapamil was used as a reference compound with an average of Ki values being 2.3 μM. A series of drugs from different therapeutic classes have been tested using the same protocol (see table). From these data it appears that compounds that inhibit HERG channels with a Ki value below 1 μM in this particular protocol has a great risk of prolonging the QT interval in patients. E.g. Astemizole (0.08 μM) and terfenadine (0.11 μM) have been withdrawn from market.

Results:

Compounds in this invention typically inhibit HERG channels with Ki values above 1 μM. For example

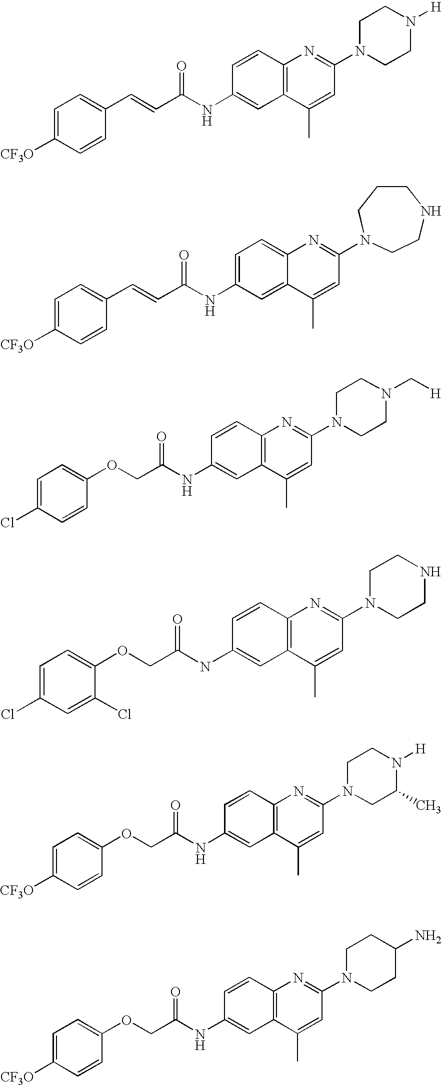

have Ki values between 1 and 5 μM.

TABLE
Kᵢ* values obtained with this protocol

| | | | | Risk |
|---|---|---|---|---|
| | | | Kᵢ in μM ± S.D. (#) | Group** |
| Anti- | (class) | | | |
| arrhyth- | Ic | Flecainide | 5.9 ± 1.2 (2) | 2 |
| mics | III | E-4031 | 0.2 ± 0.1 (20) | |
| | | Amiodarone | 2.8 ± 0.4 (2) | 1 |
| | | (±) sotalol | No effect at 100 (5) | 1 |
| | IV | Verapamil | 2.3 ± 0.8 (175) | |

| | | | |
|---|---|---|---|
| Antihistamines | Astemizole | 0.08 ± 0.02 (6) | Off |
| | | | market |
| | Terfenadine | 0.11 ± 0.03 (4) | Off |
| | | | market |
| | Cinnarizine | 0.36 ± 0.045 (3) | Not |
| | | | listed |
| | Meclizine | 1.2 (1) | Not |
| | | | listed |
| | Clemastine | 1.6 ± 1.0 (4) | Not |
| | | | listed |
| | Chlorcyclizine | 4.0 ± 1.4 (3) | Not |
| | | | listed |
| Antipsychotics | Pimozide | 0.06 ± 0.002 (2) | 1 |
| | Haloperidole | 0.17 ± 0.035 (3) | 1 |
| | Risperidone | 1.1 | 2 |
| | Chlorpromazine | 1.4 ± 0.57 (2) | 1 |
| | Perphenazine | 1.9 ± 0.28 (2) | Not |
| | | | listed |
| | Fluphenazine | 2.5 ± 0.57 (2) | Not |
| | | | listed |
| | Prochlorperazine | 4.0 ± 2.4 (3) | Not |
| | | | listed |
| | Cis-Thiothixene | 8.6 ± 2.7 (2) | Not |
| | | | listed |
| | Clozapine | 17 ± 4.1 (3) | Not |
| | | | listed |
| Antidepressives | Clomipramine | 6.5 ± 1.9 (2) | 4 |
| | Fluoxetine | 6.7 ± 2.2 (2) | 4 |
| | Amitriptyline | 18 ± 4.9 (2) | 4 |
| | Doxepine | 24 | 4 |
| | Amoxapine | 31 ± 7.1 (2) | 4 |
| | Imipramine | 31 | 4 |
| | Desipramine | 35 ± 9.2 (3) | 4 |
| | Trimipramine | No effect at 10 | 4 |
| Miscellaneous | Tamoxifen | 0.24 ± 0.08 (3) | 2 |
| | Bepridil | 0.36 ± 0.26 (2) | 1 |
| | Ketoconazole | 7.6 ± 1.6 (4) | 4 |

*Kᵢ values were calculated as described in the methods
**The risk groups have been obtained from the home page www.torsades.org and are defined as: 1: Drugs that are generally accepted by authorities to have a risk of Torsades de Pointes (TdP) 2: Drugs that in some reports may be associated with TdP but at this time lack substantial evidence

1: Drugs that are generally accepted by authorities to have a risk of Torsades de Pointes (TdP)
2: Drugs that in some reports may be associated with TdP but at this time lack substantial evidence
4: Drugs that, in some reports, have been weakly associated with TdP but that when used in usual dosages are unlikely to be a risk for TdP.
   Solubility
   Method:

The compound is dissolved as a 10 mM DMSO solution and added in small increments to 2.0 ml of a pH 7 phosphate buffer at room temperature. The additions of the DMSO solution are made with about one minute apart. The appearance of opalescence or precipitate is visually observed or measured via change in UV absorbance from light scattering.

Results:

The following compounds having a terminal aliphatic nitrogen in the side chain were found to have solubilities of about 75 μM or more. For example, the five compounds below have solubilities of about 75-100 μM according to this protocol.

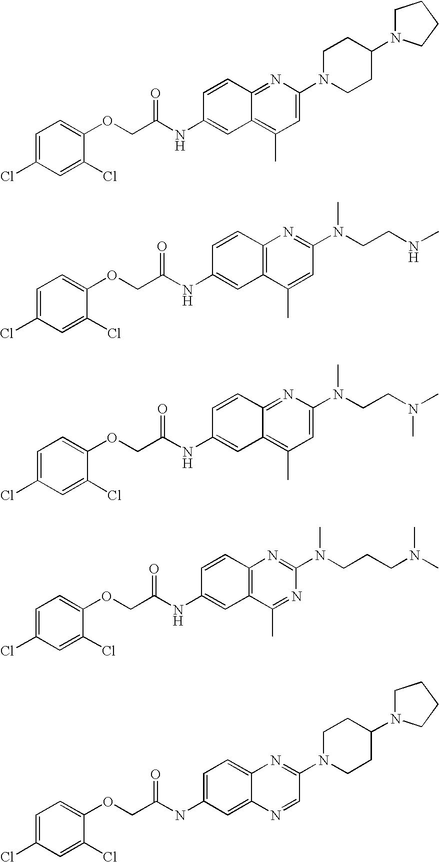

Similarily, the two compounds below also having a terminal aliphatic nitrogen in the side chain have solubilities of about 150 μM.

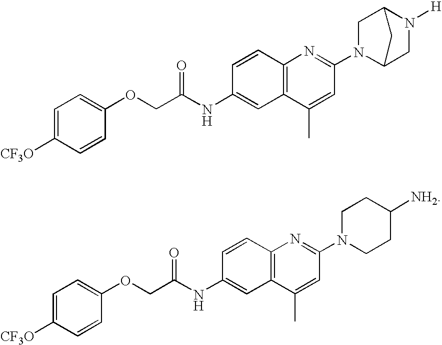

In contrast, the related di-chloro derivatives below lacking such an ionisable moiety displayed a solubility of about 5 μM or less.

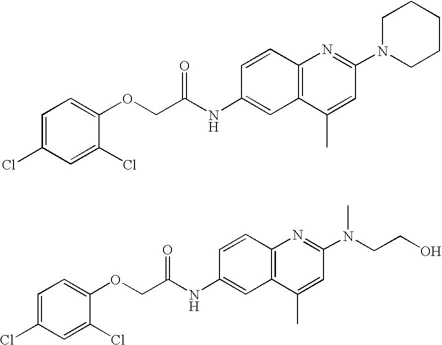

### SYNTHESIS EXAMPLES

General comments:

¹H NMR data are given either in full detail or with selected characteristic peaks. LC/MS was performed on an Agilent 1100-series instrument. LC/MS methods are as follows:

an20p10: Column: Agilent Zorbax Eclipse XDB-C18 (4.6×150 mm, 5μ); Flow: 0.8 mL/min; Gradient: 0-8 min: 20-95% MeCN in water, 8-10 min: 95% MeCN in water;

Modifier: 0.1% formic acid; MS-ionisation mode: API-ES (pos.).

an20p15: As an20p10, but Gradient: 0-10 min: 20-95% MeCN in water, 10-15 min 95% MeCN in water.

an05p7: Column: Waters XTerra MS C18 (2.1×5 mm, 5μ); Flow: 1.2 mL/min; Gradient: 0-4 min: 5-96% MeCN in water, 44.5 min: 96% MeCN in water, 4.5-6.5 min: 100% MeCN in water; 1% NH3 was added to the solvent as modifier; MS-ionisation mode: API-ES (pos.).

an07n7: As an05p7, but MS-ionisation mode: API-ES (neg.).

an10p8: Column: Waters XTerra MS C18 (2.1×5 mm, 5μ); Flow: 1.0 mL/min; Gradient: 0-5 min: 10-100% MeCN, 5-7.5 min: 100% MeCN; MS-ionisation mode: API-ES (pos.).
General Synthetic Route I:

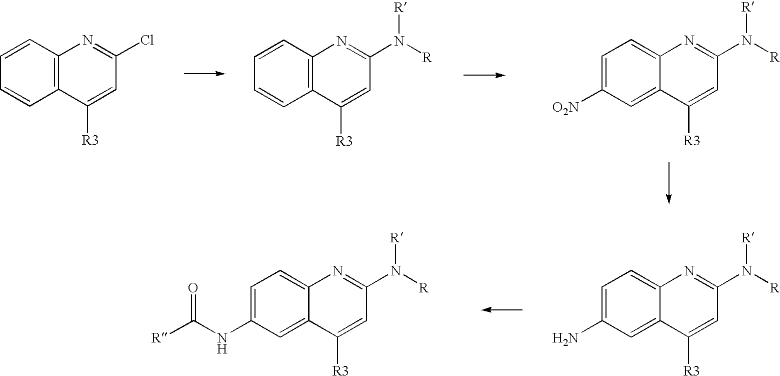

### Example 1

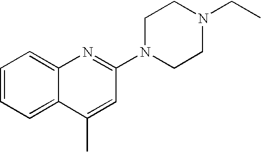

2-(4-Ethylpiperazin-1-yl)-4-methylquinoline. A stirred mixture of 2-chlorolepidine (22 g, 124 mmol) and N-ethylpiperazine (50 mL, 395 mmol) was heated to 150° C. for 2 hours. Excess N-ethylpiperazine was removed in vacuo. The residue was taken up in 3% aqueous HCl (800 mL). The aqueous phase was washed twice with DCM, 4 N NaOH added until pH 8 was reached, and extracted with DCM (4×200 mL). The extract was concentrated, and the residue was dissolved in Et₂O (500 mL). Insoluble material was filtered off, and the organic phase was concentrated in vacuo to give 28 g (88%) of the pure title compound as a light yellow oil. The product was used without further purification. LC/MS (an20p10): Rt 1.90, m/z 256.1 [MH⁻]; ¹H NMR (300 MHz, CDCl₃): δ 1.18 (t, 3H, J=7.2 Hz), 2.52 (q, 2H, J=7.2 Hz,), 2.61 (s, 3H,), 2.63 (“t”, 4H, J=5.1 Hz), 3.81 (“t”, 4H, J=5.1 Hz), 6.85 (d, 1H, J=0.8 Hz), 7.26 (m, 1H), 7.54 (m, 1H), 7.75 (ddd, 1H, J=14.9, 8.3, 1.3 Hz); ¹³C NMR(300 MHz, CDCl₃): δ 12.3, 19.5, 45.4, 52.8, 53.2, 110.1, 122.4, 123.8, 123.8, 127.5, 129.5, 145.3, 148.2, 157.6.

### Example 2

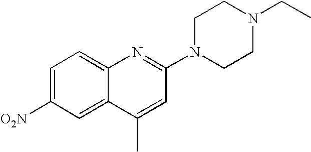

2-(4-Ethylpiperazin-1-yl)-4-methyl-6-nitroquinoline. The quinoline from Example 1 (6.0 g, 24 mmol) at was added in small portions to stirred cold (0° C.) fuming HNO₃ (>90%, 60 mL). The reaction was stirred at 0° C. for 1 h, then poured into an ice/water mixture. To the acid was added 4 N NaOH until pH 12 was reached, and the solution was left to precipitate overnight. The yellow precipitate was filtered off, washed with water (4×100 mL) and dried in vacuo to give 7.0 g (99%) of the pure title compound as a yellow solid. The product was used without further purification. LC/MS (an20p10): Rt 4.59, m/z 301.1 [MH⁺]; ¹H NMR (300 MHz, CDCl₃): δ 1.18 (t, 3H, J=7.1 Hz), 2.52 (q, 2H, J=7.2 Hz), 2.62 (“t”, 4H, J=5.3 Hz), 2.67 (s, 3H), 3.90 (“t”, 4H, J=5.3 Hz), 6.92 (d, 1H, J=0.8 Hz), 7.67 (d, 1H, J=9.2 Hz), 8.30 (dd, 1H, J=9.2, 2.6 Hz), 8.72 (d, 1H, J=2.5 Hz).

### Example 3

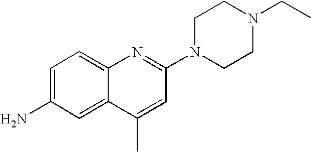

2-(4-Ethylpiperazin-1-yl)-4-methyl-6-aminoquinoline. The quinoline from Example 2 (5.3 g, 17 mmol) in THF (150 mL) under argon was added to a suspension of 5% Pd/C (1.0 g) in THF (10 mL). The argon atmosphere was substituted with hydrogen, and the reaction was stirred under 1 atm H₂ for 12 h. The reaction mixture was filtered through a pad of Celite and concentrated in vacuo to give 4.65 g (97%) of the pure title product as a greenish oil. LC/MS (an20p10): Rt 1.60, m/z 171.1 [MH⁺]; ¹H NMR (300 MHz, CDCl₃): δ1.15 (t, 3H, J=7.3 Hz), 2.02 (s, 2H), 2.49 (q, 2H, 7.2 Hz), 2.50 (s, 3H), 2.60 (“t”, 4H, J=5.3 Hz), 3.69 (“t”, 4H, J=5.1 Hz), 6.79 (d, 1H, J=0.9 Hz), 6.96 (d, 1H, J=2.4 Hz), 7.02 (dd, 1H, J=8.7, 2.6 Hz), 7.58 (dd, 1H, J=8.9, 0.6 Hz); ¹³C NMR (300 MHz, CDCl₃): δ 12.1, 19.6, 45.7, 52.8, 53.1, 106.1, 110.8, 121.1, 124.8, 128.7, 128.7, 133.6, 141.7, 142.6, 143.7, 156.2.

### Example 4

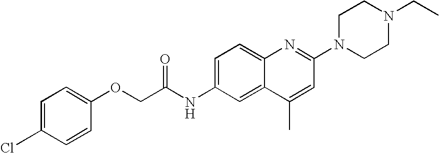

2-(4-Chlorophenoxy)-N-[2-(4-ethylpiperazin-1-yl)-4-methylquinolin-6-yl]-acetamide. Aminoquinoline from Example 3 (1.54 g, 5.7 mmol) in dry DCM (15 ml) u/Ar was added to freshly prepared 4-chlorophenoxyacetyl chloride (1.2 g, 6.0 mmol) in DCM dropwise at r.t. The resulting green slurry was stirred for 3 h. The reaction was diluted with DCM (200 ml), washed with 1 N NaOH and brine, dried and concentrated to 2.40 g (96%) of the pure title compound as a light brown solid. The crude product was recrystallized from MeOH to give 1.65 g of the title compound as light brown needles. LC/MS (an20p10): Rt 4.37 min, m/z 439.1 [MH⁺]; ¹H NMR (300 MHz, CDCl₃): δ 1.15 (t, 3H, J=7.3 Hz), 2.49 (q, 2H, 7.2 Hz), 2.59 (s, 3H), 2.59 (m, 4H), 3.77 (m, 4H), 6.85 (d, 1H, J=0.8 Hz), 6.95 (m, 2H), 7.31 (m, 2H), 7.53 (dd, 1H, J=8.9, 2.3 Hz), 7.69 (d, 1H, J=8.9 Hz), 8.20 (d, 1H, J=2.5 Hz), 8.31 (s, 1H); ¹³C NMR (300 MHz, CDCl₃): δ 12.1, 19.6, 45.7, 52.8, 53.1, 106.1, 110.8, 121.1, 124.8, 128.7, 128.7, 133.6, 141.7, 142.6, 143.7, 156.2.

### Example 5

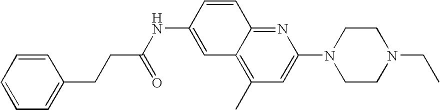

N-[2-(4-Ethyl-piperazin-1-yl)-4-methyl-quinolin-6-yl]-3-phenyl-propionamide. A solution of aminoquinoline from Example 3 (0.10 mmol) in dry DCM (1.5 ml) was added to a 0.25 M solution of 3-phenylpropionyl chloride (0.5 mL, 0.125 mmol) in dry DMS. The reaction was stirred u/Ar over night, then extracted with Na₂CO₃ (sat.). The organic phase was concentrated and the residue was purified by chromatography (SiO₂, [MeOH w/5% NH₃]: EtOAc, 1:9 to 1:5) to give the title compound. LC/MS (an20p10): Rt 4.460 min, m/z 403.2 [MH⁺].

### Example 6

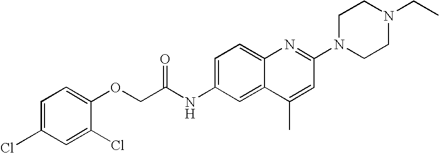

2-(2,4-Dichloro-phenoxy)-N-[2-(4-ethyl-piperazin-1-yl)-4-methyl-quinolin-6-yl]-acetamide. The title compound was made according to a procedure similar to the one described for Example 5. LC/MS (an20p10): Rt 4.925 min, m/z 473.1 [MH⁺].

### Example 7

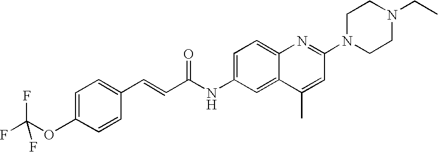

(E)-N-[2-(4-Ethyl-piperazin-1-yl)-4-methyl-quinolin-6-yl]-3-(4-trifluoromethoxy-phenyl)-acrylamide. The title compound was made according to a procedure similar to the one described for Example 5. LC/MS (an20p10): Rt 5.283 min, m/z 485.2 [MH⁺].

### Example 8

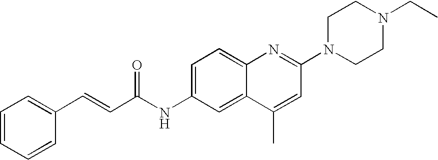

(E)-N-[2-(4-Ethyl-piperazin-1-yl)-4-methyl-quinolin-6-yl]-3-phenyl-acrylamide. The title compound was made according to a procedure similar to the one described for Example 5. LC/MS (an20p10): Rt 4.610 min, m/z 401.2 [MH⁺].

### Example 9

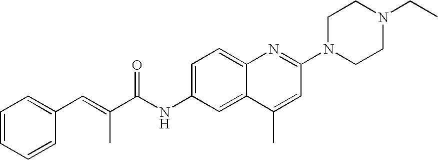

(E)-N-[2-(4-Ethyl-piperazin-1-yl)-4-methyl-quinolin-6-yl]-2-methyl-3-phenyl-acrylamide. The title compound was made according to a procedure similar to the one described for Example 5. LC/MS (an20p10): Rt 5.730 min, m/z 415.2 [MH⁺].

### Example 10

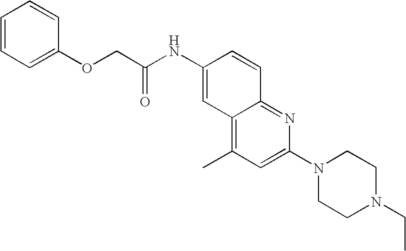

N-[2-(4-Ethyl-piperazin-1-yl)-4-methyl-quinolin-6-yl]-2-phenoxy-acetamide. The title compound was made according to a procedure similar to the one described for Example 5. LC/MS (an20p10): Rt 5.165 min, m/z 405.2 [MH⁺].

### Example 11

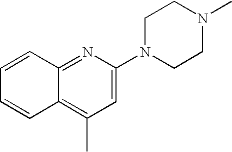

4-Methyl-2-(4-methyl-piperazin-1-yl)-quinoline. The title compound was made according to a procedure similar to the one described for Example 1. LC/MS (an20p10): Rt 2.49 min, m/z 242.1[MH⁺]; ¹H NMR (300 MHz, CDCl₃): δ 2.37 (s, 3H), 2.56 (“t”, 4H, J=4.9 Hz), 2.60 (s, 3H), 3.77 (“t”, 4H, J=4.0 Hz), 6.85 (d, 1H, 0.8 Hz), 7.25 (m, 1H), 7.53 (m, 1H), 7.72 (ddd, 1H, J=8.3, 1.1, 0.6 Hz), 7.77 (dd, 1H, J=8.3, 1.3 Hz); ¹³C NMR(75 MHz, CDCl₃): δ 19.6, 45.4, 46.5, 55.4, 110.2, 122.5, 123.8, 123.8, 127.5, 129.6, 145.4, 148.2, 157.6.

### Example 12

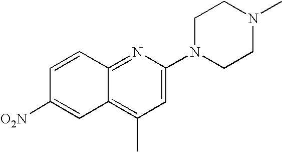

4-Methyl-2-(4-methyl-piperazin-1-yl)-6-nitro-quinoline. The title compound was made according to a procedure similar to the one described for Example 2. LC/MS (an20p10): Rt 4.31 min, m/z 287.1[MH⁺]; ¹H NMR (300 MHz, CDCl₃): δ 2.38 (s, 3H), 2.59 (m, 4H), 2.65 (s, 3H), 3.88 (m, 4H), 6.92 (s, 1H), 7.66 (d, 1H, J=9.2 Hz), 8.30 (dd, 1H, J=9.2, 2.4 Hz), 8.70 (d, 1H, J=2.4 Hz); ¹³C NMR(75 MHz, CDCl₃): δ 19.6, 44.9, 46.5, 55.3, 111.2, 121.3, 122.2, 123.8, 127.9, 142.1, 127.1, 152.1, 158.7.

### Example 13

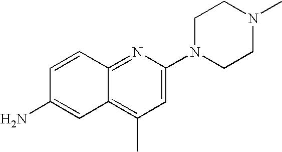

4-Methyl-2-(4-methyl-piperazin-1-yl)-quinolin-6-ylamine. The title compound was made according to a procedure similar to the one described for Example 3. LC/MS (an20p10): Rt 1.57 min, m/z 257.1 [MH⁺]; ¹H NMR (300 MHz, CDCl₃): δ 2.38 (s, 3H), 2.52 (s, 3H), 2.58 (m, 4H), 3.70 (m, 4H), 3.73 (br s, 2H), 6.81 (d, 1H, J=0.8 Hz), 6.98 (d, 1H, J=2.5 Hz), 7.04 (dd, 1H, J=8.9, 2.6 Hz), 7.60 (d, 1H, J=8.7 Hz); ¹³C NMR(75 MHz, CDCl₃): δ 19.6, 45.8, 46.4, 55.4, 106.1, 110.9, 121.1, 124.8, 128.7, 141.7, 142.6, 143.7, 156.2.

### Example 14

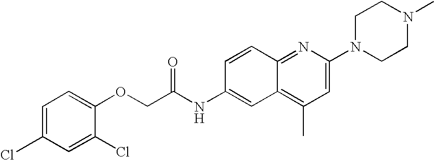

2-(2,4-Dichloro-phenoxy)-N-[4-methyl-2-(4-methyl-piperazin-1-yl)-quinolin-6-yl]-acetamide. The title compound was made according to a procedure similar to the one described for Example 4. LC/MS (an20p10): Rt 4.83 min, m/z 459.0 (³⁵CI-isotope) [MH⁺]; ¹H NMR (300 MHz, CDCl₃): δ 2.37 (s, 3H), 2.56 (m, 4H), 2.59 (s, 3H), 3.76 (m, 4H), 4.65 (s, 2H), 6.86 (s, 1H), 6.91 (d, 1H, J=8.9 Hz), 7.25 (dd, 1H, J=8.9, 2.5 Hz), 7.46 (d, 1H, J=2.5 Hz), 7.51 (dd, 1H, J=8.9, 2.4 Hz), 7.69 (d, 1H, J=8.9 Hz), 8.27 (d, 1H, J=2.3 Hz); ¹³C NMR (75 MHz, CDCl₃): δ 19.7, 45.4, 46.6, 55.4, 68.9, 110.8, 114.0, 115.4, 123.1, 123.9, 124.3, 128.2, 128.3, 128.5, 130.7, 131.5, 145.3, 145.9, 151.9, 157.5, 165.2.

### Example 15

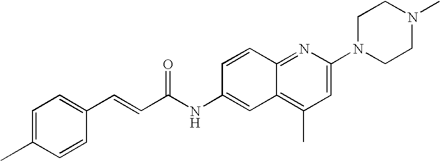

(E)-N-[4-Methyl-2-(4-methyl-piperazin-1-yl)-quinolin-6-yl]-3-p-tolyl-acrylamide. The title compound was made according to a procedure similar to the one described for Example 5. LC/MS (an20p10): Rt 4.87 min, m/z 401.2 [MH⁺].

### Example 16

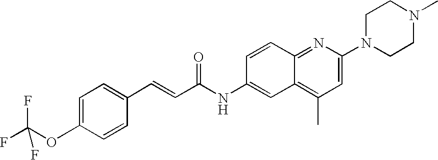

(E)-N-[4-Methyl-2-(4-methyl-piperazin-1-yl)-quinolin-6-yl]-3-(4-trifluoromethoxy-phenyl)-acrylamide. The title compound was made according to a procedure similar to the one described for Example 5. LC/MS (an20p10): Rt 5.409 min, m/z 471.1 [MH⁺].

### Example 17

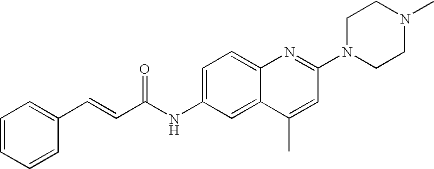

(E)-N-[4-Methyl-2-(4-methyl-piperazin-1-yl)-quinolin-6-yl]-3-phenyl-acrylamide.

The title compound was made according to a procedure similar to the one described for Example 5. LC/MS (an20p10): Rt 4.137 min, m/z 387.2 [MH⁺].

### Example 18

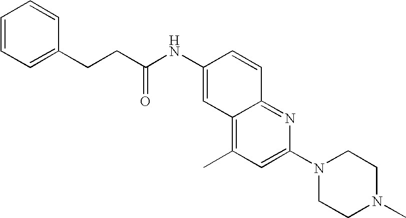

N-[4-Methyl-2-(4-methyl-piperazin-1-yl)-quinolin-6-yl]-3-phenyl-propionamide. The title compound was made according to a procedure similar to the one described for Example 5. LC/MS (an20p10): Rt 4.676 min, m/z 389.2 [MH⁺].

### Example 19

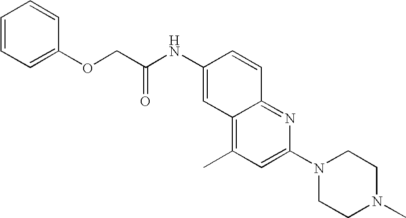

N-[4-Methyl-2-(4-methyl-piperazin-1-yl)-quinolin-6-yl]-2-phenoxy-acetamide. The title compound was made according to a procedure similar to the one described for Example 5. LC/MS (an20p10): Rt 4.695 min, m/z 391.2 [MH⁺].
General Synthetic Route II

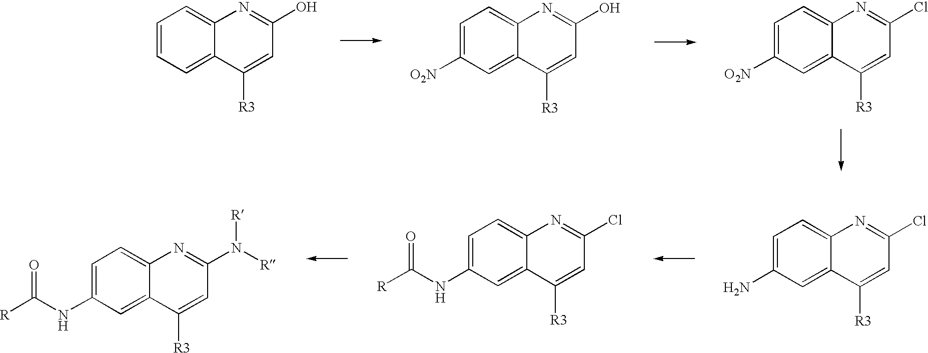

### Example 20

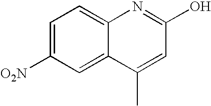

2-Hydroxy-4-methyl-6-nitroquinoline. 2-Hydroxylepidine (10 g, 63 mmol) was added slowly to cooled (0° C.), stirred fuming HNO₃ (>90%, 75 ml). After 2 h the reaction mixture was poured over ice, and 4 M NaOH (aq.) was added until pH>7. The precipitate was filtered off and washed with water. The crude product was recrystallized from ethanol to give 9.86 g (82%) of the pure title compound. ¹H NMR (300 MHz, DMSO-d₆): δ 2.50 (s, 3H), 6.57 (d, 1H, J=1.1 Hz), 7.43 (d, 1H, J=9.0 Hz), 8.34 (dd, 1H, J=9.2, 2.6 Hz), 8.50 (d, 1H, J=2.5 Hz), 12.18 (br s, 1H).

### Example 21

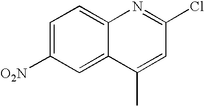

2-Chloro-4-methyl-6-nitroquinoline. 2-Hydroxy-4-methyl-6-nitroquinoline (503 mg, 2.46 mmol) was added to POCl₃ (3 ml, 32 mmol) and mixture was heated by microwaves to 150° C. for 5 min. The violet reaction mixture was poured into water and stirred until excess POCl₃ was destroyed. 4 N NaOH was carefully added to the aqueous phase until pH 7 was reached, and the precipitate was filtered off and dried to give 530 mg (97%) of the pure title compound as a violet solid. The product was used without further purification. LC/MS (an20p10): Rt 10.51, m/z 222.9 (³⁵CI-isotope) [MH⁺]; ¹H NMR (300 MHz, DMSO-d₆): δ 2.81 (s, Me), 7.73 (s, H-3), 8.15 (d, J=9.2 Hz), 8.52 (dd, J=9.2, 2.6 Hz), 8.96 (d, J=2.4 Hz, H-5).

### Example 22

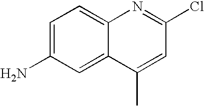 2-Chloro-4-methyl-6-aminoquinoline. To a suspension of 5% Pt/C (85 mg) in MeOH (35 ml) was added 2-Chloro-4-methyl-6-nitroquinoline (508 mg, 2.28 mmol). The reaction was stirred under 1 atm H₂ at room temperature for 2 h. Filtration through a pad of Celite and concentration gave 360 mg (82%) of the title compound as a yellow solid. The product was used without further purification. LC/MS (an20p10): Rt 7.69 min, m/z 193.0 (³⁵CI-isotope) [MH⁺]; ¹H NMR (300 MHz, DMSO-d₆): δ 2.49 (d, 3H, J=0.9 Hz), 5.85 (br s, 2H), 6.90 (d, 1H, J=2.5 Hz), 7.17 (dd, 1H, J=9.0, 2.4 Hz), 7.22 (d, 1H, J=0.8 Hz), 7.61 (d, 1H, J=8.9 Hz); ¹³C NMR (300 MHz, DMSO-d₈): δ 19.0, 102.8, 122.5, 122.8, 129.3, 130.0, 141.4, 144.7, 145.9, 148.4.

### Example 23

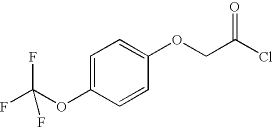

### (4-Trifluoromethoxy-phenoxy)-acetyl chloride

To trifluoromethoxyphenol (1.53 g, 8.6 mmol) and ethyl acetate (0.95 ml, 8.6 mmol) in acetone (5 ml) was added K₂CO₃ (1.2 g, 8.6 mmol), and the reaction was stirred at r.t. for 1 h, then concentrated. The residue was partitioned between EtOAc and water. The organic phase was washed with brine, dried (MgSO₄) and concentrated. The residue was purified by flash chromatography (SiO₂, EtOAc:heptane, 1:5) to give 1.8 g (80%) Ethyl (4-Trifluoromethoxy-phenoxy)-acetate as pale yellow oil. Ethyl (4-Trifluoromethoxy-phenoxy)-acetate (1.67 g, 6.32 mmol) in THF (10 mL) was added LiOH (477 mmol, 11.4 mmol) dissolved in water (40 mL), and the reaction was stirred vigorously for 12 h. 3% HCl was then added until pH<1, and the mixture was extracted with DCM. The organic phase was washed with brine, dried (MgSO₄) and concentrated to give 1.43 g (96%) (4-trifluoromethoxy-phenoxy)-acetic acid as a white solid.

To (4-trifluoromethoxy-phenoxy)-acetic acid (1.25 g, 5.29 mmol) in dry DCM (10 Ml) at 0° C. was added oxalyl chloride (470 μL, 5.3 mmol). DMF (1 drop) was added, and the reaction was stirred at r.t. for 1 h. The solution was used directly in the next step. TLC (SiO₂, EtOAc: heptane, 1:1): Rf 0.3.

### Example 24

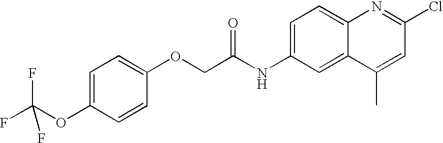

N-(2-Chloro-4-methyl-quinolin-6-yl)-2-(4-trifluoromethoxyphenoxy)-acetamide. To chloroquinoline (4.33 g, 22.4 mmol) in dry DCM (120 mL) was added dropwise 4-trifluoromethoxyphenoxyacetyl chloride (6.32 g, 24.8 mmol). The reaction was stirred at room temperature for 2 hours, and then poured into MeOH (380 mL) to give a homogenous solution. Water (250 mL) was added in small portions, and the mixture was left to precipitate. The precipitate was filtered off, washed with MeOH/water (1:1, 200 mL). The solution was concentrated until precipitation started, and left to give a second crop, that was collected and washed like the first crop. The combined product was dried to give 8.72 g (95%) of the pure title compound as a white solid. The product was used without further purification. LC/MS (an20p10): Rt 4.22 min, m/z 411.0 (³⁵CI-isotope) [MH+]; ¹H NMR (300 MHz, DMSO-d₆): δ 2.62 (s, 3H), 4.84 (s, 2H), 7.13 (d, 2H, =9.0 Hz), 7.34 (d, 2H, J=9.1 Hz), 7.91 (d, 1H, J=9.2 Hz), 7.98 (dd, 1H, 9.0, 2.3 Hz), 8.49 (d, 1H, 1.9 Hz), 10.55 (s, 1H); ¹³C NMR (300 MHz, DMSO-d₆): δ 18.9, 68.2, 113.1, 116.8, 123.4, 123.4, 125.1, 127.9, 129.9, 137.8, 143.1, 144.7, 148.8, 157.5, 167.7.

### Example 25

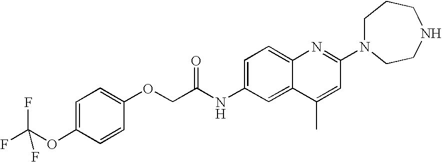

N-(2-[1,4]Diazepan-1-yl-4-methylquinolin-6-yl)-2-(4-trifluoromethoxyphenoxy)-acetamide. To N-(2-chloro-4-methyl-quinolin-6-yl)-2-(4-hydroxy-phenoxy)-acetamide (400 mg, 0.97 mmol) was added homopiperazine (4 mL, 40 mmol) and the mixture was heated to 140° C. under argon. After 2 h, excess homopiperazine was distilled off in vacuo, and the residue was purified by flash chromatography (SiO₂, [MeOH w/5% NH₄OH]:EtOAc, 1:5) to give the title product. LC/MS (an05p7): Rt 3.94 min, m/z 475.1 [MH⁺].

### Example 26

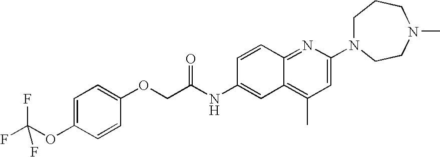

N-[4-Methyl-2-(4-methyl-[1,4]diazepan-1-yl)-quinolin-6-yl]-2-(4-trifluoromethoxy-phenoxy)-acetamide. To N-(2-chloro-4-methyl-quinolin-6-yl)-2-(4-hydroxy-phenoxy)-acetamide (33 mg, 0.08 mmol) was added 2-methylpiperazine (1.0 mL, 8 mmol), and the mixture was heated to 100° C. under argon over night. Excess amine was evaporated off in vacuo. The residue was dissolved in DCM. The organic phase was washed with Na₂CO₃ (sat.), dried (MgSO₄) and concentrated. The residue was purified by flash chromatography (SiO₂, [MeOH w/5% NH₄OH]:EtOAc, 1:5) to give 23.6 mg (60%) the title compound. LC/MS (an20p10): Rt 5.397 min, m/z 489.1 [MH⁺].

### Example 27

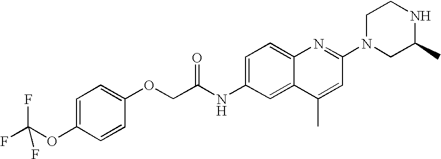

N-[4-Methyl-2-((S)-3-methyl-piperazin-1-yl)-quinolin-6-yl]-2-(4-trifluoromethoxy-phenoxy)-acetamide. To N-(2-Chloro-4-methyl-quinolin-6-yl)-2-(4-hydroxy-phenoxy)-acetamide (150 mg, 0.367 mmol) was added 2-methylpiperazine (180 mg, 0.84 mmol) and the mixture was heated to 130° C. under argon fro 45-50 min. Excess amine was evaporated off in vacuo and DCM (2 mL) was added. The organic phase was washed with Na₂CO₃ (sat.) and concentrated. The residue was purified by flash chromatography (SiO₂, [MeOH w/5% NH₄OH]:EtOAc, 1:5) to give the title compound. LC/MS (an05p7): Rt 3.868 min, m/z 475.2 [MH⁺].

### Example 28

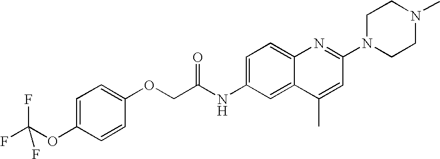

N-[4-Methyl-2-(4-methyl-piperazin-1-yl)-quinolin-6-yl]-2-(4-trifluoromethoxy-phenoxy)-acetamide. The title compound was made according to a procedure similar to the one described for Example 27. LC/MS (an20p10): Rt 6.061 min, m/z 475.1 [MH⁺].

### Example 29

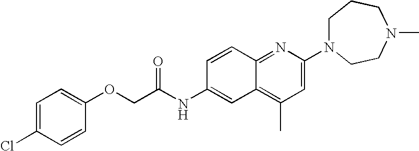

2-(4-Chloro-phenoxy)-N-[4-methyl-2-(4-methyl-[1,4]diazepan-1-yl)-quinolin-6-yl]-acetamide. The title compound was made according to a procedure similar to the one described for Example 27. LC/MS (an20p10): Rt 4.869 min, m/z 439.1 [MH⁺].

### Example 30

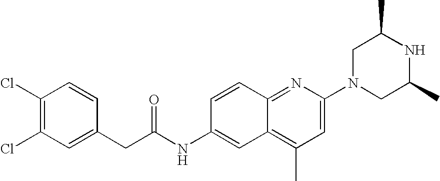

2-(3,4-Dichloro-phenyl)-N-[2-((3R,5S)-3,5-dimethyl-piperazin-1-yl)-4-methyl-quinolin-6-yl]-acetamide. The title compound was made according to a procedure similar to the one described for Example 27. ¹H NMR (300 MHz, CDCl3): 1.16 (s, CH3), 1.18 (s, CH3), 2.53 (s, CH3), 3.65 (s, CH2), 8.15 (d, —CONH—)

### Example 31

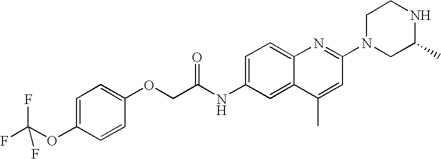

N-[4-Methyl-2-((R)-3-methyl-piperazin-1-yl)-quinolin-6-yl]-2-(4-trifluoromethoxy-phenoxy)-acetamide. The title compound was made according to a procedure similar to the one described for Example 27. LC/MS (an05p7): Rt 3,86 min, m/z 475,2 [MH⁺].

### Example 32

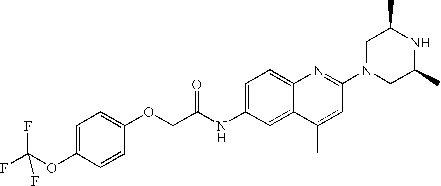

N-[2-((3R,5S)-3,5-Dimethyl-piperazin-1-yl)-4-methyl-quinolin-6-yl]-2-(4-trifluoromethoxy-phenoxy)-acetamide. The title compound was made according to a procedure similar to the one described for Example 27. LC/MS (an05p7): Rt 3,974 min, m/z 489,2 [MH⁺].

### Example 33

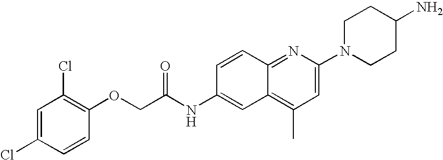

N-[2-(4-Amino-piperidin-1-yl)-4-methyl-quinolin-6-yl]-2-(2,4-dichloro-phenoxy)-acetamide. The title compound was made according to a procedure similar to the one described for Example 27. LC/MS (an10p8): Rt 5.40 min, m/z 461.1 [MH⁺].

### Example 34

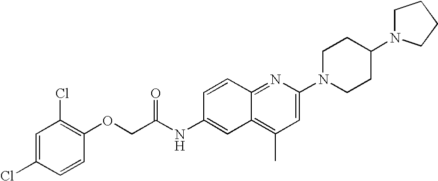

2-(2,4-Dichloro-phenoxy)-N-[4-methyl-2-(4-pyrrolidin-1-yl-piperidin-1-yl)-quinolin-6-yl]-acetamide. The title compound was made according to a procedure similar to the one described for Example 27. LC/MS (an10p8): Rt 6.40 min, m/z 513.2 [M⁺].

### Example 35

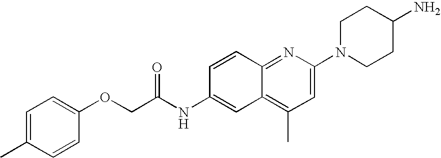

N-[2-(4-Amino-piperidin-1-yl)-4-methyl-quinolin-6-yl]-2-p-tolyloxy-acetamide. The title compound was made according to a procedure similar to the one described for Example 27. LC/MS (an10p8): Rt 4.64 min, m/z 405.2 [M⁺].

### Example 36

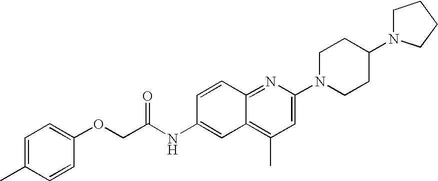

N-[4-Methyl-2-(4-pyrrolidin-1-yl-piperidin-1-yl)-quinolin-6-yl]-2-p-tolyloxy-acetamide. The title compound was made according to a procedure similar to the one described for Example 27. LC/MS (an10p8): Rt 5.36 min, m/z 459.2 [MH⁺].

### Example 37

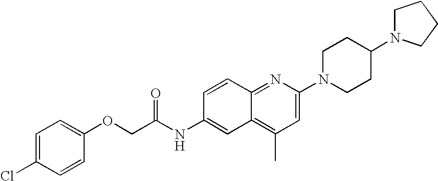

2-(4-Chloro-phenoxy)-N-[4-methyl-2-(4-pyrrolidin-1-yl-piperidin-1-yl)-quinolin-6-yl]-acetamide. The title compound was made according to a procedure similar to the one described for Example 27. LC/MS (an10p8): Rt 5.43 min, m/z 480.2 [MH⁺].

### Example 38

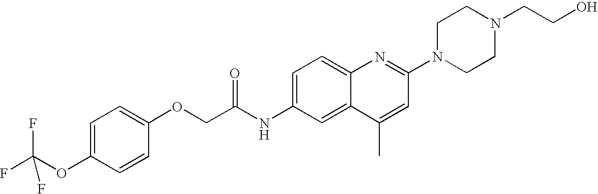

N-{2-[4-(2-Hydroxy-ethyl)-piperazin-1-yl]-4-methyl-quinolin-6-yl}-2-(4-trifluoromethoxy-phenoxy)-acetamide. The title compound was made according to a procedure similar to the one described for Example 27. LC/MS (an05p7): Rt 4,069 min, m/z 505.2 [MH⁺].
General Synthetic Route III

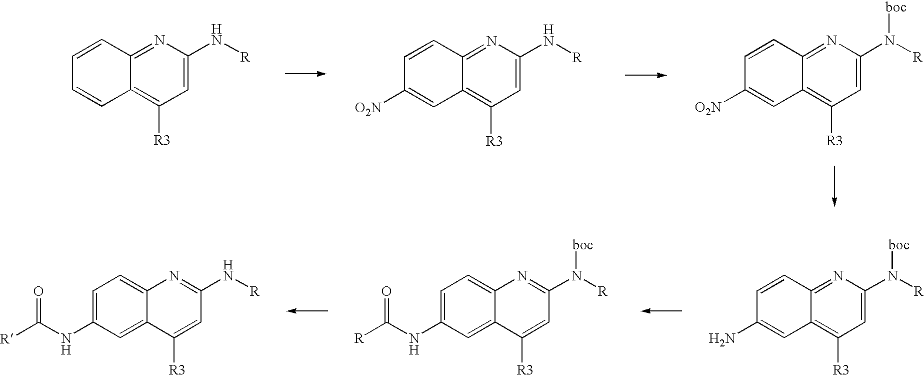

### Example 39

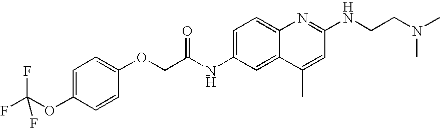

N-[2-(2-Dimethylamino-ethylamino)-4-methyl-quinolin-6-yl]-2-(4-trifluoromethoxy-phenoxy)-acetamide. 2-N-Boc-N-(dimethylaminoethyl)-4-methyl-6-aminoquinoline (50 mg, 0.15 mmol) was added 0.3 M solution of 4-trifluoromethoxyphenoxyacetyl chloride in DCM (0.50 mL, 0.15 mmol) and TFA (40 μL, 0.3 mmol). The reaction were stirred for 12 h under argon. TFA (100 uL) was added, and the stirring was continued for 2 days.

The reaction was concentrated. The residue was dissolved in DCM and washed with Na₂CO₃ (sat.), dried (MgSO₄) and concentrated. The residue was purified by chromatography (SiO₂, [MeOH w/5% NH₃]: EtOAc, 1:9 to 1:5) to give the title compound. LC/MS (an05p7): Rt 4.081 min, m/z 463.1 [MH⁺].

### Example 40

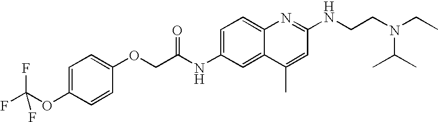

N-[2-(2-Diisopropylamino-ethylamino)-4-methyl-quinolin-6-yl]-2-(4-trifluoromethoxy-phenoxy)-acetamide. The title compound was made according to a procedure similar to the one described for Example 39. LC/MS (an05p7): Rt 5.364 min, m/z 519.2 [MH⁺]

### Example 41

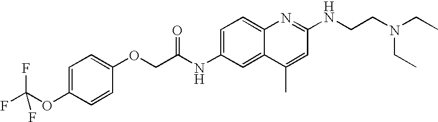

N-[2-(2-Diethylamino-ethylamino)-4-methyl-quinolin-6-yl]-2-(4-trifluoromethoxy-phenoxy)-acetamide. The title compound was made according to a procedure similar to the one described for Example 39. LC/MS (an05p7): Rt 4.529 min, m/z 491.1 [MH⁺]

### Example 42

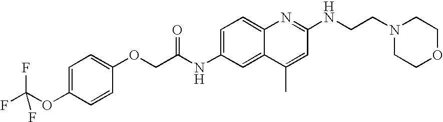

N-[4-Methyl-2-(2-morpholin-4-yl-ethylamino)-quinolin-6-yl]-2-(4-trifluoromethoxy-phenoxy)-acetamide. The title compound was made according to a procedure similar to the one described for Example 39. LC/MS (an05p7): Rt 3.671 min, m/z 505.1 [MH⁺].

### Example 43

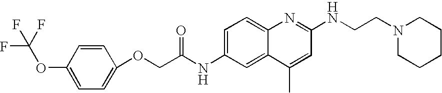

N-[4-Methyl-2-(2-piperidin-1-yl-ethylamino)-quinolin-6-yl]-2-(4-trifluoromethoxy-phenoxy)-acetamide. The title compound was made according to a procedure similar to the one described for Example 39. LC/MS (an05p7): Rt 4.388 min, m/z 503.1 [MH⁺].

### Example 44

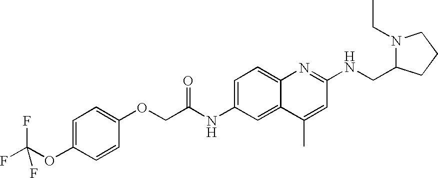

N-{2-[(1-Ethyl-pyrrolidin-2-ylmethyl)-amino]-4-methyl-quinolin-6-yl}-2-(4-trifluoromethoxy-phenoxy)-acetamide. The title compound was made according to a procedure similar to the one described for Example 39. LC/MS (an05p7): Rt 4.910 min, m/z 503.1 [MH⁺].

### Example 45

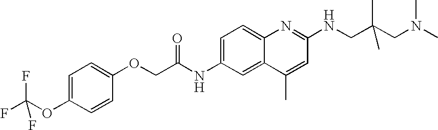

N-[2-(3-Dimethylamino-2,2-dimethyl-propylamino)-4-methyl-quinolin-6-yl]-2-(4-trifluoromethoxy-phenoxy)-acetamide. The title compound was made according to a procedure similar to the one described for Example 39. LC/MS (an05p7): Rt 4.593 min, m/z 505.1 [MH⁺].

### Example 46

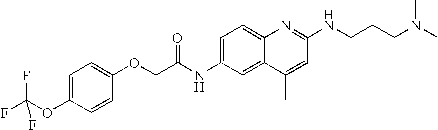

N-[2-(3-Dimethylamino-propylamino)-4-methyl-quinolin-6-yl]-2-(4-trifluoromethoxy-phenoxy)-acetamide. The title compound was made according to a procedure similar to the one described for Example 39. LC/MS (an05p7): Rt 4.101 min, m/z 477.1 [MH⁺].

### Example 47

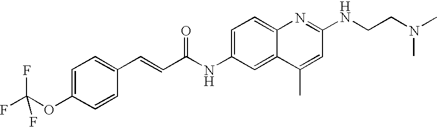

(E)-N-[2-(2-Dimethylamino-ethylamino)-4-methyl-quinolin-6-yl]-3-(4-trifluoromethoxy-phenyl)-acrylamide. The title compound was made according to a procedure similar to the one described for Example 39. LC/MS (an20p10): Rt 4.52 min, m/z 459.2 [MH⁺].

### Example 48

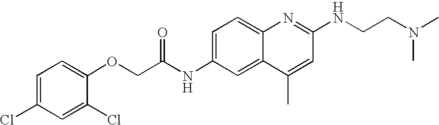

2-(2,4-Dichloro-phenoxy)-N-[2-(2-dimethylamino-ethylamino)-4-methyl-quinolin-6-yl]-acetamide. The title compound was made according to a procedure similar to the one described for Example 39. LC/MS (an20p10): Rt 5.16 min, m/z 447.1 [MH⁺].

### Example 49

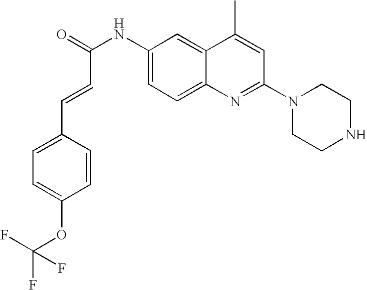

(E)-N-(4-Methyl-2-piperazin-1-yl-quinolin-6-yl)-3-(4-trifluoromethoxy-phenyl)-acrylamide. The title compound was made according to a procedure similar to the one described for Example 51. LC/MS (an20p10): Rt 5,030 min, m/z 457,1 [MH⁺].

### Example 50

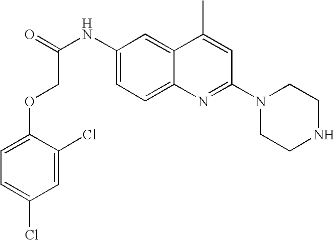

2-(2,4-Dichloro-phenoxy)-N-(4-methyl-2-piperazin-1-yl-quinolin-6-yl)-acetamide. The title compound was made according to a procedure similar to the one described for Example 51. LC/MS (an20p10): Rt 5,118 min, m/z 445,1 [MH⁺].

### Example 51

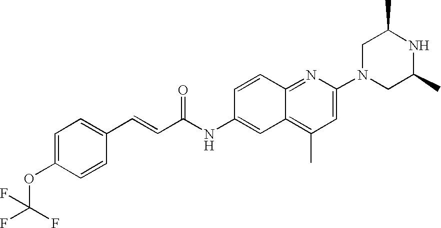

(E)-N-[2-((3R,5S)-3,5-Dimethyl-piperazin-1-yl)-4-methyl-quinolin-6-yl]-3-(4-trifluoromethoxy-phenyl)-acrylamide. The N-Boc analog (0.6 g, 1.0 mmol) was added 10% TFA in DCM (30 mL), and the mixture was stirred at r.t. for 2 h. The reaction was basified with Na₂CO₃. Sat. NaHCO₃ (50 mL) was added the the mixture was extracted with DCM (3×50 mL). The organic phase was dried (MgSO₄) and contcentrated. The residue was washed with Et₂O and dried to give 324 mg (65%) of the title compound. LC/MS (an20p10): Rt 5.85 min, m/z 485.2 [MH⁺].

### Example 52

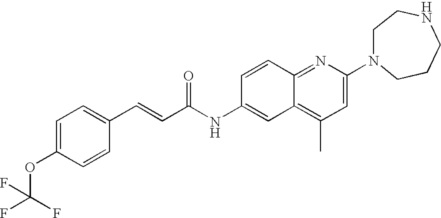

(E)-N-(2-[1,4]Diazepan-1-yl-4-methyl-quinolin-6-yl)-3-(4-trifluoromethoxy-phenyl)-acrylamide. The title compound was made according to a procedure similar to the one described for Example 51. LC/MS (an20p10): Rt 5.42 min, m/z 471.1 [MH⁺].

### Example 53

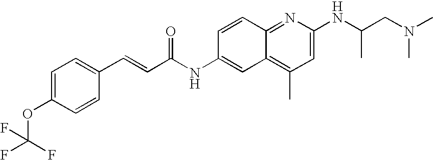

(E)-N-[2-(2-Dimethylamino-1-methyl-ethylamino)-4-methyl-quinolin-6-yl]-3-(4-trifluoromethoxy-phenyl)-acrylamide. The title compound was made according to a procedure similar to the one described for Example 39. LC/MS (an20p10): Rt 5.75 min, m/z 473.1 [MH⁺].

### Example 54

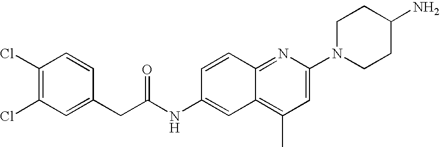

N-[2-(4-Amino-piperidin-1-yl)-4-methyl-quinolin-6-yl]-2-(3,4-dichloro-phenyl)-acetamide. The title compound was made according to a procedure similar to the one described for Example 27. ¹H NMR (300 MHz, DMSO): 3.16 (s, ArCH3), 3.35 (s, —NCH3), 3.73 (s, ArCH2-), 8.14 (d, —CONH—).

### Example 55

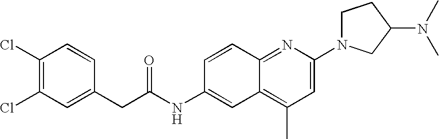

2-(3,4-Dichloro-phenyl)-N-[2-(3-dimethylamino-pyrrolidin-1-yl)-4-methyl-quinolin-6-yl]-acetamide. The title compound was made according to a procedure similar to the one described for Example 27. ¹H NMR (300 MHz, CDCl3): 2.35 (s, N(CH3)₂), 2.56 (s, CH3), 3.71 (s, CH2), 8.18 (d, —CONH—).

### Example 56

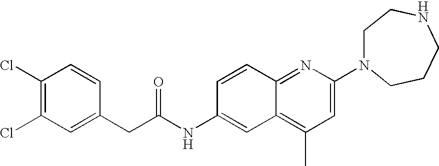

N-(2-[1,4]Diazepan-1-yl-4-methyl-quinolin-6-yl)-2-(3,4-dichloro-phenyl)-acetamide.

The title compound was made according to a procedure similar to the one described for Example 27. LC/MS (an05n7): Rt 3.81 min, m/z 443.0 [M].

### Example 57

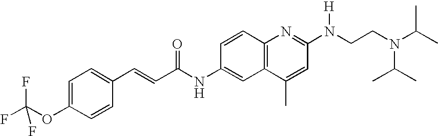

(E)-N-[2-(2-Diisopropylamino-ethylamino)-4-methyl-quinolin-6-yl]-3-(4-trifluoromethoxy-phenyl)-acrylamide. The title compound was made according to a procedure similar to the one described for Example 39. LC/MS (an20p15): Rt 8,612 min, m/z 515,2 [MH⁺].

### Example 58

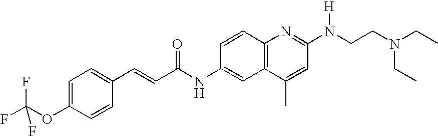

(E)-N-[2-(2-Diethylamino-ethylamino)-4-methyl-quinolin-6-yl]-3-(4-trifluoromethoxy-phenyl)-acrylamide. The title compound was made according to a procedure similar to the one described for Example 39. LC/MS (an20p15): Rt 7,424 min, m/z 487,2 [MH⁺].

### Example 59

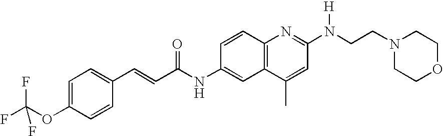

(E)-N-[4-Methyl-2-(2-morpholin-4-yl-ethylamino)-quinolin-6-yl]-3-(4-trifluoromethoxy-phenyl)-acrylamide. The title compound was made according to a procedure similar to the one described for Example 39. LC/MS (an20p15): Rt 7,186 min, m/z 501,1 [MH⁺].

### Example 60

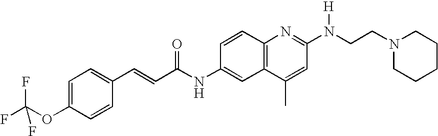

(E)-N-[4-Methyl-2-(2-piperidin-1-yl-ethylamino)-quinolin-6-yl]-3-(4-trifluoromethoxy-phenyl)-acrylamide. The title compound was made according to a procedure similar to the one described for Example 39. LC/MS (an20p15): Rt 7,455 min, m/z 499.2 [MH⁺].

### Example 61

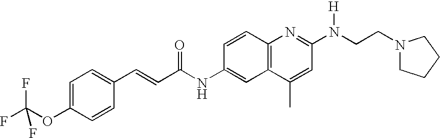

(E)-N-[4-Methyl-2-(2-pyrrolidin-1-yl-ethylamino)-quinolin-6-yl]-3-(4-trifluoromethoxy-phenyl)-acrylamide. The title compound was made according to a procedure similar to the one described for Example 39. LC/MS (an20p15): Rt 7,551 min, m/z 485.1 [MH⁺].

### Example 62

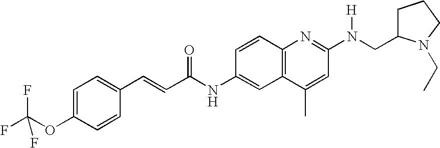

(E)-N-{2-[(1-Ethyl-pyrrolidin-2-ylmethyl)-amino]-4-methyl-quinolin-6-yl}-3-(4-trifluoromethoxy-phenyl)-acrylamide. The title compound was made according to a procedure similar to the one described for Example 39. LC/MS (an20p15): Rt 8,767 min, m/z 499.2 [MH⁺].

### Example 63

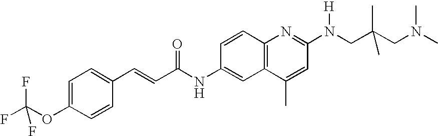

(E)-N-[2-(3-Dimethylamino-2,2-dimethyl-propylamino)-4-methyl-quinolin-6-yl]-3-(4-trifluoromethoxy-phenyl)-acrylamide. The title compound was made according to a procedure similar to the one described for Example 39. LC/MS (an20p15): Rt 7,475 min, m/z 501.2 [MH⁺].

### Example 64

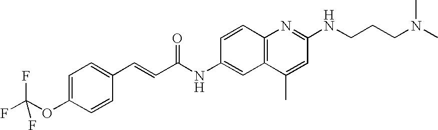

(E)-N-[2-(3-Dimethylamino-propylamino)-4-methyl-quinolin-6-yl]-3-(4-trifluoromethoxy-phenyl)-acrylamide. The title compound was made according to a procedure similar to the one described for Example 39. LC/MS (an20p15): Rt 7,179 min, m/z 473.1 [MH⁺].

### Example 65

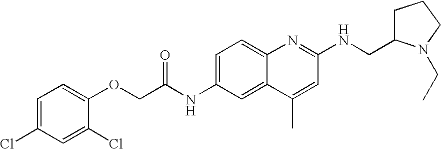 2-(2,4-Dichloro-phenoxy)-N-{2-[(1-ethyl-pyrrolidin-2-ylmethyl)-amino]-4-methyl-quinolin-6-yl}-acetamide. The title compound was made according to a procedure similar to the one described for Example 39. LC/MS (an10p8): Rt 6.425 min, m/z 487.2 [MH⁺].

### Example 66

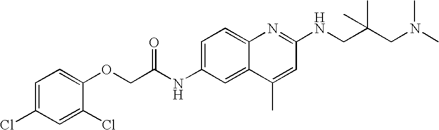

2-(2,4-Dichloro-phenoxy)-N-[2-(3-dimethylamino-2,2-dimethyl-propylamino)-4-methyl-quinolin-6-yl]-acetamide. The title compound was made according to a procedure similar to the one described for Example 39. LC/MS (an10p8): Rt 5.641 min, m/z 489.2 [MH⁺].

### Example 67

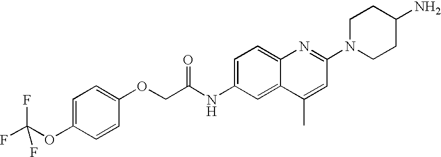

N-[2-(4-Amino-piperidin-1-yl)-4-methyl-quinolin-6-yl]-2-(4-trifluoromethoxy-phenoxy)-acetamide. The title compound was made according to a procedure similar to the one described for Example 27. LC/MS (an05p7): Rt 3.88 min, m/z 475.2 [MH⁺].

### Example 68

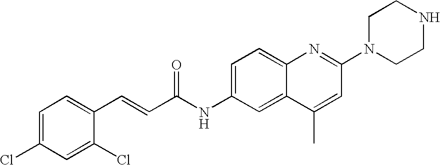

(E)-3-(2,4-Dichloro-phenyl)-N-(4-methyl-2-piperazin-1-yl-quinolin-6-yl)-acrylamide.

The title compound was made according to a procedure similar to the one described for Example 51. LC/MS (an10p8): Rt 4.620 min, m/z 441.1 [MH⁺].

### Example 69

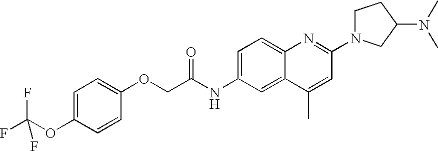

N-[2-(4-Amino-piperidin-1-yl)-4-methyl-quinolin-6-yl]-2-(4-trifluoromethoxy-phenoxy)-acetamide. The title compound was made according to a procedure similar to the one described for Example 27. LC/MS (an05p7): Rt 4.78 min, m/z 489.2 [MH⁺].

### Example 70

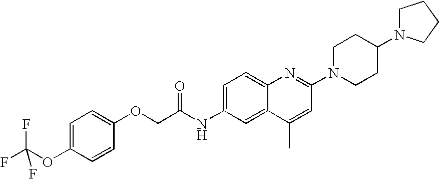

N-[4-Methyl-2-(4-pyrrolidin-1-yl-piperidin-1-yl)-quinolin-6-yl]-2-(4-trifluoromethoxy-phenoxy)-acetamide. The title compound was made according to a procedure similar to the one described for Example 27. LC/MS (an20p15): Rt 4.731 min, m/z 529.2 [MH⁺].

### Example 71

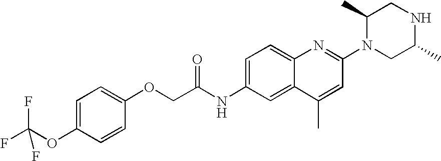

N-[2-((2S,5R)-2,5-Dimethyl-piperazin-1-yl)-4-methyl-quinolin-6-yl]-2-(4-trifluoromethoxy-phenoxy)-acetamide. The title compound was made according to a procedure similar to the one described for Example 27. LC/MS (an10p8): Rt 4.306 min, m/z 489.2 [MH⁺].

### Example 72

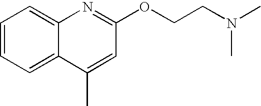

Dimethyl-[2-(4-methyl-6-nitro-quinolin-2-yloxy)-ethyl]-amine. A solution of 2-chlorolepidine (3.0 g, 17 mmol) in dry dioxane (5 mL) was added N,N-dimethylethanolamine (1.9 mL, 19 mmol) and NaH (60% in mineral oil, 600 mg, 25 mmol). The mixture was heated to reflux under inert atmosphere for 12 h. The reaction was cooled and added and sat NH₄Cl (70 mL). The aqueous phase was extracted with EtOAc. The organic phase was dried (MgSO₄) and concentrated. The residue was purified by chromatography (SiO₂, [MeOH w/10% NH₄OH]:DCM, 1:10) to give 1.02 g (26%). LC/MS (an20p10): Rt 4.27 min, m/z 231.1 [MH⁺].

### Example 73

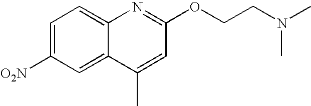

Dimethyl-[2-(4-methyl-6-nitro-quinolin-2-yloxy)-ethyl]-amine. The title compound was made according to a procedure similar to the one described for Example 2. ¹H NMR (300 MHz, CDCl₃): d 2.37 (s, 6H), 2.70 (s, 3H), 2.78 (m, 2H), 4.62 (m, 2H), 6.95 (s, 1H), 7.91 (d, 1H), 8.39 (dd, 1H), 8.82 (d, 1H).

### Example 74

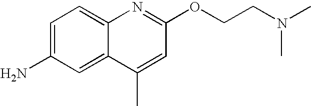

2-(2-Dimethylamino-ethoxy)-4-methyl-quinolin-6-ylamine. The title compound was made according to a procedure similar to the one described for Example 3. LC/MS (an20p10): Rt 1.94 min, m/z 246.1 [MH⁺].

### Example 75

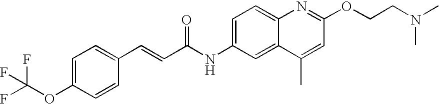

(E)-N-[2-(2-Dimethylamino-ethoxy)-4-methyl-quinolin-6-yl]-3-(4-trifluoromethoxy-phenyl)-acrylamide. The title compound was made according to a procedure similar to the one described for Example 4. LC/MS (an20p10): Rt 6.76 min, m/z 460.1 [MH⁺]; ¹H NMR (300 MHz, DMSO-d₆): δ 2.57 (s, 3H), 2.82 (d, 6H), 5.76 (s, 2H), 10.39 (br s, 1H).

### Example 76

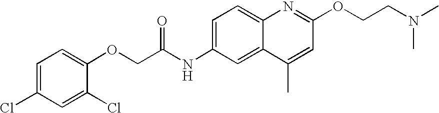

2-(2,4-Dichloro-phenoxy)-N-[2-(2-dimethylamino-ethoxy)-4-methyl-quinolin-6-yl]-acetamide. The title compound was made according to a procedure similar to the one described for Example 4. LC/MS (an20p10): Rt 7.16 min, m/z 448.1 and 450.1.

### Example 77

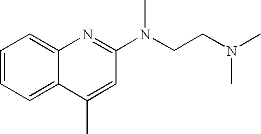

N,N,N′-Trimethyl-N′-(4-methyl-quinolin-2-yl)-ethane-1,2-diamine. The title compound was made according to a procedure similar to the one described for Example 1. LC/MS (an20p10): Rt 1.78 min, m/z 244.1 [MH⁺].

### Example 78

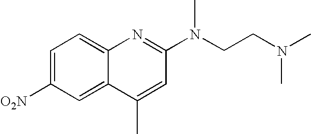

N,N,N′-Trimethyl-N′-(4-methyl-6-nitro-quinolin-2-yl)-ethane-1,2-diamine. The title compound was made according to a procedure similar to the one described for Example 2. LC/MS (an20p10): Rt 4.52 min, m/z 289.1 [MH⁺].

### Example 79

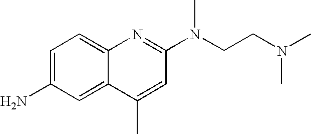

N*2*-(2-Dimethylamino-ethyl)-4,N*2*-dimethyl-quinoline-2,6-diamine. The title compound was made according to a procedure similar to the one described for Example 3. LC/MS (an20p10): Rt 1.54 min, m/z 259.1 [MH⁺].

### Example 80

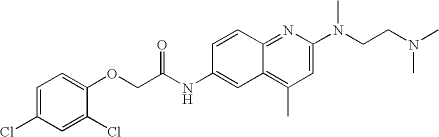

2-(2,4-Dichloro-phenoxy)-N-{2-[(2-dimethylamino-ethyl)-methyl-amino]-4-methyl-quinolin-6-yl}-acetamide. To the aniline from Example 79 (25 mg, 0.1 mmol) in dry DCM (1.5 mL) was added the acid chloride in dry DCM (0.5 mL, 0.25 M, 0.125 mmol). The reaction was stirred u/Ar for 12 h, then washed with Na₂CO₃ (sat.) and concentrated. The residue was purified by chromatography (SiO₂, [MeOH w/5% NH₃]: EtOAc, 1:9 to 1:5) to give the title compound. LC/MS (an20p10): Rt 5.435 min, m/z 461.1 [MH⁺].

### Example 81

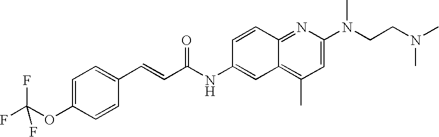

(E)-N-{2-[(2-Dimethylamino-ethyl)-methyl-amino]-4-methyl-quinolin-6-yl}-3-(4-trifluoromethoxy-phenyl)-acrylamide. The title compound was made according to a procedure similar to the one described for Example 80. LC/MS (an20p10): Rt 5.424 min, m/z 473.2 [MH⁺].

### Example 82

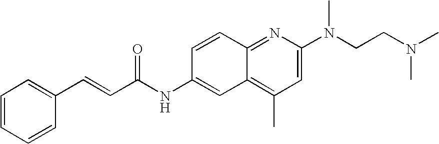

(E)-N-{2-[(2-Dimethylamino-ethyl)-methyl-amino]-4-methyl-quinolin-6-yl}-3-phenyl-acrylamide. The title compound was made according to a procedure similar to the one described for Example 80. LC/MS (an20p10): Rt 4.175 min, m/z 389.2 [MH⁺].

### Example 83

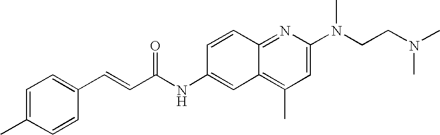

(E)-N-{2-[(2-Dimethylamino-ethyl)-methyl-amino]-4-methyl-quinolin-6-yl}-3-p-tolyl-acrylamide. The title compound was made according to a procedure similar to the one described for Example 80. LC/MS (an20p10): Rt 4.692 min, m/z 403.2 [MH⁺].

### Example 84

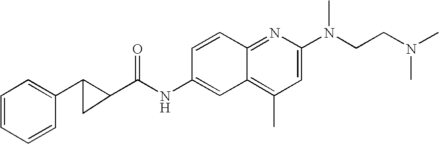

2-Phenyl-cyclopropanecarboxylic acid {2-[(2-dimethylamino-ethyl)-methyl-amino]-4-methyl-quinolin-6-yl}-amide. The title compound was made according to a procedure similar to the one described for Example 80. LC/MS (an20p10): Rt 4.312 min, m/z 403.2 [MH⁺].

### Example 85

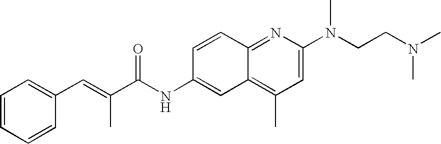

(E)-N-{2-[(2-Dimethylamino-ethyl)-methyl-amino]-4-methyl-quinolin-6-yl}-2-methyl-3-phenyl-acrylamide. The title compound was made according to a procedure similar to the one described for Example 80. LC/MS (an20p10): Rt 4.480 min, m/z 403.2 [MH⁺].

### Example 86

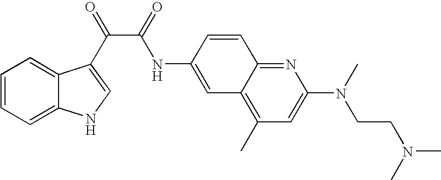

N-{2-[(2-Dimethylamino-ethyl)-methyl-amino]-4-methyl-quinolin-6-yl}-2-(H-indol-3-yl)-2-oxo-acetamide. The title compound was made according to a procedure similar to the one described for Example 80. LC/MS (an20p10): Rt 4.168 min, m/z 430.1 [MH⁺].

### Example 87

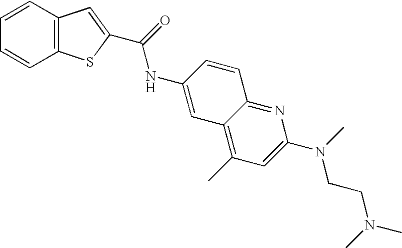

Benzo[b]thiophene-2-carboxylic acid {2-[(2-dimethylamino-ethyl)-methyl-amino]-4-methyl-quinolin-6-yl}-amide. The title compound was made according to a procedure similar to the one described for Example 80. LC/MS (an20p10): Rt 4.589 min, m/z 419.1 [MH⁺].

### Example 88

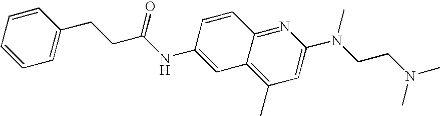

N-{2-[(2-Dimethylamino-ethyl)-methyl-amino]-4-methyl-quinolin-6-yl}-3-phenyl-propionamide. The title compound was made according to a procedure similar to the one described for Example 80. LC/MS (an20p10): Rt 3.871 min, m/z 391.2 [MH⁺].

### Example 89

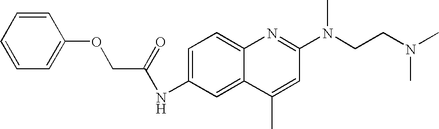

N-{2-[(2-Dimethylamino-ethyl)-methyl-amino]-4-methyl-quinolin-6-yl}-2-phenoxy-acetamide. The title compound was made according to a procedure similar to the one described for Example 80. LC/MS (an20p10): Rt 4.525 min, m/z 393.2 [MH⁺].

### Example 90

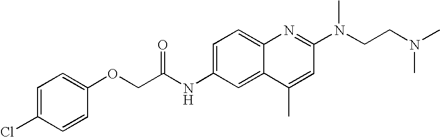

2-(4-Chloro-phenoxy)-N-{2-[(2-dimethylamino-ethyl)-methyl-amino]-4-methyl-quinolin-6-yl}-acetamide. The title compound was made according to a procedure similar to the one described for Example 80. LC/MS (an20p10): Rt 4.714 min, m/z 427.1 [MH⁺].

### Example 91

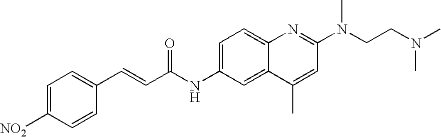

(E)-3-(4-Nitro-phenyl)-N-{2-[(2-dimethylamino-ethyl)-methyl-amino]-4-methyl-quinolin-6-yl}-acrylamide. The title compound was made according to a procedure similar to the one described for Example 80. LC/MS (an20p10): Rt 4.354 min, m/z 434.1 [MH⁺].

### Example 92

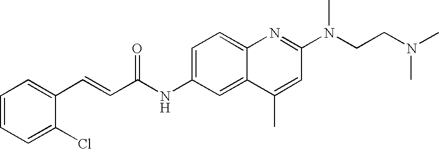

(E)-3-(2-Chloro-phenyl)-N-{2-[(2-dimethylamino-ethyl)-methyl-amino]-4-methyl-quinolin-6-yl}-acrylamide. The title compound was made according to a procedure similar to the one described for Example 80. LC/MS (an20p10): Rt 5.411 min, m/z 423.1 [MH⁺].

### Example 93

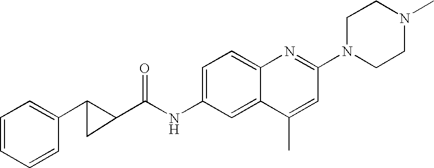

2-Phenyl-cyclopropanecarboxylic acid [4-methyl-2-(4-methyl-piperazin-1-yl)-quinolin-6-yl]-amide. The title compound was made according to a procedure similar to the one described for Example 80. LC/MS (an20p10): Rt 4.256 min, m/z 401.2 [MH⁺].

### Example 94

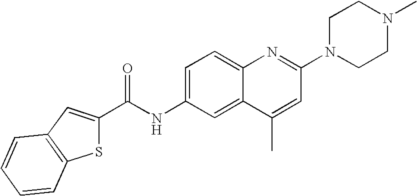

Benzo[b]thiophene-2-carboxylic acid [4-methyl-2-(4-methyl-piperazin-1-yl)-quinolin-6-yl]-amide. The title compound was made according to a procedure similar to the one described for Example 80. LC/MS (an20p10): Rt 4.499 min, m/z 417.1 [MH⁺].

### Example 95

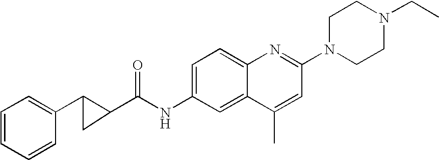

2-Phenyl-cyclopropanecarboxylic acid [2-(4-ethyl-piperazin-1-yl)-4-methyl-quinolin-6-yl]-amide. The title compound was made according to a procedure similar to the one described for Example 80. LC/MS (an20p10): Rt 4.557 min, m/z 415.2 [MH⁺].

### Example 96

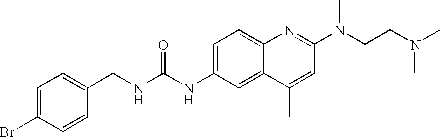

1-(4-Bromo-benzyl)-3-{2-[(2-dimethylamino-ethyl)-methyl-amino]-4-methyl-quinolin-6-yl}-urea. The title compound was made according to a procedure similar to the one described for Example 80. LC/MS (an20p10): Rt 5.510 min, m/z 470.1 [MH⁺].

### Example 97

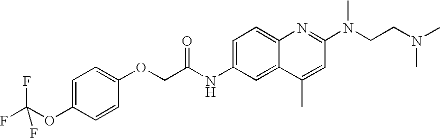

N-{2-[(2-Dimethylamino-ethyl)-methyl-amino]-4-methyl-quinolin-6-yl}-2-(4-trifluoromethoxy-phenoxy)-acetamide. The title compound was made according to a procedure similar to the one described for Example 80. LC/MS (an05p10): Rt 9.968 min, m/z 477.2 [MH⁺].

### Example 98

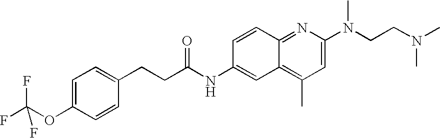

N-{2-[(2-Dimethylamino-ethyl)-methyl-amino]-4-methyl-quinolin-6-yl}-3-(4-trifluoromethoxy-phenyl)-propionamide. The title compound was made according to a procedure similar to the one described for Example 80. LC/MS (an20p10): Rt 5.480 min, m/z 475.2 [MH⁺].

### Example 99

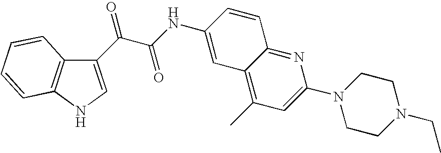

N-[2-(4-Ethyl-piperazin-1-yl)-4-methyl-quinolin-6-yl]-2-(1H-indol-3-yl)-2-oxo-acetamide. The title compound was made according to a procedure similar to the one described for Example 80. LC/MS (an20p10): Rt 4.232 min, m/z 442.1 [MH⁺].

### Example 100

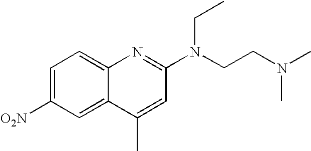

N-Ethyl-N′,N′-dimethyl-N-(4-methyl-6-nitro-quinolin-2-yl)-ethane-1,2-diamine. The title compound was made according to a procedure similar to the one described for Example 2. LC/MS (an20p15): Rt 5.82 min, m/z 303 [MH⁺].

### Example 101

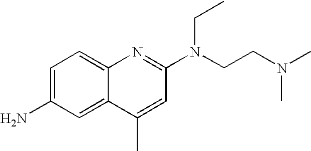

N*2*-(2-Dimethylamino-ethyl)-N*2*-ethyl-4-methyl-quinoline-2,6-diamine. The title compound was made according to a procedure similar to the one described for Example 3. LC/MS (an20p15): Rt 1.68 min, m/z 273 [MH⁺].

### Example 102

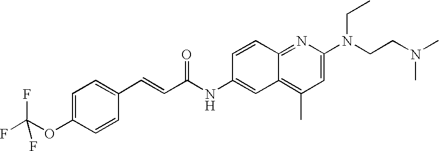

(E)-N-{2-[(2-Dimethylamino-ethyl)-ethyl-amino]-4-methyl-quinolin-6-yl}-3-(4-trifluoromethoxy-phenyl)-acrylamide. The title compound was made according to a procedure similar to the one described for Example 80. LC/MS (an20p15): Rt 7.4 min, m/z 487 [MH⁺].

### Example 103

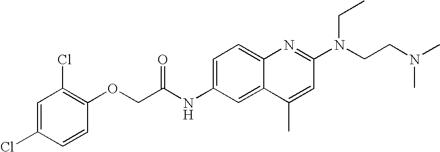

2-(2,4-Dichloro-phenoxy)-N-{2-[(2-dimethylamino-ethyl)-ethyl-amino]-4-methyl-quinolin-6-yl}-acetamide. The title compound was made according to a procedure similar to the one described for Example 80. LC/MS (an20p15): Rt 6.1 min, m/z 475 [MH⁺].

### Example 104

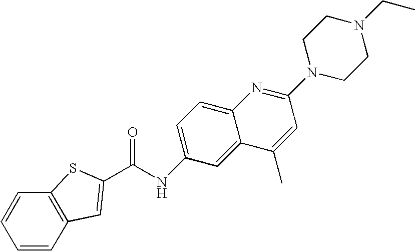

Benzo[b]thiophene-2-carboxylic acid [2-(4-ethyl-piperazin-1-yl)-4-methyl-quinolin-6-yl]-amide. The title compound was made according to a procedure similar to the one described for Example 80. LC/MS (an20p10): Rt 5.597 min, m/z 431.1 [MH⁺].

### Example 105

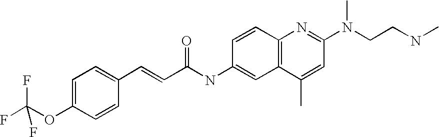

(E)-N-{4-Methyl-2-[methyl-(2-methylamino-ethyl)-amino]-quinolin-6-yl}-3-(4-trifluoromethoxy-phenyl)-acrylamide. The N-Boc protected compound (2.12 mmol) was dissolved in an ethereal solution of hydrogen chloride (2M, 15 mL) and allowed to stand for 90 min, during which time a precipitate was formed which was collected by filtration and dried in vacuo to yield the title compound (814 mg, 82% over 2 steps). LCMS (an20p10): Rt 5.622 min, m/z 459.2 [MH⁺]; ¹H NMR (300 MHz, CDCl₃) 8.02 (1H, s), 7.55 (2H, m), 7.08 (2H, d), 6.98 (2H, d), 6.84 (1H, s), 6.72 (1H, d), 5.80 (1H, d), 3.68 (2H, br t), 3.12 (2H, br t), 2.95 (3H, s), 2.65 (3H, s), 2.50 (3H, s).

### Example 106

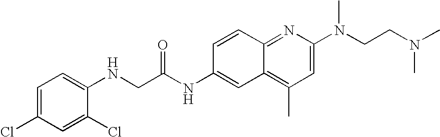

2-(2,4-Dichloro-phenylamino)-N-{2-[(2-dimethylamino-ethyl)-methyl-amino]-4-methyl-quinolin-6-yl}-acetamide. To a suspension of 6-amino-2-(N-2-dimethylaminoethyl-N-methylamino)-4-methylquinoline from Example 79 (0.50 g, 2.05 mmol) and HOBt (0.4 g, 3.1 mmol) in DCM (5 mL) was added PS-CDI (3.0 g, loading 1.35 mmol/g, 4.1 mmol), and the mixture was stirred for 20 min. 2,4-Dichlorophenylaminoacetic acid (0.45 g, 2.05 mmol) was added, and the reaction was stirred at r.t. over night. The resin was removed by filtration and extracted with DCM. The organc phase was added trisamine (2 g) and stirred for 2 h. The resin was removed by filtration and the organic phase was concentrated. The residue was purified on an SCX-column (Eluent: MeOH, then MeOH w/5% NH₄OH) to give the title compound. LC/MS (an20p10): Rt 5.47 min, m/z 460.41 [M⁺].

### Example 107

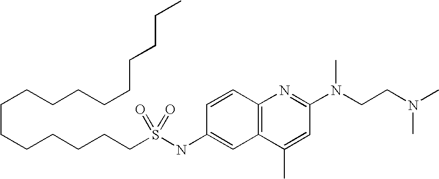

Hexadecane-1-sulfonic acid {2-[(2-dimethylamino-ethyl)-methyl-amino]-4-methyl-quinolin-6-yl}-amide. A vial was charged with dichloromethane (1 mL), aniline (50 mg, 0.19 mmol) and sulfonyl chloride (0.25 mmol). Dimethylformamide (0.2 mL) was added after 15 min. The reaction mixture was allowed to stand overnight before being purified on LCMS to give 7.1 mg of the title compound. ¹H NMR (300 MHz, CDCl₃) 8.56 (1H, br s), 7.70 (1H, d), 7.48 (1H, dd), 6.98 (1H, s), 3.92 (2H, t), 3.22 (3H, s), 3.00 (2H, t), 2.70 (6H, s), 2.62 (3H, s), 1.80 (2H, m), 1.42-1.18 (31H, m).

### Example 108

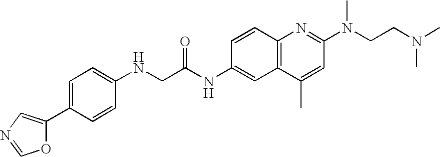

N-{2-[(2-Dimethylamino-ethyl)-methyl-amino]-4-methyl-quinolin-6-yl}-4-oxazol-5-yl-benzenesulfonamide. The title compound was made according to a procedure similar to the one described for Example 107. (3.6 mg); ¹H NMR (300 MHz, CDCl₃) 8.45 (1H, s), 7.98 (1H, s), 7.78 (2H, d), 7.65 (2H, d), 7.32-7.22 (2H, m), 6.78 (1H, s), 4.08 (2H, t), 3.22 (3H, s), 3.12 (2H, t), 2.78 (6H, s), 2.50 (3H, s).

### Example 109

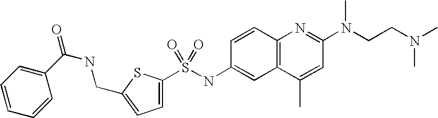

N-(5-{2-[(2-Dimethylamino-ethyl)-methyl-amino]-4-methyl-quinolin-6-ylsulfamoyl}-thiophen-2-ylmethyl)-benzamide. The title compound was made according to a procedure similar to the one described for Example 107. (14.5 mg); ¹H NMR (300 MHz, CDCl₃) 8.50 (1H, br s), 7.77 (2H, d), 7.60-7.32 (7H, m), 6.98-6.90 (2H, m), 4.63 (2H, s), 4.00 (2H, br t), 3.38 (2H, br t), 3.20 (2H, s), 3.00 (6H, s), 2.50 (3H, s).

### Example 110

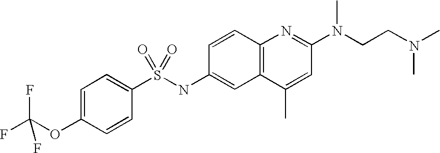

N-{2-[(2-Dimethylamino-ethyl)-methyl-amino]-4-methyl-quinolin-6-yl}-4-trifluoromethoxy-benzenesulfonamide. The title compound was made according to a procedure similar to the one described for example 107. (16.5 mg); ¹H NMR (300 MHz, CDCl₃) 8.51 (1H, br s), 7.78 (2H, d), 7.46 (1H, d), 7.35 (1H, d), 7.20 (2H, d), 6.70 (1H, s), 4.08 (2H, t), 3.20-3.10 (5H, m), 2.80 (6H, s), 2.42 (3H, s).

### Example 111

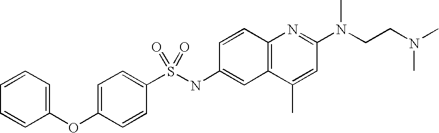

N-{2-[(2-Dimethylamino-ethyl)-methyl-amino]-4-methyl-quinolin-6-yl}-4-phenoxy-benzenesulfonamide. The title compound was made according to a procedure similar to the one described for Example 107. (13.5 mg); ¹H NMR (300 MHz, CDCl₃) 8.52 (1H, br s), 7.68 (2H, d), 7.50-7.32 (3H, m), 7.20-7.15 (2H, m), 7.0 (2H, d), 7.90 (2H, d), 6.70 (1H, s), 4.06 (2H, br app t), 3.20-3.10 (5H, m), 2.79 (6H, s), 2.49 (3H, s).

### Example 112

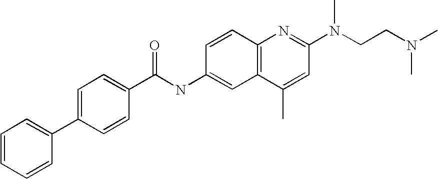

Biphenyl-4-carboxylic acid {2-[(2-dimethylamino-ethyl)-methyl-amino]-4-methyl-quinolin-6-yl}-amide. A vial was charged with dichloromethane (2 mL), dimethylformamide (0.2 mL), aniline (50 mg, 0.19 mmol), PS-carbodiimide (280 mg), hydroxybenzotriazole monohydrate (27 mg, 0.20 mmol) and carboxylic acid (0.25 mmol). The shaker was set to full for 16 h before the addition of PS-trisamine (100 mg), PS-isocyanate (100 mg), followed by shaking for 2 h. The reaction mixture was filtered directly onto SCX-plugs utilizing the stacker capability. The reaction mixture residue was washed with dichloromethane (2 mL) and methanol (5 mL). The collection rack was then charged with clean numbered vials and the product eluted from the SCX using a 5% ammonia in ethanol solution (5 mL). The volatiles were removed in vacuo to give the title compound. LCMS (an20p10): Rt 4.915 min, m/z 439.2 [MH⁺].

### Example 113

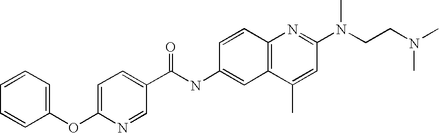

N-{2-[(2-Dimethylamino-ethyl)-methyl-amino]-4-methyl-quinolin-6-yl}-6-phenoxy-nicotinamide. The title compound was made according to a procedure similar to the one described for Example 112. LCMS (an20p10): Rt 4.443 min, m/z 456.2 [MH⁺].

### Example 114

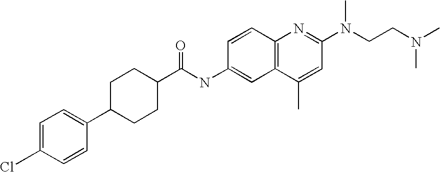

4-(4-Chloro-phenyl)-cyclohexanecarboxylic acid {2-[(2-dimethylamino-ethyl)-methyl-amino]-4-methyl-quinolin-6-yl}-amide. The title compound was made according to a procedure similar to the one described for Example 112. LCMS (an20p10): Rt 5.450 min, m/z 481.2 [MH⁺].

### Example 115

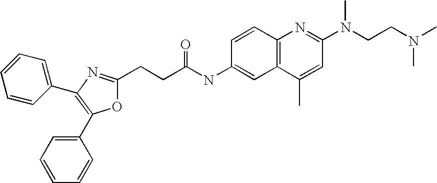

N-{2-[(2-Dimethylamino-ethyl)-methyl-amino]-4-methyl-quinolin-6-yl}-3-(4,5-diphenyl-oxazol-2-yl)-propionamide. The title compound was made according to a procedure similar to the one described for Example 112. LCMS (an20p10), 5.290 min, M⁺H 534.2 (90%), 267.6 (100%).

### Example 116

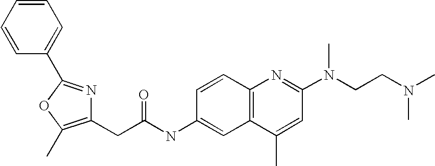

N-{2-[(2-Dimethylamino-ethyl)-methyl-amino]-4-methyl-quinolin-6-yl}-2-(5-methyl-2-phenyl-oxazol-4-yl)-acetamide. The title compound was made according to a procedure similar to the one described for Example 112. LCMS (an20p10), 4.459 min, M⁺H 458.2 (100%), 207.1 (90%).

### Example 117

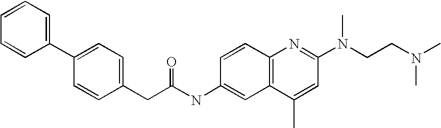

2-Biphenyl-4-yl-N-{2-[(2-dimethylamino-ethyl)-methyl-amino]-4-methyl-quinolin-6-yl}-acetamide. The title compound was made according to a procedure similar to the one described for Example 112. LCMS (an20p10), 5.077 min, M⁺H 453.2 (50%), 227.1 (100%).

### Example 118

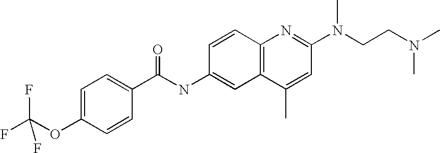

N-{2-[(2-Dimethylamino-ethyl)-methyl-amino]-4-methyl-quinolin-6-yl}-4-trifluoromethoxy-benzamide. The title compound was made according to a procedure similar to the one described for Example 112. LCMS (an20p10): Rt 6.094 min, m/z 447.1 [MH⁺].

### Example 119

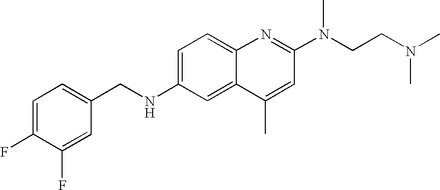

N*6*-(3,4-Difluoro-benzyl)-N*2*-(2-dimethylamino-ethyl)-4,N*2*-dimethyl-quinoline-2,6-diamine. 6-Amino-2-(N-2-dimethylaminoethyl-N-methylamino)-4-methylquinoline from Example 79 (2 g, 7.8 mmol), 3,4-difluorobenzaldehyde (1.66 g, 11.7 mmol, 1.3 mL), and sodium methoxide (2.1 g, 38.8 mmol) were stirred together in methanol (150 mL) at 40° C. for 48 h before sodium borohydride (653 mg, 17.2 mmol) was added and the mixture heated to 50° C. for a further 48 h. The reaction mixture was allowed to cool before the volatiles were removed in vacuo. The residue was taken up in hydrochloric acid (4M, 50 mL) and washed with dichloromethane (30 mL). The aqueous layer was then basified with sodium hydroxide (4M) to pH 10 and extracted with dichloromethane (3×30 mL). These organic extracts were combined and washed with brine (50 mL), dried over sodium sulfate and reduced in vacuo to yield the title compound as a brown oil (1.69 g, 4.4 mmol, 56%) which was used without further purification.

### Example 120

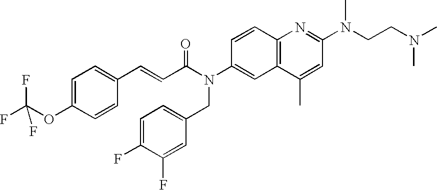

(E)-N-(3,4-Difluoro-benzyl)-N-{2-[(2-dimethylamino-ethyl)-methyl-am in o]4-methyl-quinolin-6-yl}-3-(4-trifluoromethoxy-phenyl)-acrylamide. Secondary aniline form Example 119 (563 mg, 1.46 mL) was dissolved in dichloromethane (20 mL) and cooled to 0° C. under nitrogen atmosphere before 4-trifluoromethoxycinnamoyl chloride (364 mg, 1.46 mmol) was added. After stirring for 15 min, the ice bath was removed and the reaction was allowed to stir until complete. A solid residue was formed in the reaction and the whole reaction mixture was dissolved in hydrochloric acid (1M, 50 mL) and washed with dichloromethane. The aqueous layer was basified with sodium hydroxide (4M) and extracted with dichloromethane. These extracts were reduced in vacuo and a sample of product was purified on LC/MS to yield the title compound (16 mg, 0.026 mmol). LCMS (an20p10): Rt 6.500 min, m/z 599.2 [MH⁺]; ¹H NMR (300 MHz, CDCl₃) 7.72 (1H, d), 7.60 (1H, d), 7.40 (1H, s), 7.32-6.97 (8H, m), 6.80 (1H, s), 6.32 (1H, d), 5.01 (2H, br app s), 4.15 (2H, br app s), 3.30 (2H, br app s), 3.22 (3H, br s), 2.91 (6H, br s), 2.51 (3H, br s).

### Example 121

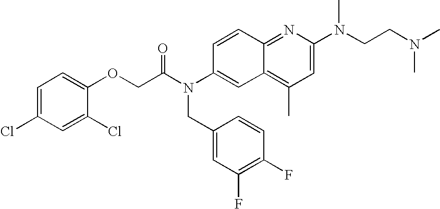 2-(2,4-Dichloro-phenoxy)-N-(3,4-difluoro-benzyl)-N-{2-[(2-di methylamino-ethyl)-methyl-amino]-4-methyl-quinolin-6-yl}-acetamide. The title compound was made according to a procedure similar to the one described for Example 112. (4.6 mg); LC/MS (an20p10): Rt 6.777 min, m/z 587.1 [MH⁺]; ¹H NMR (300 MHz, CDCl₃) 7.62 (1H, d), 7.32 (2H, m), 7.18-6.88 (5H, m), 6.80 (1H, s), 6.68 (1H, d), 4.88 (2H, s), 4.49 (2H, s), 4.12 (2H, br t), 3.22 (3H, s), 3.15 (2H, br t), 2.78 (6H, s), 2.47 (3H, s).

### Example 122

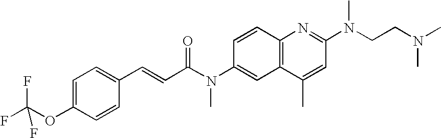

(E)-N-{2-[(2-Dimethylamino-ethyl)-methyl-amino]-4-methyl-quinolin-6-yl}-N-methyl-3-(4-trifluoromethoxy-phenyl)-acrylamide. The title compound was made from N*2*-(2-dimethylamino-ethyl)-4,N*2*,N*6*-trimethyl-quinoline-2,6-diamine and 4-trifluoromethoxyphenylacryloys chloride according to a procedure similar to the one described for Example 5. LC/MS (an20p10): Rt 5.37 min, m/z 487 [MH⁺].

### Example 123

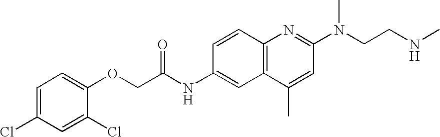

2-(2,4-Dichloro-phenoxy)-N-{4-methyl-2-[methyl-(2-methylamino-ethyl)-amino]-quinolin-6-yl}-acetamide. The title compound was made according to a procedure similar to the one described for Example 39. LC/MS (an20p10): Rt 5.11 min, m/z 447, 449 [MH⁺].

### Example 124

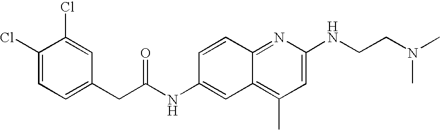

2-(3,4-Dichloro-phenyl)-N-[2-(2-dimethylamino-ethylamino)-4-methyl-quinolin-6-yl]-acetamide. The title compound was made according to a procedure similar to the one described for Example 39. LC/MS (an20p10): Rt 6.34 min, m/z 431.1 [MH⁺].

### Example 125

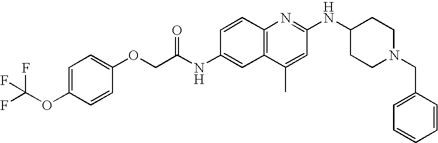

N-[2-(1-Benzyl-piperidin-4-ylamino)-4-methyl-quinolin-6-yl]-2-(4-trifluoromethoxy-phenoxy)-acetamide. The title compound was made according to a procedure similar to the one described for Example 39. LC/MS (an050p7): Rt 4.40 min, m/z 565 [MH⁺].

### Example 126

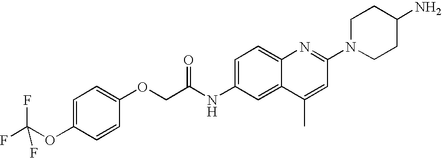

N-[2-(4-Amino-piperidin-1-yl)-4-methyl-quinolin-6-yl]-2-(4-trifluoromethoxy-phenoxy)-acetamide. The title compound was made according to a procedure similar to the one described for Example 27. LC/MS (an050p7): Rt 3.87 min, m/z 475 [MH⁺].

### Example 127

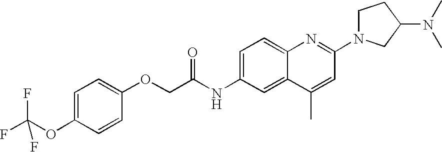

N-[2-(3-Dimethylamino-pyrrolidin-1-yl)-4-methyl-quinolin-6-yl]-2-(4-trifluoromethoxy-phenoxy)-acetamide. The title compound was made according to a procedure similar to the one described for Example 27. LC/MS (an050p7): Rt 4.78 min, m/z 489 [MH⁺].

### Example 128

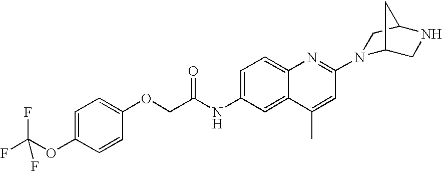

N-[2-(2,5-Diaza-bicyclo[2.2.1]hept-2-yl)-4-methyl-quinolin-6-yl]-2-(4-trifluoromethoxy-phenoxy)-acetamide. The title compound was made according to a procedure similar to the one described for Example 27. LC/MS (an050p7): Rt 5.78 min, m/z 473 [MH⁺].

### Example 129

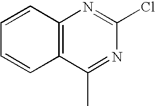

2-Chloro-4-methyl-quinazoline. To a solution of 2,4-dichloroquinazoline (200 mg, 1 mmol) and Pd(PPh₃)-4 (81 mg, 0.07eq) in dry THF (3 ml) was added a 2N solution of trimethylaluminum in hexane (0.17 ml, 0.33 mmol). The reaction mixture was stirred for 20h00 at 75° C. under an argon atmosphere. The reaction was quenched by the addition of water, extracted with ethyl acetate. The organic phase was washed with water, dried over MgSO₄ and concentrated in vacuo to give Example 129 (235 mg, weigh>theoretical w=179 mg, 1.0 mmol) which was used without further purification. LC-MS (an10p8): Rt 2.49 min; MW+1=179

### Example 130

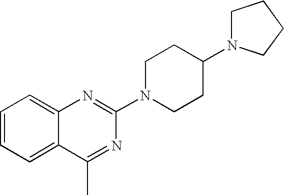

4-Methyl-2-(4-pyrrolidin-1-yl-piperidin-1-yl)-quinazoline. A mixture of Example 129 (120 mg, 0.6 mmol) and 4-(1-pyrrolidinyl)-piperidine (130 mg, 0.8 mmol) were stirred for 5 minutes at 150° C. in the microwave. After cooling, the residue was diluted with EtOAc and washed with water. The organic phase was dried over MgSO₄ and concentrated in vacuo to give Example 130 (199 mg, 0.6 mmol, 100%) as a pale yellow oil which was used without further purification. 300 MHz ¹HNMR (CDCl₃): δ ppm 7.2 (t, 1H); 7.6 (m, 2H); 7.85 (d, 1H)

### Example 131

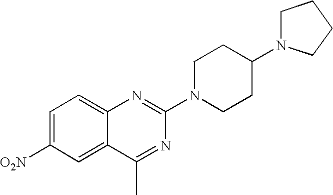

4-Methyl-6-nitro-2-(4-pyrrolidin-1-yl-piperidin-1-yl)-quinazoline. To cooled (−5° C.-0° C.) fumic acid (2 ml) was slowly added Example 130 (199 mg, 0.6 mmol) over a period of 15 minutes. After completion, the reaction mixture was stirred for a further 30 minutes at (−5° C.-0° C.). The mixture was then poured onto ice/water and basified with 30% aq.NaOH, extracted with ethyl acetate (2×), dried over MgSO₄ and concentrated in vacuo. The residue was purified over silica gel chromatography (eluent:CH₂Cl₂/MeOH/NH₃:100/0/0 up to 90/9/1) to give Example 131 (229 mg, 0.6 mmol, 100%, estimated yield as ¹HNMR showed required compound+impurities) which was used without further purification. 300 MHz ¹HNMR (CDCl₃): δ ppm 7.55 (d, 1H); 8.35 (d, 1H); 8.75 (s, 1H).

### Example 132

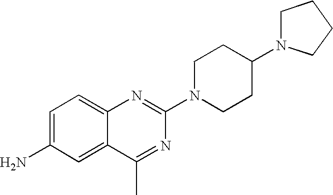

4-Methyl-2-(4-pyrrolidin-1-yl-piperidin-1-yl)-quinazolin-6-ylamine. To a solution of Example 131 (229 mg, 0.6 mmol) in methanol (10 ml) was added catalytic amount of 10% wtPd/C. The reaction mixture was stirred under a hydrogen atmosphere at RT for 2 hours. Catalyst was filtered off and the filtrate was concentrated in vacuo to give Example 132 (208 mg, 0.6 mmol, 100%, estimated yield) which was used without further purification and characterization in Example 133.

### Example 133

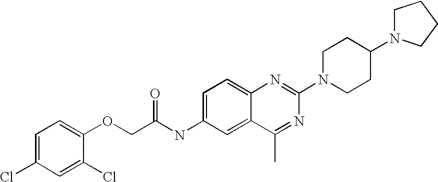

2-(2,4-Dichloro-phenoxy)-N-[4-methyl-2-(4-pyrrolidin-1-yl-piperidin-1-yl)-quinazolin-6-yl]-acetamide. To a cooled (0C) solution of Example 132 (208 mg, 0.6 mmol) in CH₂Cl₂ (10 ml) was added, under an inert atmosphere, 2,4-dichlorophenoxy-acetyl chloride (200 mg, 0.8 mmol). The reaction mixture was stirred at RT overnight. The mixture was then partitioned between water and EtOAc. The aqueous phase was basified and extracted with EtOAc. The organic phase was dried over MgSO₄ and concentrated in vacuo. The residue was purified over silica gel chromatography to give Example 133 (4.4 mg, 0.0085 mmol, 1.4%). LC-MS (an10p8): Rt=7.0 min; MW+1=514

### Example 134

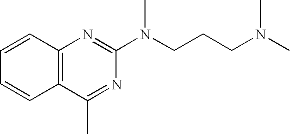

N,N,N′-Trimethyl-N′-(4-methyl-quinazolin-2-yl)-propane-1,3-diamine. A mixture of Example 129 (235 mg, theoretical w=179 mg, 1.0 mmol) and N,N,N′-trimethyl-1,3-propane diamine (0.29 ml, 2.0 mmol) was stirred for 5 minutes at 150° C. in the microwave. After cooling, the residue was diluted with CH₂Cl₂ and washed with sat.aq. NaHCO₃. The organic phase was dried over MgSO₄ and concentrated in vacuo to give an oil which was purified over SCX column (1 g) to give Example 134 (170 mg, 0.66 mmol, 66%) as an pale brownish-orange oil. LC-MS (an10p8): Rt=4.55 min; MW+1=259

### Example 135′

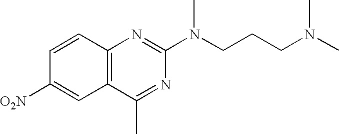

N,N,N′-Trimethyl-N′-(4-methyl-6-nitro-quinazolin-2-yl)-propane-1,3-diamine. To cooled (−5° C.-0° C.) fumic acid (2 ml) was slowly added Example 134 (120 mg, 0.46 mmol) over a period of 15 minutes. After completion, the reaction mixture was stirred for a further 45 minutes at (−5° C.-0° C.). The mixture was then poured onto ice/water and basified with 30% aq.NaOH, extracted with ethyl acetate (2×), dried over MgSO₄ and concentrated in vacuo. The residue was purified over silica gel chromatography (eluent:CH₂Cl₂/MeOH/NH₃:100/0/0 up to 90/9/1) to give Example 135 as a yellow oil (13 mg, 0.042 mmol, 21%). 300 MHz ¹HNMR (CDCl₃): δ ppm 2.2 (s, 6H); 2.8 (s, 3H); 3.3 (s, 3H)

### Example 136

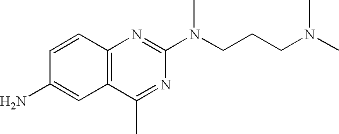

N*2*-(3-Dimethylamino-propyl)-4,N*2*-dimethyl-quinazoline-2,6-diamine.

To a solution of Example 135 (13 mg, 0.042 mmol) in methanol (5 ml) was added 10% wtPd/C (1.3 mg, 10% w/w). The reaction mixture was stirred under a hydrogen atmosphere at 35° C. for 30 minutes. Catalyst was filtered off and the filtrate was concentrated in vacuo to give Example 136 as a yellow oil (11.7 mg, 0.042 mmol, 100%) which was used without further purification and characterization in Example 133.

### Example 137

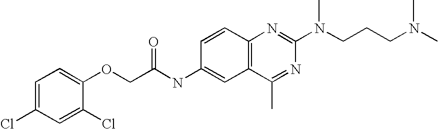

2(2,4-Dichlorophenoxy)N{2[(3-dimethylaminopropyl)methyl-amino]-4-methyl-quinazolin-6-yl}-acetamide. To a solution of Example 136 (11.7 mg, 0.042 mmol) in CH₂CO₂ (1 ml) was added 2,4-dichlorophenoxy-acetyl chloride (12.3 mg, 0.050 mmol). The reaction mixture was stirred at RT overnight. The mixture was then loaded on a 1 g silica column and elution was carried out with pure CH₂Cl₂ followed by CH₂Cl₂/MeOH/NH₃ 95/4.5/0.5 to give Example 137 as a yellow solid (4.3 mg, 0.009 mmol, 21%). 300 MHz ¹HNMR (CDCl₃): δ ppm 2.35 (s, 6H); 2.75 (s, 3H); 3.25 (s, 3H); 4.2 (s, 2H).

### Example 138

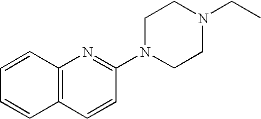

2-(4-Ethyl-piperazin-1-yl)-quinoline. A mixture of 2-chloroquinoline (650 mg, 3.97 mmol) and N-ethylpiperazine (1.27 ml, 10 mmol) was heated at 90° C. overnight under an inert atmosphere. After cooling, the mixture was partitioned between CH₂Cl₂ and 1N aq.HCl. The phases were separated. pH of the aqueous phase was adjusted to 7 with aq.NaHCO₃. The aqueous phase was extracted with CH₂Cl₂ (2×5 ml). The combined organic phases were dried over MgSO₄ and concentrated in vacuo to give Example 138 (1.06 g, weigh>theoretical w=958 mg, 3.97 mmol) which was used without further purification. LC-MS (an20p10): Rt=3.80 min; MW+1=242.

### Example 139

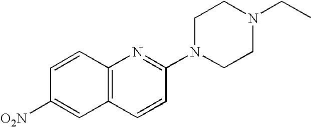

2-(4-Ethyl-piperazin-1-yl)-6-nitro-quinoline. To cooled (−10° C.) fuming nitric acid (50 ml) was slowly added Example 138 (958 mg, 3.97 mmol). After completion, the mixture was stirred for three days at RT. The mixture was cooled to 0° C. and pH was adjusted to 10 by addition of solid Na₂CO₃. The aqueous phase was extracted with CH₂Cl₂ (2×20 ml). The combined organic phases were dried over MgSO₄ and concentrated in vacuo to give Example 139 (530 mg, 1.85 mmol, 46%) which was used without further purification. 300 MHz ¹HNMR (CDCl₃): δ ppm 1.13 (s, 3H); 2.51 (q, 2H); 2.59 (t, 4H)

### Example 140

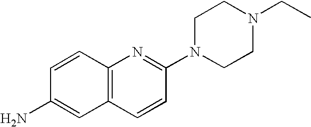

2-(4-Ethyl-piperazin-1-yl)-quinolin-6-ylamine. To a solution of Example 139 (530 mg, 1.85 mmol) in ethanol (20 ml) was added 10% wtPd/C (53 mg, 10% w/w). The reaction mixture was stirred under a hydrogen atmosphere at RT overnight. Catalyst was filtered off and the filtrate was concentrated in vacuo to give Example 140 (474 mg, 1.85 mmol, 100%) which was used without further purification. LC-MS (an20p10): Rt=1.69 min; MW+1=257.

### Example 141

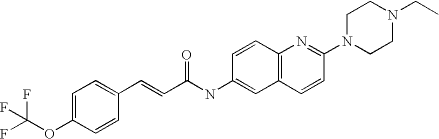

N-[2-(4-Ethyl-piperazin-1-yl)-quinolin-6-yl]-3-(4-trifluoromethoxy-phenyl)-acrylamide. To a solution of Example 140 (20 mg, 0.078 mmol) in CH₂Cl₂ (1 ml) was added, under an inert atmosphere, 3-(4-trifluoromethoxyphenyl)prop-2-enoylchloride (25 mg, 0.097 mmol). The reaction mixture was stirred overnight at RT. Ethyl acetate (2 ml) was added. The organic phase was washed with sat.aq.NaHCO₃ (2 ml), dried over MgSO₄ and concentrated in vacuo. The residue was purified over silica gel chromatography (1 g silica; eluent: CH₂Cl₂, then CH₂Cl₂/MeOH/NH₃:300/10/1 to 100/10/1) to give Example 141 (18 mg, 0.036, 49%). LC-MS (an20p10): Rt=5.57 min; MW+1=471

### Example 142

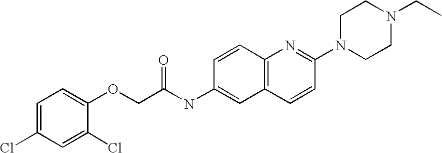

2-(2,4-Dichloro-phenoxy)-N-[2-(4-ethyl-piperazin-1-yl)-quinolin-6-yl]-acetamide. According to a similar procedure to the one described in Example 141 was synthesised Example 142, using Example 140 as starting material. LC-MS (an20p10): Rt=5.73 min; MW=459.

### Example 143

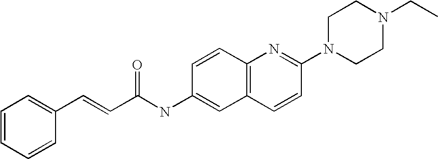

N-[2-(4-Ethyl-piperazin-1-yl)-quinolin-6-yl]-3-phenyl-acrylamide. According to a similar procedure to the one described in Example 141 was synthesised Example 143, using Example 140 as starting material. 300 MHz ¹HNMR (CDCl₃): δ ppm 1.16(t, 3H); 2.49(q, 2H); 8.27(s, 1H).

### Example 144

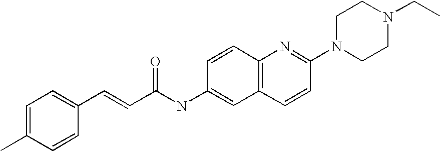

N-[2-(4-Ethyl-piperazin-1-yl)-quinolin-6-yl]-3-p-tolyl-acrylamide. To a cooled (0° C.) suspension of 4-methylcinnamic acid (15.8 mg, 0.097 mmol) were successively added oxalyl chloride (6 μl) and DMF (2 μl). The reaction mixture was stirred for 30 minutes at 0° C. and then for 1 hour at RT. Triethylamine (16 μl) was then added to give mixture A.

To a solution of Example 140 (20 mg, 0.078 mmol) in dichloromethane (1 ml) was added mixture A. The resulting reaction mixture was stirred overnight at RT. Ethyl acetate (2 ml) was added. The organic phase was washed with sat.aq.NaHCO₃ (2 ml). The aqueous phase was extracted with ethyl acetate (2×1 ml). The combined organic phases were dried over MgSO₄ and concentrated in vacuo. The residue was chromatographed on silica (1 g, eluent: CH₂Cl₂, then CH₂Cl₂/MeOH/NH₃: 300/10/1 to 100/10/1) to give Example 144 (7.3 mg, 0.018 mmol, 23%). 300 MHz ¹HNMR (CDCl₃): δ ppm 1.16(t, 3H); 2.39(s, 3H); 2.48(q, 2H); 8.25(s, 1H).

### Example 145

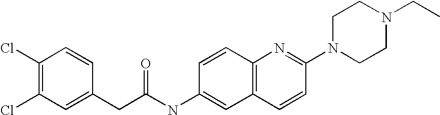

2-(3,4-Dichloro-phenyl)-N-[2-(4-ethyl-piperazin-1-yl)-quinolin-6-yl]-acetamide. According to a similar procedure to the one described in Example 144, Example 145 was synthesised using Example 140 as starting material. 300 MHz ¹HNMR (CDCl₃): δ ppm 1.15(t, 3H); 2.48(q, 2H); 3.71(s, 2H); 8.04(s, 1H).

### Example 146

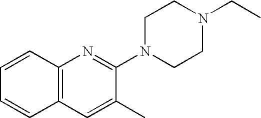

2-(4-Ethyl-piperazin-1-yl)-3-methyl-quinoline. According to a similar procedure to the one described in Example 138, Example 146 was synthesised using 2-chloro-3-methylquinoline and N-ethylpiperazine as starting materials. Example 146 was used as crude without analytical characterization in the synthesis of Example 147

### Example 147

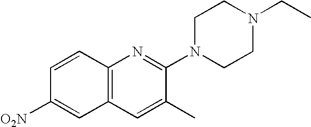

2-(4-Ethyl-piperazin-1-yl)-3-methyl-6-nitro-quinoline. According to a similar procedure to the one described in Example 139, Example 147 was synthesised using Example 146 as starting material. 300 MHz ¹HNMR (CDCl₃): δ ppm 1.16 (t, 3H); 2.45 (s, 3H); 3.52 (t, 4H).

### Example 148

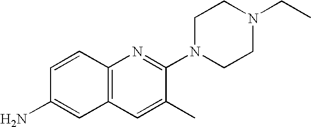

2-(4-Ethyl-piperazin-1-yl)-3-methyl-quinolin-6-ylamine. According to a similar procedure to the one described in Example 140, Example 148 was synthesised using Example 147 as starting material. LC-MS (an20p10): Rt=1.81 min; MW+1=271; 300 MHz ¹HNMR (CDCl₃): δ ppm 1.38 (t, 3H); 2.34 (s, 3H); 3.62 (t, 4H).

### Example 149

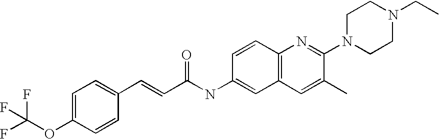

N-[2-(4-Ethyl-piperazin-1-yl)-3-methyl-quinolin-6-yl]-3-(4-trifluoromethoxy-phenyl)-acrylamide. According to a similar procedure to the one described in Example 141, Example 149 was synthesised using Example 148 as starting material. LC-MS (an20p10): Rt=7.35 min; MW+1=485.

### Example 150

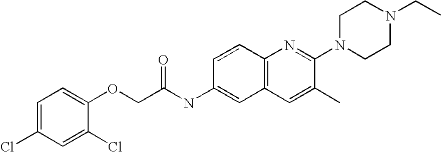

2-(2,4-Dichloro-phenoxy)-N-[2-(4-ethyl-piperazin-1-yl)-3-methyl-quinolin-6-yl]-acetamide. According to a similar procedure to the one described in Example 141, Example 150 was synthesised using Example 148 as starting material. LC-MS (an20p10): Rt=7.35 min; MW=473.

### Example 151

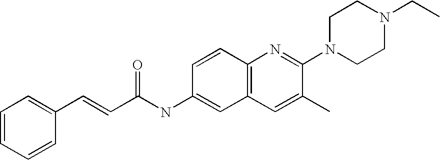

N-[2-(4-Ethyl-piperazin-1-yl)-3-methyl-quinolin-6-yl]-3-phenyl-acrylamide. According to a similar procedure to the one described in Example 141, Example 151 was synthesised using Example 148 as starting material. 300 MHz ¹HNMR (CDCl₃): δ ppm 1.18 (t, 3H); 2.44 (s, 3H); 3.62 (t, 4H); 8.28(s, 1H).

### Example 152

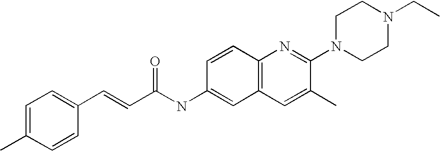

N-[2-(4-Ethyl-piperazin-1-yl)-3-methyl-quinolin-6-yl]-3-p-tolyl-acrylamide. According to a similar procedure to the one described in Example 144, Example 151 was synthesised, using Example 148 as starting material. 300 MHz ¹HNMR (CDCl₃): δ ppm 1.16 (t, 3H); 2.394 (s, 3H); 2.43 (s, 34H); 8.27(s, 1H).

### Example 153

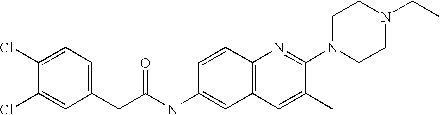

2-(3,4-Dichloro-phenyl)-N-[2-(4-ethyl-piperazin-1-yl)-3-methyl-quinolin-6-yl]-acetamide. According to a similar procedure to the one described in Example 144, Example 153 was synthesised using Example 148 as starting material. 300 MHz ¹HNMR (CDCl₃): δ ppm 1.15 (t, 3H); 2.40 (s, 3H); 3.71 (s, 2H); 8.068(s, 1H).

### Example 154

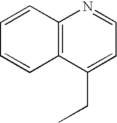

4-Ethyl-quinoline. To a cooled (−78° C.) solution of lepidine (3.3 ml, 25 mmol) in dry THF (35 ml) was slowly added over a period of 30 minutes, under an inert atmosphere, a 2M solution of LDA in heptane (15 ml, 30 mmol). After stirring for 1 h 30 minutes at −78° C., methyl iodide (1.9 ml, 30 mmol) was added and stirring was continued at −78° C. for a further 2 hours. The mixture was then allowed to warm up to RT over 1 hour. The reaction mixture was quenched by addition of sat.aq.NH₄Cl (100 ml) and extracted with EtOAc (200 ml). The organic phase was dried over MgSO₄ and concentrated in vacuo. The residue was purified by flash chromatography (silica, eluent: CH₂Cl₂/MeOH: 10/0 to 10/1) to give Example 154(3.2 g, 20.3 mmol, 81%). LC-MS (an20p15): Rt=4.0 min; MW+1=158.

### Example 155

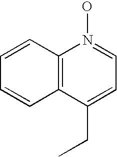

4-Ethyl-quinoline-N-oxide. To a solution of Example 154(3.2 g, 20.3 mmol) in chloroform (50 ml) was added meta-chloroperbenzoic acid (6.7 g, 30 mmol). After stirring for 3 hours at RT, sat.aq.Na₂CO₃ (150 ml) was added and the mixture was extracted with CH₂Cl₂ (200 ml). The organic phase was dried over MgSO₄ and concentrated in vacuo. The residue was purified by chromatography (silica, eluent: CH₂Cl₂/MeOH: 10/0 to 10/0.5) to give Example 155 (2.1 g, 12.1 mmol, 61%). LC-MS (an20p15): Rt 7.3 min; MW+1=174.

### Example 156

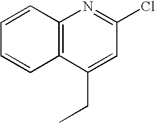

2-Chloro-4-ethyl-quinoline. To a solution of Example 155 (2 g, 11.5 mmol) in toluene (40 ml) was added diisopropylethylamine (2.4 ml, 13.8 mmol). The mixture was then cooled to 0° C. and phosphoryl oxychloride (5.4 ml, 57.5 mmol) was added at such a rate to keep internal temperature below 40° C. After completion, stirring was continued for a further 4 hours. Sat.aq.NaHCO₃ (100 ml) was added and the mixture was extracted with EtOAc (2×100 ml). The organic phases were combined, dried over MgSO₄ and concentrated in vacuo. The residue was chromatographed (silica, eluent: CH₂Cl₂) to give Example 156 (840 mg, 4.38 mmol, 38%). LC-MS (an20p15): Rt=14.1 min; MW+1 =192

### Example 157

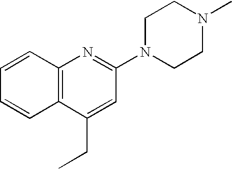

4-Ethyl-2-(4-methyl-piperazin-1-yl)-quinoline. According to a similar procedure to the one described in Example 138, Example 157 was synthesised using Example 156 and N-methylpiperazine as starting materials. 300 MHz ¹HNMR (CDCl₃): δ ppm 1.38 (t, 3H); 2.38 (s, 3H); 2.58 (t, 4H); 3.0 (q, 2H); 3.79 (t, 4H); 6.87 (s, 1H).

### Example 158

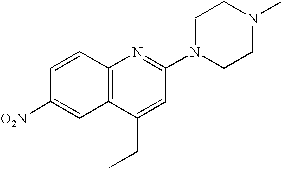

4-Ethyl-2-(4-methyl-piperazin-1-yl)-6-nitro-quinoline. According to a similar procedure to the one described in Example 139, Example 158 was synthesised using Example 157 as starting material. LC-MS (an20p15): Rt 6.96 min; MW+1=300.

### Example 159

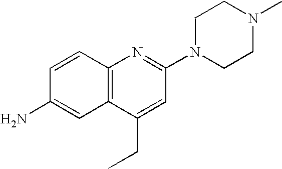

4-Ethyl-2-(4-methyl-piperazin-1-yl)-quinolin-6-ylamine. According to a similar procedure to the one described in Example 140, Example 159 was synthesised using Example 158 as starting material. 300 MHz ¹HNMR (CDCl₃): δ ppm 1.35 (t, 3H); 2.37 (s, 3H); 2.58 (t, 4H); 2.9 (q, 2H); 3.69 (t, 4H); 6.82 (s, 1H); 7.02 (m, 2H); 7.6 (d, 1H).

### Example 160

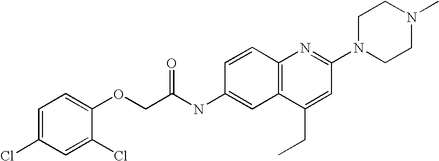

2-(2,4-Dichloro-phenoxy)-N-[4-ethyl-2-(4-methyl-piperazin-1-yl)-quinolin-6-yl]-acetamide. According to a similar procedure to the one described in Example 141, Example 160 was synthesised using Example 159 as starting material. 300 MHz

¹HNMR (CDCl₃): δ ppm 1.39(t, 3H); 2.39(s, 3H); 2.59(m, 4H); 3(q, 2H); 3.78(m, 4H); 4.67(s, 2H); 6.88(s, 1H); 6.95(d, 1H); 7.28(m, 1H); 7.5(m, 2H); 7.72(d, 1H); 8.35(s, 1H); 8.64(s, 1H).

### Example 161

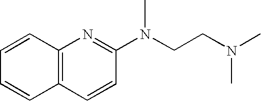

N,N,N′-Trimethyl-N′-quinolin-2-yl-ethane-1,2-diamine. According to a similar procedure to the one described in Example 138, Example 161 was synthesised using 2-chloroquinoline and N,N,N′-trimethylethylenediamine as starting materials. LC-MS (an20p10): Rt=3.80 min; MW+1=230.

### Example 162

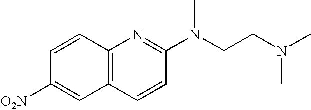

N,N,N′-Trimethyl-N′-(6-nitro-quinolin-2-yl)-ethane-1,2-diamine. According to a similar procedure to the one described in Example 139, Example 162 was synthesised using Example 161 as starting material. 300 MHz ¹HNMR (CDCl₃): δ ppm 2.39 (s, 6H); 3.26 (s, 3H).

### Example 163

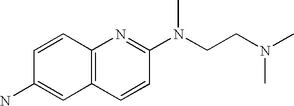

N*2*-(2-Dimethylamino-ethyl)-N*2*-methyl-quinoline-2,6-diamine. According to a similar procedure to the one described in Example 140, Example 163 was synthesised using Example 162 as starting material. LC-MS (an20p10): Rt=1.55 min; MW+1=245; 300 MHz ¹HNMR (CDCl₃): δ ppm 2.51 (s, 6H); 3.17 (s, 3H).

### Example 164

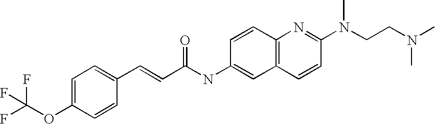

N-{2-[(2-Dimethylamino-ethyl)-methyl-amino]-quinolin-6-yl}-3-(4-trifluoromethoxy-phenyl)-acrylamide. According to a similar procedure to the one described in Example 141, Example 164 was synthesised using Example 163 as starting material. LC-MS (an20p10): Rt=6.10 min; MW+1=459.

### Example 165

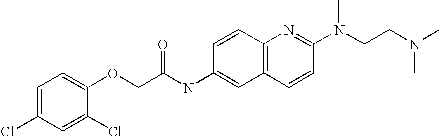

2-(2,4-Dichloro-phenoxy)-N{2-[(2-dimethylamino-ethyl)-methyl-amino]-quinolin-6-yl}-acetamide. According to a similar procedure to the one described in Example 141, Example 165 was synthesised using Example 163 as starting material. LC-MS (an20p10): Rt 6.02 min; MW=447.

### Example 166

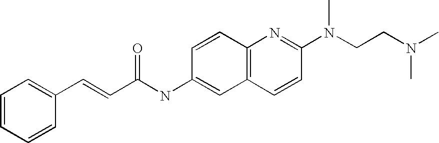

N-{2-[(2-Dimethylamino-ethyl)-methyl-amino]-quinolin-6-yl}-3-phenyl-acrylamide. According to a similar procedure to the one described in Example 141, Example 166 was synthesised using Example 163 as starting material. LC-MS (an20p10): Rt=4.67 min; MW+1=375; 300 MHz ¹HNMR (CDCl₃): δ ppm 2.36 (s, 6H); 3.21 (s, 3H); 8.23(s, 1H).

### Example 167

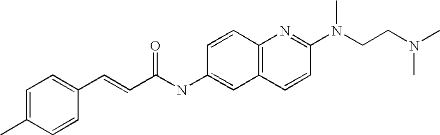

N-{2-[(2-Dimethylamino-ethyl)-methyl-amino]-quinolin-6-yl}-3-p-tolyl-acrylamide. According to a similar procedure to the one described in Example 144, Example 167 was synthesised using Example 163 as starting material. LC-MS (an20p10): Rt=5.11 min; MW+1=389; 300 MHz ¹HNMR (CDCl₃): δ ppm 2.36 (s, 6H); 2.39(s, 3H); 3.21 (s, 3H); 8.21 (s, 1H).

### Example 168

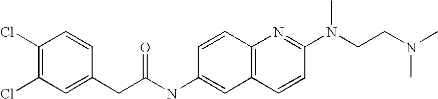

2-(3,4-Dichloro-phenyl)-N-{2-[2-dimethylamino-ethyl)-methyl-amino]-quinolin-6-yl}-acetamide. According to a similar procedure to the one described in Example 144, Example 168 was synthesised using Example 163 as starting material. LC-MS (an20p10): Rt=5.33 min; MW=431; 300 MHz ¹HNMR (CDCl₃): δ ppm 2.36 (s, 6H); 3.20 (s, 3H); 3.71 (s, 2H); 8.23(s, 1H).

### Example 169

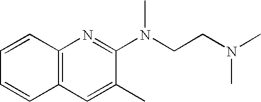

N,N,N′-Trimethyl-N′-(3-methyl-quinolin-2-yl)-ethane-1,2-diamine. According to a similar procedure to the one described in Example 138, Example 169 was synthesised using 2-chloro-3-methylquinoline and N,N,N′-trimethylethylenediamine as starting materials. LC-MS (an20p10): Rt=6.71 min; MW+1=244.

### Example 170

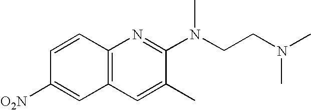

N,N,N′-Trimethyl-N′-(3-methyl-6-nitro-quinolin-2-yl)-ethane-1,2-diamine. According to a similar procedure to the one described in Example 139, Example 170 was synthesised using Example 169 as starting material. 300 MHz ¹HNMR (CDCl₃): δ ppm 2.35 (s, 6H); 2.49 (s, 3H); 3.17 (s, 3H).

### Example 171

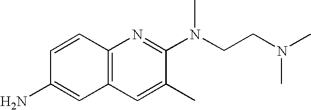

N*2*-(2-Dimethylamino-ethyl)-3,N*2*-dimethyl-quinoline-2,6-diamine. According to a similar procedure to the one described in Example 140, Example 171 was synthesised using Example 170 as starting material. LC-MS (an20p10): Rt=1.70 min; MW+1=259.

### Example 172

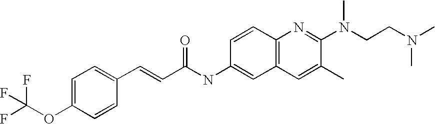

N-{2-[(2-Dimethylamino-ethyl)-methyl-amino]-3-methyl-quinolin-6-yl}-3-(4-trifluoromethoxy-phenyl)-acrylamide. According to a similar procedure to the one described in Example 141, Example 172 was synthesised using Example 171 as starting material. LC-MS (an20p10): Rt=7.34 min; MW+1=473.

### Example 173

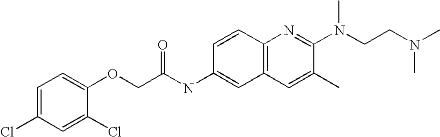

2-(2,4-Dichloro-phenoxy)-N-{2-[(2-dimethylamino-ethyl)-methyl-amino]-3-methyl-quinolin-6-yl}-acetamide. According to a similar procedure to the one described in Example 141, Example 173 was synthesised using Example 171 as starting material. LC-MS (an20p10): Rt=6.84 min; MW=461.

### Example 174

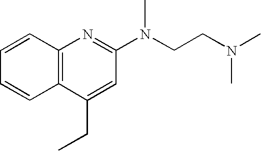

N-(4-Ethyl-quinolin-2-yl)-N,N′,N′-trimethyl-ethane-1,2-diamine. According to a similar procedure to the one described in Example 138, Example 174 was synthesised using Example 156 and N,N,N′-trimethylethylenediamine as starting materials. 300 MHz ¹HNMR (CDCl₃): δ ppm 1.38 (t, 3H); 2.39 (s, 6H); 2.62 (t, 2H); 3.0 (q, 2H); 3.237(s, 3H); 3.85 (t, 2H); 6.75 (s, 1H).

### Example 175

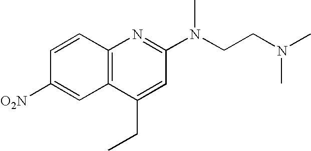

N-(4-Ethyl-6-nitro-quinolin-2-yl)-N,N′,N′-trimethyl-ethane-1,2-diamine. According to a similar procedure to the one described in Example 139, Example 175 was synthesised using Example 174 as starting material. 300 MHz ¹HNMR (CDCl₃): δ ppm 1.40 (t, 3H); 2.36 (s, 6H); 2.60 (t, 2H); 3.03 (q, 2H); 3.27(s, 3H); 3.87 (t, 2H); 6.83 (s, 1H); (s, 1H); 7.67 (d, 2H); 8.26 (dd, 1H); 8.74 (s, 1H).

### Example 176

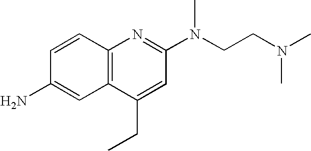

N*2*-(2-Dimethylamino-ethyl)-4-ethyl-N*2*-methyl-quinoline-2,6-diamine. According to a similar procedure to the one described in Example 140, Example 176 was synthesised using Example 175 as starting material. 300 MHz ¹HNMR (CDCl₃): δ ppm 1.35 (t, 3H); 2.35 (s, 6H); 2.56 (t, 2H); 2.90 (q, 2H); 3.18 (s, 3H); 3.77 (t, 2H); 6.71 (s, 1H); 7.03 (m, 2H); 7.56 (d, 1H).

### Example 177

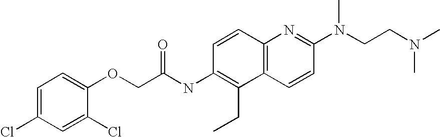

2-(2,4-Dichloro-phenoxy)-N-{2-[(2-dimethylamino-ethyl)-methyl-amino]-4-ethyl-quinolin-6-yl}-acetamide. According to a similar procedure to the one described in Example 141, Example 177 was synthesised using Example 176 as starting material. 300 MHz ¹HNMR (CDCl₃): δ ppm 1.39 (t, 3H); 2.39 (s, 6H); 2.63 (q, 2H); 3.23 (s, 3H); 3.83 (t, 2H); 4.68 (s, 2H); 6.78 (s, 1H); 8.62 (s; 1H).

### Example 178

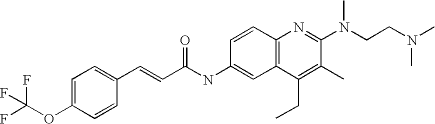

N-{2-[(2-Dimethylamino-ethyl)-methyl-amino]-4-ethyl-quinolin-6-yl}-3-(4-trifluoromethoxy-phenyl)-acrylamide. According to a similar procedure to the one described in Example 141, Example 178 was synthesised using Example 176 as starting material. 300 MHz ¹HNMR (CDCl₃): δ ppm 1.37 (t, 3H); 2.36 (s, 6H); 2.59 (q, 2H); 3.21 (s, 3H); 3.81 (t, 2H); 8.44 (s; 1H).

### Example 179

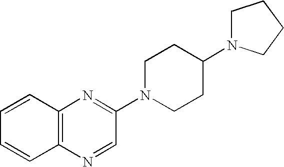

### 2-(4-Pyrrolidin-1-yl-piperidin-1-yl)-quinoxaline

A mixture of 2-chloroquinoxaline (150 mg, 0.9 mmol) and 4-(1-pyrrolidinyl)-piperidine (300 mg, 1.9 mmol) was heated at 130° C. for 2h00. After cooling, the solid residue was washed with water, filtered and dried in vacuo to give Example 179 (233 mg, 0.82 mmol, 92%) which was used without further purification. LC-MS (an05p7): Rt=3.3 min; MW+1=283.

### Example 180

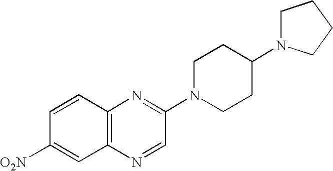

6-Nitro-2-(4-pyrrolidin-1-yl-piperidin-1-yl)-quinoxaline. To a solution of Example 179 (50 mg, 0.18 mmol) in concentrated sulphuric acid (3 ml) was dropwise added a solution of potassium nitrate (20 mg, 0.2 mmol) in concentrated sulphuric acid (1 ml). After stirring at RT for 18h00, the mixture was poured into ice and basified to pH=12 with aq. NaOH. The mixture was extracted with EtOAc. The organic phase was dried over MgSO₄ and concentrated in vacuo to give Example 180 (58.8 mg, 0.18 mmol, 100%) which was used without further purification. LC-MS (an10p8): Rt=3.8 min; MW+1=328.

### Example 181

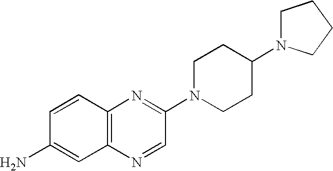

2-(4-Pyrrolidin-1-yl-piperidin-1-yl)-quinoxalin-6-ylamine. To a solution of Example 180 (58.8 mg, 0.18 mmol) in ethanol (25 ml) was added a catalytic amount of Pd/C (10% wt). The reaction mixture was stirred for 1h00 at RT under a hydrogen atmosphere. The catalyst was filtered off and the filtrate was concentrated in vacuo to give Example 181 (53.4 mg, 0.18 mmol, 100%) which was used without further purification. LC-MS (an10p8): Rt=4.7 min; MW+1=298.

### Example 182

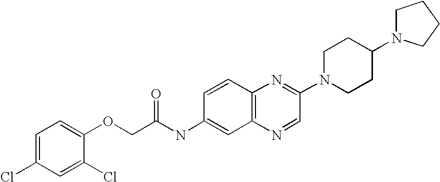

2-(2,4-Dichloro-phenoxy)-N-[2-(4-pyrrolidin-1-yl-piperidin-1-yl)-quinoxalin-6-yl]-acetamide. To a cooled (0° C.) solution of Example 181 (53.4 mg, 0.18 mmol) in CH₂Cl₂ (20 ml), 2,4-dichlorophenoxyacetyl chloride (52 mg, 0.2 mmol) was added, under an inert atmosphere. The reaction mixture was then allowed to stir at RT overnight. The mixture was washed with aq. NaHCO₃. The organic phase was dried over MgSO₄ and concentrated in vacuo. The residue was purified over silica gel chromatography to give Example 182 (30 mg, 0.06 mmol, 33%). LC-MS (an10p8): Rt=4.45 min; MW=500.

### Example 183

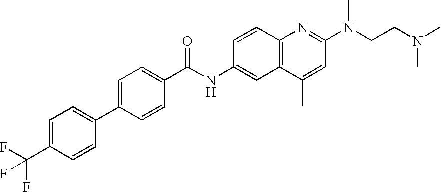

4′-Trifluoromethyl-biphenyl-4-carboxylic acid {2-[(2-dimethylamino-ethyl)-methyl-amino]-4-methyl-quinolin-6-yl}-amide. The title compound was made according to a procedure similar to the one described for Example 112. LC/MS (an10p8.m): Rt 5.566 min, m/z 507.2 [MH⁺].

### Example 184

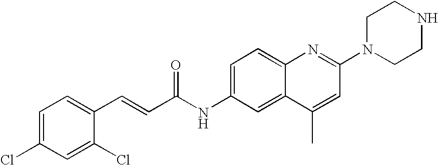

(E)-3-(2,4-Dichloro-phenyl)-N-(4-methyl-2-piperazin-1-yl-quinolin-6-yl)-acrylamide. The title compound was made according to a procedure similar to the one described for Example 39. LCMS (an10p8.m): Rt 4.620 min, m/z 441.1 [MH⁺].

In Vitro Tests of Compounds According to the Invention

Receptor Binding Data

| | | Receptor | IP3 |
|---|---|---|---|
| | | binding | IC50 |
| Compound | Example | IC50 nM | nM |
| 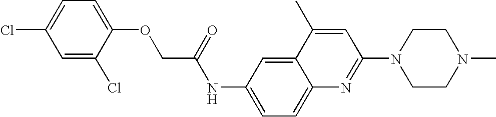 | Example 14 | 12 | 72 |
| 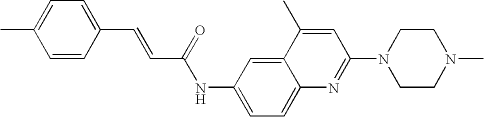 | Example 15 | 20 | |
| 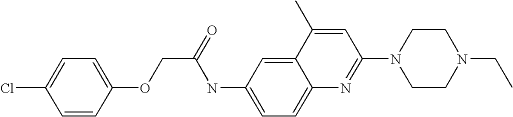 | Example 4 | 24 | 140 |
| 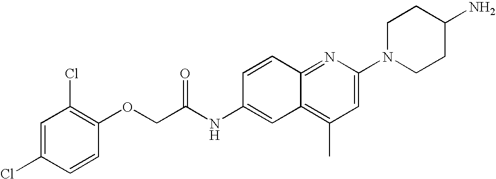 | Example 33 | 41 | 63 |
| 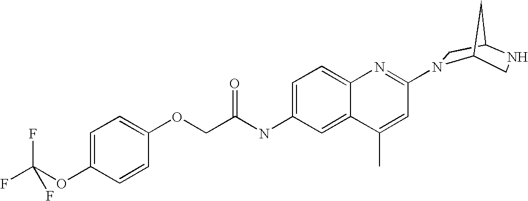 | Example 128 | 10 | 13 |
| 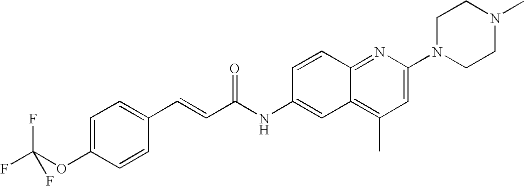 | Example 16 | 11 | 42 |
| 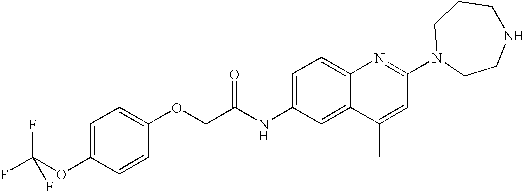 | Example 25 | 12 | 71 |
| 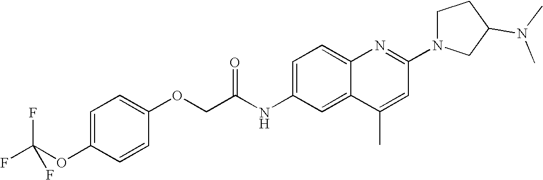 | Example 69 | 6 | 82 |
| 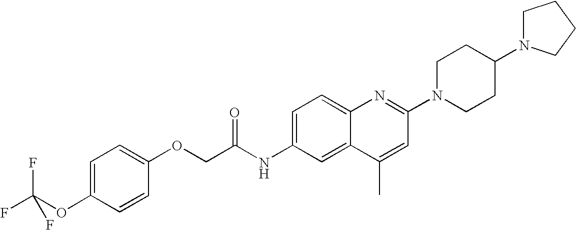 | Example 70 | 5 | 8 |
| 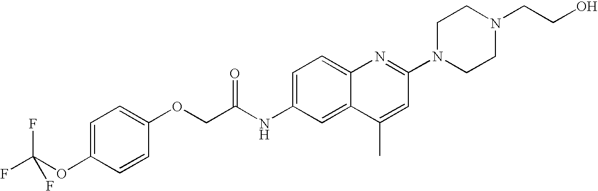 | Example 38 | 50 | 23 |
| 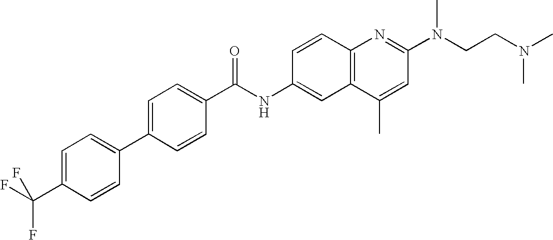 | Example 183 | 63 | 474 |
| 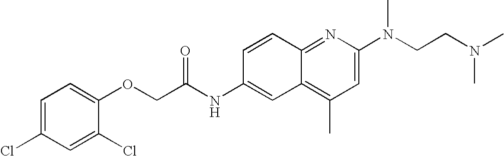 | Example 80 | 8 | 65 |
| 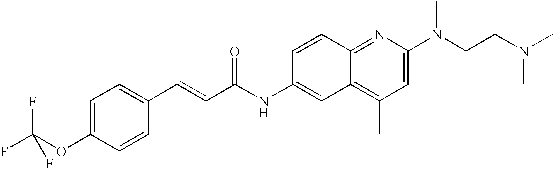 | Example 81 | 20 | 140 |
| 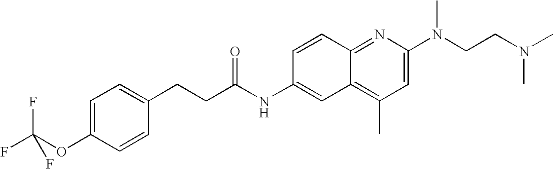 | Example 98 | 37 | 720 |
| 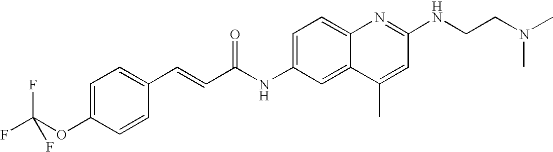 | Example 47 | 11 | 42 |
| 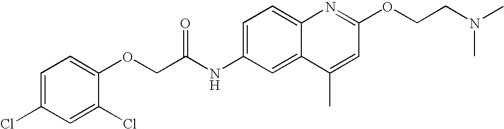 | Example 76 | 180 | 135 |
| 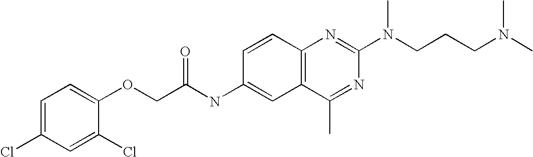 | Example 137 | 10 | 63 |
| 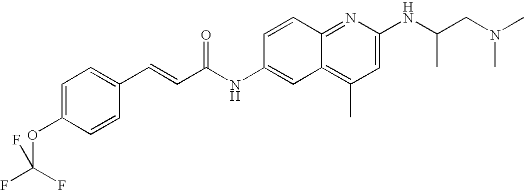 | Example 53 | 22 | 98 |
| 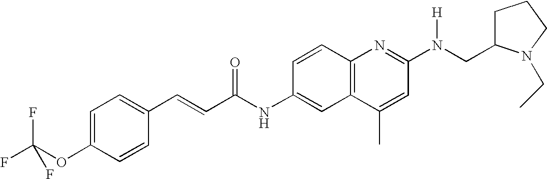 | Example 62 | 13 | 10 |
| 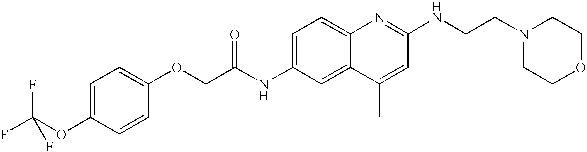 | Example 42 | 10 | 16 |
| 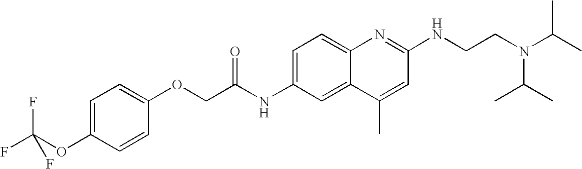 | Example 40 | 22 | |
| 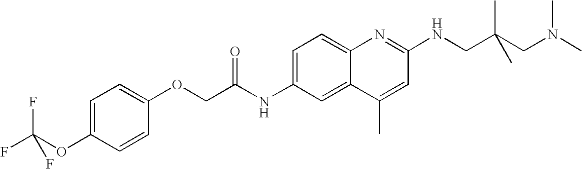 | Example 45 | 21 | 190 |
| 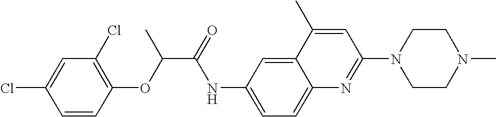 | — | 280 | 2000 |
| 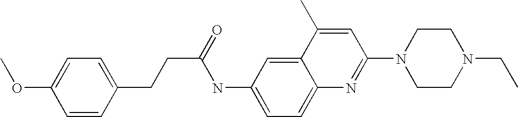 | — | 250 | 420 |

In Vivo model measuring effects on food intake—The effects of test compounds on food intake were studied in male Sprague Dawley rats (250 g at entrance). Animals are single housed in conventional cages. 10 days before dosing, the animals will be accustomed to the reversed day night cycle (lights off 8:00 am till 20:00 pm). During this period, the animals will also be accustomed to the administration procedures (2 times, 1 h before dark, water, 2 ml p.o.). They have access to food (normal rat chow) and water ad. lib., 24 h per day, unless otherwise stated. Test compounds are dissolved in lactic acid 0.01%, with an administration volume of 10 ml/kg. 24 h before the test, food is taken away from the animals. (i.e. just before lights off). At the test day, the animals are given with the test compound orally 1 h prior to lights off. At lights off, the animals are given food again. Food and water intake are registered hourly over the first 3 hours at 6 h, and at 24 h after re-access to food. The animals are randomly assigned to the groups after weighing at the test day. The control group is given the vehicle of 0.01% lactic acid. The compounds are given in doses between 5 and 50 mg/kg. Results are analyzed by one-way ANOVA followed by post hoc Bonferroni test.

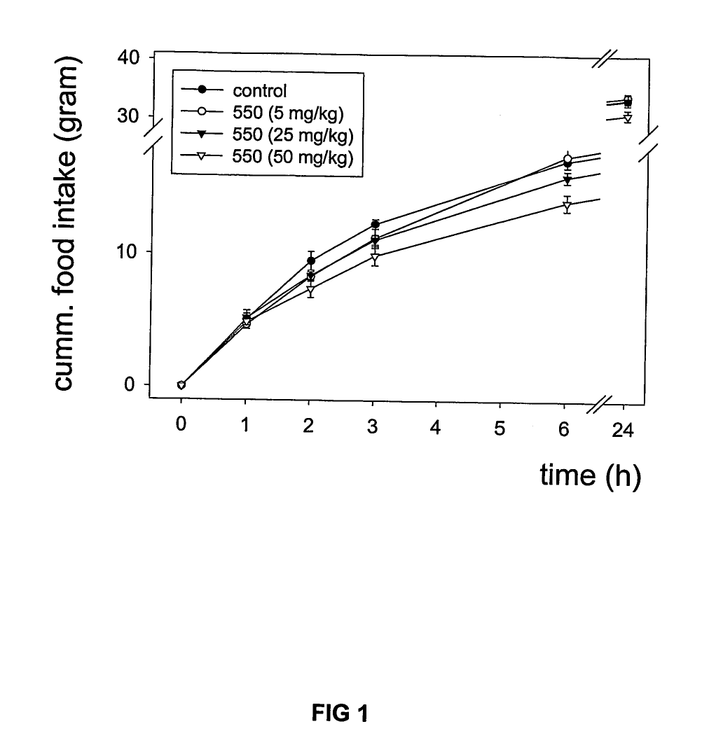

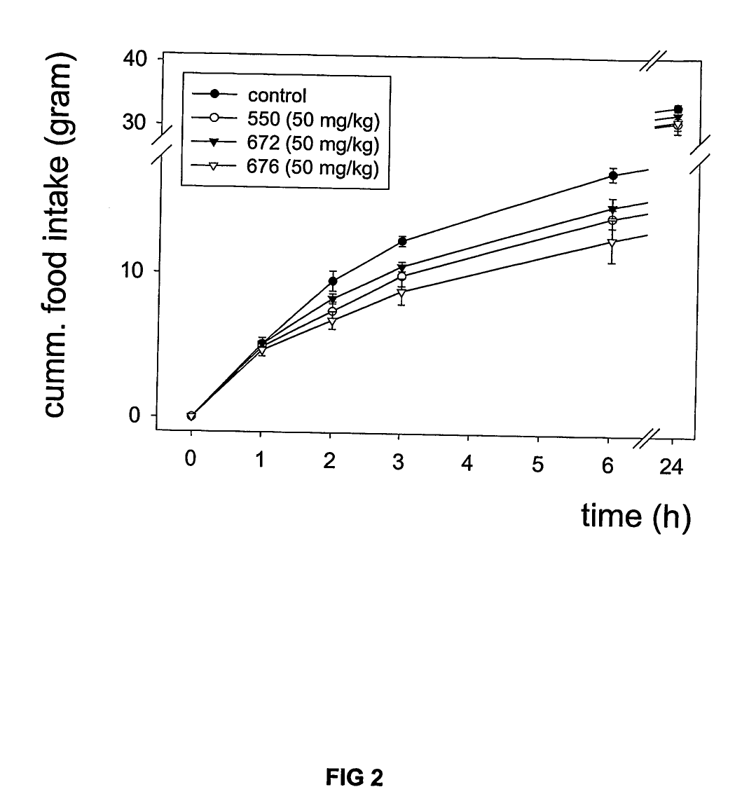

FIGS. 1 and 2 show the effect on food intake after oral administration of compounds in Example 4 (550), Example 80 (672) and Example 81 (676).

1-142. (canceled)

143. A compound with the following structure (Formula Ia) 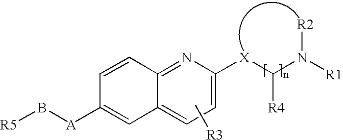
wherein the quinoline moiety may contain more than one nitrogen atom such as, e.g. 2 or 3 nitrogen atoms,
and wherein -A- is a linker, which when the quinoline moiety contains 2 or 3 nitrogen atoms, is selected from the group consisting of 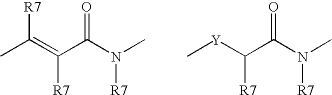
and wherein -A- is a linker, which when the quinoline moiety contains 1 nitrogen atom, is 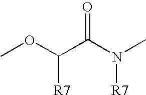
and Y being CHR7, O, S, NR7;
wherein the linker may be attached via either of the two free bonds to the B group;
and R7 is the same or different and is hydrogen or a straight or branched C₁-C₄ alkyl or alkenyl group; R7 can be linked direct or via hetero atoms to B or the quinoline ring system when chemically feasible;
and X being nitrogen;
B is an aryl or heteroaryl group such as, e.g. phenyl, pyridine, pyrimidine, pyrazine, thiophene;
R1 and R2 are the same or different selected from hydrogen, straight or branched alkyl groups with 1-4 carbon atoms; cycloalkyl groups with 3-7 carbons; alkylcycloalkyl with 4-8 carbons atoms; alkylaryl groups such as benzyl; alkylheteroaryl groups; the alkyl, aryl and heteroaryl groups may be substituted with substituents such as Alk-CONH—, Alk-O—, HO—, NC—; AlkNH—, Alk₂N—, —CONH₂, —CONHAlk, —CONAlk₂, or the aryl and heteroaryl groups fused with moieties such as —O—CH₂—O—, —N═CH—NH—, —O—CH═N—; R2 may be further substituted with one or more R4 groups in any position;
Alk is the same or a different alkyl group;
R4 is the same or different and is hydrogen or a straight or branched C₁-C₄ alkyl group; and may be substituted with one or two C₁-C₄ alkyl groups;
R3 may be selected from hydrogen and alkyl groups;
R1, R2, R3 or R4 may optionally be linked to each other, or to the carbon chain linking the two nitrogen atoms, when possible; and O or NR1 may be inserted in the chain or ring in a chemically stable position; R4 may optionally be linked to X;
R5 is hydrogen, halogen atoms, alkyl groups, cycloalkyl groups with 3-7 carbons, alkoxy groups (AlkO—), —CONH₂, —CONHAlk, —CONHAr—CONAlk₂, —NHCO-Alk, —NHCO—Ar, —CO-Alk, —N(CF₃)₂, —SCH₃, partially or fully fluorinated alkyl, alkoxy or thioalkoxy groups such as —CH₂CF₃, —CF₃, —OCF₃, —SCF₃;
optionally, one or more R5 may be present on B; and
n is 2 or 3;

144. A compound according to claim 143, wherein the nitrogen-containing chain has the structure: 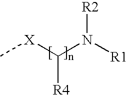
and the quinoline moiety has one of the following structures: 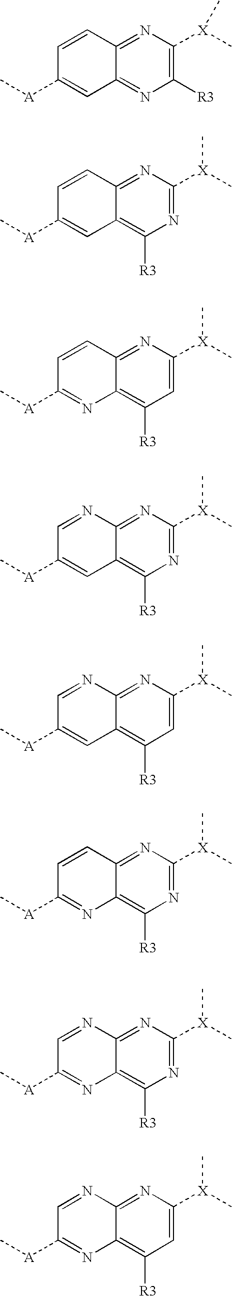
wherein A, B, R1, R2, R3, R4, R5, R7, Y, X and n are as defined in claim 143.

145. A compound according to claim 144, wherein Y is oxygen; R3 is methyl; R7 is hydrogen; and A, B, R1, R2, R4, R5, X and n are as defined in claim 143.

146. A compound according to claim 143, wherein the nitrogen-containing chain has the structure: 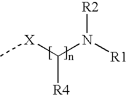
and the quinoline moiety has the following structure: 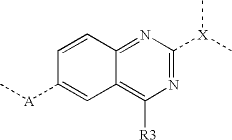
wherein A, R1, R2, R3, R4, X and n are as defined in claim 143.

147. A compound according to claim 146, wherein Y is oxygen; R3 is methyl; R7 is hydrogen; and A, B, R1, R2, R4, R5, X and n are as defined in claim 143.

148. A compound according to claim 143, wherein the nitrogen-containing chain has the structure: 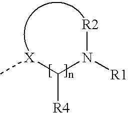
wherein X, R1, R2, R4 and n are as defined in claim 143.

149. A compound according to claim 143, wherein the nitrogen-containing chain has the structure: 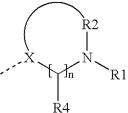
and the quinoline moiety has one of the following structures: 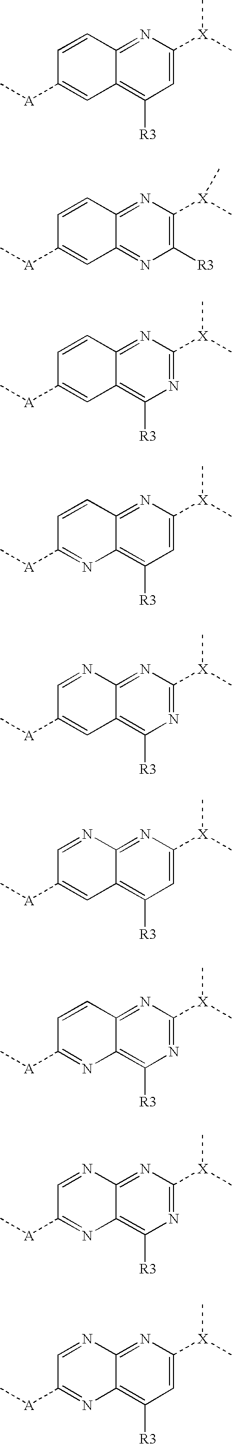
wherein A, B, R1, R2, R3, R4, R5, R7, Y, X and n are as defined in claim 143.

150. A compound according to claim 143, wherein the nitrogen-containing chain has the structure: 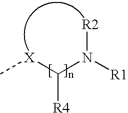
and the quinoline moiety has the following structure: 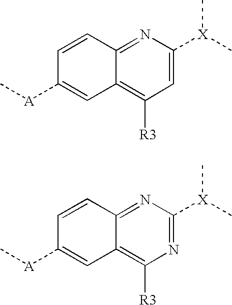
wherein A, R1, R2, R3, R4, X and n are as defined in claim 143.

151. A compound according to claim 148, wherein the nitrogen-containing chain has the structure: 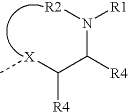
wherein X, R1, R2 and R4 are as defined in claim 143.

152. A compound according to claim 148, wherein the nitrogen-containing chain has the structure: 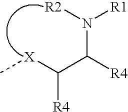
and the quinoline moiety has one of the following structures: 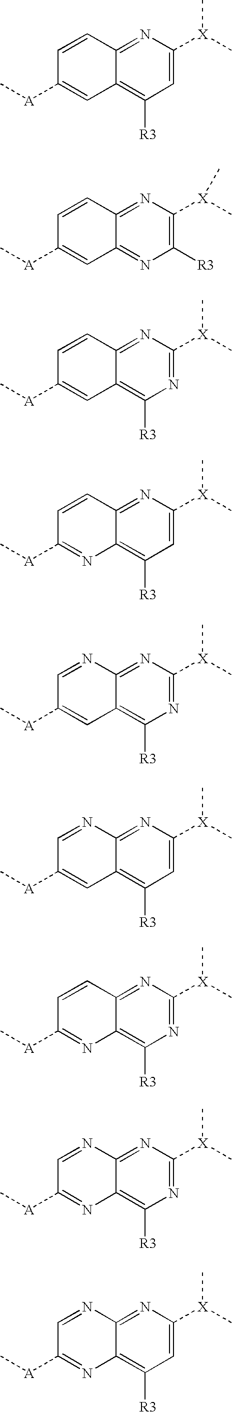
wherein A, B, R1, R2, R3, R4, R5, R7, Y, X and n are as defined in claim 143.

153. A compound according to claim 148, wherein the nitrogen-containing chain has the structure: 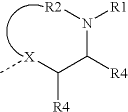
and the quinoline moiety has one of the following structures: 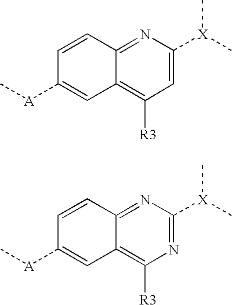
wherein A, B, R1, R2, R3, R4, R5, R7, Y, X and n are as defined in claim 143.

154. A compound according to claim 148, wherein the nitrogen-containing chain has the structure: 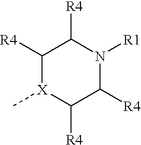
wherein X, R1 and R4 are as defined in claim 143.

155. A compound according to claim 148, wherein the nitrogen-containing chain has the structure: 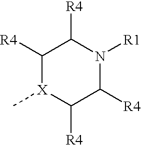
and the quinoline moiety has one of the following structures: 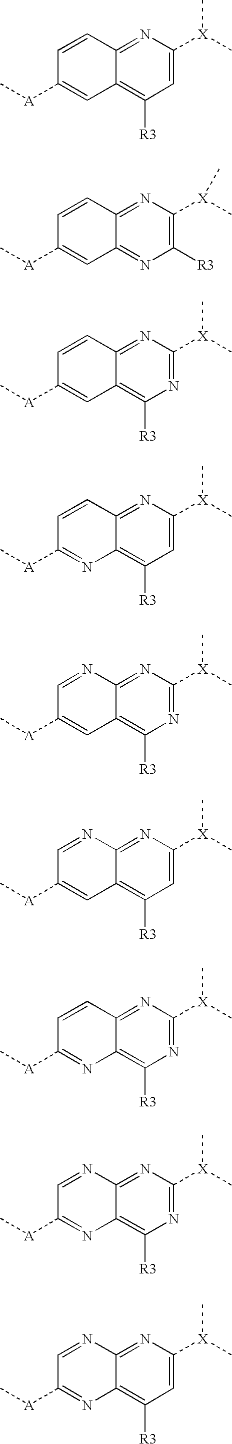
wherein A, B, R1, R2, R3, R4, R5, R7, Y, X and n are as defined in claim 143.

156. A compound according to claim 148, wherein the nitrogen-containing chain has the structure: 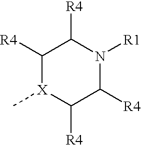
and the quinoline moiety has one of the following structures: 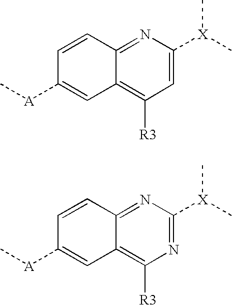
wherein A, B, R1, R2, R3, R4, R5, R7, Y, X and n are as defined in claim 143.

157. A compound according to claim 143, wherein the nitrogen-containing chain has the structure: 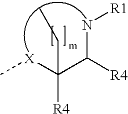
wherein X, R1 and R4 are as defined in claim 143 and m is 1 or 2.

158. A compound according to claim 143, wherein the nitrogen-containing chain has the structure: 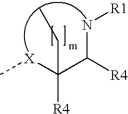
and the quinoline moiety has one of the following structures: 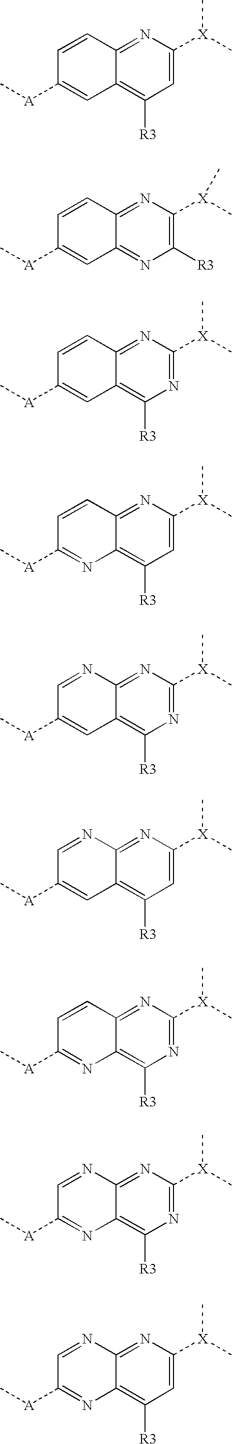
wherein A, B, R1, R2, R3, R4, R5, R7, Y, X and n are as defined in claim 143, and m is 1 or 2.

159. A compound according to claim 143, wherein the nitrogen-containing chain has the structure: 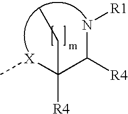
and the quinoline moiety has one of the following structures: 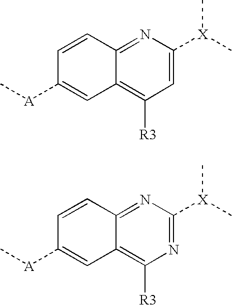
wherein A, B, R1, R2, R3, R4, R5, R7, Y, X and n are as defined in claim 143, and m is 1 or 2.

160. A compound according to claim 143, wherein the nitrogen-containing chain has the structure: 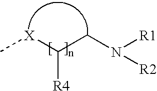
wherein X, R1, R2, R4 and n are as defined in claim 143.

161. A compound according to claim 143, wherein the nitrogen-containing chain has the structure: 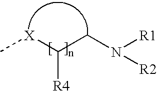
and the quinoline moiety has one of the following structures: 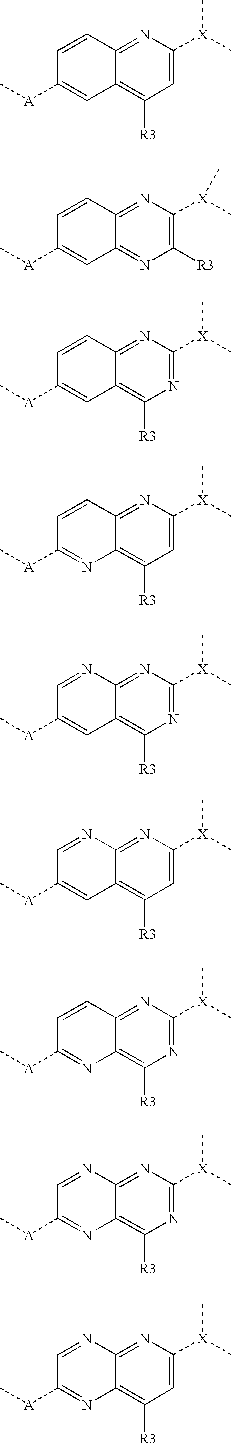
wherein A, B, R1, R2, R3, R4, R5, R7, Y, X and n are as defined in claim 143.

162. A compound according to claim 143, wherein the nitrogen-containing chain has the structure: 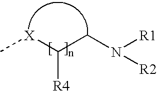
and the quinoline moiety has one of the following structures: 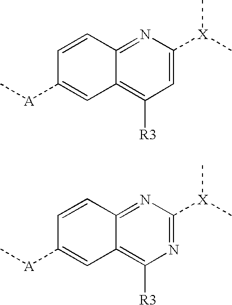
wherein A, B, R1, R2, R3, R4, R5, R7, Y, X and n are as defined in claim 143.

163. A compound according to claim 160, wherein the nitrogen-containing chain has the structure: 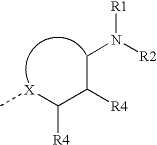
wherein X, R1, R2 and R4 are as defined in claim 143.

164. A compound according to claim 160, wherein the nitrogen-containing chain has the structure: 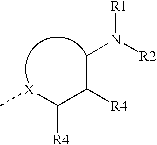
and the quinoline moiety has one of the following structures: 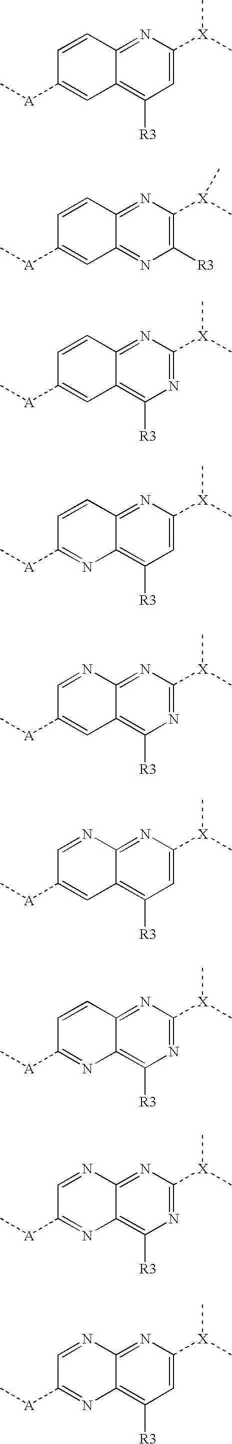
wherein A, B, R1, R2, R3, R4, R5, R7, Y, X and n are as defined in claim 143.

165. A compound according to claim 160, wherein the nitrogen-containing chain has the structure: 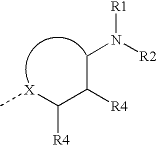
and the quinoline moiety has one of the following structures: 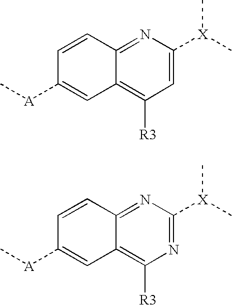
wherein A, B, R1, R2, R3, R4, R5, R7, Y, X and n are as defined in claim 143.

166. A compound according to claim 143, wherein the nitrogen-containing chain has the structure: 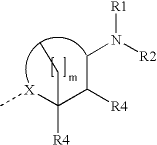
wherein X, R1, R2 and R4 are as defined in claim 143 and m is 1 or 2.

167. A compound according to claim 160, wherein the nitrogen-containing chain has the structure: 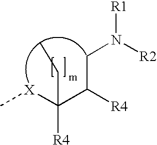
and the quinoline moiety has one of the following structures: 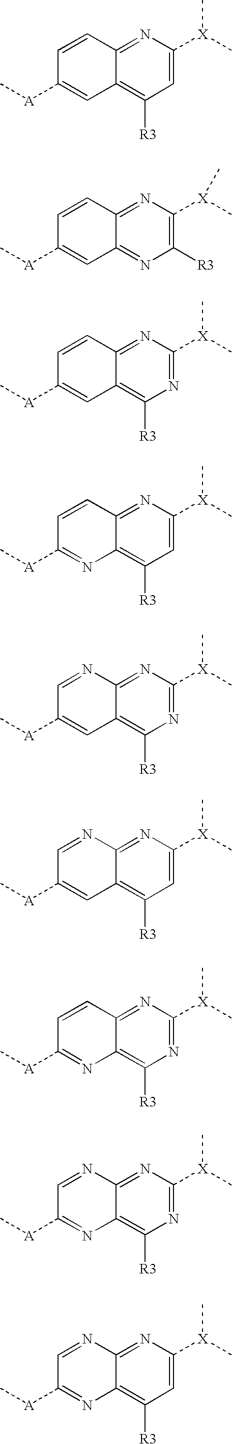
wherein A, B, R1, R2, R3, R4, R5, R7, Y, X and n are as defined in claim 143 and m is 1 or 2.

167. A compound according to claim 160, wherein the nitrogen-containing chain has the structure: 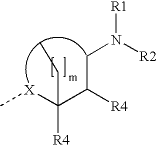
and the quinoline moiety has one of the following structures: 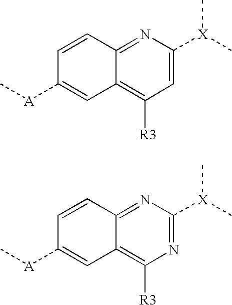
wherein A, B, R1, R2, R3, R4, R5, R7, Y, X and n are as defined in claim 143 and m is 1 or 2.

169. A compound according to claim 160, wherein the nitrogen-containing chain has the structure: 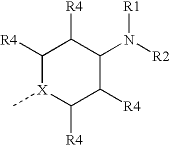
wherein X, R1, R2 and R4 are as defined in claim 143.

170. A compound according to claim 160, wherein the nitrogen-containing chain has the structure: 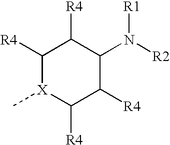
and the quinoline moiety has one of the following structures: 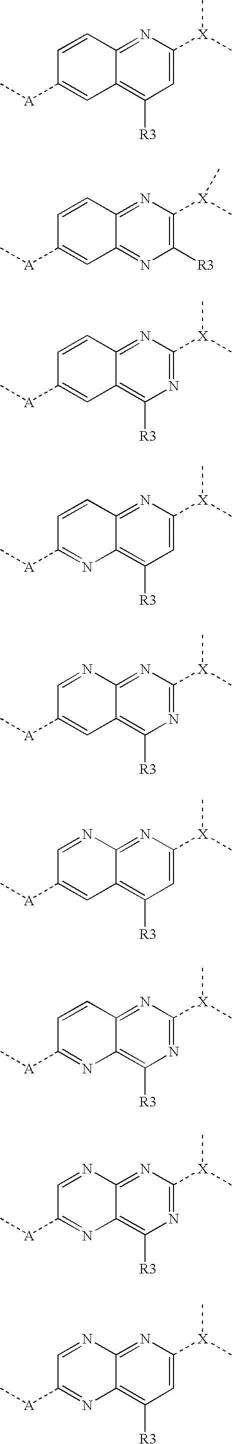
wherein A, B, R1, R2, R3, R4, R5, R7, Y, X and n are as defined in claim 143.

171. A compound according to claim 160, wherein the nitrogen-containing chain has the structure: 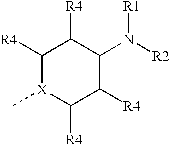
and the quinoline moiety has one of the following structures: 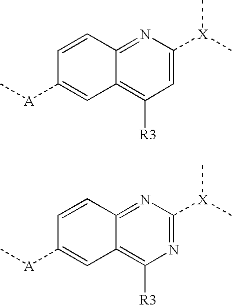
wherein A, B, R1, R2, R3, R4, R5, R7, Y, X and n are as defined in claim 143.

172. A compound according to claim 143, wherein R7 is hydrogen.

173. A compound according to claim 143, wherein A is: 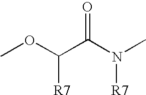
wherein R7 is hydrogen.

174. A compound according to claim 143, wherein A has the structure 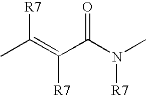
and the nitrogen-containing chain has the structure: 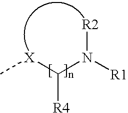
wherein R7 is hydrogen and R1, R2, R4, X and n are as defined in claim 143.

175. A compound according to claim 143, wherein the compound has one of the following structures: 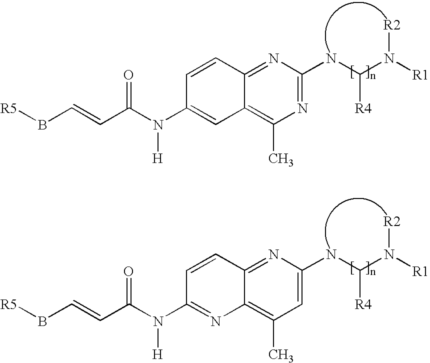
wherein B, R1, R2, R4, R5 and n are as defined in claim 143.

176. A compound according to claim 143, wherein the compound has the following structure: 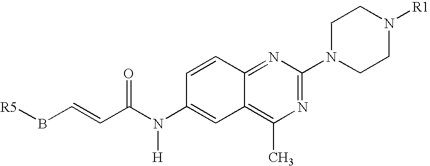
wherein B, R1, and R5 are as defined in claim 143.

177. A compound according to claim 143, wherein A has the structure 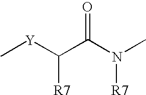
and the nitrogen-containing chain has the structure: 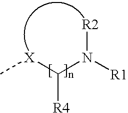
wherein R7 is hydrogen; Y is oxygen, and R1, R2, R4, Y, X and n are as defined in claim 143.

178. A compound according to claim 143, wherein the compound has one of the following structures: 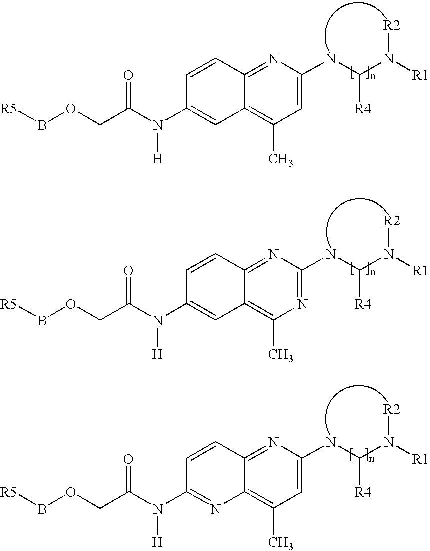
wherein B, R1, R2, R4, R5, and n are as defined in claim 143.

179. A compound according to claim 143, wherein the compound has one of the following structures: 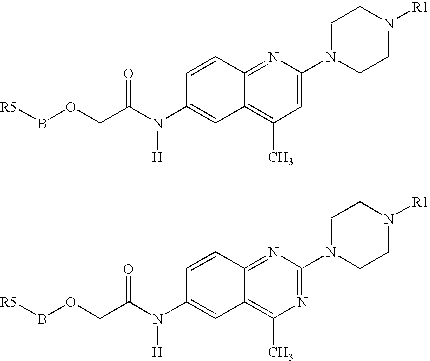
wherein B, R1, and R5, are as defined in claim 143.

180. A compound according to claim 143, wherein A has the structure 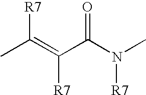
and the nitrogen-containing chain has the structure: 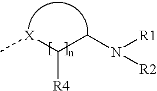
wherein R7 is hydrogen and R1, R2, R4 and n are as defined in claim 143.

181. A compound according to claim 143, wherein the compound has one of the following structures: 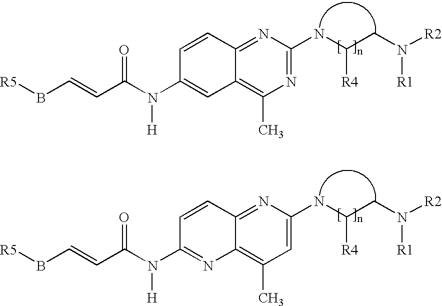
wherein B, R1, R2, R4, R5, and n are as defined in claim 143.

182. A compound according to claim 143, wherein the compound has the following structure: 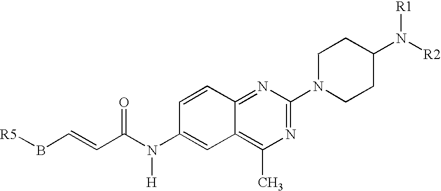
wherein B, R1, R2, and R5 are as defined in claim 143.

183. A compound according to claim 143, wherein A has the structure 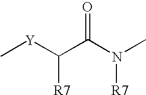
and the nitrogen-containing chain has the structure: 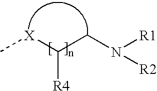
wherein R7 is hydrogen; Y is oxygen; and R1, R2, R4, and n are as defined in claim 143.

184. A compound according to claim 143, wherein the compound has one of the following structures: 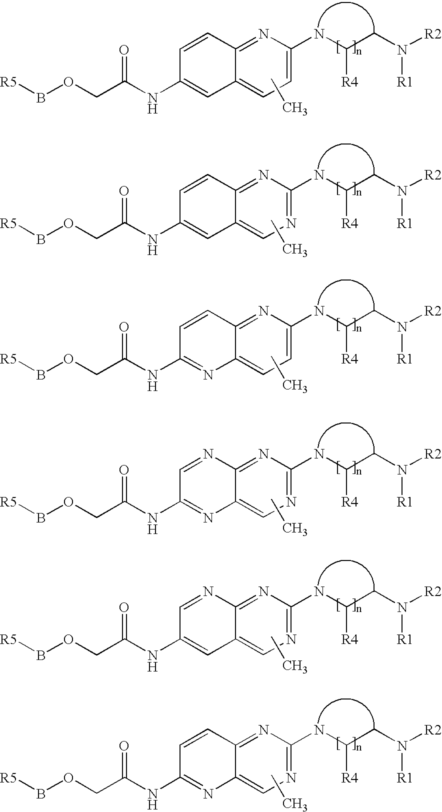
wherein B, R1, R2, R4, R5, and n are as defined in claim 143.

185. A compound according to claim 143, wherein the compound has one of the following structures: 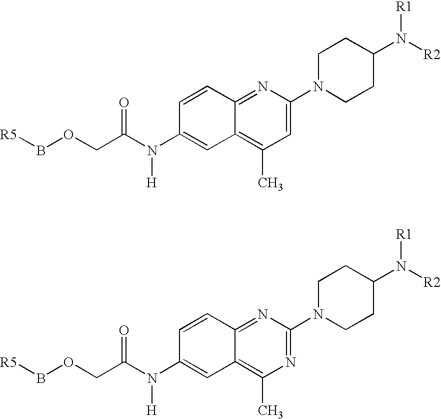
wherein B, R1, R2, and R5, are as defined in claim 143.

186. A compound according to claim 143, wherein the compound has the following structure: 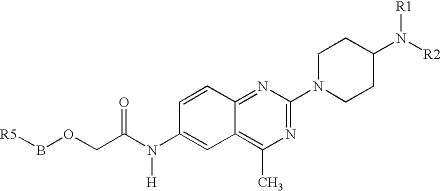
wherein B is phenyl or pyridine;
R5 is halogen atoms, alkyl groups, partially or fully fluorinated alkyl or alkoxy groups such as —CF₃, —OCF₃;
and R1 and R2 are as defined in claim 143.

187. A compound according to claim 143, wherein R3 is methyl.

188. A compound according to claim 143, wherein R7 is hydrogen.

189. A compound according to claim 143, wherein R4 is hydrogen

190. A compound according to claim 143, wherein R1 is hydrogen a lower straight or branched alkyl group with 1-4 carbon atoms or a cycloalkyl group with 3-7 carbon atoms such as, e.g., methyl, ethyl, propyl, isopropyl, cyclopentyl, which may be substituted with OH.

191. A compound according to claim 143, wherein R1 is hydrogen, methyl, ethyl, propyl, iso-propyl, or 2-hydroxyethyl.

192. A compound according to claim 143, wherein R1 is methyl, ethyl or 2-hydroxyethyl.

193. A compound according to claim 143, wherein Y is oxygen.

194. A compound according to claim 143, wherein B is phenyl or pyridine.

195. A compound according to claim 143, wherein R5 is halogen atoms, alkyl groups, —SCH₃, partially or fully fluorinated alkyl, alkoxy or thioalkoxy groups such as —CH₂CF₃, —CF₃, —OCF₃, —SCF₃.

196. A compound according to claim 143, wherein the compound is in amorphous or crystalline form.

197. A compound according to claim 143, wherein the compound is in racemic or enantiomeric form.

198. A compound according to claim 143, wherein the compound is in the form of a physiologically acceptable salt, complex, solvate or prodrug thereof.

199. A compound according to claim 143 for use in medicine.

200. A compound according to claim 143 for preventing or treating diseases caused by or involving a melanin-concentrating hormone.

201. A compound according to claim 143 for modulating the activity of a MCH receptor.

202. A compound according to claim 201 that has antagonistic activity against a MCH receptor.

203. A compound according to claim 201 that exhibits agonistic, inverse agonistic or allosteric activity against a MCH receptor.

204. A compound according to claim 201, wherein the MCH receptor has at least about 80% such as, e.g. at least about 85% or at least about 90% homology to the amino acid sequence CTLITAMDAN or CTIITSLDTC

205. A compound according to claim 201, wherein the MCH receptor comprises the amino acid sequence CTLITAMDAN or CTIITSLDTC.

206. A compound according to claim 201, wherein the MCH receptor is a MCH1 or MCH2 receptor.

207. A compound according to claim 201, wherein the MCH receptor is a MCH1 receptor.

208. A compound according to claim 201, wherein the MCH receptor is a mammalian receptor such as human receptor.

209. A cosmetic method for reducing overweight and/or for treating of and/or preventing overweight, bulimia, bulimia nervosa, obesity and/or complications thereto, the method comprising administering to an animal such as, e.g. a human in need thereof, an effective amount of a compound defined in claim 143.

210. A method for the treatment and/or prophylaxis of diseases caused by a melanin-concentrating hormone, the method comprising administering to a mammal in need thereof an efficient amount of a compound defined in claim 143.

211. A method for the treatment and/or prophylaxis of diseases caused by feeding disorders, the method comprising administering to a mammal in need thereof an efficient amount of a compound defined in claim 143.

212. A method for modifying the feeding behaviour of a mammal, the method comprising administering to a mammal in need thereof an efficient amount of a compound defined in claim 143.

213. A method for the reduction of body mass, the method comprising administering to a mammal in need thereof an efficient amount of a compound defined in claim 143.

214. A method for the treatment and/or prophylaxis of Syndrome X (metabolic syndrome) or any combination of obesity, insulin resistance, dyslipidemia, impaired glucose tolerance and hypertension, the method comprising administering to a mammal in need thereof an efficient amount of a compound defined in claim 143.

215. A method for the treatment and/or prophylaxis of Type II diabetes or Non Insulin Dependent Diabetes Mellitus (NIDDM), the method comprising administering to a mammal in need thereof an efficient amount of a compound defined in claim 143.

216. A method for the treatment and/or prophylaxis of bulimia, bulimia nervosa and/or obesity, the method comprising administering to a mammal in need thereof an efficient amount of a compound defined in claim 143.

217. A method for the treatment and/or prophylaxis of depression and/or anxiety, the method comprising administering to a mammal in need thereof an efficient amount of a compound defined in claim 143.

218. A pharmaceutical composition comprising a compound as defined in claim 143, together with one or more physiologically acceptable excipients.

219. A pharmaceutical composition according to claim 218, wherein the compound is present in the form of a physiologically acceptable salt such as a salt formed between the compound and an inorganic acid such as e.g., a hydrochloride, a hydrobromide, a hydroiodide, a nitrate, a nitrite, a H₃PO₃ salt, a H₃PO₄ salt, a H₂SO₃ salt, a sulfate, a H₂SO₅ salt, or a salt formed between the compound and an organic acid such as organic acids like e.g. H₂CO₃, acetic acid, C₂H₅COOH, C₃H₇COOH, C₄H₉COOH, longer saturated or unsaturated fatty acids, (COOH)₂, CH₂(COOH)₂, C₂H₄(COOH)₂, C₃H₆(COOH)₂, C₄H₈(COOH)₂, C₅H₁₀(COOH)₂, fumaric acid, maleic acid, malic acid, lactic acid, citric acid, tartaric acid, ascorbic acid, benzoic acid, salicylic acid, phthalic acid, palmoic acid, trifluoroacetic acid, p-toluenesulfonic acid, methanesulfonic acid.

220. A pharmaceutical composition according to claim 218 for enteral and/or parenteral use.

221. A pharmaceutical composition according to claim 218 for oral, buccal, rectal, nasal, topical, vaginal or ocular use.

222. A pharmaceutical composition according to claim 218 in the form of a solid, semi-solid or fluid composition.

223. A pharmaceutical composition according to claim 222 in solid form, wherein the composition is in the form of tablets such as, e.g. conventional tablets, effervescent tablets, coated tablets, melt tablets or sublingual tablets, pellets, powders, granules, or particulate material.

224. A pharmaceutical composition according to claim 222 in semi-solid form, wherein the composition is in the form of a chewing gum, an ointment, a cream, a liniment, a paste, a gel or a hydrogel.

225. A pharmaceutical composition according to claim 222 in fluid form, wherein the composition is in the form of a solution, an emulsion, a suspension, a dispersion, a liposomal composition, a spray, a mixture, or a syrup.

226. A pharmaceutical composition according to claim 218 comprising a therapeutically effective amount of a compound according to claim 143.

227. A pharmaceutical composition according to claim 226, wherein the amount is from about 0.001 mg to about 1 g such as, e.g. from about 0.005 to about 750 mg, from about 0.01 to about 500 mg, from about 0.05 to about 500 mg, from about 0.1 to about 250 mg, from about 0.1 to about 100 mg or from about 0.5 to about 50 mg.
